SEQUENCE LISTING (2 sequences)

SEQ ID NO: 1 - protein, 163 aa, Homo sapiens
MADEEKLPPGWEKRMSRSSGRVYYFNHITNASQWERPSGNSSSGGKNGQGEPARVRCSHLLVKHSQSRRPSSWRQEKITRTKEEALELINGYIQKIKSGEEDFESLASQFSDCSSAKARGDLGAFSRGQMQKPFEDASFALRTGEMSGPVFTDSGIHIILRTE

SEQ ID NO: 2 - protein, 163 aa, Homo sapiens
MADEEKLPPGWEKAMSRSSGRVYYFNHITNASQWERPSGNSSSGGKNGQGEPARVRCSHLLVKHSQSRRPSSWRQEKITRTKEEALELINGYIQKIKSGEEDFESLASQFSDCSSAKARGDLGAFSRGQMQKPFEDASFALRTGEMSGPVFTDSGIHIILRTE

(19) United States
(12) Patent Application Publication
(10) Pub. No.: US 2006/0074222 A1
(43) Pub. Date: Apr. 6, 2006

(54) Determination of a phosphorylation site in PPIase domain of Pin1 and uses therefor

(75) Inventors: Kun Ping Lu, Newton, MA (US); Janusz M. Sowadski, Boston, MA (US); Robert K. Suto, Maynard, MA (US); Timothy D. McKee, Waltham, MA (US); Amy L. Kimzey, Hudson, MA (US); Futoshi Suizu, Auburndale, MA (US)

Correspondence Address: LAHIVE & COCKFIELD, LLP., 28 STATE STREET, BOSTON, MA, 02109, US

(73) Assignees: VERNALIS PLC, Winnersh (GB); BETH ISRAEL DEACONESS MEDICAL CENTER, Boston, MA (US)

(21) Appl. No.: 11/183,254

(22) Filed: Jul. 15, 2005

Related U.S. Application Data
(60) Provisional application No. 60/588,469, filed on Jul. 15, 2004.
(60) Provisional application No. 60/617,006, filed on Oct. 7, 2004.

(51) Int. Cl. C07K 14/705; G06F 19/00

(52) U.S. Cl. 530/352; 702/19

(57) ABSTRACT

This invention relates to methods of determining the prognosis of a subject with a Pin1 associated disorder by evaluating the levels of phosphorylated Pin1 in a biological sample. The invention also provides Pin1 polypeptides phosphorylated position 16, position 71, or both. The invention further relates to a crystal structure of a pPin1(71) polypeptide and a pPin1(16)(71) polypeptide. The invention also provides constitutively active mutants of Pin1 and modulators that are specific for the molecules. The invention further provides methods for determining if a subject is at risk of developing a Pin1 associated state by determining if a the subject has a mutation in Pin1 that renders the polypeptide constitutively active.

### RELATED APPLICATIONS

This application claims the benefit of and priority to U.S. Provisional Application No. 60/588,469 filed Jul. 15, 2004 and U.S. Provisional Application No. 60/617,006 filed Oct. 7, 2004, the entire contents each of which are incorporated herein by reference.

### BACKGROUND OF THE INVENTION

The peptidyl-prolyl cis-trans isomerases (PPIases), or rotamases, are a family of ubiquitous enzymes that catalyze the cis/trans isomerization of the peptide bond on the n-terminal side of proline residues in proteins (Hunter, (1998) Cell 92:141-142). PPIases are divided into three classes, cyclophilins (Cyps), FK-506 binding proteins (FKBPs) and the Pin1/parvulin class.

Cyclophilins and FKBPs are distinguished by their ability to bind the clinically immunosuppressive drugs cyclosporin and FK506, respectively (Schreiber, (1991) Science 251:283-7, Hunter, supra). Upon binding of these drugs, there are two common outcomes: inhibition of the PPIase activity and inhibition of the common target calcineurin. The inhibition of calcineurin phosphatase activity prevents lymphocytes from responding to antigen-induced mitogenic signals, thus resulting in immunosuppression. However, the inhibition of the PPIase activity is apparently unrelated to the immunosuppressive property of the drug/PPIase complexes. Even more surprisingly, deletion of all 8 known cyclophilins and 4 FKBPs in the same cells does not result in any significant phenotype (Dolinski et al., (1997) Proc. Natl. Acad. Sci. USA 94:13093-131098).

In contrast, members of the Pin1/parvulin class of PPIases bind neither of these immunosuppressive drugs, and are structurally unrelated to the other two classes of PPIases. Known members of the Pin1/parvulin class include Pins1-3 (Lu et al., (1996) Nature 380;544-547), Pin-1 (Campbell et al., (1997) Genomics 44:157-162), parvulin (Rahfeld, et al., (1996) Proc. Natl. Acad. Sci. USA 93:447-451) and Ess1/Pft1 (Hanes et al., (1989) Yeast 5:55-72; and Hani, et al., (1995) FEBS Lett. 365:198-202).

Pin1 is a highly conserved protein that catalyzes the isomerization of only phosphorylated Ser/Thr-Pro bonds (Rananathan et al., (1997) Cell 89:875-86; Yaffe et al., (1997) Science 278:1957-1960; Shen, et al., (1998) Genes Dev. 12:706-720; Lu et al., (1999) Science 283:1325-1328; Crenshaw et al., (1998) EMBO J. 17:1315-1327; Lu et al., (1999) Nature 399:784-788; Zhou, et al., (1999) Cell Mol. Life Sci. 56:788-806). In addition, Pin1 contains an N-terminal WW domain, which functions as a phosphorylated Ser/Thr-Pro binding module (Sudol, (1996) Prog. Biophys. Mol. Biol. 65:113-32). This phosphorylation-dependent interaction targets Pin1 to a subset of phosphorylated substrates, including Cdc25, Wee 1, Myt1, Tau-Rad4, and the C-terminal domain of RNA polymerase II large domain (Crenshaw et al., (1998) EMBO J. 17:1315-27; Shen, (1998) Genes Dev. 12:706-20; Wells, (1999) J. Cell. Sci. 112: 3861-71).

The specificity of Pin1 activity is essential for cell growth; depletion or mutations of Pin1 cause growth arrest, affect cell cycle checkpoints and induce premature mitotic entry, mitotic arrest and apoptosis in human tumor cells, yeast or Xenopus extracts (Lu et al., (1996) Nature 380:544-547; Winkler et al., (2000) Science 287:1644-1647; Hani et al., (1999) J. Biol. Chem. 274:108-116). In addition, Pin1 is dramatically overexpressed in human cancer samples. Moreover, inhibition of Pin1 by various approaches, including Pin1 antisense polynucleotides or genetic depletion, kills human and yeast dividing cells by inducing premature mitotic entry and apoptosis.

Thus, Pin1-catalyzed prolyl isomerization regulates the conformation and function of these phosphoprotein substrates and facilitates dephosphorylation because of the conformational specificity of some phosphatases. Thus, Pin1 -dependent peptide bond isomerization is a critical post-phosphorylation regulatory mechanism, allowing cells to turn phosphoprotein function on or off with high efficiency and specificity during temporally regulated events, including the cell cycle (Lu et al., supra).

It has previously been demonstrated that the phosphorylation of Pin1 at serine 16 inhibits the ability of Pin1 to isomerize substrate.

These results indicate that the Pin1 subfamily of enzymes is a novel target for diseases characterized by uncontrolled cell proliferation, primarily malignancies, and further can be used as a diagnostic marker for the onset and progression of cell proliferative disorders.

### SUMMARY OF THE INVENTION

The present invention is based, at least in part, on the discovery that phosphorylating Pin1 at position 71 leads to inactivation of the enzyme, and that mutations at this position that cannot be phosphorylated retain isomerase activity. This discovery provides a novel diagnostic and therapeutic target for subjects having a Pin1 associated disorder.

The invention provides prognostic and diagnostic methods using antibodies specific for Pin1 phosphorylated at position 16 and/or 71. In a related embodiment, the invention provides methods for determining the prognosis or diagnosis of a subject using more than one phosphorylation specific antibody, or an antibody that recognizes more than one phosphorylation site. For example, the invention provides a method using an antibody specific for pPin1(71) in combination with one that is specific for pPin1(16) to determine the prognosis or diagnosis of a subject.

In one embodiment, the invention pertains, at least in part, to a Pin1 polypeptide that is phosphorylated at position 16 and 71.

In one embodiment, the Pin1 that is phosphorylated at position 71 is inactive, i.e., cannot isomerize a substrate.

In one aspect, the invention pertains to a Pin1 polypeptide in the crystallized form having a mutation that disrupts that ability of Pin1 to be phosphorylated at position 16. In one embodiment, the mutant Pin1 is phosphorylated at position 71.

In one embodiment, the invention provides Pin1 polypeptides having a mutation in the phosphokinase A (PKA) recognition site for phosphorylating serine 16, wherein said polypeptide is phosphorylated at position 71. In a related embodiment, the mutation is at position 13, 14, 15, or 16. In a specific embodiment, the mutation is at position 14.

In another aspect, the invention provides the Pin1 R14A phosphorylated at position 71.

In yet another aspect, the invention provides a crystallized Pin1 polypeptide that is phosphorylated at position 71. In a related embodiment, the peptide has one or more mutations in the phosphokinase A (PKA) recognition site for PKA phosphorylation at position 16, e.g., residues 13-16 of SEQ ID NO:1. In a related embodiment, the one or more mutations are selected from the group consisting of residues: 13, 14, 15, and 16. In a specific embodiment, the mutation is at position 14. In a further specific embodiment, the mutation at position 14 is an arginine to alanine mutation.

In another aspect, the invention provides a Pin1 polypeptide having a mutation in the phosphokinase A (PKA) recognition site for phosphorylating serine 71. In certain embodiments, the mutated Pin1 polypeptide is not capable of being phosphorylated at position 71 and is constitutively active. In certain embodiments, the mutation is S71A, S71P, S71L, S71T, or S71W. In a further embodiment, the S71T mutant retains the ability to be phosphorylated at position 71.

In one specific embodiment, the crystallized polypeptide has the coordinates set forth in Table 2.

In yet another aspect, the invention provides a crystallized Pin1 polypeptide that is phosphorylated at position 16 and position 71. In one specific embodiment, the crystallized polypeptide has the coordinates set forth in Table 3.

In another embodiment, the invention provides a method of determining if a subject has a cell proliferative disorder comprising the steps of: obtaining a biological sample from a subject; evaluating the sample for the presence of pPin1(71), wherein a decreased level of pPin1 (71) as compared to a control sample is indicative that the subject has a cell proliferative disorder.

In another aspect, the invention provides a method for determining the prognosis of a subject having a cell proliferative disorder comprising the steps of: determining the levels of pPin1(71) in a biological sample; wherein an elevated level of pPin1 (71) in the sample compared to the statistical mean of a population having a cell proliferative disorder is indicative of a good prognosis.

In another aspect, the invention provides a method for determining the prognosis of a subject having a cell proliferative disorder comprising the steps of: determining the levels of pPin1(71) in a biological sample, wherein an decreased level of pPin1(71) in the sample compared to the statistical mean of a population having a cell proliferative disorder is indicative of a poor prognosis.

In related embodiments, the methods of determining the diagnosis and/or prognosis of a subject by evaluating the level of pPin1 use an antibody specific for pPin1 (71). In related embodiments, the methods may further comprise evaluating the levels of Pin1 phosphorylated at position 16. In related embodiments the levels of pPin1 are determined by FISH or immunohistochemistry (IHC).

In another aspect, the invention provides a method of determining the prognosis of a subject having a cell proliferative disorder comprising: obtaining a first biological sample from the subject and determining the level of pPin1(71) in the sample; obtaining a second biological sample from the subject at a time after collection of the first biological sample and determining the level of pPin1 (71) in the sample, wherein an increase in the level of pPin1(71) is indicative of good prognosis.

In another aspect, the invention provides a method of determining the prognosis of a subject having a cell proliferative disorder comprising: obtaining a first biological sample from the subject and determining the level of pPin1(71) in the sample; obtaining a second biological sample from the subject at a time after collection of the first biological sample and determining the level of pPin1(71) in said sample, wherein a decrease in the level of pPin1(71) is indicative of poor prognosis.

In related embodiments, the methods of the invention comprise isolating a biological sample selected from the group consisting of, for example, breast tissue, uterine tissue, ovarian tissue, brain tissue, endometrium tissue, cervical tissue, colon tissue, esophagus tissue, hepatocellular tissue, kidney tissue, mouth tissue, prostate tissue, liver tissue, lung tissue, skin tissue, or testicular, endocrine tissue, thyroid tissue, blood, ascites or brain fluid.

In another aspect, the invention provides a kit for determining the prognosis of a subject having a cell proliferative disorder comprising an antibody specific for pPin1(71) and instructions for use. In a related embodiment, the kit further comprises an antibody specific for pPin1(16). In related embodiments, the antibody is a monoclonal antibody or a polyclonal antibody. In a related embodiment, the kit further comprises an antibody specific for a second cancer marker.

In another aspect, the invention provides a method for determining the course of treatment for a subject having a cell proliferative disorder comprising determining the level of pPin1(71) in a biological sample from the subject, wherein the lower the level of pPin1(71 ) the more aggressive the treatment of the subject with an anticancer agent.

In related embodiments, the cell proliferative disorder is cancer. Exemplary cancers include oligodendroglioma, astrocytoma, glioblastomamultiforme, cervical carcinoma, endometriod carcinoma, endometrium serous carcinoma, ovary endometroid cancer, ovary Brenner tumor, ovary mucinous cancer, ovary serous cancer, uterus carcinosarcoma, breast lobular cancer, breast ductal cancer, breast medullary cancer, breast mucinous cancer, breast tubular cancer, thyroid adenocarcinoma, thyroid follicular cancer, thyroid medullary cancer, thyroid papillary carcinoma, parathyroid adenocarcinoma, adrenal gland adenoma, adrenal gland cancer, pheochromocytoma, colon adenoma mild displasia, colon adenoma moderate displasia, colon adenoma severe displasia, colon adenocarcinoma, esophagus adenocarcinoma, hepatocelluar carcinoma, mouth cancer, gall bladder adenocarcinoma, pancreatic adenocarcinoma, small intestine adenocarcinoma, stomach diffuse adenocarcinoma, prostate (hormone-refract), prostate (untreated), kidney chromophobic carcinoma, kidney clear cell carcinoma, kidney oncocytoma, kidney papillary carcinoma, testis non-seminomatous cancer, testis seminoma, urinary bladder transitional carcinoma, lung adenocarcinoma, lung large cell cancer, lung small cell cancer, lung squamous cell carcinoma, Hodgkin lymphoma, MALT lymphoma, non-hodgkins lymphoma (NHL) diffuse large B, NHL, thymoma, skin malignant melanoma, skin basolioma, skin squamous cell cancer, skin merkel cell cancer, skin benign nevus, lipoma, and liposarcoma abnormal cell growth.

In related embodiments, anticancer agents are Pin1 modulators.

The invention further provides methods of designing compounds that inhibit Pin1 by interacting with serine 71. In a further embodiment, the invention provides a method of designing compounds that interact with pPin1(71) and inhibit the dephosphorylation of Pin1. Alternatively, the invention provides screening methods to identify compounds that interact with pPin1(71) and/or inhibit the dephosphorylation of pPin1(71).

### BRIEF DESCRIPTION OF THE FIGURES

FIG. 1 depicts the amino acid sequence of Pin1 (SEQ ID NO:1).

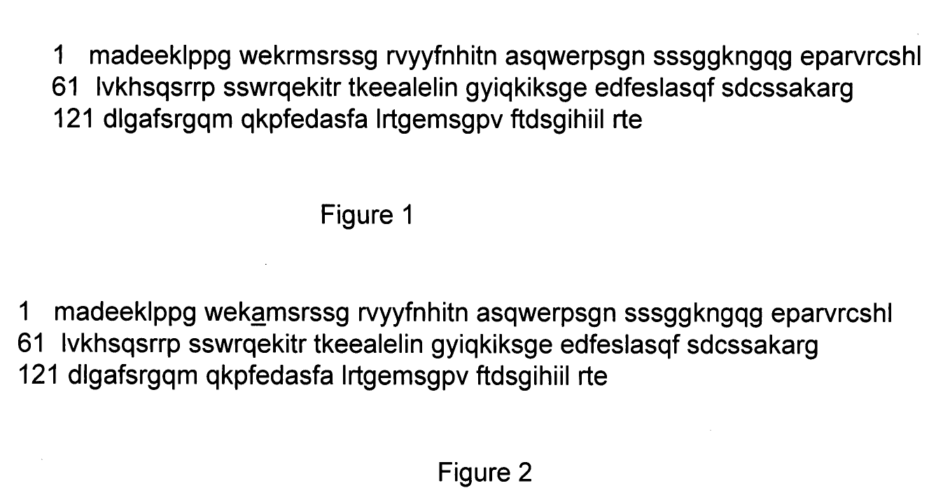

FIG. 2 depicts the amino acid sequence of Pin1 R14A (SEQ ID NO:2).

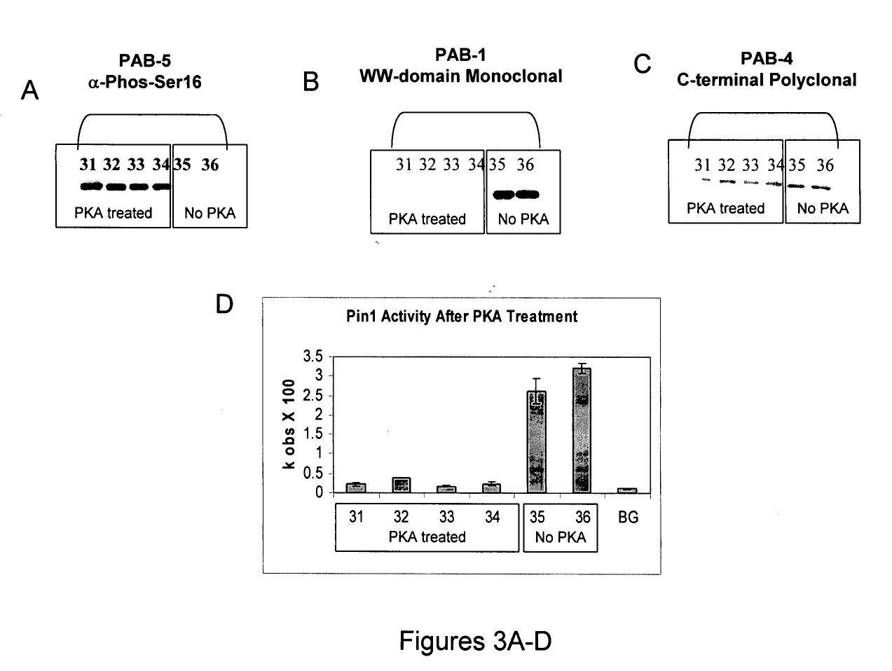

FIGS. 3A-D depict the effects of phosphorylation of Pin1 by PKA. FIGS. 3A-C depict western blots using Pin1 specific antibodies. FIG. 3D depicts an activity graph of PKA treated Pin1 and PKA un-treated Pin1 R14A versus the observed rate constant for isomerization of substrate.

FIGS. 4A-D depict MALDI-TOF spectra. FIG. 4A depicts the mass spectrum of PKA treated R14A. FIG. 4B depicts the mass spectrum of PKA untreated R14A. FIG. 4C depicts the mass spectrum of PKA un-treated wild-type Pin1. FIG. 4D depicts the mass spectrum of PKA treated wild-type Pin1.

FIG. 5A-B depict MALDI-TOF spectra of partial tryptic digests of Pin1. FIG. 5A depicts the mass spectrum of a Pin1 tryptic digest. FIG. 5B depicts a mass spectrum of a partial digest of PKA treated Pin1.

### DETAILED DESCRIPTION

The features and other details of the invention will now be more particularly described and pointed out in the claims. It will be understood that the particular embodiments of the invention are shown by way of illustration and not as limitations of the invention. The principle features of this invention can be employed in various embodiments without departing from the scope of the invention.

The present invention is based, at least in part, on the discovery of a novel phosphorylation site on the Pin1 enzyme. The instant invention provides Pin1 polypeptides, crystals, and crystal structures of Pin1 phosphorylated at position 71 (pPin1(71)). The invention further provides diagnostic and prognostic methods using antibodies specific for phosphorylated Pin1, e.g., Pin1 phosphorylated at position 16, position 71, or both. The invention is also based, at least in part, on the discovery that Pin1 molecules with mutations at position 71 that can not be phosphorylated, are constitutively active. The invention provides modulators of Pin1 specific to mutant Pin1 molecules that have non-wild type residues at position 71. The invention also provides methods for determining if a subject is predisposed to developing a Pin1 associated condition by determining if said subject has a mutation in Pin1 that renders the polypeptide constitutively active.

The term “biological sample” includes solid and body fluid samples. The biological samples of the present invention may include cells, protein or membrane extracts of cells, blood or biological fluids such as ascites fluid or brain fluid (e.g., cerebrospinal fluid). Examples of solid biological samples include samples taken from feces, the rectum, central nervous system, bone, breast tissue, renal tissue, the uterine cervix, the endometrium, the head/neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, and the thymus. Examples of “body fluid samples” include samples taken from the blood, serum, cerebrospinal fluid, semen, prostate fluid, seminal fluid, urine, saliva, sputum, mucus, bone marrow, lymph, and tears. Samples for use in the methods of the invention can be obtained by standard methods including venous puncture and surgical biopsy. In certain embodiments, the biological sample is a breast, lung, colon, or prostate tissue sample obtained by needle biopsy.

The term “nuclear Pin1” is intended to include Pin1 polypeptide that is localized to the nucleus of a cell. In certain embodiments, nuclear Pin1 is predominantly phosphorylated, e.g., at position 16, 71, or both.

The term “cytoplasmic Pin1” is intended to include Pin1 polypeptide that is localized to the cytoplasm of a cell. In certain embodiments, cytoplasmic Pin1 is predominantly unphosphorylated.

The term “phosphorylation state” is intended to denote that the Pin1 polypeptide can exist in either a phosphorylated or unphosphorylated state. The phosphorylation state denotes whether the Pin1 in a biological sample is phosphorylated or unphosphorylated, or the relative ratios of phosphorylated to unphosphorylated Pin1 in a sample. For example, Lu et al. (2002, J. Biol. Chem. 277:2381-4) demonstrated the importance of the phosphorylation of serine 16 on the ability of Pin1 to bind phosphorylated substrate. The experiments described herein, indicate that Pin1 is also capable of being phosphorylated at position 71.

The term “Pin1-associated state” or “Pin1-associated disorder” includes disorders and states (e.g., a disease state) that are associated with the abnormal activity of Pin1. This abnormal activity can be as a result of the misexpression or misregulation of the production, degradation, or regulation of Pin1, e.g., the phosphorylation/dephosphorylation of Pin1 at position 16 and/or 71. Without being bound by theory, Pin1-associated disorders that are related to higher than necessary levels of Pin1 can be caused by (1) an increase in the level of transcription or translation, or a decrease in the level of degradation of Pin1 such that an abnormally high amount of Pin1 polypeptide is present in a cell, or (2) the amount Pin1 that is present in the unphosphorylated (i.e., active form) is abnormally high due to either an increase in the dephosphorylation of Pin1 or a decrease in the phosphorylation of Pin1. Pin-associated states can also be a result of a mutation in the Pin1 nucleic acid sequence that leads to misregulation or misexpression of Pin1 production, degradation or activity, e.g., a mutation at position 16 or 71 that leads to constitutive Pin1 activity. Pin1 disorders are often associated with abnormal cell growth, abnormal cell proliferation, or misexpression of Pin1 (e.g., Pin1 protein or nucleic acid). Pin1-associated states include states resulting from an elevation in the expression of cyclin D1 and/or Pin1. Pin1-associated states also include states resulting from an elevation in the phosphorylation level of c-Jun, particularly phosphorylation of c-Jun on Ser 63/73-Pro and/or from an elevation in the level of c-Jun amino terminal kinases (JNKs) present in a cell. Pin1-associated states include neoplasia, cancer, undesirable cell growth, and/or tumor growth. Pin1-associated states include states caused by DNA damage, an oncogenic protein (i.e., Ha-Ras), loss of or reduced expression of a tumor suppressor (i.e., Brca1), and/or growth factors. Pin1-associated state is also intended to include diseases or disorders caused by, or associated with, deregulation of genes and/or gene products involved in a biological pathway that includes Pin1 and/or cyclin D1 (e.g. beta-catenin, APC or WNT). Beta-catenin, APC and WNT have been linked to cancer development as demonstrated in Biochim. Biophys. Acta 1653:1-24 (2003) and Eur J Surg Oncol. 29:107-117 (2003). Pin1-associated states further include disorders and states associated with regulation or activity of Pin1 in the brain, e.g., Alzheimer's disease, wherein the phosphorylation state of tau is influenced by the activity of Pin1.

The term “misexpression” includes a non-wild type pattern of gene expression or the misregulation of the control of Pin1, e.g., the phosphorylation and/or dephosphorylation of Pin1. Expression as used herein includes transcriptional, post transcriptional, e.g., mRNA stability, translational, and post translational stages. Misexpression includes: expression at non-wild type levels, i.e., over or under expression; a pattern of expression that differs from wild type in terms of the time or stage at which the gene is expressed, e.g., increased or decreased expression (as compared with wild type) at a predetermined developmental period or stage; a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus. Misexpression includes any expression from a transgenic nucleic acid. Misexpression includes the lack or non-expression of a gene or transgene, e.g., that can be caused by a deletion of all or part of the gene or its control sequences. Misregulation includes any non-wild type level of Pin1 phosphorylation and/or dephosphorylation when compared to Pin1 in normal tissue. For example, misregulation of Pin1 can result in higher or lower levels of phosphorylated or unphosphorylated Pin1 at, for example, serine 16 or serine 71.

The term “carcinoma” includes malignancies of epithelial or endocrine tissues, including respiratory system carcinomas, gastrointestinal system carcinomas, genitourinary system carcinomas, testicular carcinomas, breast carcinomas, prostate carcinomas, endocrine system carcinomas, melanomas, choriocarcinoma, and carcinomas of the cervix, lung, head and neck, colon, and ovary. The term “carcinoma” also includes carcinosarcomas, which include malignant tumors composed of carcinomatous and sarcomatous tissues. The term “adenocarcinoma” includes carcinomas derived from glandular tissue or a tumor in which the tumor cells form recognizable glandular structures.

For example, the therapeutic methods of the present invention can be applied to cancerous cells of mesenchymal origin, such as those producing sarcomas (e.g., fibrosarcoma, myxosarcoma, liosarcoma, chondrosarcoma, osteogenic sarcoma or chordosarcoma, angiosarcoma, endotheliosardcoma, lympangiosarcoma, synoviosarcoma or mesothelisosarcoma); leukemias and lymphomas such as granulocytic leukemia, monocytic leukemia, lymphocytic leukemia, malignant lymphoma, plasmocytoma, reticulum cell sarcoma, or Hodgkin's disease; sarcomas such as leiomysarcoma or rhabdomysarcoma, tumors of epithelial origin such as squamous cell carcinoma, basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, chorioaencinoma, semonoma, or embryonal carcinoma; and tumors of the nervous system including gioma, menigoma, medulloblastoma, schwannoma or epidymoma. Additional cell types amenable to treatment according to the methods described herein include those giving rise to mammary carcinomas, gastrointestinal carcinoma, such as colonic carcinomas, bladder carcinoma, prostate carcinoma, and squamous cell carcinoma of the neck and head region. Examples of cancers amenable to treatment according to the methods described herein include colorectal cancers, e.g., colon cancer.

The language “inhibiting undesirable cell growth” is intended to include the inhibition of undesirable or inappropriate cell growth. The inhibition is intended to include inhibition of cell proliferation, including rapid proliferation. For example, undesirable cell growth can result in benign masses or malignant tumors. Examples of benign conditions which result from inappropriate cell growth or angiogenesis are diabetic retinopathy, retrolental fibrioplasia, neovascular glaucoma, psoriasis, angiofibromas, rheumatoid arthritis, hemangiomas, Karposi's sarcoma, and other conditions or dysfunctions characterized by disregulated endothelial cell division.

The language “inhibiting tumor growth” or “inhibiting neoplasia” includes the prevention of the growth of a tumor in a subject or a reduction in the growth of a pre-existing tumor in a subject, or can be the inhibition of the metastasis of a tumor from one site to another. In particular, the language “tumor” is intended to encompass both in vitro, e.g., in cell culture, and in vivo tumors that form in any organ or body part of the subject. The tumors preferably are tumors sensitive to the Pin1-modulating compounds of the present invention.

The term “cancer” includes malignancies characterized by deregulated or uncontrolled cell growth, for instance carcinomas, sarcomas, leukemias, and lymphomas. The terms “cancer” and “tumor” may be used interchangeably herein. The term “cancer” includes primary malignant tumors, e.g., those whose cells have not migrated to sites in the subject's body other than the site of the original tumor, and secondary malignant tumors, e.g., those arising from metastasis, the migration of tumor cells to secondary sites that are different from the site of the original tumor.

Pin1 modulating agents may be used to treat, inhibit, and/or prevent undesirable cell growth, neoplasia, neurodegenerative diseases, and/or cancer in a subject. Pin1 modulating compounds may be used to inhibit Pin1 activity in a subject. In one embodiment, Pin1 modulating compounds may be used to inhibit cyclin D1 expression in a subject. In a further embodiment, Pin1 modulating compounds of the invention can be used to treat subjects having Alzheimer's disease.

The language “Pin1 modulating compound” refers to compounds that modulate, e.g., inhibit, promote, or otherwise alter, the activity of Pin1. Pin1 modulating compounds include both Pin1 agonists and antagonists. In certain embodiments, the Pin1 modulating compounds include compounds that interact with the peptidyl prolyl isomerase domain (PPI) and/or the WW domain of Pin1. In certain embodiments, the Pin1 modulating compound is substantially specific to Pin1. The phrase “substantially specific for Pin1” is intended to include inhibitors of the invention that have a Kᵢ or K_{d} that is at least 2, 3, 4, 5, 10, 15, or 20 times less than the Kᵢ or K_{d} for other peptidyl prolyl isomerases, e.g., hCyP-A, hCyP-B, hCyP-C, NKCA, hFKBP-12, hFKBP-13, and hFKBP-25. In one embodiment, the Pin1 inhibitor is specific for Pin1 that is phosphorylated, e.g., at position 16 and/or 71, thereby inhibiting dephosphorylation of Pin1. In another embodiment, the Pin1 inhibitor of the invention is specific for constitutively active Pin1, e.g., a Pin1 with a mutation at position 16 or 71.

Examples of Pin1 modulating compounds include compounds described in PCT Publication No's: WO 03074550 A2, WO 03073999 A2, WO 03074497 A1, WO 04028535A1, WO 03074001A2, WO 03074002A2, W005007123A2 and WO 04093803A2. The compounds described in these applications can be altered such that they have the ability to covalently interact with residues in the active site of Pin1. The entire contents of each of the aforementioned applications are hereby expressly incorporated herein by reference in their entireties. In certain embodiments, the Pin1 inhibiting compounds include compounds that interact with the PPI and/or the WW domain of Pin1.

The term “preselected” is intended to mean that a subject has been identified based on their level and/or phosphorylation state of Pin1, e.g., phosphorylation at position 16, 71, or both, to be likely to benefit from treatment with a Pin1 modulator. In certain embodiments, a subject is preselected based on the levels of unphosphorylated Pin1, phosphorylated Pin1, or the relative amounts of phosphorylated and unphosphorylated Pin1.

The term “prognosis” is intended to mean the probable course and outcome of a disease, e.g., a Pin1 associated disease. In certain embodiments, the term is intended to mean the likelihood that a subject will live longer than the average length of time that a population of subjects with a similar disease will live. In related embodiments, a subject's prognosis is indicative of the aggressiveness and course of treatment that a subject will receive.

Polypeptides

In one embodiment, the invention provides a mutant Pin1 in which there is a mutation in the phosphokinase A (PKA) recognition site at residues 13-16 of SEQ ID NO:1. In one embodiment, the invention provides a phosphorylated Pin1 polypeptide in the crystallized form, e.g., pPin1(71). In another embodiment, the invention provides protein crystals and structural coordinates of pPin1(71). In yet another embodiment, this invention relates to methods of diagnosis, treatment and prognosis of subjects with a Pin1 associated disorder. Pin1 is a highly conserved protein (SEQ ID NO:1) that catalyzes the isomerization of only phosphorylated Ser/Thr-Pro bonds (Rananathan et al., (1997) Cell 89:875-86; Yaffe et al., (1997) Science 278:1957-1960,; Shen et al., (1998) Genes Dev. 12:706-720; Lu et al., (1999) Science 283:1325-1328; Crenshaw et al., (1998) EMBO J. 17:1315-1327; Lu et al., (1999) Nature 399:784-788; Zhou et al., (1999) Cell Mol. Life Sci. 56:788-806). In addition, Pin1 contains an N-terminal WW domain, which functions as a phosphorylated Ser/Thr-Pro binding module (Sudol, (1996) Prog. Biophys. Mol. Biol. 65:113-32). This phosphorylation-dependent interaction targets Pin1 to a subset of phosphorylated substrates, including Cdc25, Wee 1, Myt1, Tau-Rad4, and the C-terminal domain of RNA polymerase II large domain (Crenshaw et al., (1998) EMBO J. 17:1315-27; Shen, (1998) Genes Dev. 12:706-20; Wells, (1999) J. Cell. Sci. 112: 3861-71).

The specificity of Pin1 activity is essential for cell growth; depletion or mutations of Pin1 cause growth arrest, affect cell cycle checkpoints and induce premature mitotic entry, mitotic arrest and apoptosis in human tumor cells, yeast or Xenopus extracts (Lu et al., (1996) Nature 380:544-547; Winkler et al., (2000) Science 287:1644-1647; Hani et al., (1999) J Biol. Chem. 274:108-116). In addition, Pin1 is dramatically misexpressed in human cancer samples and the total level or concentration of Pin1 is correlated with the aggressiveness of tumors. Moreover, inhibition of Pin1 by various approaches, including Pin1 antisense polynucleotides or genetic depletion, kills human and yeast dividing cells by inducing premature mitotic entry and apoptosis.

Thus, Pin1-catalyzed prolyl isomerization regulates the conformation and function of these phosphoprotein substrates and facilitates dephosphorylation because of the conformational specificity of some phosphatases. Thus, Pin1-dependent peptide bond isomerization is an important post-phosphorylation regulatory mechanism, allowing cells to turn phosphoprotein function on or off with high efficiency and specificity during temporally regulated events, including the cell cycle (Lu et al., supra).

In one embodiment, the invention provides a mutant Pin1 polypeptide in which there is a mutation of an amino acid residue in the PKA recognition site for PKA phosphorylation of serine 16, thereby rendering PKA unable to phosphorylate Pin1 at position 16. In one embodiment, the mutation is at position 13, 14, 15, or 16. In one embodiment, the mutation is at position 14. In another embodiment, the mutation is an arginine to alanine substitution. In a specific embodiment, the mutant Pin1 is phosphorylated at position 71.

In another embodiment, the invention provides a Pin1 mutant that has lost the ability to be phosphorylated at position 71, e.g., Pin1 S71A. In one embodiment, this Pin1 mutant is constitutively active.

The polypeptides of the invention can be produced by art recognized methods using recombinant DNA technology. For example, the mutant polypeptides of the invention can be produced by using a commercially available mutagenesis kit, e.g., the QuikChange® Site-Directed Mutagenesis Kit (Stratagene, La Jolla, Calif.) to mutate the DNA encoding Pin1. This DNA can then be incorporated into an expression vector and produced in an expression system, e.g., a bacterial expression system. An exemplary detailed protocol for the expression of a specific Pin1 mutant, R14A, is described in the Examples.

pPin1 Crystals

The present invention is based, at least in part, on the crystal structure of Pin1 phosphorylated at position 16 and 71. The present invention is also based, at least in part, on the discovery that crystals of Pin1 polypeptide containing an alanine at position 14 of the amino acid sequence (Pin1 R14A) inhibit phosphorylation of serine 16 of Pin1 by phosphokinase A (PKA). Further, the crystal structure of Pin1 R14A treated with phosphokinase A indicates that Pin1 is phosphorylated at position 71. The Arg at position 14 is located in the WW domain (Ranganathan et al., (1997) Cell 89:875-86) and is part of a PKA recognition sequence. Mutation of the Arg14 residue to an alanine abolishes the ability of PKA to phosphorylate serine 16. The atomic coordinates of Pin1 R14A are listed in Table I, and the atomic coordinates of pPin1(71) R14A are listed in Table II.

Accordingly, in one aspect the invention features a crystallized Pin1 polypeptide containing a non-native amino acid at position 13, 14, 15 or 16 of the polypeptide sequence, wherein said non-native amino acid inhibits the ability of PKA to phosphorylate Pin1 at position 16. In certain embodiments, the non-native amino acid is at position 14. In one embodiment the amino acid at position 14 is alanine. In related embodiments, the invention pertains to crystals pPin1(71) of any part or fragment of a Pin1 polypeptide of the invention that contains the residues that comprise the isomerase active site (e.g., fragments of Pin1 R14A).

In one aspect the invention provides Pin1 crystals phosphorylated at position 16 and 71. The atomic coordinates of this structure are presented in Table III.

In another aspect, the invention provides crystal structures of Pin1 with a mutation at position 71 which is not phosphorylated and is enzymatically active. In certain embodiment, the mutation is S71A, S71P, S71L, S71T, or S71W. In one embodiment, the crystal structure of Pin1 with a mutation at position 71 is phosphorylated at position 16.

The term “appropriate conditions” include those conditions which result in the formation of a crystal which can be analyzed to a resolution of 5.0 Å, 4.0 Å, 3.0 Å, 2.0 Å or greater. In one embodiment, the temperature of crystallization of the Pin1 polypeptide is from about 1° C. to about 30° C., from about 1° C. to about 25° C., from about 1° C. to about 15° C., from about 1° C. to 10° C., or about 4° C. In a further embodiment, the conditions are selected such that crystals of the Pin1 polypeptide grow within an acceptable time and reach dimensions that are suitable for structural determination, e.g., by using X-ray diffraction. In certain embodiments, the acceptable time for crystal growth is 1 week or less, 5 days or less, 4 days or less, 3 days or less, or, advantageously, 2 days or less. In related embodiments, the dimensions of the crystal are 0.05 mm or greater per side, 0.1 mm or greater per side, 0.2 mm or greater per side, or approximately 0.3 mm per side or greater.

Crystals can be produced by one of skill in the art using routine techniques. For example, a skilled artisan can use a commercial crystal screening kit to determine conditions suitable for crystal growth. Screening kits are available from, for example, Hampton Research (Aliso Viejo, Calif.). Further, conditions in which the crystals of the instant invention were grown can be found in the Examples section.

Once Pin1 crystals have been obtained, the structure of the polypeptide that form the crystal can be solved by X-ray crystallography.

Antibodies

The invention provides a method of detecting the presence and amount of phosphorylated Pin1, e.g., pPin1(71) and/or pPin1(16) and/or pPin1(71) and (16) in a biological sample. The invention also provides methods of determining the amount of phosphorylated Pin1 relative to the amount of unphosphorylated Pin1 in a sample. Accordingly, the methods of the invention may use antibodies that recognize phosphorylated Pin1 and unphosphorylated Pin1, antibodies that are specific for phosphorylated Pin1 e.g., at position 16 and/or 71, or both, or antibodies that are specific for unphosphorylated Pin1. Further, this application provides antibodies and methods of detecting constitutively active Pin1 molecules. Antibodies that are specific for Pin1 are described in U.S. Pat. No. 6,596,848, the entire contents of which is incorporated herein by reference.

The phrase “antibodies specific for phosphorylated Pin1” is intended to include antibodies that preferentially bind to an antigen of Pin1 that contains a phosphorylated residue, e.g., at position 16 and/or 71. In a preferred embodiment, the antibodies of the invention specifically recognize pPin1(71). Antibodies that are specific for phosphorylated Pin1 bind to Pin1 with at least twice the affinity that they bind to a nonspecific antigen (e.g., BSA or casein). Further, an antibody that is specific for phosphorylated Pin1 has more affinity for phosphorylated Pin1 than it does for unphosphorylated Pin1. In certain embodiments, the antibody specific for phosphorylated Pin1 binds with at least 2, 3, 4, 5, 10, 20, 50, 100, 500, or 1000 times the affinity to phosphorylated Pin1 as it does to unphosphorylated Pin1. In at least one embodiment, the antibody specific for phosphorylated Pin1 recognizes a Pin1 molecule that is phosphorylated on serine 16 of Pin1. In another embodiment, the invention provides an antibody specific for phosphorylated Pin1 wherein said Pin1 is phosphorylated at position 71. In another embodiment, the invention provides antibodies that specifically recognize Pin1 mutants that are constitutively active, e.g., mutants at position 71 such as S71A.

The phrase “antibodies specific for unphosphorylated Pin1” is intended to include antibodies that preferentially bind a Pin1 polypeptide that is not phosphorylated. Antibodies that are specific for unphosphorylated Pin1 bind to Pin1 with at least twice the affinity that they bind to a nonspecific antigen (e.g., BSA or casein). Further, an antibody that is specific for unphosphorylated Pin1 has more affinity for unphosphorylated Pin1 than it does phosphorylated Pin1. In certain embodiments, the antibody specific for unphosphorylated Pin1 binds with at least 2, 3, 4, 5, 10, 20, 50, 100, 500, or 1000 times the affinity to unphosphorylated Pin1 as it does phosphorylated Pin1. In at least one embodiment, the antibody specific for unphosphorylated Pin1 recognizes an epitope of Pin1 comprising a residue that is capable of being phosphorylated, e.g., serine 71 of SEQ ID NO:1.

Polyclonal antibodies are produced by immunizing animals, usually a mammal, by multiple subcutaneous or intraperitoneal injections of an immunogen (antigen) and an adjuvant as appropriate. As an illustrative embodiment, animals are typically immunized against a protein, peptide or derivative by combining about 1 μg 10 to 1 mg of protein capable of eliciting an immune response, along with an enhancing carrier preparation, such as Freund's complete adjuvant, or an aggregating agent such as alum, and injecting the composition intradermally at multiple sites. Animals are later boosted with at least one subsequent administration of a lower amount, as 1/5 to 1/10 the original amount of immunogen in Freund's complete adjuvant (or other suitable adjuvant) by subcutaneous injection at multiple sites. Animals are subsequently bled, serum assayed to determine the specific antibody titer, and the animals are again boosted and assayed until the titer of antibody no longer increases (i.e., plateaus).

Such populations of antibody molecules are referred to as “polyclonal” because the population comprises a large set of antibodies each of which is specific for one of the many differing epitopes found in the immunogen, and each of which is characterized by a specific affinity for that epitope. An epitope is the smallest determinant of antigenicity, which for a protein, comprises a peptide of six to eight residues in length (Berzofsky, J. and I. Berkower, (1993) in Paul, W., Ed., Fundamental Immunology, Raven Press, N.Y., p.246). Affinities range from low, e.g 10⁻⁶ M, to high, e.g., 10⁻¹¹ M. The polyclonal antibody fraction collected from mammalian serum is isolated by well known techniques, e.g. by chromatography with an affinity matrix that selectively binds immunoglobulin molecules such as protein A, to obtain the IgG fraction. To enhance the purity and specificity of the antibody, the specific antibodies may be further purified by immunoaffinity chromatography using solid phase-affixed immunogen. The antibody is contacted with the solid phase-affixed immunogen for a period of time sufficient for the immunogen to immunoreact with the antibody molecules to form a solid phase-affixed immunocomplex. Bound antibodies are eluted from the solid phase by standard techniques, such as by use of buffers of decreasing pH or increasing ionic strength, the eluted fractions are assayed, and those containing the specific antibodies are combined.

“Monoclonal antibody” or “monoclonal antibody composition” as used herein refers to a preparation of antibody molecules of single molecular composition. A monoclonal antibody composition displays a single binding specificity and affinity for a particular epitope. Monoclonal antibodies can be prepared using a technique which provides for the production of antibody molecules by continuous growth of cells in culture. These include but are not limited to the hybridoma technique originally described by Kohler and Milstein (1975, Nature 256:495-497; see also Brown et al., (1981) J. Immunol 127:539-46; Brown et al., (1980) J Biol Chem 255:4980-83; Yeh et al., (1976) PNAS 76:2927-31; and Yeh et al., (1982) Int. J. Cancer 29:269-75) and the more recent human B cell hybridoma technique (Kozbor et al., (1983) Immunol Today 4:72), EBV-hybridoma technique (Cole et al., (1985) Monoclonal Antibodies and Cancer Therapy, Alan R. Liss, Inc., pp. 77-96), and trioma techniques. The technology for producing hybridomas is well known (see generally Current Protocols in Immunology, Coligan et al. ed., John Wiley & Sons, New York, 1994). Hybridoma cells producing a monoclonal antibody of the invention are detected by screening the hybridoma culture supernatants for antibodies that bind the polypeptide of interest, e.g., using a standard ELISA assay.

A monoclonal antibody can be produced by the following steps. In all procedures, an animal is immunized with an antigen such as a protein (or peptide thereof) as described above for preparation of a polyclonal antibody. The immunization is typically accomplished by administering the immunogen to an immunologically competent mammal in an immunologically effective amount, i.e., an amount sufficient to produce an immune response. Preferably, the mammal is a rodent such as a rabbit, rat or mouse. The mammal is then maintained on a booster schedule for a time period sufficient for the mammal to generate high affinity antibody molecules as described. A suspension of antibody-producing cells is removed from each immunized mammal secreting the desired antibody. After a sufficient time to generate high affinity antibodies, the animal (e.g., mouse) is sacrificed and antibody-producing lymphocytes are obtained from one or more of the lymph nodes, spleens and peripheral blood. Spleen cells are preferred, and can be mechanically separated into individual cells in a physiological medium using methods well known to one of skill in the art. The antibody-producing cells are immortalized by fusion to cells of a mouse myeloma line. Mouse lymphocytes give a high percentage of stable fusions with mouse homologous myelomas, however rat, rabbit and frog somatic cells can also be used. Spleen cells of the desired antibody-producing animals are immortalized by fusing with myeloma cells, generally in the presence of a fusing agent such as polyethylene glycol. Any of a number of myeloma cell lines suitable as a fusion partner are used with to standard techniques, for example, the P3-NS1/1-Ag4-1, P3-x63-Ag8.653 or Sp2/O-Ag14 myeloma lines, available from the American Type Culture Collection (ATCC), Rockville, Md.

The fusion-product cells, which include the desired hybridomas, are cultured in selective medium such as HAT medium, designed to eliminate unfused parental myeloma or lymphocyte or spleen cells. Hybridoma cells are selected and are grown under limiting dilution conditions to obtain isolated clones. The supernatants of each clonal hybridoma is screened for production of antibody of desired specificity and affinity, e.g., by immunoassay techniques to determine the desired antigen such as that used for immunization. Monoclonal antibody is isolated from cultures of producing cells by conventional methods, such as ammonium sulfate precipitation, ion exchange chromatography, and affinity chromatography (Zola et al., Monoclonal Hybridoma Antibodies: Techniques And Applications, Hurell (ed.), pp. 51-52, CRC Press, 1982). Hybridomas produced according to these methods can be propagated in culture in vitro or in vivo (in ascites fluid) using techniques well known to those with skill in the art.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening an antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., (1989) Science 246:1275- 1281; Griffiths et al., (1993) EMBO J. 12:725-734.

Additionally, recombinant antibodies, such as chimeric, diabodies, and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al., (1988) Science 240:1041-1043; Liu et al., (1987) Proc. Natl. Acad. Sci. USA 84:3439-3443; Liu et al, (1987) J. Immunol. 139:3521-3526; Sun et al., (1987) Proc. Natl. Acad Sci. USA 84:214-218; Nishimura et al., (1987) Cancer Res. 47:999-1005; Wood et al., (1985) Nature 314:446-449; and Shaw et al., (1988) J. Natl. Cancer Inst. 80:1553-1559); Morrison (1985) Science 229:1202-1207; Oi et al., (1986) Bio/Techniques 4:214; U.S. Pat. No. 5,225,539; Jones et al., (1986) Nature 321:552-525; Verhoeyan et al., (1988) Science 239:1534; and Beidler et al., (1988) J. Immunol. 141:4053-4060.

The term “diabodies” refers to a small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites, e.g., one specific for phosphorylation at position 16 and one specific for phosphorylation at position 71 of Pin1. Diabodies are described more fully in, for example, EP 404,097; WO 93/11161; and Hollinger et al., (1993) Proc. Natl. Acad Sci. USA 90: 6444-6448.

“Labeled antibody” as used herein includes antibodies that are labeled by a detectable means and includes enzymatically, radioactively, fluorescently, chemiluminescently, and/or bioluminescently labeled antibodies.

One of the ways in which an antibody can be detectably labeled is by linking the same to an enzyme. This enzyme, in turn, when later exposed to its substrate, will react with the substrate in such a manner as to produce a chemical moiety which can be detected, for example, by spectrophotometric, fluorometric or by visual means. Enzymes which can be used to detectably label the Pin1-specific or a cancer associated polypeptide-specific antibody include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase.

Detection may be accomplished using any of a variety of immunoassays. For example, by radioactively labeling an antibody, it is possible to detect the antibody through the use of radioimmune assays. A description of a radioimmune assay (RIA) may be found in Laboratory Techniques and Biochemistry in Molecular Biology, by Work, T. S., et al., North Holland Publishing Company, NY (1978), with particular reference to the chapter entitled “An Introduction to Radioimmune Assay and Related Techniques” by Chard, T.

The radioactive isotope can be detected by such means as the use of a gamma counter or a scintillation counter or by audioradiography. Isotopes which are particularly useful for the purpose of the present invention are: ³H, ¹³¹I, ³⁵S, ¹⁴C, and preferably ¹²⁵I.

It is also possible to label an antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerytherin, phycocyanin, allophycocyanin, o-phthaldehyde and fluorescamine.

An antibody can also be detectably labeled using fluorescence emitting metals such as ¹⁵²Eu, or others of the lanthanide series. These metals can be attached to the antibody using such metal chelating groups as diethylenetriaminepentaacetic acid (DTPA) or ethylenediaminetetraacetic acid (EDTA).

An antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, luciferin, isoluminol, theromatic acridinium ester, imidazole, acridinium salt and oxalate ester.

Likewise, a bioluminescent compound may be used to label an antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

### METHODS OF THE INVENTION

In one embodiment, the invention provides methods for evaluating subjects for the level and phosphorylation state of Pin1, e.g., at position 16, 71, or both. In certain embodiments, the invention provides methods for evaluating subjects for the level of Pin1 in combination with other cancer markers. These results can be used to preselect a subject for treatment with a Pin1 modulator.

The amount of phosphorylated and unphosphorylated Pin1 in a biological sample may be determined by a radioimmunoassay, an immunoradiometric assay, enzyme immunoassay, and/or by immunohistochemistry using antibodies specific for phosphorylated Pin1 and unphosphorylated Pin1, respectively.

“Radioimmunoassay” is a technique for detecting and measuring the concentration of an antigen using a labeled (i.e. radioactively labeled) form of the antigen. Examples of radioactive labels for antigens include ³H, ¹⁴C, and ¹²⁵I. The concentration of phosphorylated and unphosphorylated Pin1 in a sample (i.e. biological sample) is measured by having the antigen in the sample compete with a labeled (i.e. radioactively) antigen for binding to an antibody to the antigen. To ensure competitive binding between the labeled antigen and the unlabeled antigen, the labeled antigen is present in a concentration sufficient to saturate the binding sites of the antibody. The higher the concentration of antigen in the sample, the lower the concentration of labeled antigen that will bind to the antibody.

In a radioimmunoassay, to determine the concentration of labeled antigen bound to antibody, the antigen-antibody complex must be separated from the free antigen. One method for separating the antigen-antibody complex from the free antigen is by precipitating the antigen-antibody complex with an anti-isotype antiserum. Another method for separating the antigen-antibody complex from the free antigen is by precipitating the antigen-antibody complex with formalin-killed S. aureus. Yet another method for separating the antigen-antibody complex from the free antigen is by performing a “solid-phase radioimmunoassay” where the antibody is linked (i.e. covalently) to Sepharose beads, polystyrene wells, polyvinylchloride wells, or microtiter wells. By comparing the concentration of labeled antigen bound to antibody to a standard curve based on samples having a known concentration of antigen, the concentration of antigen in the biological sample can be determined.

A “Immunoradiometric assay” (IRMA) is an immunoassay in which the antibody reagent is radioactively labeled. An IRMA requires the production of a multivalent antigen conjugate, by techniques such as conjugation to a protein e.g., rabbit serum albumin (RSA). The multivalent antigen conjugate must have at least 2 antigen residues per molecule and the antigen residues must be of sufficient distance apart to allow binding by at least two antibodies to the antigen. For example, in an IRMA the multivalent antigen conjugate can be attached to a solid surface such as a plastic sphere. Unlabeled “sample” antigen and antibody to antigen which is radioactively labeled are added to a test tube containing the multivalent antigen conjugate coated sphere. The antigen in the sample competes with the multivalent antigen conjugate for antigen antibody binding sites. After an appropriate incubation period, the unbound reactants are removed by washing and the amount of radioactivity on the solid phase is determined. The amount of bound radioactive antibody is inversely proportional to the concentration of antigen in the sample.

The most common enzyme immunoassay is the “Enzyme-Linked Immunosorbent Assay (ELISA).” The “Enzyme-Linked Immunosorbent Assay (ELISA)” is a technique for detecting and measuring the concentration of an antigen using a labeled (i.e. enzyme linked) form of the antibody.

In a “sandwich ELISA”, an antibody (i.e. to phosphorylated and unphosphorylated Pin1) is linked to a solid phase (i.e. a microtiter plate) and exposed to a biological sample containing phosphorylated and/or unphosphorylated Pin1. The solid phase is then washed to remove unbound antigen. A labeled (i.e. enzyme linked) is then bound to the bound-antigen (if present) forming an antibody-antigen-antibody sandwich. Examples of enzymes that can be linked to the antibody are alkaline phosphatase, horseradish peroxidase, luciferase, urease, and β-galactosidase. The enzyme linked antibody reacts with a substrate to generate a colored reaction product that can be assayed for.

In a “competitive ELISA”, antibody is incubated with a sample containing phosphorylated and unphosphorylated Pin1. The antigen-antibody mixture is then contacted with an antigen-coated solid phase (i.e. a microtiter plate). The more antigen present in the sample, the less free antibody that will be available to bind to the solid phase. A labeled (i.e. enzyme linked) secondary antibody is then added to the solid phase to determine the amount of primary antibody bound to the solid phase.

In an “immunohistochemistry assay” a section of tissue for is tested for specific proteins by exposing the tissue to antibodies that are specific for the type of Pin1 protein that is being assayed (e.g., phosphorylated and unphosphorylated Pin1.

The antibodies are then visualized by any of a number of methods to determine the presence and amount of the protein present. Examples of methods used to visualize antibodies are, for example, through enzymes linked to the antibodies (e.g., luciferase, alkaline phosphatase, horseradish peroxidase, or β-galactosidase), or chemical methods (e.g., DAB/Substrate chromagen). Examples of immunohistochemistry assays are provided in the Examples.

Once the levels of phosphorylated and/or unphosphorylated Pin1 in a biological sample are determined the subject can be classified based on the level and/or ratio of phosphorylated and/or unphosphorylated Pin1.

In one embodiment, subjects with high levels of unphosphorylated Pin1 localized in the cytoplasm are preselected for treatment with a Pin1 modulator.

Methods of Prognosis

The instant invention provides method of determining the prognosis of a subject with a Pin1 associated disorder, e.g., a cell proliferative disorder such as. In certain embodiments, the Pin1 associated disorder is a type of cancer, e.g., colon, breast, or lung cancer. The instant invention provides for the determination of the prognosis of a subject by evaluating the levels of pPin1(71) alone, or in combination with pPin1(16) in the subject at one or more points in time.

In one embodiment, the level of pPin1 in a subject can be compared to the statistical mean level in a population of subjects with similar diseases and the prognosis of the subject can be determined based on the level of pPin1 relative to the statistical mean. If the level of pPin1 in a subject is lower than the mean, the prognosis of the individual is considered poor, e.g., the subject will likely not survive for as long as the mean length of survival of the population. If the level of pPin1 in a subject is higher than the mean, the prognosis of the individual is considered good, e.g., the subject will survive for as long as the mean length of survival of the population, or longer.

The term “statistical mean” is used herein in a manner consistent with the well-understood definitions in the art of statistics. The statistical mean can be determined by quantitating the level of pPin1 in a statistically significant number of subjects and determining the mean value of that population.

In another embodiment, the prognosis of an individual can be determined by evaluating the level of pPin1 in biological samples isolated at different time points. If the levels of pPin1 decrease from a first sample to a second sample, the prognosis of said subject is poor. If the levels of pPin1 in a sample stay the same, or increase, the prognosis is good.

The levels of pPin1 can be determined and compared with a survival curve generated with data from a statistically significant number of subjects having a similar disease. The comparison of the pPin1 levels in a subject to the survival curve will determine the prognosis of a subject, i.e., the chance the subject has to survive for a given amount of time.

In other embodiments, the levels of pPin1 in a biological sample can be determined and used in combination with the levels of other known prognostic markers to determine the prognosis of a subject. For example, the levels of pPin1 and one or more known cancer markers can be evaluated and together used to determine the prognosis of a subject. The use of pPin1 and one or more additional makers allows for a more accurate determination of a subject's prognosis.

The prognosis of a subject as determined by the methods disclosed herein, can aide in the determination of what course of treatment to provide a subject. Further, the prognosis can indicate the aggressiveness of treatment that is required.

In a further embodiment, the invention provides a method for determining if a subject is at risk or predisposed to developing a Pin1-associated state by obtaining a biological sample from the subject and determining if the subject has a Pin1 mutation that will lead to misregulation, or misexpression of Pin1. In one embodiment the invention provides a method of determining if a subject has a Pin1 mutation that would lead to a constitutively active Pin1. In one particular embodiment, the constitutively active Pin1 has a mutation such that it is unable to be phosphorylated at position 71. In one embodiment, the mutation is the result of a single nucleotide polymorphism (SNP). Ser71 in human Pin1 is encoded by the codon TCG, and SNPs that can occur at this position which could result in a change of the amino acid include Thr (ACG), Pro (CCG), Ala (GCG), Trp (TGG) and Leu (TTG).

One of skill in the art can determine the presence of SNPs using methods that are routine in the art such as the methods described by, for example, Sapolsky et al. (1999) U.S. Pat. No. 5,858,659; Shuber (1997) U.S. Pat. No. 5,633,134; Dahlberg (1998) U.S. Pat. No. 5,719,028; Murigneux (1998) WO98/30717; Shuber (1997) WO97/10366; Murphy et al. (1998) WO98/44157; Lander et al. (1998) WO98/20165; Goelet et al. (1995) WO95/12607, Cronin et al. (1998) WO98/30883; ligase based methods are described by Barany et al. (1997) WO97/31256 and Chen et al., (1998) Genome Res. 8(5):549-56; mass-spectroscopy-based methods described by Monforte (1998) WO98/12355, Turano et al. (1998) WO98/14616 and Ross et al., (1997) Anal Chem. 15, 4197-202; PCR-based methods described by Hauser et al., (1998) Plant J. 16,117-25; exonuclease-based methods described by Mundy U.S. Pat. No. 4,656,127; dideoxynucleotide-based methods by Cohen et al. WO91/02087; Genetic Bit Analysis or GBA described by Goelet et al. WO92/15712; Oligonucleotide Ligation Assays or OLAs described by Landegren et al., (1988) Science 241:1077-1080 and Nickerson et al., (1990) Proc. Natl. Acad Sci. USA. 87:8923-8927; or primer-guided nucleotide incorporation procedures described by Prezant et al., (1992) Hum. Mutat. 1:159-164; Ugozzoli et al., (1992) GATA 9:107-112; Nyreen et al., (1993) Anal. Biochem. 208:171-175. the entire contents of each of the aforementioned application is hereby incorporated herein by reference.

Methods of Treatment

The term “subject” is intended to include living organisms, e.g., prokaryotes and eukaryotes. Examples of subjects include mammals, e.g., humans, dogs, cows, horses, pigs, sheep, goats, cats, mice, rabbits, rats, and transgenic non-human animals. Most preferably the subject is a human.

The term “subject that would benefit from treatment with a Pin1 modulator” is intended to include subjects having a Pin1 associated disorder wherein treatment of said subject with a Pin1 modulator would alleviate, reduce or eliminate one or more symptoms of the Pin1 disorder.

The language “effective amount” of the compound is that amount necessary or sufficient to treat or prevent a Pin1 associated state, e.g. prevent the various morphological and somatic symptoms of a Pin1 associated state. In an example, an effective amount of a Pin1 modulator of the invention is the amount sufficient to inhibit undesirable cell growth in a subject. In another example, an effective amount of the Pin1 modulator compound is the amount sufficient to reduce the size of a pre-existing benign cell mass or malignant tumor in a subject. The effective amount can vary depending on such factors as the size and weight of the subject, the type of illness, or the particular Pin1 binding compound. For example, the choice of the Pin1 modulator compound can affect what constitutes an “effective amount”. One of ordinary skill in the art would be able to study the aforementioned factors and make the determination regarding the effective amount of the Pin1 modulating compound without undue experimentation.

The regimen of administration can affect what constitutes an effective amount. A Pin1 modulator compound can be administered to the subject either prior to or after the onset of a Pin1 associated state. Further, several divided dosages, as well as staggered dosages, can be administered daily or sequentially, or the dose can be continuously infused, or can be a bolus injection. Further, the dosages of the Pin1 modulator can be proportionally increased or decreased as indicated by the exigencies of the therapeutic or prophylactic situation.

The term “treated,” “treating” or “treatment” includes the diminishment or alleviation of at least one symptom associated or caused by the state, disorder or disease being treated. For example, treatment can be diminishment of one or several symptoms of a disorder or complete eradication of a disorder.

While it is possible for a compound of the present invention to be administered alone, it is preferable to administer the compound as a pharmaceutical composition.

The language “pharmaceutical composition” includes preparations suitable for administration to mammals, e.g., humans. When the modulators are administered as pharmaceuticals to mammals, e.g., humans, they can be given per se or as a pharmaceutical composition containing, for example, 0.1 to 99.5% (more preferably, 0.5 to 90%) of active ingredient in combination with a pharmaceutically acceptable carrier.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, transdermal, buccal, sublingual, rectal, vaginal and/or parenteral administration. The formulations may conveniently be presented in unit dosage form and may be prepared by any methods well known in the art of pharmacy. The amount of active ingredient which can be combined with a carrier material to produce a single dosage form will generally be that amount of the compound which produces a therapeutic effect. Generally, out of one hundred per cent, this amount will range from about 1 percent to about ninety-nine percent of active ingredient, preferably from about 5 percent to about 70 percent, most preferably from about 10 per cent to about 30 per cent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

In solid dosage forms for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of pharmaceutical compositions for use in the methods of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Dosage forms for the topical or transdermal administration of a compound include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Transdermal patches have the added advantage of providing controlled delivery of a compound to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Pharmaceutical compositions suitable for parenteral administration comprise one or more compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions for use in the methods of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

For the methods of treatment of the instant invention, formulations may be given orally, parenterally, topically, or rectally. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrastemal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracistemally and topically, as by powders, ointments or drops, including buccally and sublingually.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, intravenous and subcutaneous doses of the compounds of this invention for a patient, when used for the indicated analgesic effects, will range from about 0.0001 to about 100 mg per kilogram of body weight per day, more preferably from about 0.01 to about 50 mg per kg per day, and still more preferably from about 1.0 to about 100 mg per kg per day. An effective amount is that amount treats an Pin1 associated state. If desired, the effective daily dose of the active compound may be administered as two, three, four, five, six or more sub-doses administered separately at appropriate intervals throughout the day, optionally, in unit dosage forms.

Methods of Designing Modulators of Pin1

Based on the structural and functional data presented herein, one of skill in the art can use the newly presented data to design modulators of Pin1 activity. For example, modulators of Pin1 can be designed to interact with serine 71, e.g., have a moiety that interacts with serine 71 in a manner similar to a phosphate moiety.

Alternatively, modulators can be designed that bind to pPin1(71) and inhibit the dephosphorylation of Pin1 thereby keeping the enzyme in the inactive state.

Pin1 modulators can be designed as a whole or “de novo” using either an empty active site or optionally including some portion(s) of a known inhibitor(s), such as those described in, for example, WO 03074550 A2, WO 03073999 A2, or WO 03074002 A2. Programs which can aid in these methods include:

1. LUDI (Bohm, H.-J., “The Computer Program LUDI: A New Method for the De Novo Design of Enzyme Inhibitors”, J. Comp. Aid. Molec. Design, 6, pp. 61-78 (1992)). LUDI is available from Biosym Technologies, San Diego, Calif.

2. LEGEND (Nishibata, Y. and A. Itai, Tetrahedron, 47, p. 8985 (1991)). LEGEND is available from Molecular Simulations, Burlington, Mass.

3. LeapFrog (available from Tripos Associates, St. Louis, Mo.).

Other molecular modeling techniques may also be employed in accordance with the design methods of this invention. See, e.g., Cohen, N. C. et al., “Molecular Modeling Software and Methods for Medicinal Chemistry”, J. Med. Chem., 33, pp. 883-894 (1990). See also, Navia, M. A. and M. A. Murcko, “The Use of Structural Information in Drug Design”, Current Opinions in Structural Biology, 2, pp. 202-210 (1992).

Once a modulator has been designed, the activity can be tested using the protease-coupled PPIase assay developed by Fischer et al. (Biomed. Biochim. Acta, 1984, 43: 1101-1111). In a related embodiment, molecules that block the dephosphorylation of pPin1, e.g., pPin1 (16) and/or pPin1(71) can be evaluated using a modified protease-coupled PPIase assay. The methods of Fisher et al. can be modified to include a dephosphatase and the activity of Pin1 monitored in the presence of one or more modulators. The slower the rate of return of PPIase activity the better the modulator of PPI dephosphorylation.

The invention is further illustrated by the following examples, which should not be construed as further limiting. The contents of all references, pending patent applications and published patents, cited throughout this application are hereby expressly incorporated by reference. The animal models used throughout the Examples are accepted animal models and the demonstration of efficacy in these animal models is predictive of efficacy in humans.

### EXAMPLES

### Example 1

### Crystallization and Structural Determination of Pin1 R14A Production of Pin1 R14A:

An N-terminal histidine tagged fusion protein of the Pin1 R14A mutant is expressed in E. coil. The bacteria is grown using Terrific Broth at 37° C. The expression vector (a pET28a derivative) contains Kanamycin resistance, and the protein is induced by lowering the temperature to 20° C. and adding IPTG to 50 μM. After 6-8 hours the bacteria is harvested by centrifugation and stored at −80° C.

Purification of Pin1 R14A:

The purification of Pin1 R14A is based on the modified method of Ranganathan et al. (Cell (1997), 89, 875-886). The E. coli pellet is thawed and suspended at 5 mL per gram in 50 mM Tris (pH 8.0), 500 mM NaCl, 20 mM Imidazole (pH 8.0), 10% glycerol, 1% Tween 20, and 25 mM β-mercaptoethanol. Lysozyme (hen egg white) is added at 0.5 mg/mL and the solution stirred for 30 min at room temperature. The cells were disrupted by sonication, and the solution clarified by centrifugation. The extract was then applied to a 5 mL column of Qiagen Ni-NTA resin at a 1-2 mL/min flow rate. The column was washed with three column volumes of the above buffer, and then bound protein was eluted from the resin with 50 mM Tris (pH 8.0), 500 mM NaCl, 250 mM Imidazole (pH 8.0), and 10% glycerol. Fractions containing protein were pooled and thrombin was added at 0.5 μL/mL of 1 U/μL. The material was placed in a dialysis bag and dialyzed overnight against 50 mM Tris (pH 8.0), 500 mM NaCl, and 25 mM β-mercaptoethanol. The material is then passed through a 0.5 mL column of Qiagen Ni-NTA resin at 1 mL/min, and 0.25 mL column of Benzamidine-Sepharose at 1 mL/min. After concentration, the material was loaded onto a FPLC size-exclusion column (Superdex 75, HiLoad 16/60, Pharmacia). Fractions containing Pin1 R14A were concentrated and dialyzed against 10 mM HEPES (pH 7.5), 20 mM NaCl, 1 mM DTT. After dialysis, the material was concentrated to 15-20 mg/mL. Material not used immediately was stored at −80° C. Milligram quantities of purified Pin1 R14A are obtained from a liter of bacterial culture.

Crystallization of Pin1 R14A:

The protein is crystallized by screening against 1.8-2.05 M Ammonium sulfate, 1% PEG 400, 0.1 M HEPES (pH 7.5), 1 mM DTT. Crystallization is performed using the hanging drop method in a Linbro style plate, with 1 mL solution in the reservoir. Siliconized glass cover slips are inverted over the reservoir after mixing 2 μL protein solution and 2 μL reservoir solution. Trays are wrapped in foil and placed at 4° C. Crystal growth is observable within two days. Crystal growth was shortened to 2-3 days rather than 1-2 weeks for the wild-type protein, and crystal seeding techniques were not required to obtain sufficiently large crystals.

Crystals can be grown from 0.9-1.2 M Sodium citrate, 1% PEG 400, 0.1 M HEPES (pH 7.5), 5 mM DTT, with 0-5% glycerol.

Transfer of Pin1 R14A Crystals to Cryoprotectant:

The crystals are transferred directly into cryoprotection solutions (i) 30% PEG 400, 100 mM HEPES (pH 7.5), 1 mM DTT, or (ii) 25% MPD, 100 mM HEPES (pH 7.5) or (iii) 30% PEP, 100 mM HEPES (pH 7.5). Crystal transfer to the cryoprotection solution is necessary to preserve the crystal during cooling to −180° C. for data collection, and also to allow compounds to bind under low salt conditions, which is important to increase the solubility of the compounds and to remove the sulfate ion bound in the active site (placed by crystallization conditions, but removable upon soaking for several days).

Data Collection and Processing:

Data collection on a rotating anode X-ray generator is typically 6 hours, using 1 degree of oscillation and 5 minute exposure times. An oscillation range of 60 degrees is required for a complete data set. The conversion of Arg 14 to Ala is located in WW domain of Pin1. This mutation results in an altered and favorable crystallization space group of P3₁21. The crystals are mechanically robust and diffract to high resolution (˜1.9 Å). The images were processed and scaled using Denzo and Scalepack (Otwinowski et al., (1996) Meth. Enzymol., 276, 307-326). Molecular replacement (using PDB entry 1PIN as the original search model) and refinement was done with CNX (Brunger et al., (1990) Acta Cryst. D54, 905-921), model building was done with O (Jones et al., (1991) Acta Cryst. A 47, 110-119). The set of atomic coordinates of Pin1 R14A as determined by X-ray crystallography is provided as Table 1.

TABLE 1
Set of atomic coordinates of Pin1 R14A as determined by X-ray crystallography

| | | | | | | | | | | |
|---|---|---|---|---|---|---|---|---|---|---|
| ATOM | 1 | CB | LYS | 6 | 26.317 | 49.386 | −27.408 | 1.00 | 75.75 | C |
| ATOM | 2 | CG | LYS | 6 | 25.999 | 49.158 | −25.938 | 1.00 | 76.44 | C |
| ATOM | 3 | CD | LYS | 6 | 26.255 | 50.416 | −25.125 | 1.00 | 77.48 | C |
| ATOM | 4 | CE | LYS | 6 | 25.985 | 50.186 | −23.650 | 1.00 | 77.75 | C |
| ATOM | 5 | NZ | LYS | 6 | 26.269 | 51.405 | −22.845 | 1.00 | 78.45 | N |
| ATOM | 6 | C | LYS | 6 | 24.620 | 47.817 | −28.371 | 1.00 | 74.39 | C |
| ATOM | 7 | O | LYS | 6 | 23.834 | 48.207 | −27.507 | 1.00 | 74.98 | O |
| ATOM | 8 | N | LYS | 6 | 26.644 | 48.422 | −29.663 | 1.00 | 75.06 | N |
| ATOM | 9 | CA | LYS | 6 | 26.106 | 48.159 | −28.298 | 1.00 | 75.05 | C |
| ATOM | 10 | N | LEU | 7 | 24.249 | 47.086 | −29.418 | 1.00 | 73.33 | N |
| ATOM | 11 | CA | LED | 7 | 22.871 | 46.659 | −29.644 | 1.00 | 71.57 | C |
| ATOM | 12 | CB | LED | 7 | 22.295 | 46.010 | −28.379 | 1.00 | 71.92 | C |
| ATOM | 13 | CG | LED | 7 | 22.952 | 44.689 | −27.979 | 1.00 | 71.84 | C |
| ATOM | 14 | CD1 | LED | 7 | 22.289 | 44.121 | −26.733 | 1.00 | 71.98 | C |
| ATOM | 15 | CD2 | LED | 7 | 22.841 | 43.718 | −29.142 | 1.00 | 71.81 | C |
| ATOM | 16 | C | LED | 7 | 21.935 | 47.763 | −30.123 | 1.00 | 69.78 | C |
| ATOM | 17 | O | LED | 7 | 21.756 | 48.780 | −29.454 | 1.00 | 69.52 | O |
| ATOM | 18 | N | PRO | 8 | 21.327 | 47.567 | −31.303 | 1.00 | 68.04 | N |
| ATOM | 19 | CD | PRO | 8 | 21.626 | 46.449 | −32.214 | 1.00 | 67.76 | C |
| ATOM | 20 | CA | PRO | 8 | 20.391 | 48.502 | −31.933 | 1.00 | 66.26 | C |
| ATOM | 21 | CB | PRO | 8 | 20.024 | 47.787 | −33.233 | 1.00 | 66.72 | C |
| ATOM | 22 | CG | PRO | 8 | 21.263 | 47.031 | −33.555 | 1.00 | 67.32 | C |
| ATOM | 23 | C | PRO | 8 | 19.169 | 48.756 | −31.045 | 1.00 | 64.59 | C |
| ATOM | 24 | O | PRO | 8 | 19.171 | 48.411 | −29.861 | 1.00 | 64.16 | O |
| ATOM | 25 | N | PRO | 9 | 18.114 | 49.374 | −31.605 | 1.00 | 62.73 | N |
| ATOM | 26 | CD | PRO | 9 | 18.120 | 50.167 | −32.846 | 1.00 | 63.09 | C |
| ATOM | 27 | CA | PRO | 9 | 16.901 | 49.656 | −30.830 | 1.00 | 59.43 | C |
| ATOM | 28 | CB | PRO | 9 | 16.207 | 50.746 | −31.652 | 1.00 | 61.30 | C |
| ATOM | 29 | CG | PRO | 9 | 17.323 | 51.369 | −32.440 | 1.00 | 63.30 | C |
| ATOM | 30 | C | PRO | 9 | 15.999 | 48.438 | −30.656 | 1.00 | 54.44 | C |
| ATOM | 31 | O | PRO | 9 | 15.606 | 47.803 | −31.636 | 1.00 | 54.75 | O |
| ATOM | 32 | N | GLY | 10 | 15.673 | 48.114 | −29.408 | 1.00 | 48.14 | N |
| ATOM | 33 | CA | GLY | 10 | 14.791 | 46.987 | −29.158 | 1.00 | 41.85 | C |
| ATOM | 34 | C | GLY | 10 | 15.451 | 45.633 | −28.957 | 1.00 | 36.53 | C |
| ATOM | 35 | O | GLY | 10 | 14.763 | 44.648 | −28.689 | 1.00 | 33.03 | O |
| ATOM | 36 | N | TRP | 11 | 16.773 | 45.579 | −29.077 | 1.00 | 34.92 | N |
| ATOM | 37 | CA | TRP | 11 | 17.505 | 44.327 | −28.904 | 1.00 | 33.37 | C |
| ATOM | 38 | CB | TRP | 11 | 18.664 | 44.241 | −29.898 | 1.00 | 31.56 | C |
| ATOM | 39 | CG | TRP | 11 | 18.240 | 43.993 | −31.302 | 1.00 | 30.03 | C |
| ATOM | 40 | CD2 | TEP | 11 | 17.911 | 42.727 | −31.885 | 1.00 | 29.07 | C |
| ATOM | 41 | CE2 | TRP | 11 | 17.564 | 42.967 | −33.235 | 1.00 | 28.10 | C |
| ATOM | 42 | CE3 | TRP | 11 | 17.875 | 41.411 | −31.399 | 1.00 | 26.09 | C |
| ATOM | 43 | CD1 | TRP | 11 | 18.082 | 44.923 | −32.292 | 1.00 | 30.05 | C |
| ATOM | 44 | NE1 | TRP | 11 | 17.678 | 44.315 | −33.453 | 1.00 | 29.35 | N |
| ATOM | 45 | CZ2 | TRP | 11 | 17.187 | 41.941 | −34.105 | 1.00 | 26.58 | C |
| ATOM | 46 | CZ3 | TRP | 11 | 17.500 | 40.391 | −32.264 | 1.00 | 25.29 | C |
| ATOM | 47 | CH2 | TRP | 11 | 17.161 | 40.663 | −33.604 | 1.00 | 26.24 | C |
| ATOM | 48 | C | TRP | 11 | 18.060 | 44.130 | −27.499 | 1.00 | 33.25 | C |
| ATOM | 49 | O | TRP | 11 | 18.412 | 45.089 | −26.811 | 1.00 | 32.43 | O |
| ATOM | 50 | N | GLU | 12 | 18.137 | 42.871 | −27.078 | 1.00 | 31.52 | N |
| ATOM | 51 | CA | GLU | 12 | 18.677 | 42.542 | −25.769 | 1.00 | 30.58 | C |
| ATOM | 52 | CB | GLU | 12 | 17.559 | 42.419 | −24.734 | 1.00 | 30.62 | C |
| ATOM | 53 | CG | GLU | 12 | 16.673 | 41.201 | −24.889 | 1.00 | 32.61 | C |
| ATOM | 54 | CD | GLU | 12 | 15.547 | 41.189 | −23.878 | 1.00 | 34.53 | C |
| ATOM | 55 | OE1 | GLU | 12 | 14.612 | 42.008 | −24.013 | 1.00 | 33.83 | O |
| ATOM | 56 | OE2 | GLU | 12 | 15.606 | 40.372 | −22.937 | 1.00 | 34.83 | O |
| ATOM | 57 | C | GLU | 12 | 19.437 | 41.229 | −25.880 | 1.00 | 29.52 | C |
| ATOM | 58 | O | GLO | 12 | 19.170 | 40.417 | −26.768 | 1.00 | 27.64 | O |
| ATOM | 59 | N | LYS | 13 | 20.392 | 41.031 | −24.985 | 1.00 | 28.84 | N |
| ATOM | 60 | CA | LYS | 13 | 21.183 | 39.811 | −24.993 | 1.00 | 30.55 | C |
| ATOM | 61 | CB | LYS | 13 | 22.525 | 40.053 | −24.299 | 1.00 | 32.86 | C |
| ATOM | 62 | CG | LYS | 13 | 23.505 | 38.895 | −24.393 | 1.00 | 36.09 | C |
| ATOM | 63 | CD | LYS | 13 | 24.783 | 39.201 | −23.614 | 1.00 | 38.49 | C |
| ATOM | 64 | CE | LYS | 13 | 25.499 | 40.428 | −24.175 | 1.00 | 39.37 | C |
| ATOM | 65 | NZ | LYS | 13 | 26.694 | 40.807 | −23.364 | 1.00 | 40.68 | N |
| ATOM | 66 | C | LYS | 13 | 20.394 | 38.745 | −24.257 | 1.00 | 30.06 | C |
| ATOM | 67 | O | LYS | 13 | 19.679 | 39.040 | −23.294 | 1.00 | 28.85 | O |
| ATOM | 68 | N | ALA | 14 | 20.510 | 37.504 | −24.714 | 1.00 | 29.08 | N |
| ATOM | 69 | CA | ALA | 14 | 19.798 | 36.413 | −24.073 | 1.00 | 29.26 | C |
| ATOM | 70 | CB | ALA | 14 | 18.550 | 36.049 | −24.882 | 1.00 | 29.89 | C |
| ATOM | 71 | C | ALA | 14 | 20.705 | 35.199 | −23.915 | 1.00 | 28.49 | C |
| ATOM | 72 | O | ALA | 14 | 21.813 | 35.161 | −24.448 | 1.00 | 26.85 | O |
| ATOM | 73 | N | MET | 15 | 20.220 | 34.208 | −23.183 | 1.00 | 29.72 | N |
| ATOM | 74 | CA | MET | 15 | 20.984 | 32.995 | −22.943 | 1.00 | 29.88 | C |
| ATOM | 75 | CB | MET | 15 | 21.318 | 32.895 | −21.456 | 1.00 | 33.30 | C |
| ATOM | 76 | CG | MET | 15 | 22.022 | 31.619 | −21.070 | 1.00 | 36.15 | C |
| ATOM | 77 | SD | MET | 15 | 23.597 | 31.529 | −21.892 | 1.00 | 39.84 | S |
| ATOM | 78 | CE | MET | 15 | 24.542 | 32.702 | −20.899 | 1.00 | 37.57 | C |
| ATOM | 79 | C | MET | 15 | 20.180 | 31.775 | −23.362 | 1.00 | 29.22 | C |
| ATOM | 80 | O | MET | 15 | 19.071 | 31.573 | −22.883 | 1.00 | 27.04 | O |
| ATOM | 81 | N | SER | 16 | 20.738 | 30.963 | −24.254 | 1.00 | 28.77 | N |
| ATOM | 82 | CA | SER | 16 | 20.048 | 29.757 | −24.693 | 1.00 | 28.24 | C |
| ATOM | 83 | CB | SER | 16 | 20.804 | 29.085 | −25.834 | 1.00 | 28.93 | C |
| ATOM | 84 | OG | SER | 16 | 20.279 | 27.788 | −26.061 | 1.00 | 30.01 | O |
| ATOM | 85 | C | SER | 16 | 19.932 | 28.776 | −23.532 | 1.00 | 28.89 | C |
| ATOM | 86 | O | SER | 16 | 20.932 | 28.418 | −22.910 | 1.00 | 27.90 | O |
| ATOM | 87 | N | AEG | 17 | 18.706 | 28.352 | −23.244 | 1.00 | 29.91 | N |
| ATOM | 88 | CA | ARG | 17 | 18.444 | 27.403 | −22.166 | 1.00 | 32.62 | C |
| ATOM | 89 | CB | ARG | 17 | 16.939 | 27.317 | −21.895 | 1.00 | 34.74 | C |
| ATOM | 90 | CG | ARG | 17 | 16.323 | 28.597 | −21.357 | 1.00 | 37.67 | C |
| ATOM | 91 | CD | ARG | 17 | 16.886 | 28.960 | −19.994 | 1.00 | 38.10 | C |
| ATOM | 92 | NE | ARO | 17 | 16.386 | 30.253 | −19.533 | 1.00 | 40.92 | N |
| ATOM | 93 | CZ | ARO | 17 | 15.132 | 30.484 | −19.153 | 1.00 | 41.40 | C |
| ATOM | 94 | NH1 | ARG | 17 | 14.235 | 29.508 | −19.167 | 1.00 | 41.19 | N |
| ATOM | 95 | NH2 | ARG | 17 | 14.773 | 31.700 | −18.769 | 1.00 | 41.62 | N |
| ATOM | 96 | C | ARO | 17 | 18.963 | 26.011 | −22.511 | 1.00 | 33.46 | C |
| ATOM | 97 | O | ARG | 17 | 19.246 | 25.207 | −21.621 | 1.00 | 32.53 | O |
| ATOM | 98 | N | SER | 18 | 19.086 | 25.730 | −23.804 | 1.00 | 34.96 | N |
| ATOM | 99 | CA | SER | 18 | 19.552 | 24.424 | −24.259 | 1.00 | 37.48 | C |
| ATOM | 100 | CB | SER | 18 | 18.902 | 24.070 | −25.599 | 1.00 | 38.32 | C |
| ATOM | 101 | OG | SER | 18 | 17.506 | 23.886 | −25.458 | 1.00 | 43.49 | O |
| ATOM | 102 | C | SER | 18 | 21.061 | 24.307 | −24.404 | 1.00 | 37.45 | C |
| ATOM | 103 | O | SER | 18 | 21.662 | 23.349 | −23.920 | 1.00 | 40.02 | O |
| ATOM | 104 | N | SER | 19 | 21.669 | 25.286 | −25.066 | 1.00 | 36.22 | N |
| ATOM | 105 | CA | SER | 19 | 23.102 | 25.271 | −25.311 | 1.00 | 35.77 | C |
| ATOM | 106 | CB | SER | 19 | 23.377 | 25.684 | −26.759 | 1.00 | 38.18 | C |
| ATOM | 107 | OG | SER | 19 | 22.862 | 26.978 | −27.025 | 1.00 | 39.68 | O |
| ATOM | 108 | C | SER | 19 | 23.938 | 26.137 | −24.386 | 1.00 | 35.38 | C |
| ATOM | 109 | O | SER | 19 | 25.120 | 25.870 | −24.188 | 1.00 | 35.14 | O |
| ATOM | 110 | N | GLY | 20 | 23.332 | 27.177 | −23.825 | 1.00 | 35.10 | N |
| ATOM | 111 | CA | GLY | 20 | 24.074 | 28.060 | −22.947 | 1.00 | 33.07 | C |
| ATOM | 112 | C | GLY | 20 | 24.807 | 29.116 | −23.756 | 1.00 | 31.89 | C |
| ATOM | 113 | O | GLY | 20 | 25.558 | 29.923 | −23.215 | 1.00 | 31.35 | O |
| ATOM | 114 | N | ARG | 21 | 24.590 | 29.112 | −25.064 | 1.00 | 30.69 | N |
| ATOM | 115 | CA | ARG | 21 | 25.241 | 30.083 | −25.929 | 1.00 | 30.10 | C |
| ATOM | 116 | CB | ARG | 21 | 25.371 | 29.524 | −27.343 | 1.00 | 33.05 | C |
| ATOM | 117 | CG | ARG | 21 | 26.334 | 30.296 | −28.229 | 1.00 | 37.69 | C |
| ATOM | 118 | CD | ARG | 21 | 26.025 | 30.027 | −29.687 | 1.00 | 40.08 | C |
| ATOM | 119 | NE | ARG | 21 | 25.742 | 28.615 | −29.899 | 1.00 | 40.15 | N |
| ATOM | 120 | CZ | ARG | 21 | 25.137 | 28.125 | −30.973 | 1.00 | 40.11 | C |
| ATOM | 121 | NH1 | ARG | 21 | 24.745 | 28.932 | −31.951 | 1.00 | 38.33 | N |
| ATOM | 122 | NH2 | ARG | 21 | 24.915 | 26.821 | −31.059 | 1.00 | 42.15 | N |
| ATOM | 123 | C | ARG | 21 | 24.403 | 31.360 | −25.966 | 1.00 | 27.27 | C |
| ATOM | 124 | O | ARG | 21 | 23.173 | 31.308 | −25.940 | 1.00 | 24.40 | O |
| ATOM | 125 | N | VAL | 22 | 25.071 | 32.502 | −26.033 | 1.00 | 26.38 | N |
| ATOM | 126 | CA | VAL | 22 | 24.364 | 33.773 | −26.072 | 1.00 | 25.85 | C |
| ATOM | 127 | CB | VAL | 22 | 25.328 | 34.963 | −25.832 | 1.00 | 28.19 | C |
| ATOM | 128 | CG1 | VAL | 22 | 26.054 | 34.787 | −24.507 | 1.00 | 31.21 | C |
| ATOM | 129 | CG2 | VAL | 22 | 26.329 | 35.067 | −26.972 | 1.00 | 29.97 | C |
| ATOM | 130 | C | VAL | 22 | 23.679 | 33.968 | −27.422 | 1.00 | 23.85 | C |
| ATOM | 131 | O | VAL | 22 | 24.131 | 33.456 | −28.443 | 1.00 | 22.20 | O |
| ATOM | 132 | N | TYR | 23 | 22.563 | 34.684 | −27.415 | 1.00 | 23.69 | N |
| ATOM | 133 | CA | TYR | 23 | 21.856 | 34.991 | −28.649 | 1.00 | 22.30 | C |
| ATOM | 134 | CB | TYR | 23 | 20.838 | 33.901 | −29.014 | 1.00 | 22.14 | C |
| ATOM | 135 | CG | TYR | 23 | 19.604 | 33.801 | −28.143 | 1.00 | 23.90 | C |
| ATOM | 136 | CD1 | TYR | 23 | 18.394 | 34.384 | −28.536 | 1.00 | 24.48 | C |
| ATOM | 137 | CE1T | YR | 23 | 17.236 | 34.223 | −27.777 | 1.00 | 23.42 | C |
| ATOM | 138 | CD2 | TYR | 23 | 19.624 | 33.062 | −26.960 | 1.00 | 25.02 | C |
| ATOM | 139 | CE2 | TYR | 23 | 18.478 | 32.896 | −26.196 | 1.00 | 27.09 | C |
| ATOM | 140 | CZ | TYR | 23 | 17.286 | 33.475 | −26.610 | 1.00 | 26.36 | C |
| ATOM | 141 | OH | TYR | 23 | 16.151 | 33.282 | −25.852 | 1.00 | 26.92 | O |
| ATOM | 142 | C | TYR | 23 | 21.179 | 36.322 | −28.409 | 1.00 | 22.90 | C |
| ATOM | 143 | O | TYR | 23 | 21.224 | 36.850 | −27.298 | 1.00 | 22.70 | O |
| ATOM | 144 | N | TYR | 24 | 20.561 | 36.873 | −29.441 | 1.00 | 23.42 | N |
| ATOM | 145 | CA | TYR | 24 | 19.916 | 38.161 | −29.289 | 1.00 | 23.78 | C |
| ATOM | 146 | CB | TYR | 24 | 20.641 | 39.189 | −30.156 | 1.00 | 26.76 | C |
| ATOM | 147 | CG | TYR | 24 | 22.094 | 39.296 | −29.751 | 1.00 | 31.08 | C |
| ATOM | 148 | CD1 | TYR | 24 | 23.035 | 38.359 | −30.191 | 1.00 | 33.27 | C |
| ATOM | 149 | CE1 | TYR | 24 | 24.353 | 38.387 | −29.729 | 1.00 | 34.88 | C |
| ATOM | 150 | CD2 | TYR | 24 | 22.512 | 40.269 | −28.843 | 1.00 | 33.85 | C |
| ATOM | 151 | CE2 | TYR | 24 | 23.825 | 40.308 | −28.375 | 1.00 | 34.75 | C |
| ATOM | 152 | CZ | TYR | 24 | 24.740 | 39.366 | −28.820 | 1.00 | 36.76 | C |
| ATOM | 153 | OH | TYR | 24 | 26.037 | 39.402 | −28.354 | 1.00 | 38.29 | O |
| ATOM | 154 | C | TYR | 24 | 18.431 | 38.094 | −29.588 | 1.00 | 23.08 | C |
| ATOM | 155 | O | TYR | 24 | 17.989 | 37.360 | −30.466 | 1.00 | 21.07 | O |
| ATOM | 156 | N | PHE | 25 | 17.668 | 38.857 | −28.821 | 1.00 | 23.04 | N |
| ATOM | 157 | CA | PHE | 25 | 16.224 | 38.887 | −28.942 | 1.00 | 23.59 | C |
| ATOM | 158 | CB | PHE | 25 | 15.625 | 38.214 | −27.706 | 1.00 | 23.65 | C |
| ATOM | 159 | CG | PHE | 25 | 14.153 | 38.413 | −27.550 | 1.00 | 24.29 | C |
| ATOM | 160 | CD1 | PHE | 25 | 13.252 | 37.731 | −28.360 | 1.00 | 24.38 | C |
| ATOM | 161 | CD2 | PHE | 25 | 13.663 | 39.279 | −26.575 | 1.00 | 24.35 | C |
| ATOM | 162 | CE1 | PHE | 25 | 11.874 | 37.910 | −28.200 | 1.00 | 23.65 | C |
| ATOM | 163 | CE2 | PHE | 25 | 12.296 | 39.464 | −26.409 | 1.00 | 24.27 | C |
| ATOM | 164 | CZ | PHE | 25 | 11.400 | 38.776 | −27.225 | 1.00 | 24.79 | C |
| ATOM | 165 | C | PHE | 25 | 15.756 | 40.333 | −29.055 | 1.00 | 24.00 | C |
| ATOM | 166 | O | PHE | 25 | 16.287 | 41.219 | −28.386 | 1.00 | 24.15 | O |
| ATOM | 167 | N | ASN | 26 | 14.773 | 40.564 | −29.921 | 1.00 | 23.32 | N |
| ATOM | 168 | CA | ASN | 26 | 14.215 | 41.900 | −30.122 | 1.00 | 23.05 | C |
| ATOM | 169 | CB | ASN | 26 | 14.169 | 42.231 | −31.621 | 1.00 | 22.84 | C |
| ATOM | 170 | CG | ASN | 26 | 13.816 | 43.686 | −31.889 | 1.00 | 20.90 | C |
| ATOM | 171 | OD1 | ASN | 26 | 12.734 | 44.146 | −31.542 | 1.00 | 20.54 | O |
| ATOM | 172 | ND2 | ASN | 26 | 14.734 | 44.412 | −32.511 | 1.00 | 21.46 | N |
| ATOM | 173 | C | ASN | 26 | 12.810 | 41.891 | −29.536 | 1.00 | 20.66 | C |
| ATOM | 174 | O | ASN | 26 | 11.950 | 41.139 | −29.986 | 1.00 | 20.34 | O |
| ATOM | 175 | N | HIS | 27 | 12.572 | 42.725 | −28.528 | 1.00 | 21.02 | N |
| ATOM | 176 | CA | HIS | 27 | 11.266 | 42.747 | −27.880 | 1.00 | 20.66 | C |
| ATOM | 177 | CB | HIS | 27 | 11.398 | 43.280 | −26.442 | 1.00 | 20.66 | C |
| ATOM | 178 | CG | HIS | 27 | 11.962 | 44.665 | −26.348 | 1.00 | 23.34 | C |
| ATOM | 179 | CD2 | HIS | 27 | 13.198 | 45.103 | −26.012 | 1.00 | 23.87 | C |
| ATOM | 180 | ND1 | HIS | 27 | 11.211 | 45.795 | −26.597 | 1.00 | 25.84 | N |
| ATOM | 181 | CE1 | HIS | 27 | 11.959 | 46.868 | −26.416 | 1.00 | 25.11 | C |
| ATOM | 182 | NE2 | HIS | 27 | 13.170 | 46.476 | −26.062 | 1.00 | 27.78 | N |
| ATOM | 183 | C | HIS | 27 | 10.170 | 43.498 | −28.637 | 1.00 | 20.96 | C |
| ATOM | 184 | O | HIS | 27 | 9.031 | 43.556 | −28.187 | 1.00 | 21.38 | O |
| ATOM | 185 | N | ILE | 28 | 10.507 | 44.063 | −29.786 | 1.00 | 22.76 | N |
| ATOM | 186 | CA | ILE | 28 | 9.506 | 44.769 | −30.579 | 1.00 | 23.22 | C |
| ATOM | 187 | CB | ILE | 28 | 10.077 | 46.083 | −31.149 | 1.00 | 23.38 | C |
| ATOM | 188 | CG2 | ILE | 28 | 9.017 | 46.788 | −31.990 | 1.00 | 22.95 | C |
| ATOM | 189 | CG1 | ILE | 28 | 10.515 | 46.990 | −29.993 | 1.00 | 23.21 | C |
| ATOM | 190 | CD1 | ILE | 28 | 11.319 | 48.213 | −30.425 | 1.00 | 24.17 | C |
| ATOM | 191 | C | ILE | 28 | 9.011 | 43.872 | −31.720 | 1.00 | 22.19 | C |
| ATOM | 192 | O | ILE | 28 | 7.810 | 43.823 | −32.001 | 1.00 | 22.10 | O |
| ATOM | 193 | N | THR | 29 | 9.931 | 43.138 | −32.347 | 1.00 | 21.09 | N |
| ATOM | 194 | CA | THR | 29 | 9.582 | 42.237 | −33.452 | 1.00 | 21.14 | C |
| ATOM | 195 | CB | THR | 29 | 10.655 | 42.249 | −34.551 | 1.00 | 21.56 | C |
| ATOM | 196 | OG1 | THR | 29 | 11.887 | 41.750 | −34.008 | 1.00 | 18.52 | O |
| ATOM | 197 | CG2 | THR | 29 | 10.877 | 43.657 | −35.075 | 1.00 | 21.14 | C |
| ATOM | 198 | C | TER | 29 | 9.454 | 40.785 | −33.002 | 1.00 | 21.23 | C |
| ATOM | 199 | O | THR | 29 | 8.899 | 39.949 | −33.725 | 1.00 | 19.43 | O |
| ATOM | 200 | N | ASN | 30 | 9.977 | 40.499 | −31.810 | 1.00 | 21.08 | N |
| ATOM | 201 | CA | ASN | 30 | 9.985 | 39.155 | −31.243 | 1.00 | 20.34 | C |
| ATOM | 202 | CB | ASN | 30 | 8.563 | 38.590 | −31.168 | 1.00 | 20.23 | C |
| ATOM | 203 | CG | ASN | 30 | 7.787 | 39.129 | −29.968 | 1.00 | 20.49 | C |
| ATOM | 204 | OD1 | ASN | 30 | 6.559 | 39.252 | −29.999 | 1.00 | 20.76 | O |
| ATOM | 205 | ND2 | ASN | 30 | 8.508 | 39.444 | −28.905 | 1.00 | 19.52 | N |
| ATOM | 206 | C | ASN | 30 | 10.913 | 38.229 | −32.037 | 1.00 | 20.74 | C |
| ATOM | 207 | O | ASN | 30 | 10.803 | 37.001 | −31.975 | 1.00 | 20.80 | O |
| ATOM | 208 | N | ALA | 31 | 11.836 | 38.824 | −32.785 | 1.00 | 19.68 | N |
| ATOM | 209 | CA | ALA | 31 | 12.798 | 38.032 | −33.548 | 1.00 | 20.16 | C |
| ATOM | 210 | CB | ALA | 31 | 13.345 | 38.841 | −34.720 | 1.00 | 19.74 | C |
| ATOM | 211 | C | ALA | 31 | 13.947 | 37.605 | −32.637 | 1.00 | 20.68 | C |
| ATOM | 212 | O | ALA | 31 | 14.253 | 38.270 | −31.646 | 1.00 | 18.54 | O |
| ATOM | 213 | N | SER | 32 | 14.566 | 36.477 | −32.969 | 1.00 | 19.88 | N |
| ATOM | 214 | CA | SER | 32 | 15.709 | 35.983 | −32.220 | 1.00 | 21.20 | C |
| ATOM | 215 | CB | SER | 32 | 15.278 | 34.911 | −31.207 | 1.00 | 19.65 | C |
| ATOM | 216 | OG | SER | 32 | 14.486 | 33.901 | −31.803 | 1.00 | 25.48 | O |
| ATOM | 217 | C | SER | 32 | 16.716 | 35.433 | −33.236 | 1.00 | 22.14 | C |
| ATOM | 218 | O | SER | 32 | 16.332 | 34.801 | −34.219 | 1.00 | 21.52 | O |
| ATOM | 219 | N | GLN | 33 | 17.999 | 35.702 | −33.004 | 1.00 | 22.52 | N |
| ATOM | 220 | CA | GLN | 33 | 19.072 | 35.271 | −33.902 | 1.00 | 23.90 | C |
| ATOM | 221 | CB | GLN | 33 | 19.341 | 36.356 | −34.951 | 1.00 | 25.32 | C |
| ATOM | 222 | CG | GLN | 33 | 19.529 | 37.743 | −34.344 | 1.00 | 28.58 | C |
| ATOM | 223 | CD | GLN | 33 | 19.893 | 38.811 | −35.367 | 1.00 | 32.66 | C |
| ATOM | 224 | OE1 | GLN | 33 | 21.068 | 39.118 | −35.572 | 1.00 | 36.83 | O |
| ATOM | 225 | NE2 | GLN | 33 | 18.886 | 39.375 | −36.016 | 1.00 | 29.57 | N |
| ATOM | 226 | C | GLN | 33 | 20.347 | 35.036 | −33.094 | 1.00 | 24.47 | C |
| ATOM | 227 | O | GLN | 33 | 20.493 | 35.574 | −31.999 | 1.00 | 23.29 | O |
| ATOM | 228 | N | TRP | 34 | 21.276 | 34.254 | −33.644 | 1.00 | 25.01 | N |
| ATOM | 229 | CA | TRP | 34 | 22.531 | 33.973 | −32.948 | 1.00 | 25.47 | C |
| ATOM | 230 | CB | TRP | 34 | 23.213 | 32.726 | −33.537 | 1.00 | 23.87 | C |
| ATOM | 231 | CG | TRP | 34 | 22.521 | 31.438 | −33.188 | 1.00 | 21.81 | C |
| ATOM | 232 | CD2 | TEP | 34 | 22.341 | 30.889 | −31.877 | 1.00 | 22.45 | C |
| ATOM | 233 | CE2 | TRP | 34 | 21.590 | 29.702 | −32.021 | 1.00 | 22.25 | C |
| ATOM | 234 | CE3 | TRP | 34 | 22.742 | 31.286 | −30.594 | 1.00 | 22.29 | C |
| ATOM | 235 | CG1 | TRP | 34 | 21.896 | 30.582 | −34.049 | 1.00 | 21.58 | C |
| ATOM | 236 | NE1 | TRP | 34 | 21.333 | 29.540 | −33.357 | 1.00 | 21.07 | N |
| ATOM | 237 | CZ2 | TRP | 34 | 21.228 | 28.906 | −30.929 | 1.00 | 23.45 | C |
| ATOM | 238 | CZ3 | TRP | 34 | 22.386 | 30.493 | −29.508 | 1.00 | 23.48 | C |
| ATOM | 239 | CH2 | TRP | 34 | 21.635 | 29.317 | −29.683 | 1.00 | 23.38 | C |
| ATOM | 240 | C | TRP | 34 | 23.518 | 35.140 | −32.965 | 1.00 | 27.36 | C |
| ATOM | 241 | O | TRP | 34 | 24.168 | 35.417 | −31.954 | 1.00 | 26.42 | O |
| ATOM | 242 | N | GLU | 35 | 23.624 | 35.827 | −34.103 | 1.00 | 28.79 | N |
| ATOM | 243 | CA | GLU | 35 | 24.558 | 36.938 | −34.228 | 1.00 | 32.29 | C |
| ATOM | 244 | CB | GLU | 35 | 24.885 | 37.205 | −35.694 | 1.00 | 32.69 | C |
| ATOM | 245 | CG | GLU | 35 | 25.387 | 36.002 | −36.457 | 1.00 | 33.59 | C |
| ATOM | 246 | CD | GLU | 35 | 26.098 | 36.391 | −37.735 | 1.00 | 34.42 | C |
| ATOM | 247 | OE1 | GLU | 35 | 26.165 | 35.550 | −38.653 | 1.00 | 34.44 | O |
| ATOM | 248 | OE2 | GLU | 35 | 26.607 | 37.534 | −37.815 | 1.00 | 33.46 | O |
| ATOM | 249 | C | GLU | 35 | 24.084 | 38.238 | −33.604 | 1.00 | 36.01 | C |
| ATOM | 250 | O | GLU | 35 | 22.887 | 38.518 | −33.549 | 1.00 | 34.67 | O |
| ATOM | 251 | N | ARG | 36 | 25.043 | 39.033 | −33.142 | 1.00 | 39.87 | N |
| ATOM | 252 | CA | ARG | 36 | 24.739 | 40.319 | −32.539 | 1.00 | 45.62 | C |
| ATOM | 253 | CB | ARG | 36 | 25.973 | 40.886 | −31.837 | 1.00 | 47.70 | C |
| ATOM | 254 | CG | ARG | 36 | 25.677 | 42.099 | −30.977 | 1.00 | 52.10 | C |
| ATOM | 255 | CD | ARG | 36 | 26.885 | 42.506 | −30.154 | 1.00 | 55.23 | C |
| ATOM | 256 | NE | ARG | 36 | 26.518 | 43.444 | −29.099 | 1.00 | 57.97 | N |
| ATOM | 257 | CZ | ARG | 36 | 27.353 | 43.885 | −28.166 | 1.00 | 59.67 | C |
| ATOM | 258 | NH1 | ARG | 36 | 28.615 | 43.475 | −28.155 | 1.00 | 60.43 | N |
| ATOM | 259 | NH2 | ARG | 36 | 26.924 | 44.730 | −27.238 | 1.00 | 60.07 | N |
| ATOM | 260 | C | ARC | 36 | 24.325 | 41.219 | −33.693 | 1.00 | 47.90 | C |
| ATOM | 261 | O | ARG | 36 | 25.099 | 41.438 | −34.625 | 1.00 | 48.07 | O |
| ATOM | 262 | N | PRO | 37 | 23.094 | 41.746 | −33.647 | 1.00 | 49.97 | N |
| ATOM | 263 | CD | PRO | 37 | 22.208 | 41.730 | −32.471 | 1.00 | 50.02 | C |
| ATOM | 264 | CA | PRO | 37 | 22.547 | 42.624 | −34.685 | 1.00 | 52.82 | C |
| ATOM | 265 | CB | PRO | 37 | 21.156 | 42.947 | −34.153 | 1.00 | 51.49 | C |
| ATOM | 266 | CG | PRO | 37 | 21.381 | 42.976 | −32.682 | 1.00 | 50.64 | C |
| ATOM | 267 | C | PRO | 37 | 23.367 | 43.882 | −34.952 | 1.00 | 55.66 | C |
| ATOM | 268 | O | PRO | 37 | 23.945 | 44.467 | −34.034 | 1.00 | 55.81 | O |
| ATOM | 269 | N | SER | 38 | 23.403 | 44.284 | −36.220 | 1.00 | 58.07 | N |
| ATOM | 270 | CA | SER | 38 | 24.126 | 45.474 | −36.658 | 1.00 | 60.06 | C |
| ATOM | 271 | CB | SER | 38 | 23.196 | 46.689 | −36.607 | 1.00 | 59.17 | C |
| ATOM | 272 | OC | SER | 38 | 22.011 | 46.444 | −37.347 | 1.00 | 57.19 | O |
| ATOM | 273 | C | SER | 38 | 25.377 | 45.750 | −35.831 | 1.00 | 61.25 | C |
| ATOM | 274 | O | SER | 38 | 25.633 | 46.888 | −35.437 | 1.00 | 62.84 | O |
| ATOM | 275 | N | GLU | 51 | 14.438 | 56.608 | −39.353 | 1.00 | 57.69 | N |
| ATOM | 276 | CA | GLU | 51 | 13.791 | 55.417 | −39.887 | 1.00 | 57.34 | C |
| ATOM | 277 | CB | GLU | 51 | 12.787 | 54.856 | −38.872 | 1.00 | 57.35 | C |
| ATOM | 278 | CG | GLU | 51 | 11.476 | 55.629 | −38.793 | 1.00 | 56.42 | C |
| ATOM | 279 | CD | GLU | 51 | 10.543 | 55.101 | −37.718 | 1.00 | 56.15 | C |
| ATOM | 280 | OE1 | GLU | 51 | 9.364 | 55.509 | −37.704 | 1.00 | 55.24 | O |
| ATOM | 281 | OE2 | GLU | 51 | 10.987 | 54.285 | −36.884 | 1.00 | 56.66 | O |
| ATOM | 282 | C | GLU | 51 | 13.066 | 55.776 | −41.180 | 1.00 | 56.81 | C |
| ATOM | 283 | O | GLU | 51 | 12.765 | 56.944 | −41.425 | 1.00 | 57.20 | O |
| ATOM | 284 | N | PRO | 52 | 12.776 | 54.774 | −42.026 | 1.00 | 55.89 | N |
| ATOM | 285 | CD | PRO | 52 | 13.163 | 53.358 | −41.904 | 1.00 | 55.54 | C |
| ATOM | 286 | CA | PRO | 52 | 12.081 | 55.013 | −43.294 | 1.00 | 54.05 | C |
| ATOM | 287 | CB | PRO | 52 | 11.985 | 53.617 | −43.903 | 1.00 | 54.81 | C |
| ATOM | 288 | CG | PRO | 52 | 13.186 | 52.919 | −43.344 | 1.00 | 55.46 | C |
| ATOM | 289 | C | PRO | 52 | 10.706 | 55.626 | −43.066 | 1.00 | 52.40 | C |
| ATOM | 290 | O | PRO | 52 | 10.054 | 55.342 | −42.064 | 1.00 | 53.30 | O |
| ATOM | 291 | N | ALA | 53 | 10.269 | 56.467 | −43.995 | 1.00 | 50.01 | N |
| ATOM | 292 | CA | ALA | 53 | 8.962 | 57.096 | −43.882 | 1.00 | 47.50 | C |
| ATOM | 293 | CB | ALA | 53 | 8.839 | 58.233 | −44.888 | 1.00 | 47.78 | C |
| ATOM | 294 | C | ALA | 53 | 7.902 | 56.037 | −44.151 | 1.00 | 45.45 | C |
| ATOM | 295 | O | ALA | 53 | 6.777 | 56.115 | −43.654 | 1.00 | 44.38 | O |
| ATOM | 296 | N | ARC | 54 | 8.277 | 55.044 | −44.949 | 1.00 | 43.10 | N |
| ATOM | 297 | CA | ARC | 54 | 7.380 | 53.954 | −45.301 | 1.00 | 39.82 | C |
| ATOM | 298 | CB | ARC | 54 | 6.669 | 54.259 | −46.623 | 1.00 | 42.74 | C |
| ATOM | 299 | CG | ARC | 54 | 5.840 | 55.532 | −46.615 | 1.00 | 46.81 | C |
| ATOM | 300 | CD | ARC | 54 | 5.537 | 56.006 | −48.032 | 1.00 | 49.87 | C |
| ATOM | 301 | NE | ARG | 54 | 4.699 | 55.078 | −48.790 | 1.00 | 51.01 | N |
| ATOM | 302 | CZ | ARC | 54 | 3.428 | 54.811 | −48.507 | 1.00 | 52.78 | C |
| ATOM | 303 | NH1 | ARC | 54 | 2.834 | 55.398 | −47.476 | 1.00 | 53.46 | N |
| ATOM | 304 | NH2 | ARC | 54 | 2.743 | 53.965 | −49.266 | 1.00 | 53.28 | N |
| ATOM | 305 | C | ARC | 54 | 8.171 | 52.661 | −45.450 | 1.00 | 35.96 | C |
| ATOM | 306 | O | ARC | 54 | 9.373 | 52.676 | −45.712 | 1.00 | 35.46 | O |
| ATOM | 307 | N | VAL | 55 | 7.489 | 51.540 | −45.266 | 1.00 | 32.57 | N |
| ATOM | 308 | CA | VAL | 55 | 8.110 | 50.235 | −45.429 | 1.00 | 28.54 | C |
| ATOM | 309 | CB | VAL | 55 | 8.445 | 49.557 | −44.071 | 1.00 | 27.23 | C |
| ATOM | 310 | CG1 | VAL | 55 | 9.463 | 50.390 | −43.307 | 1.00 | 24.86 | C |
| ATOM | 311 | CG2 | VAL | 55 | 7.170 | 49.373 | −43.251 | 1.00 | 24.27 | C |
| ATOM | 312 | C | VAL | 55 | 7.094 | 49.373 | −46.149 | 1.00 | 27.45 | C |
| ATOM | 313 | O | VAL | 55 | 5.902 | 49.681 | −46.163 | 1.00 | 26.43 | O |
| ATOM | 314 | N | ARG | 56 | 7.571 | 48.301 | −46.764 | 1.00 | 25.29 | N |
| ATOM | 315 | CA | ARG | 56 | 6.687 | 47.379 | −47.449 | 1.00 | 23.95 | C |
| ATOM | 316 | CB | ARG | 56 | 7.073 | 47.251 | −48.924 | 1.00 | 23.09 | C |
| ATOM | 317 | CG | ARG | 56 | 6.146 | 46.336 | −49.702 | 1.00 | 24.25 | C |
| ATOM | 318 | CD | ARG | 56 | 6.558 | 46.212 | −51.166 | 1.00 | 25.63 | C |
| ATOM | 319 | NE | ARG | 56 | 5.648 | 45.336 | −51.900 | 1.00 | 26.11 | N |
| ATOM | 320 | CZ | ARG | 56 | 5.716 | 45.108 | −53.210 | 1.00 | 27.42 | C |
| ATOM | 321 | NH1 | ARG | 56 | 6.656 | 45.691 | −53.944 | 1.00 | 25.82 | N |
| ATOM | 322 | NH2 | ARG | 56 | 4.841 | 44.297 | −53.789 | 1.00 | 26.73 | N |
| ATOM | 323 | C | ARG | 56 | 6.852 | 46.041 | −46.742 | 1.00 | 22.60 | C |
| ATOM | 324 | O | ARG | 56 | 7.973 | 45.599 | −46.497 | 1.00 | 22.92 | O |
| ATOM | 325 | N | CYS | 57 | 5.740 | 45.405 | −46.401 | 1.00 | 22.50 | N |
| ATOM | 326 | CA | CYS | 57 | 5.797 | 44.124 | −45.715 | 1.00 | 21.96 | C |
| ATOM | 327 | CB | CYS | 57 | 5.526 | 44.299 | −44.216 | 1.00 | 21.34 | C |
| ATOM | 328 | SG | CYS | 57 | 6.684 | 45.319 | −43.331 | 1.00 | 20.11 | S |
| ATOM | 329 | C | CYS | 57 | 4.773 | 43.148 | −46.240 | 1.00 | 22.55 | C |
| ATOM | 330 | O | CYS | 57 | 3.781 | 43.534 | −46.871 | 1.00 | 22.64 | O |
| ATOM | 331 | N | SER | 58 | 5.037 | 41.875 | −45.972 | 1.00 | 20.32 | N |
| ATOM | 332 | CA | SER | 58 | 4.123 | 40.796 | −46.298 | 1.00 | 20.21 | C |
| ATOM | 333 | CB | SER | 58 | 4.763 | 39.753 | −47.217 | 1.00 | 20.04 | C |
| ATOM | 334 | OG | SER | 58 | 5.040 | 40.301 | −48.486 | 1.00 | 19.12 | O |
| ATOM | 335 | C | SER | 58 | 3.893 | 40.179 | −44.932 | 1.00 | 20.61 | C |
| ATOM | 336 | O | SER | 58 | 4.712 | 40.362 | −44.019 | 1.00 | 20.53 | O |
| ATOM | 337 | N | HIS | 59 | 2.788 | 39.462 | −44.770 | 1.00 | 20.02 | N |
| ATOM | 338 | CA | HIS | 59 | 2.544 | 38.821 | −43.499 | 1.00 | 19.93 | C |
| ATOM | 339 | CB | HIS | 59 | 1.860 | 39.802 | −42.515 | 1.00 | 21.80 | C |
| ATOM | 340 | CG | HIS | 59 | 0.362 | 39.853 | −42.617 | 1.00 | 24.62 | C |
| ATOM | 341 | CD2 | HIS | 59 | −0.592 | 39.810 | −41.657 | 1.00 | 22.95 | C |
| ATOM | 342 | ND1 | HIS | 59 | −0.307 | 40.000 | −43.813 | 1.00 | 24.31 | N |
| ATOM | 343 | CE1 | HIS | 59 | −1.609 | 40.041 | −43.586 | 1.00 | 21.97 | C |
| ATOM | 344 | NE2 | HIS | 59 | −1.809 | 39.930 | −42.286 | 1.00 | 26.77 | N |
| ATOM | 345 | C | HIS | 59 | 1.735 | 37.555 | −43.672 | 1.00 | 20.16 | C |
| ATOM | 346 | O | HIS | 59 | 1.118 | 37.315 | −44.720 | 1.00 | 18.78 | O |
| ATOM | 347 | N | LEU | 60 | 1.789 | 36.720 | −42.647 | 1.00 | 18.36 | N |
| ATOM | 348 | CA | LEU | 60 | 1.043 | 35.482 | −42.616 | 1.00 | 19.49 | C |
| ATOM | 349 | CB | LEU | 60 | 1.976 | 34.278 | −42.576 | 1.00 | 18.97 | C |
| ATOM | 350 | CG | LEU | 60 | 1.345 | 32.883 | −42.703 | 1.00 | 18.12 | C |
| ATOM | 351 | CD1 | LEU | 60 | 2.445 | 31.884 | −43.007 | 1.00 | 18.41 | C |
| ATOM | 352 | CD2 | LEU | 60 | 0.605 | 32.495 | −41.431 | 1.00 | 17.46 | C |
| ATOM | 353 | C | LEU | 60 | 0.293 | 35.625 | −41.305 | 1.00 | 20.43 | C |
| ATOM | 354 | O | LEU | 60 | 0.894 | 35.803 | −40.240 | 1.00 | 19.13 | O |
| ATOM | 355 | N | LEU | 61 | −1.026 | 35.592 | −41.391 | 1.00 | 20.07 | N |
| ATOM | 356 | CA | LEU | 61 | −1.850 | 35.751 | −40.210 | 1.00 | 21.15 | C |
| ATOM | 357 | CB | LEU | 61 | −2.889 | 36.849 | −40.457 | 1.00 | 22.48 | C |
| ATOM | 358 | CG | LEU | 61 | −4.014 | 37.017 | −39.429 | 1.00 | 22.65 | C |
| ATOM | 359 | CG1 | LEU | 61 | −3.441 | 37.533 | −38.115 | 1.00 | 24.95 | C |
| ATOM | 360 | CD2 | LEU | 61 | −5.060 | 37.990 | −39.972 | 1.00 | 24.74 | C |
| ATOM | 361 | C | LEU | 61 | −2.556 | 34.460 | −39.843 | 1.00 | 20.90 | C |
| ATOM | 362 | O | LEU | 61 | −3.067 | 33.749 | −40.705 | 1.00 | 18.95 | O |
| ATOM | 363 | N | VAL | 62 | −2.557 | 34.147 | −38.554 | 1.00 | 20.75 | N |
| ATOM | 364 | CA | VAL | 62 | −3.261 | 32.978 | −38.075 | 1.00 | 20.88 | C |
| ATOM | 365 | CB | VAL | 62 | −2.324 | 31.949 | −37.415 | 1.00 | 21.62 | C |
| ATOM | 366 | CG1 | VAL | 62 | −3.155 | 30.825 | −36.786 | 1.00 | 21.94 | C |
| ATOM | 367 | CG2 | VAL | 62 | −1.382 | 31.361 | −38.461 | 1.00 | 22.40 | C |
| ATOM | 368 | C | VAL | 62 | −4.234 | 33.540 | −37.047 | 1.00 | 21.88 | C |
| ATOM | 369 | O | VAL | 62 | −3.827 | 34.113 | −36.037 | 1.00 | 19.51 | O |
| ATOM | 370 | N | LYS | 63 | −5.523 | 33.411 | −37.334 | 1.00 | 22.77 | N |
| ATOM | 371 | CA | LYS | 63 | −6.543 | 33.923 | −36.434 | 1.00 | 25.20 | C |
| ATOM | 372 | CB | LYS | 63 | −7.782 | 34.376 | −37.218 | 1.00 | 26.39 | C |
| ATOM | 373 | CG | LYS | 63 | −7.604 | 35.675 | −37.987 | 1.00 | 28.45 | C |
| ATOM | 374 | CD | LYS | 63 | −8.925 | 36.124 | −38.611 | 1.00 | 29.09 | C |
| ATOM | 375 | CE | LYS | 63 | −8.822 | 37.513 | −39.201 | 1.00 | 27.74 | C |
| ATOM | 376 | NZ | LYS | 63 | −10.093 | 37.921 | −39.861 | 1.00 | 28.12 | N |
| ATOM | 377 | C | LYS | 63 | −6.950 | 32.873 | −35.415 | 1.00 | 25.23 | C |
| ATOM | 378 | O | LYS | 63 | −6.698 | 31.681 | −35.591 | 1.00 | 24.50 | O |
| ATOM | 379 | N | HIS | 64 | −7.573 | 33.335 | −34.339 | 1.00 | 26.37 | N |
| ATOM | 380 | CA | HIS | 64 | −8.046 | 32.449 | −33.288 | 1.00 | 26.91 | C |
| ATOM | 381 | CB | HIS | 64 | −7.077 | 32.470 | −32.098 | 1.00 | 24.05 | C |
| ATOM | 382 | CG | HIS | 64 | −6.700 | 33.845 | −31.646 | 1.00 | 25.82 | C |
| ATOM | 383 | CD2 | HIS | 64 | −5.538 | 34.531 | −31.753 | 1.00 | 26.61 | C |
| ATOM | 384 | ND1 | HIS | 64 | −7.584 | 34.688 | −31.005 | 1.00 | 25.59 | N |
| ATOM | 385 | CE1 | HIS | 64 | −6.982 | 35.832 | −30.738 | 1.00 | 28.49 | C |
| ATOM | 386 | NE2 | HIS | 64 | −5.739 | 35.763 | −31.182 | 1.00 | 27.26 | N |
| ATOM | 387 | C | HIS | 64 | −9.448 | 32.903 | −32.882 | 1.00 | 27.07 | C |
| ATOM | 388 | O | HIS | 64 | −9.980 | 33.858 | −33.446 | 1.00 | 26.03 | O |
| ATOM | 389 | N | SER | 65 | −10.044 | 32.223 | −31.910 | 1.00 | 30.47 | N |
| ATOM | 390 | CA | SER | 65 | −11.395 | 32.555 | −31.472 | 1.00 | 30.56 | C |
| ATOM | 391 | CB | SER | 65 | −11.891 | 31.508 | −30.473 | 1.00 | 33.30 | C |
| ATOM | 392 | OC | SER | 65 | −11.143 | 31.559 | −29.272 | 1.00 | 34.85 | O |
| ATOM | 393 | C | SER | 65 | −11.551 | 33.952 | −30.868 | 1.00 | 31.48 | C |
| ATOM | 394 | O | SER | 65 | −12.665 | 34.473 | −30.796 | 1.00 | 30.78 | O |
| ATOM | 395 | N | GLN | 66 | −10.455 | 34.564 | −30.430 | 1.00 | 29.16 | N |
| ATOM | 396 | CA | GLN | 66 | −10.554 | 35.901 | −29.846 | 1.00 | 29.95 | C |
| ATOM | 397 | CB | GLN | 66 | −9.703 | 36.005 | −28.567 | 1.00 | 30.78 | C |
| ATOM | 398 | CG | GLN | 66 | −10.244 | 35.180 | −27.394 | 1.00 | 31.19 | C |
| ATOM | 399 | CD | GLN | 66 | −9.508 | 35.439 | −26.078 | 1.00 | 34.58 | C |
| ATOM | 400 | OE1 | GLN | 66 | −9.476 | 36.568 | −25.577 | 1.00 | 32.21 | O |
| ATOM | 401 | NE2 | GLN | 66 | −8.921 | 34.388 | −25.512 | 1.00 | 34.03 | N |
| ATOM | 402 | C | GLN | 66 | −10.192 | 37.025 | −30.814 | 1.00 | 29.66 | C |
| ATOM | 403 | O | GLN | 66 | −10.088 | 38.187 | −30.414 | 1.00 | 32.44 | O |
| ATOM | 404 | N | SER | 67 | −10.002 | 36.688 | −32.087 | 1.00 | 30.14 | N |
| ATOM | 405 | CA | SER | 67 | −9.676 | 37.694 | −33.096 | 1.00 | 31.52 | C |
| ATOM | 406 | CB | SER | 67 | −9.325 | 37.024 | −34.432 | 1.00 | 29.84 | C |
| ATOM | 407 | OG | SER | 67 | −8.112 | 36.294 | −34.338 | 1.00 | 26.70 | O |
| ATOM | 408 | C | SER | 67 | −10.902 | 38.585 | −33.271 | 1.00 | 33.64 | C |
| ATOM | 409 | O | SER | 67 | −12.031 | 38.094 | −33.203 | 1.00 | 33.54 | O |
| ATOM | 410 | N | ARC | 68 | −10.687 | 39.880 | −33.499 | 1.00 | 36.87 | N |
| ATOM | 411 | CA | ARG | 68 | −11.808 | 40.805 | −33.658 | 1.00 | 41.36 | C |
| ATOM | 412 | CB | ARC | 68 | −11.325 | 42.189 | −34.103 | 1.00 | 45.22 | C |
| ATOM | 413 | CG | ARC | 68 | −10.498 | 42.197 | −35.362 | 1.00 | 52.20 | C |
| ATOM | 414 | CD | ARC | 68 | −10.198 | 43.620 | −35.816 | 1.00 | 57.28 | C |
| ATOM | 415 | NE | ARG | 68 | −9.482 | 44.391 | −34.805 | 1.00 | 61.00 | N |
| ATOM | 416 | CZ | ARG | 68 | −8.980 | 45.604 | −35.011 | 1.00 | 63.18 | C |
| ATOM | 417 | NH1 | ARG | 68 | −9.112 | 46.186 | −36.197 | 1.00 | 63.39 | N |
| ATOM | 418 | NH2 | ARC | 68 | −8.347 | 46.238 | −34.032 | 1.00 | 64.64 | N |
| ATOM | 419 | C | ARC | 68 | −12.832 | 40.252 | −34.641 | 1.00 | 41.19 | C |
| ATOM | 420 | O | ARC | 68 | −14.035 | 40.431 | −34.454 | 1.00 | 41.37 | O |
| ATOM | 421 | N | ARC | 69 | −12.350 | 39.572 | −35.678 | 1.00 | 40.86 | N |
| ATOM | 422 | CA | ARC | 69 | −13.217 | 38.955 | −36.679 | 1.00 | 39.53 | C |
| ATOM | 423 | CB | ARC | 69 | −13.147 | 39.729 | −37.995 | 1.00 | 43.41 | C |
| ATOM | 424 | CG | ARC | 69 | −14.046 | 40.953 | −38.043 | 1.00 | 47.58 | C |
| ATOM | 425 | CD | ARC | 69 | −13.773 | 41.777 | −39.287 | 1.00 | 52.27 | C |
| ATOM | 426 | NE | ARC | 69 | −13.590 | 40.936 | −40.466 | 1.00 | 56.08 | N |
| ATOM | 427 | CZ | ARC | 69 | −13.277 | 41.396 | −41.673 | 1.00 | 58.81 | C |
| ATOM | 428 | NH1 | ARC | 69 | −13.114 | 42.698 | −41.865 | 1.00 | 60.54 | N |
| ATOM | 429 | NH2 | ARC | 69 | −13.119 | 40.556 | −42.688 | 1.00 | 59.95 | N |
| ATOM | 430 | C | ARC | 69 | −12.772 | 37.508 | −36.893 | 1.00 | 37.96 | C |
| ATOM | 431 | O | ARC | 69 | −11.934 | 37.227 | −37.751 | 1.00 | 36.44 | O |
| ATOM | 432 | N | PRO | 70 | −13.330 | 36.570 | −36.106 | 1.00 | 36.89 | N |
| ATOM | 433 | CD | PRO | 70 | −14.289 | 36.845 | −35.021 | 1.00 | 38.20 | C |
| ATOM | 434 | CA | PRO | 70 | −13.019 | 35.137 | −36.166 | 1.00 | 35.97 | C |
| ATOM | 435 | CB | PRO | 70 | −13.655 | 34.598 | −34.887 | 1.00 | 37.31 | C |
| ATOM | 436 | CG | PRO | 70 | −14.851 | 35.471 | −34.729 | 1.00 | 37.53 | C |
| ATOM | 437 | C | PRO | 70 | −13.515 | 34.415 | −37.414 | 1.00 | 34.97 | C |
| ATOM | 438 | O | PRO | 70 | −14.282 | 33.447 | −37.332 | 1.00 | 34.38 | O |
| ATOM | 439 | N | SER | 71 | −13.047 | 34.874 | −38.570 | 1.00 | 32.94 | N |
| ATOM | 440 | CA | SER | 71 | −13.435 | 34.287 | −39.848 | 1.00 | 30.60 | C |
| ATOM | 441 | CB | SER | 71 | −14.826 | 34.797 | −40.245 | 1.00 | 32.20 | C |
| ATOM | 442 | OG | SER | 71 | −15.220 | 34.309 | −41.516 | 1.00 | 37.57 | O |
| ATOM | 443 | C | SER | 71 | −12.411 | 34.703 | −40.898 | 1.00 | 28.59 | C |
| ATOM | 444 | O | SER | 71 | −11.703 | 35.691 | −40.710 | 1.00 | 27.43 | O |
| ATOM | 445 | N | SER | 72 | −12.328 | 33.941 | −41.985 | 1.00 | 27.81 | N |
| ATOM | 446 | CA | SER | 72 | −11.406 | 34.240 | −43.086 | 1.00 | 28.26 | C |
| ATOM | 447 | CB | SER | 72 | −9.952 | 33.918 | −42.703 | 1.00 | 25.68 | C |
| ATOM | 448 | OG | SER | 72 | −9.714 | 32.518 | −42.730 | 1.00 | 21.94 | O |
| ATOM | 449 | C | SER | 72 | −11.797 | 33.400 | −44.295 | 1.00 | 27.75 | C |
| ATOM | 450 | O | SER | 72 | −12.712 | 32.586 | −44.215 | 1.00 | 27.95 | O |
| ATOM | 451 | N | TRP | 73 | −11.087 | 33.584 | −45.405 | 1.00 | 28.49 | N |
| ATOM | 452 | CA | TRP | 73 | −11.375 | 32.838 | −46.621 | 1.00 | 27.17 | C |
| ATOM | 453 | CB | TRP | 73 | −10.521 | 33.351 | −47.792 | 1.00 | 27.62 | C |
| ATOM | 454 | CG | TRP | 73 | −9.035 | 33.087 | −47.648 | 1.00 | 28.44 | C |
| ATOM | 455 | CD2 | TRP | 73 | −8.326 | 31.921 | −48.093 | 1.00 | 27.51 | C |
| ATOM | 456 | CE2 | TRP | 73 | −6.975 | 32.082 | −47.714 | 1.00 | 26.68 | C |
| ATOM | 457 | CE3 | TRP | 73 | −8.703 | 30.755 | −48.773 | 1.00 | 27.78 | C |
| ATOM | 458 | CD1 | TRP | 73 | −8.107 | 33.887 | −47.035 | 1.00 | 27.25 | C |
| ATOM | 459 | NE1 | TRP | 73 | −6.868 | 33.288 | −47.071 | 1.00 | 26.15 | N |
| ATOM | 460 | CZ2 | TRP | 73 | −5.997 | 31.118 | −47.994 | 1.00 | 26.40 | C |
| ATOM | 461 | CZ3 | TRP | 73 | −7.726 | 29.794 | −49.050 | 1.00 | 27.70 | C |
| ATOM | 462 | CH2 | TRP | 73 | −6.392 | 29.986 | −48.658 | 1.00 | 26.52 | C |
| ATOM | 463 | C | TRP | 73 | −11.121 | 31.352 | −46.419 | 1.00 | 26.91 | C |
| ATOM | 464 | O | TEP | 73 | −11.669 | 30.523 | −47.139 | 1.00 | 27.03 | O |
| ATOM | 465 | N | ARC | 74 | −10.298 | 31.017 | −45.429 | 1.00 | 26.23 | N |
| ATOM | 466 | CA | ARC | 74 | −9.968 | 29.624 | −45.146 | 1.00 | 25.74 | C |
| ATOM | 467 | CB | ARG | 74 | −8.696 | 29.536 | −44.300 | 1.00 | 23.12 | C |
| ATOM | 468 | CG | ARG | 74 | −7.503 | 30.297 | −44.871 | 1.00 | 22.48 | C |
| ATOM | 469 | CD | ARG | 74 | −6.217 | 29.755 | −44.266 | 1.00 | 22.09 | C |
| ATOM | 470 | NE | ARG | 74 | −6.071 | 28.352 | −44.633 | 1.00 | 22.25 | N |
| ATOM | 471 | CZ | ARC | 74 | −5.729 | 27.386 | −43.792 | 1.00 | 23.61 | C |
| ATOM | 472 | NH1 | ARC | 74 | −5.489 | 27.663 | −42.517 | 1.00 | 27.30 | N |
| ATOM | 473 | NH2 | ARG | 74 | −5.641 | 26.138 | −44.230 | 1.00 | 25.35 | N |
| ATOM | 474 | C | ARG | 74 | −11.094 | 28.882 | −44.434 | 1.00 | 26.69 | C |
| ATOM | 475 | O | ARG | 74 | −11.276 | 27.685 | −44.640 | 1.00 | 25.48 | O |
| ATOM | 476 | N | GLN | 75 | −11.825 | 29.583 | −43.573 | 1.00 | 28.87 | N |
| ATOM | 477 | CA | GLN | 75 | −12.942 | 28.970 | −42.861 | 1.00 | 31.31 | C |
| ATOM | 478 | CB | GLN | 75 | −12.450 | 28.048 | −41.740 | 1.00 | 33.76 | C |
| ATOM | 479 | CG | GLN | 75 | −11.543 | 28.688 | −40.727 | 1.00 | 37.08 | C |
| ATOM | 480 | CD | GLN | 75 | −11.212 | 27.745 | −39.581 | 1.00 | 39.40 | C |
| ATOM | 481 | OE1 | GLN | 75 | −10.375 | 28.053 | −38.731 | 1.00 | 39.41 | O |
| ATOM | 482 | NE2 | GLN | 75 | −11.875 | 26.593 | −39.549 | 1.00 | 38.99 | N |
| ATOM | 483 | C | GLN | 75 | −13.885 | 30.030 | −42.307 | 1.00 | 29.89 | C |
| ATOM | 484 | O | GLN | 75 | −13.457 | 31.011 | −41.703 | 1.00 | 28.98 | O |
| ATOM | 485 | N | GLU | 76 | −15.180 | 29.815 | −42.524 | 1.00 | 30.41 | N |
| ATOM | 486 | CA | GLU | 76 | −16.207 | 30.758 | −42.102 | 1.00 | 28.77 | C |
| ATOM | 487 | CB | GLU | 76 | −17.589 | 30.246 | −42.537 | 1.00 | 28.89 | C |
| ATOM | 488 | CG | GLU | 76 | −18.658 | 31.332 | −42.562 | 1.00 | 28.30 | C |
| ATOM | 489 | CD | GLD | 76 | −19.925 | 30.905 | −43.299 | 1.00 | 28.54 | C |
| ATOM | 490 | OE1 | GLU | 76 | −20.809 | 31.764 | −43.496 | 1.00 | 28.39 | O |
| ATOM | 491 | OE2 | GLU | 76 | −20.034 | 29.718 | −43.681 | 1.00 | 25.51 | O |
| ATOM | 492 | C | GLU | 76 | −16.211 | 31.078 | −40.610 | 1.00 | 29.59 | C |
| ATOM | 493 | O | GLU | 76 | −16.461 | 32.215 | −40.215 | 1.00 | 28.90 | O |
| ATOM | 494 | N | LYS | 77 | −15.932 | 30.082 | −39.783 | 1.00 | 31.59 | N |
| ATOM | 495 | CA | LYS | 77 | −15.918 | 30.285 | −38.341 | 1.00 | 34.34 | C |
| ATOM | 496 | CB | LYS | 77 | −17.096 | 29.536 | −37.706 | 1.00 | 35.97 | C |
| ATOM | 497 | CG | LYS | 77 | −17.260 | 29.743 | −36.208 | 1.00 | 40.71 | C |
| ATOM | 498 | CD | LYS | 77 | −17.760 | 31.143 | −35.876 | 1.00 | 43.03 | C |
| ATOM | 499 | CE | LYS | 77 | −17.985 | 31.305 | −34.376 | 1.00 | 43.51 | C |
| ATOM | 500 | NZ | LYS | 77 | −18.931 | 30.284 | −33.837 | 1.00 | 44.93 | N |
| ATOM | 501 | C | LYS | 77 | −14.602 | 29.784 | −37.745 | 1.00 | 33.93 | C |
| ATOM | 502 | O | LYS | 77 | −14.388 | 28.577 | −37.638 | 1.00 | 33.50 | O |
| ATOM | 503 | N | ILE | 78 | −13.728 | 30.715 | −37.368 | 1.00 | 34.25 | N |
| ATOM | 504 | CA | ILE | 78 | −12.437 | 30.373 | −36.764 | 1.00 | 34.01 | C |
| ATOM | 505 | CB | ILE | 78 | −11.446 | 31.564 | −36.821 | 1.00 | 34.63 | C |
| ATOM | 506 | CG2 | ILE | 78 | −10.088 | 31.135 | −36.269 | 1.00 | 33.43 | C |
| ATOM | 507 | CG1 | ILE | 78 | −11.298 | 32.074 | −38.260 | 1.00 | 35.90 | C |
| ATOM | 508 | CD1 | ILE | 78 | −10.411 | 31.231 | −39.142 | 1.00 | 35.86 | C |
| ATOM | 509 | C | ILE | 78 | −12.688 | 30.046 | −35.289 | 1.00 | 33.77 | C |
| ATOM | 510 | O | ILE | 78 | −13.233 | 30.869 | −34.559 | 1.00 | 33.99 | O |
| ATOM | 511 | N | THR | 79 | −12.280 | 28.862 | −34.846 | 1.00 | 33.32 | N |
| ATOM | 512 | CA | THR | 79 | −12.501 | 28.478 | −33.455 | 1.00 | 33.42 | C |
| ATOM | 513 | CB | THR | 79 | −13.548 | 27.360 | −33.370 | 1.00 | 34.16 | C |
| ATOM | 514 | OG1 | THR | 79 | −13.108 | 26.233 | −34.138 | 1.00 | 35.23 | O |
| ATOM | 515 | CG2 | THR | 79 | −14.874 | 27.844 | −33.915 | 1.00 | 35.13 | C |
| ATOM | 516 | C | THR | 79 | −11.257 | 28.029 | −32.685 | 1.00 | 32.98 | C |
| ATOM | 517 | O | THR | 79 | −11.339 | 27.717 | −31.496 | 1.00 | 31.73 | O |
| ATOM | 518 | N | ARG | 80 | −10.107 | 27.986 | −33.351 | 1.00 | 32.06 | N |
| ATOM | 519 | CA | ARG | 80 | −8.883 | 27.570 | −32.671 | 1.00 | 29.59 | C |
| ATOM | 520 | CB | ARG | 80 | −7.710 | 27.534 | −33.657 | 1.00 | 31.75 | C |
| ATOM | 521 | CG | ARG | 80 | −7.267 | 28.907 | −34.132 | 1.00 | 32.62 | C |
| ATOM | 522 | CD | ARG | 80 | −5.975 | 28.855 | −34.938 | 1.00 | 33.04 | C |
| ATOM | 523 | NE | ARG | 80 | −6.207 | 28.717 | −36.372 | 1.00 | 37.11 | N |
| ATOM | 524 | CZ | ARG | 80 | −6.361 | 27.562 | −37.005 | 1.00 | 37.45 | C |
| ATOM | 525 | NH1 | ARC | 80 | −6.304 | 26.421 | −36.328 | 1.00 | 39.19 | N |
| ATOM | 526 | NH2 | ARC | 80 | −6.580 | 27.552 | −38.317 | 1.00 | 35.65 | N |
| ATOM | 527 | C | ARC | 80 | −8.584 | 28.555 | −31.546 | 1.00 | 27.69 | C |
| ATOM | 528 | O | ARC | 80 | −8.962 | 29.722 | −31.620 | 1.00 | 27.39 | O |
| ATOM | 529 | N | THR | 81 | −7.908 | 28.091 | −30.502 | 1.00 | 26.96 | N |
| ATOM | 530 | CA | THR | 81 | −7.569 | 28.965 | −29.387 | 1.00 | 28.78 | C |
| ATOM | 531 | CB | THR | 81 | −7.250 | 28.160 | −28.123 | 1.00 | 29.00 | C |
| ATOM | 532 | OG1 | THR | 81 | −6.108 | 27.327 | −28.363 | 1.00 | 29.98 | O |
| ATOM | 533 | CG2 | THR | 81 | −8.441 | 27.288 | −27.736 | 1.00 | 29.94 | C |
| ATOM | 534 | C | THR | 81 | −6.338 | 29.782 | −29.749 | 1.00 | 29.24 | C |
| ATOM | 535 | O | THR | 81 | −5.668 | 29.487 | −30.737 | 1.00 | 28.92 | O |
| ATOM | 536 | N | LYS | 82 | −6.044 | 30.808 | −28.958 | 1.00 | 30.24 | N |
| ATOM | 537 | CA | LYS | 82 | −4.864 | 31.635 | −29.200 | 1.00 | 30.05 | C |
| ATOM | 538 | CB | LYS | 82 | −4.756 | 32.750 | −28.161 | 1.00 | 33.96 | C |
| ATOM | 539 | CG | LYS | 82 | −5.863 | 33.780 | −28.210 | 1.00 | 39.25 | C |
| ATOM | 540 | CD | LYS | 82 | −5.616 | 34.887 | −27.193 | 1.00 | 41.47 | C |
| ATOM | 541 | CE | LYS | 82 | −5.531 | 34.333 | −25.778 | 1.00 | 44.01 | C |
| ATOM | 542 | NZ | LYS | 82 | −5.354 | 35.413 | −24.770 | 1.00 | 44.71 | N |
| ATOM | 543 | C | LYS | 82 | −3.621 | 30.751 | −29.096 | 1.00 | 29.66 | C |
| ATOM | 544 | O | LYS | 82 | −2.681 | 30.877 | −29.884 | 1.00 | 25.85 | O |
| ATOM | 545 | N | GLU | 83 | −3.628 | 29.861 | −28.108 | 1.00 | 28.55 | N |
| ATOM | 546 | CA | GLU | 83 | −2.505 | 28.953 | −27.892 | 1.00 | 29.02 | C |
| ATOM | 547 | CB | GLU | 83 | −2.760 | 28.075 | −26.665 | 1.00 | 31.20 | C |
| ATOM | 548 | CG | GLU | 83 | −2.852 | 28.854 | −25.364 | 1.00 | 35.97 | C |
| ATOM | 549 | CD | GLU | 83 | −4.156 | 29.626 | −25.218 | 1.00 | 37.98 | C |
| ATOM | 550 | OE1 | GLU | 83 | −4.225 | 30.506 | −24.335 | 1.00 | 41.29 | O |
| ATOM | 551 | OE2 | GLU | 83 | −5.111 | 29.350 | −25.971 | 1.00 | 37.74 | O |
| ATOM | 552 | C | GLU | 83 | −2.252 | 28.070 | −29.108 | 1.00 | 26.59 | C |
| ATOM | 553 | O | GLU | 83 | −1.104 | 27.875 | −29.511 | 1.00 | 24.73 | O |
| ATOM | 554 | N | GLU | 84 | −3.321 | 27.531 | −29.685 | 1.00 | 25.40 | N |
| ATOM | 555 | CA | GLU | 84 | −3.198 | 26.677 | −30.866 | 1.00 | 25.25 | C |
| ATOM | 556 | CB | GLU | 84 | −4.553 | 26.049 | −31.223 | 1.00 | 26.42 | C |
| ATOM | 557 | CG | GLU | 84 | −5.124 | 25.124 | −30.153 | 1.00 | 29.77 | C |
| ATOM | 558 | CD | GLU | 84 | −6.481 | 24.547 | −30.539 | 1.00 | 31.27 | C |
| ATOM | 559 | OE1 | GLU | 84 | −7.409 | 25.336 | −30.816 | 1.00 | 29.51 | O |
| ATOM | 560 | OE2 | GLU | 84 | −6.618 | 23.306 | −30.564 | 1.00 | 33.67 | O |
| ATOM | 561 | C | GLU | 84 | −2.686 | 27.500 | −32.052 | 1.00 | 25.07 | C |
| ATOM | 562 | O | GLU | 84 | −1.875 | 27.024 | −32.849 | 1.00 | 24.45 | O |
| ATOM | 563 | N | ALA | 85 | −3.163 | 28.735 | −32.160 | 1.00 | 23.80 | N |
| ATOM | 564 | CA | ALA | 85 | −2.743 | 29.625 | −33.244 | 1.00 | 23.52 | C |
| ATOM | 565 | CB | ALA | 85 | −3.535 | 30.926 | −33.188 | 1.00 | 23.72 | C |
| ATOM | 566 | C | ALA | 85 | −1.244 | 29.920 | −33.146 | 1.00 | 23.46 | C |
| ATOM | 567 | O | ALA | 85 | −0.544 | 29.994 | −34.161 | 1.00 | 21.39 | O |
| ATOM | 568 | N | LEU | 86 | −0.755 | 30.097 | −31.922 | 1.00 | 22.67 | N |
| ATOM | 569 | CA | LEU | 86 | 0.662 | 30.370 | −31.715 | 1.00 | 22.05 | C |
| ATOM | 570 | CB | LEU | 86 | 0.919 | 30.763 | −30.258 | 1.00 | 22.26 | C |
| ATOM | 571 | CG | LEU | 86 | 2.363 | 31.118 | −29.870 | 1.00 | 23.67 | C |
| ATOM | 572 | CD1 | LEU | 86 | 2.868 | 32.269 | −30.740 | 1.00 | 22.88 | C |
| ATOM | 573 | CD2 | LEU | 86 | 2.419 | 31.515 | −28.397 | 1.00 | 22.97 | C |
| ATOM | 574 | C | LEU | 86 | 1.503 | 29.149 | −32.091 | 1.00 | 22.81 | C |
| ATOM | 575 | O | LEU | 86 | 2.604 | 29.286 | −32.629 | 1.00 | 20.32 | O |
| ATOM | 576 | N | GLU | 87 | 0.997 | 27.953 | −31.803 | 1.00 | 23.15 | N |
| ATOM | 577 | CA | GLU | 87 | 1.74 | 126.747 | −32.144 | 1.00 | 25.76 | C |
| ATOM | 578 | CB | GLU | 87 | 1.07 | 025.503 | −31.559 | 1.00 | 29.79 | C |
| ATOM | 579 | CG | GLU | 87 | 1.276 | 25.379 | −30.062 | 1.00 | 35.40 | C |
| ATOM | 580 | CD | GLU | 87 | 2.739 | 25.533 | −29.679 | 1.00 | 41.20 | C |
| ATOM | 581 | OE1 | GLU | 87 | 3.564 | 24.709 | −30.131 | 1.00 | 41.60 | O |
| ATOM | 582 | OE2 | GLU | 87 | 3.066 | 26.485 | −28.935 | 1.00 | 44.59 | O |
| ATOM | 583 | C | GLU | 87 | 1.869 | 26.617 | −33.653 | 1.00 | 24.62 | C |
| ATOM | 584 | O | GLU | 87 | 2.903 | 26.169 | −34.161 | 1.00 | 22.44 | O |
| ATOM | 585 | N | LEU | 88 | 0.816 | 27.007 | −34.369 | 1.00 | 22.75 | N |
| ATOM | 586 | CA | LEU | 88 | 0.826 | 26.955 | −35.829 | 1.00 | 21.50 | C |
| ATOM | 587 | CB | LEU | 88 | −0.568 | 27.284 | −36.382 | 1.00 | 20.56 | C |
| ATOM | 588 | CG | LEU | 88 | −1.572 | 26.129 | −36.310 | 1.00 | 23.77 | C |
| ATOM | 589 | CD1 | LEU | 88 | −3.008 | 26.642 | −36.453 | 1.00 | 22.19 | C |
| ATOM | 590 | CD2 | LEU | 88 | −1.232 | 25.118 | −37.401 | 1.00 | 22.20 | C |
| ATOM | 591 | C | LEU | 88 | 1.858 | 27.949 | −36.363 | 1.00 | 19.42 | C |
| ATOM | 592 | O | LEU | 88 | 2.651 | 27.617 | −37.252 | 1.00 | 19.73 | O |
| ATOM | 593 | N | ILE | 89 | 1.838 | 29.159 | −35.809 | 1.00 | 18.57 | N |
| ATOM | 594 | CA | ILE | 89 | 2.763 | 30.227 | −36.180 | 1.00 | 18.37 | C |
| ATOM | 595 | CB | ILE | 89 | 2.475 | 31.508 | −35.349 | 1.00 | 18.61 | C |
| ATOM | 596 | CG2 | ILE | 89 | 3.644 | 32.470 | −35.435 | 1.00 | 18.86 | C |
| ATOM | 597 | CG1 | ILE | 89 | 1.188 | 32.189 | −35.842 | 1.00 | 17.15 | C |
| ATOM | 598 | CD1 | ILE | 89 | 1.342 | 32.943 | −37.163 | 1.00 | 16.22 | C |
| ATOM | 599 | C | ILE | 89 | 4.211 | 29.779 | −35.932 | 1.00 | 20.19 | C |
| ATOM | 600 | O | ILE | 89 | 5.076 | 29.920 | −36.804 | 1.00 | 18.74 | O |
| ATOM | 601 | N | ASN | 90 | 4.469 | 29.238 | −34.743 | 1.00 | 18.44 | N |
| ATOM | 602 | CA | ASN | 90 | 5.813 | 28.785 | −34.409 | 1.00 | 19.72 | C |
| ATOM | 603 | CB | ASN | 90 | 5.868 | 28.276 | −32.964 | 1.00 | 21.36 | C |
| ATOM | 604 | CG | ASN | 90 | 5.759 | 29.396 | −31.947 | 1.00 | 22.39 | C |
| ATOM | 605 | OD1 | ASN | 90 | 6.183 | 30.523 | −32.199 | 1.00 | 26.11 | O |
| ATOM | 606 | ND2 | ASN | 90 | 5.209 | 29.083 | −30.781 | 1.00 | 26.54 | N |
| ATOM | 607 | C | ASN | 90 | 6.300 | 27.691 | −35.356 | 1.00 | 19.73 | C |
| ATOM | 608 | O | ASN | 90 | 7.490 | 27.608 | −35.644 | 1.00 | 19.99 | O |
| ATOM | 609 | N | GLY | 91 | 5.381 | 26.857 | −35.835 | 1.00 | 19.30 | N |
| ATOM | 610 | CA | GLY | 91 | 5.747 | 25.791 | −36.749 | 1.00 | 19.27 | C |
| ATOM | 611 | C | GLY | 91 | 6.068 | 26.341 | −38.126 | 1.00 | 20.36 | C |
| ATOM | 612 | O | GLY | 91 | 6.965 | 25.848 | −38.818 | 1.00 | 19.68 | O |
| ATOM | 613 | N | TYR | 92 | 5.330 | 27.370 | −38.534 | 1.00 | 18.26 | N |
| ATOM | 614 | CA | TYR | 92 | 5.569 | 27.991 | −39.830 | 1.00 | 17.78 | C |
| ATOM | 615 | CB | TYR | 92 | 4.483 | 29.037 | −40.136 | 1.00 | 18.19 | C |
| ATOM | 616 | CG | TYR | 92 | 3.107 | 28.437 | −40.361 | 1.00 | 19.32 | C |
| ATOM | 617 | CD1 | TYR | 92 | 1.953 | 29.162 | −40.062 | 1.00 | 19.52 | C |
| ATOM | 618 | CE1 | TYR | 92 | 0.684 | 28.618 | −40.266 | 1.00 | 20.24 | C |
| ATOM | 619 | CD2 | TYR | 92 | 2.960 | 27.146 | −40.878 | 1.00 | 18.84 | C |
| ATOM | 620 | CE2 | TYR | 92 | 1.695 | 26.592 | −41.087 | 1.00 | 21.13 | C |
| ATOM | 621 | CZ | TYR | 92 | 0.559 | 27.338 | −40.776 | 1.00 | 20.32 | C |
| ATOM | 622 | OH | TYR | 92 | −0.697 | 26.797 | −40.961 | 1.00 | 20.00 | O |
| ATOM | 623 | C | TYR | 92 | 6.936 | 28.659 | −39.787 | 1.00 | 14.87 | C |
| ATOM | 624 | O | TYR | 92 | 7.679 | 28.597 | −40.752 | 1.00 | 16.54 | O |
| ATOM | 625 | N | ILE | 93 | 7.257 | 29.293 | −38.663 | 1.00 | 14.90 | N |
| ATOM | 626 | CA | ILE | 93 | 8.545 | 29.967 | −38.500 | 1.00 | 17.95 | C |
| ATOM | 627 | CB | ILE | 93 | 8.623 | 30.724 | −37.142 | 1.00 | 15.92 | C |
| ATOM | 628 | CG2 | ILE | 93 | 10.071 | 31.164 | −36.852 | 1.00 | 16.05 | C |
| ATOM | 629 | CG1 | ILE | 93 | 7.742 | 31.979 | −37.197 | 1.00 | 16.47 | C |
| ATOM | 630 | CD1 | ILE | 93 | 7.580 | 32.684 | −35.839 | 1.00 | 16.42 | C |
| ATOM | 631 | C | ILE | 93 | 9.697 | 28.966 | −38.602 | 1.00 | 17.73 | C |
| ATOM | 632 | O | ILE | 93 | 10.721 | 29.249 | −39.234 | 1.00 | 16.34 | O |
| ATOM | 633 | N | GLN | 94 | 9.531 | 27.797 | −37.993 | 1.00 | 18.12 | N |
| ATOM | 634 | CA | GLN | 94 | 10.574 | 26.782 | −38.054 | 1.00 | 21.32 | C |
| ATOM | 635 | CB | GLN | 94 | 10.217 | 25.579 | −37.173 | 1.00 | 23.22 | C |
| ATOM | 636 | CG | GLN | 94 | 10.242 | 25.887 | −35.691 | 1.00 | 25.92 | C |
| ATOM | 637 | CD | GLN | 94 | 10.036 | 24.654 | −34.836 | 1.00 | 31.08 | C |
| ATOM | 638 | OE1 | GLN | 94 | 9.063 | 23.913 | −35.011 | 1.00 | 32.11 | O |
| ATOM | 639 | NE2 | GLN | 94 | 10.949 | 24.428 | −33.899 | 1.00 | 31.37 | N |
| ATOM | 640 | C | GLN | 94 | 10.813 | 26.317 | −39.487 | 1.00 | 21.15 | C |
| ATOM | 641 | O | GLN | 94 | 11.959 | 26.177 | −39.911 | 1.00 | 22.23 | O |
| ATOM | 642 | N | LYS | 95 | 9.739 | 26.086 | −40.236 | 1.00 | 20.91 | N |
| ATOM | 643 | CA | LYS | 95 | 9.870 | 25.634 | −41.617 | 1.00 | 20.41 | C |
| ATOM | 644 | CB | LYS | 95 | 8.511 | 25.205 | −42.173 | 1.00 | 24.55 | C |
| ATOM | 645 | CG | LYS | 95 | 7.968 | 23.914 | −41.577 | 1.00 | 29.78 | C |
| ATOM | 646 | CD | LYS | 95 | 6.720 | 23.454 | −42.324 | 1.00 | 33.95 | C |
| ATOM | 647 | CE | LYS | 95 | 6.211 | 22.119 | −41.793 | 1.00 | 36.40 | C |
| ATOM | 648 | NZ | LYS | 95 | 5.821 | 22.203 | −40.352 | 1.00 | 40.21 | N |
| ATOM | 649 | C | LYS | 95 | 10.467 | 26.716 | −42.508 | 1.00 | 19.44 | C |
| ATOM | 650 | O | LYS | 95 | 11.218 | 26.425 | −43.439 | 1.00 | 19.34 | O |
| ATOM | 651 | N | ILE | 96 | 10.125 | 27.971 | −42.234 | 1.00 | 18.25 | N |
| ATOM | 652 | CA | ILE | 96 | 10.659 | 29.070 | −43.020 | 1.00 | 17.52 | C |
| ATOM | 653 | CB | ILE | 96 | 9.915 | 30.400 | −42.704 | 1.00 | 17.35 | C |
| ATOM | 654 | CG2 | ILE | 96 | 10.606 | 31.580 | −43.412 | 1.00 | 15.30 | C |
| ATOM | 655 | CG1 | ILE | 96 | 8.446 | 30.276 | −43.144 | 1.00 | 17.27 | C |
| ATOM | 656 | CD1 | ILE | 96 | 7.558 | 31.470 | −42.767 | 1.00 | 18.12 | C |
| ATOM | 657 | C | ILE | 96 | 12.153 | 29.228 | −42.734 | 1.00 | 16.25 | C |
| ATOM | 658 | O | ILE | 96 | 12.956 | 29.408 | −43.654 | 1.00 | 17.72 | O |
| ATOM | 659 | N | LYS | 97 | 12.529 | 29.135 | −41.462 | 1.00 | 15.88 | N |
| ATOM | 660 | CA | LYS | 97 | 13.928 | 29.290 | −41.087 | 1.00 | 16.26 | C |
| ATOM | 661 | CE | LYS | 97 | 14.066 | 29.463 | −39.570 | 1.00 | 15.39 | C |
| ATOM | 662 | CG | LYS | 97 | 13.806 | 30.889 | −39.102 | 1.00 | 17.10 | C |
| ATOM | 663 | CD | LYS | 97 | 14.218 | 31.066 | −37.662 | 1.00 | 15.69 | C |
| ATOM | 664 | CE | LYS | 97 | 14.103 | 32.515 | −37.247 | 1.00 | 17.30 | C |
| ATOM | 665 | NZ | LYS | 97 | 14.552 | 32.706 | −35.842 | 1.00 | 17.34 | N |
| ATOM | 666 | C | LYS | 97 | 14.807 | 28.142 | −41.559 | 1.00 | 16.82 | C |
| ATOM | 667 | O | LYS | 97 | 15.978 | 28.352 | −41.857 | 1.00 | 16.45 | O |
| ATOM | 668 | N | SER | 98 | 14.249 | 26.937 | −41.620 | 1.00 | 17.13 | N |
| ATOM | 669 | CA | SER | 98 | 15.005 | 25.770 | −42.086 | 1.00 | 19.37 | C |
| ATOM | 670 | CB | SER | 98 | 14.311 | 24.485 | −41.647 | 1.00 | 18.74 | C |
| ATOM | 671 | OG | SER | 98 | 13.081 | 24.351 | −42.335 | 1.00 | 18.36 | O |
| ATOM | 672 | C | SER | 98 | 15.121 | 25.770 | −43.611 | 1.00 | 20.55 | C |
| ATOM | 673 | O | SER | 98 | 16.011 | 25.127 | −44.185 | 1.00 | 19.81 | O |
| ATOM | 674 | N | GLY | 99 | 14.222 | 26.496 | −44.272 | 1.00 | 19.70 | N |
| ATOM | 675 | CA | GLY | 99 | 14.248 | 26.537 | −45.721 | 1.00 | 21.29 | C |
| ATOM | 676 | C | GLY | 99 | 13.406 | 25.422 | −46.311 | 1.00 | 22.31 | C |
| ATOM | 677 | O | GLY | 99 | 13.325 | 25.270 | −47.529 | 1.00 | 22.96 | O |
| ATOM | 678 | N | GLU | 100 | 12.776 | 24.637 | −45.444 | 1.00 | 23.26 | N |
| ATOM | 679 | CA | GLU | 100 | 11.920 | 23.542 | −45.888 | 1.00 | 26.21 | C |
| ATOM | 680 | CB | GLU | 100 | 11.427 | 22.738 | −44.687 | 1.00 | 29.05 | C |
| ATOM | 681 | CG | GLU | 100 | 12.530 | 21.992 | −43.959 | 1.00 | 37.13 | C |
| ATOM | 682 | CD | GLU | 100 | 12.045 | 21.325 | −42.690 | 1.00 | 39.23 | C |
| ATOM | 683 | OE1 | GLU | 100 | 11.630 | 22.046 | −41.759 | 1.00 | 41.16 | O |
| ATOM | 684 | OE2 | GLU | 100 | 12.075 | 20.080 | −42.627 | 1.00 | 43.56 | O |
| ATOM | 685 | C | GLU | 100 | 10.728 | 24.116 | −46.647 | 1.00 | 27.02 | C |
| ATOM | 686 | O | GLU | 100 | 10.204 | 23.488 | −47.561 | 1.00 | 28.15 | O |
| ATOM | 687 | N | GLU | 101 | 10.308 | 25.316 | −46.261 | 1.00 | 26.21 | N |
| ATOM | 688 | CA | GLU | 101 | 9.188 | 25.998 | −46.907 | 1.00 | 28.61 | C |
| ATOM | 689 | CB | GLU | 101 | 7.894 | 25.785 | −46.110 | 1.00 | 31.42 | C |
| ATOM | 690 | CG | GLU | 101 | 7.207 | 24.457 | −46.362 | 1.00 | 34.52 | C |
| ATOM | 691 | CD | GLU | 101 | 6.440 | 24.445 | −47.669 | 1.00 | 36.26 | C |
| ATOM | 692 | OE1 | GLU | 101 | 7.039 | 24.748 | −48.723 | 1.00 | 39.55 | O |
| ATOM | 693 | OE2 | GLU | 101 | 5.234 | 24.130 | −47.643 | 1.00 | 39.14 | O |
| ATOM | 694 | C | GLU | 101 | 9.511 | 27.478 | −46.930 | 1.00 | 28.01 | C |
| ATOM | 695 | O | GLU | 101 | 10.175 | 27.974 | −46.027 | 1.00 | 30.29 | O |
| ATOM | 696 | N | ASP | 102 | 9.068 | 28.197 | −47.951 | 1.00 | 26.68 | N |
| ATOM | 697 | CA | ASP | 102 | 9.355 | 29.618 | −47.949 | 1.00 | 27.54 | C |
| ATOM | 698 | CB | ASP | 102 | 9.834 | 30.103 | −49.329 | 1.00 | 34.37 | C |
| ATOM | 699 | CG | ASP | 102 | 8.865 | 29.786 | −50.441 | 1.00 | 37.59 | C |
| ATOM | 700 | OD1 | ASP | 102 | 9.310 | 29.741 | −51.609 | 1.00 | 43.05 | O |
| ATOM | 701 | OD2 | ASP | 102 | 7.668 | 29.594 | −50.157 | 1.00 | 41.47 | O |
| ATOM | 702 | C | ASP | 102 | 8.138 | 30.387 | −47.462 | 1.00 | 25.40 | C |
| ATOM | 703 | O | ASP | 102 | 7.011 | 29.890 | −47.487 | 1.00 | 23.71 | O |
| ATOM | 704 | N | PHE | 103 | 8.391 | 31.588 | −46.974 | 1.00 | 23.34 | N |
| ATOM | 705 | CA | PHE | 103 | 7.353 | 32.450 | −46.446 | 1.00 | 22.31 | C |
| ATOM | 706 | CB | PHE | 103 | 7.955 | 33.825 | −46.158 | 1.00 | 22.80 | C |
| ATOM | 707 | CG | PHE | 103 | 6.962 | 34.816 | −45.672 | 1.00 | 23.87 | C |
| ATOM | 708 | CD1 | PHE | 103 | 6.597 | 34.850 | −44.335 | 1.00 | 24.41 | C |
| ATOM | 709 | CD2 | PHE | 103 | 6.317 | 35.659 | −46.570 | 1.00 | 25.76 | C |
| ATOM | 710 | CE1 | PHE | 103 | 5.595 | 35.702 | −43.897 | 1.00 | 24.83 | C |
| ATOM | 711 | CE2 | PHE | 103 | 5.313 | 36.516 | −46.140 | 1.00 | 25.51 | C |
| ATOM | 712 | CZ | PHE | 103 | 4.951 | 36.534 | −44.803 | 1.00 | 24.29 | C |
| ATOM | 713 | C | PHE | 103 | 6.151 | 32.597 | −47.379 | 1.00 | 20.99 | C |
| ATOM | 714 | O | PHE | 103 | 5.005 | 32.426 | −46.962 | 1.00 | 19.12 | O |
| ATOM | 715 | N | GLU | 104 | 6.426 | 32.915 | −48.639 | 1.00 | 19.24 | N |
| ATOM | 716 | CA | GLU | 104 | 5.388 | 33.123 | −49.645 | 1.00 | 21.00 | C |
| ATOM | 717 | CB | GLU | 104 | 6.030 | 33.509 | −50.987 | 1.00 | 21.52 | C |
| ATOM | 718 | CG | GLU | 104 | 6.796 | 34.828 | −50.940 | 1.00 | 22.25 | C |
| ATOM | 719 | CD | GLU | 104 | 8.230 | 34.677 | −50.470 | 1.00 | 23.23 | C |
| ATOM | 720 | OE1 | GLU | 104 | 8.557 | 33.677 | −49.789 | 1.00 | 23.13 | O |
| ATOM | 721 | OE2 | GLU | 104 | 9.036 | 35.575 | −50.777 | 1.00 | 26.74 | O |
| ATOM | 722 | C | GLU | 104 | 4.459 | 31.940 | −49.854 | 1.00 | 20.60 | C |
| ATOM | 723 | O | GLU | 104 | 3.247 | 32.111 | −50.029 | 1.00 | 21.04 | O |
| ATOM | 724 | N | SER | 105 | 5.023 | 30.742 | −49.851 | 1.00 | 20.47 | N |
| ATOM | 725 | CA | SER | 105 | 4.226 | 29.542 | −50.049 | 1.00 | 22.89 | C |
| ATOM | 726 | CB | SER | 105 | 5.133 | 28.328 | −50.232 | 1.00 | 23.63 | C |
| ATOM | 727 | OG | SER | 105 | 4.368 | 27.149 | −50.326 | 1.00 | 25.68 | O |
| ATOM | 728 | C | SER | 105 | 3.280 | 29.304 | −48.878 | 1.00 | 21.76 | C |
| ATOM | 729 | O | SER | 105 | 2.095 | 29.027 | −49.078 | 1.00 | 22.05 | O |
| ATOM | 730 | N | LEU | 106 | 3.801 | 29.406 | −47.660 | 1.00 | 20.34 | N |
| ATOM | 731 | CA | LEU | 106 | 2.982 | 29.198 | −46.465 | 1.00 | 18.95 | C |
| ATOM | 732 | CB | LEU | 106 | 3.861 | 29.178 | −45.208 | 1.00 | 16.50 | C |
| ATOM | 733 | CG | LEU | 106 | 4.929 | 28.075 | −45.157 | 1.00 | 17.18 | C |
| ATOM | 734 | CD1 | LEU | 106 | 5.640 | 28.119 | −43.799 | 1.00 | 15.09 | C |
| ATOM | 735 | CD2 | LEU | 106 | 4.284 | 26.711 | −45.373 | 1.00 | 16.73 | C |
| ATOM | 736 | C | LEU | 106 | 1.924 | 30.287 | −46.331 | 1.00 | 19.01 | C |
| ATOM | 737 | O | LEU | 106 | 0.802 | 30.021 | −45.882 | 1.00 | 19.62 | O |
| ATOM | 738 | N | ALA | 107 | 2.280 | 31.513 | −46.700 | 1.00 | 19.32 | N |
| ATOM | 739 | CA | ALA | 107 | 1.330 | 32.618 | −46.629 | 1.00 | 20.46 | C |
| ATOM | 740 | CB | ALA | 107 | 2.019 | 33.932 | −46.973 | 1.00 | 20.80 | C |
| ATOM | 741 | C | ALA | 107 | 0.154 | 32.378 | −47.581 | 1.00 | 21.20 | C |
| ATOM | 742 | O | ALA | 107 | −1.013 | 32.494 | −47.184 | 1.00 | 20.09 | O |
| ATOM | 743 | N | SER | 108 | 0.458 | 32.042 | −48.834 | 1.00 | 19.42 | N |
| ATOM | 744 | CA | SER | 108 | −0.586 | 31.798 | −49.827 | 1.00 | 20.67 | C |
| ATOM | 745 | CB | SER | 108 | 0.038 | 31.430 | −51.179 | 1.00 | 21.16 | C |
| ATOM | 746 | OG | SER | 108 | 0.864 | 32.479 | −51.658 | 1.00 | 26.10 | O |
| ATOM | 747 | C | SER | 108 | −1.505 | 30.667 | −49.380 | 1.00 | 21.18 | C |
| ATOM | 748 | O | SER | 108 | −2.707 | 30.671 | −49.654 | 1.00 | 21.09 | O |
| ATOM | 749 | N | GLN | 109 | −0.931 | 29.705 | −48.671 | 1.00 | 20.49 | N |
| ATOM | 750 | CA | GLN | 109 | −1.681 | 28.551 | −48.214 | 1.00 | 22.48 | C |
| ATOM | 751 | CB | GLN | 109 | −0.750 | 27.351 | −48.088 | 1.00 | 22.52 | C |
| ATOM | 752 | CG | GLN | 109 | −0.254 | 26.790 | −49.392 | 1.00 | 23.08 | C |
| ATOM | 753 | CD | GLN | 109 | 0.724 | 25.666 | −49.166 | 1.00 | 25.92 | C |
| ATOM | 754 | OE1 | GLN | 109 | 1.930 | 25.888 | −49.042 | 1.00 | 26.77 | O |
| ATOM | 755 | NE2 | GLN | 109 | 0.209 | 24.450 | −49.090 | 1.00 | 24.41 | N |
| ATOM | 756 | C | GLN | 109 | −2.427 | 28.683 | −46.900 | 1.00 | 21.83 | C |
| ATOM | 757 | O | GLN | 109 | −3.587 | 28.311 | −46.801 | 1.00 | 20.58 | O |
| ATOM | 758 | N | PHE | 110 | −1.757 | 29.221 | −45.892 | 1.00 | 21.69 | N |
| ATOM | 759 | CA | PHE | 110 | −2.353 | 29.274 | −44.574 | 1.00 | 22.41 | C |
| ATOM | 760 | CB | PHE | 110 | −1.471 | 28.442 | −43.645 | 1.00 | 21.55 | C |
| ATOM | 761 | CG | PHE | 110 | −1.139 | 27.080 | −44.201 | 1.00 | 21.62 | C |
| ATOM | 762 | CD1 | PHE | 110 | 0.174 | 26.732 | −44.499 | 1.00 | 21.24 | C |
| ATOM | 763 | CD2 | PHE | 110 | −2.149 | 26.157 | −44.455 | 1.00 | 21.95 | C |
| ATOM | 764 | CE1 | PHE | 110 | 0.474 | 25.485 | −45.044 | 1.00 | 24.45 | C |
| ATOM | 765 | CE2 | PHE | 110 | −1.864 | 24.912 | −44.996 | 1.00 | 22.45 | C |
| ATOM | 766 | CZ | PHE | 110 | −0.551 | 24.571 | −45.294 | 1.00 | 24.44 | C |
| ATOM | 767 | C | PHE | 110 | −2.687 | 30.606 | −43.928 | 1.00 | 22.56 | C |
| ATOM | 768 | O | PHE | 110 | −3.248 | 30.628 | −42.839 | 1.00 | 23.57 | O |
| ATOM | 769 | N | SER | 111 | −2.367 | 31.719 | −44.571 | 1.00 | 21.02 | N |
| ATOM | 770 | CA | SER | 111 | −2.697 | 32.997 | −43.961 | 1.00 | 21.58 | C |
| ATOM | 771 | CB | SER | 111 | −2.030 | 34.146 | −44.691 | 1.00 | 18.53 | C |
| ATOM | 772 | OC | SER | 111 | −2.347 | 35.369 | −44.036 | 1.00 | 20.21 | O |
| ATOM | 773 | C | SER | 111 | −4.206 | 33.242 | −43.951 | 1.00 | 22.85 | C |
| ATOM | 774 | O | SER | 111 | −4.876 | 33.071 | −44.975 | 1.00 | 22.36 | O |
| ATOM | 775 | N | ASP | 112 | −4.722 | 33.648 | −42.793 | 1.00 | 23.38 | N |
| ATOM | 776 | CA | ASP | 112 | −6.145 | 33.937 | −42.619 | 1.00 | 25.80 | C |
| ATOM | 777 | CB | ASP | 112 | −6.578 | 33.724 | −41.159 | 1.00 | 24.20 | C |
| ATOM | 778 | CG | ASP | 112 | −6.822 | 32.261 | −40.820 | 1.00 | 26.33 | C |
| ATOM | 779 | OD1 | ASP | 112 | −7.520 | 31.578 | −41.599 | 1.00 | 26.12 | O |
| ATOM | 780 | OD2 | ASP | 112 | −6.333 | 31.789 | −39.767 | 1.00 | 23.37 | O |
| ATOM | 781 | C | ASP | 112 | −6.436 | 35.379 | −43.008 | 1.00 | 25.72 | C |
| ATOM | 782 | O | ASP | 112 | −7.283 | 36.030 | −42.406 | 1.00 | 28.16 | O |
| ATOM | 783 | N | CYS | 113 | −5.723 | 35.884 | −44.004 | 1.00 | 26.38 | N |
| ATOM | 784 | CA | CYS | 113 | −5.914 | 37.252 | −44.458 | 1.00 | 25.54 | C |
| ATOM | 785 | CB | CYS | 113 | −4.672 | 38.090 | −44.141 | 1.00 | 24.34 | C |
| ATOM | 786 | SG | CYS | 113 | −4.765 | 39.850 | −44.586 | 1.00 | 26.84 | S |
| ATOM | 787 | C | CYS | 113 | −6.154 | 37.234 | −45.954 | 1.00 | 25.63 | C |
| ATOM | 788 | O | CYS | 113 | −5.685 | 36.337 | −46.651 | 1.00 | 25.42 | O |
| ATOM | 789 | N | SER | 114 | −6.891 | 38.224 | −46.444 | 1.00 | 24.84 | N |
| ATOM | 790 | CA | SER | 114 | −7.181 | 38.321 | −47.870 | 1.00 | 25.86 | C |
| ATOM | 791 | CB | SER | 114 | −8.167 | 39.469 | −48.132 | 1.00 | 26.75 | C |
| ATOM | 792 | OG | SER | 114 | −7.695 | 40.692 | −47.593 | 1.00 | 29.35 | O |
| ATOM | 793 | C | SER | 114 | −5.893 | 38542 | −48.667 | 1.00 | 25.32 | C |
| ATOM | 794 | O | SER | 114 | −5.840 | 38261 | −49.872 | 1.00 | 24.77 | O |
| ATOM | 795 | N | SER | 115 | −4.854 | 39.033 | −47.995 | 1.00 | 23.19 | N |
| ATOM | 796 | CA | SER | 115 | −3.572 | 39.278 | −48.664 | 1.00 | 22.81 | C |
| ATOM | 797 | CB | SER | 115 | −2.665 | 40.159 | −47.798 | 1.00 | 20.33 | C |
| ATOM | 798 | OG | SER | 115 | −2.422 | 39.568 | −46.531 | 1.00 | 20.91 | O |
| ATOM | 799 | C | SER | 115 | −2.849 | 37.975 | −49.001 | 1.00 | 21.09 | C |
| ATOM | 800 | O | SER | 115 | −1.799 | 37.989 | −49.640 | 1.00 | 20.29 | O |
| ATOM | 801 | N | ALA | 116 | −3.412 | 36.849 | −48.574 | 1.00 | 20.25 | N |
| ATOM | 802 | CA | ALA | 116 | −2.824 | 35.546 | −48.855 | 1.00 | 19.33 | C |
| ATOM | 803 | CB | ALA | 116 | −3.664 | 34.441 | −48.217 | 1.00 | 22.64 | C |
| ATOM | 804 | C | ALA | 116 | −2.750 | 35.329 | −50.368 | 1.00 | 21.09 | C |
| ATOM | 805 | O | ALA | 116 | −1.811 | 34.707 | −50.870 | 1.00 | 17.16 | O |
| ATOM | 806 | N | LYS | 117 | −3.747 | 35.845 | −51.088 | 1.00 | 18.87 | N |
| ATOM | 807 | CA | LYS | 117 | −3.806 | 35.699 | −52.542 | 1.00 | 20.20 | C |
| ATOM | 808 | CB | LYS | 117 | −5.134 | 36.257 | −53.068 | 1.00 | 23.03 | C |
| ATOM | 809 | CG | LYS | 117 | −6.366 | 35.604 | −52.451 | 1.00 | 29.17 | C |
| ATOM | 810 | CD | LYS | 117 | −6.627 | 34.220 | −53.029 | 1.00 | 31.39 | C |
| ATOM | 811 | CE | LYS | 117 | −7.717 | 33.479 | −52.245 | 1.00 | 34.23 | C |
| ATOM | 812 | NZ | LYS | 117 | −7.223 | 33.034 | −50.901 | 1.00 | 32.34 | N |
| ATOM | 813 | C | LYS | 117 | −2.640 | 36.404 | −53.228 | 1.00 | 19.22 | C |
| ATOM | 814 | O | LYS | 117 | −2.336 | 36.131 | −54.392 | 1.00 | 17.70 | O |
| ATOM | 815 | N | ALA | 118 | −1.999 | 37.319 | −52.508 | 1.00 | 17.95 | N |
| ATOM | 816 | CA | ALA | 118 | −0.854 | 38.045 | −53.038 | 1.00 | 19.01 | C |
| ATOM | 817 | CB | ALA | 118 | −1.085 | 39.551 | −52.930 | 1.00 | 17.73 | C |
| ATOM | 818 | C | ALA | 118 | 0.411 | 37.643 | −52.272 | 1.00 | 19.64 | C |
| ATOM | 819 | O | ALA | 118 | 1.288 | 38.470 | −52.015 | 1.00 | 19.72 | O |
| ATOM | 820 | N | ARC | 119 | 0.489 | 36.366 | −51.915 | 1.00 | 18.61 | N |
| ATOM | 821 | CA | ARG | 119 | 1.634 | 35.825 | −51.189 | 1.00 | 21.48 | C |
| ATOM | 822 | CB | ARC | 119 | 2.889 | 35.863 | −52.079 | 1.00 | 24.72 | C |
| ATOM | 823 | CG | ARC | 119 | 2.947 | 34.743 | −53.119 | 1.00 | 27.85 | C |
| ATOM | 824 | CD | ARG | 119 | 3.738 | 35.130 | −54.367 | 1.00 | 31.89 | C |
| ATOM | 825 | NE | ARC | 119 | 2.901 | 35.895 | −55.290 | 1.00 | 38.23 | N |
| ATOM | 826 | CZ | ARC | 119 | 2.579 | 37.180 | −55.144 | 1.00 | 38.28 | C |
| ATOM | 827 | NH1 | ARC | 119 | 3.033 | 37.886 | −54.112 | 1.00 | 39.89 | N |
| ATOM | 828 | NH2 | ARC | 119 | 1.763 | 37.753 | −56.016 | 1.00 | 33.97 | N |
| ATOM | 829 | C | ARC | 119 | 1.881 | 36.589 | −49.893 | 1.00 | 20.62 | C |
| ATOM | 830 | O | ARC | 119 | 3.008 | 36.656 | −49.407 | 1.00 | 18.56 | O |
| ATOM | 831 | N | GLY | 120 | 0.817 | 37.171 | −49.343 | 1.00 | 20.89 | N |
| ATOM | 832 | CA | GLY | 120 | 0.934 | 37.908 | −48.094 | 1.00 | 21.30 | C |
| ATOM | 833 | C | GLY | 120 | 1.363 | 39.359 | −48.201 | 1.00 | 22.05 | C |
| ATOM | 834 | O | GLY | 120 | 1.426 | 40.053 | −47.191 | 1.00 | 21.34 | O |
| ATOM | 835 | N | ASP | 121 | 1.640 | 39.832 | −49.414 | 1.00 | 22.44 | N |
| ATOM | 836 | CA | ASP | 121 | 2.073 | 41.214 | −49.608 | 1.00 | 22.42 | C |
| ATOM | 837 | CB | ASP | 121 | 2.534 | 41.429 | −51.053 | 1.00 | 22.20 | C |
| ATOM | 838 | CG | ASP | 121 | 2.921 | 42.870 | −51.334 | 1.00 | 23.17 | C |
| ATOM | 839 | OD1 | ASP | 121 | 3.600 | 43.489 | −50.487 | 1.00 | 21.32 | O |
| ATOM | 840 | OD2 | ASP | 121 | 2.553 | 43.381 | −52.410 | 1.00 | 22.33 | O |
| ATOM | 841 | C | ASP | 121 | 0.990 | 42.232 | −49.262 | 1.00 | 24.01 | C |
| ATOM | 842 | O | ASP | 121 | −0.141 | 42.132 | −49.728 | 1.00 | 22.67 | O |
| ATOM | 843 | N | LEU | 122 | 1.363 | 43.219 | −48.450 | 1.00 | 24.20 | N |
| ATOM | 844 | CA | LEU | 122 | 0.449 | 44.263 | −48.017 | 1.00 | 25.71 | C |
| ATOM | 845 | CB | LEU | 122 | 0.580 | 44.482 | −46.510 | 1.00 | 24.03 | C |
| ATOM | 846 | CG | LEU | 122 | 0.179 | 43.321 | −45.610 | 1.00 | 24.14 | C |
| ATOM | 847 | CD1 | LEU | 122 | 0.457 | 43.698 | −44.155 | 1.00 | 22.74 | C |
| ATOM | 848 | CD2 | LEU | 122 | −1.294 | 43.007 | −45.818 | 1.00 | 23.98 | C |
| ATOM | 849 | C | LEU | 122 | 0.701 | 45.589 | −48.710 | 1.00 | 26.77 | C |
| ATOM | 850 | O | LEU | 122 | −0.069 | 46.530 | −48.541 | 1.00 | 27.12 | O |
| ATOM | 851 | N | GLY | 123 | 1.777 | 45.666 | −49.481 | 1.00 | 28.17 | N |
| ATOM | 852 | CA | GLY | 123 | 2.101 | 46.913 | −50.144 | 1.00 | 28.92 | C |
| ATOM | 853 | C | GLY | 123 | 2.829 | 47.781 | −49.138 | 1.00 | 30.04 | C |
| ATOM | 854 | O | GLY | 123 | 3.054 | 47.356 | −48.003 | 1.00 | 30.87 | O |
| ATOM | 855 | N | ALA | 124 | 3.198 | 48.992 | −49.537 | 1.00 | 28.94 | N |
| ATOM | 856 | CA | ALA | 124 | 3.910 | 49.892 | −48.641 | 1.00 | 30.19 | C |
| ATOM | 857 | CB | ALA | 124 | 4.740 | 50.886 | −49.451 | 1.00 | 30.74 | C |
| ATOM | 858 | C | ALA | 124 | 2.928 | 50.635 | −47.746 | 1.00 | 31.41 | C |
| ATOM | 859 | O | ALA | 124 | 1.744 | 50.747 | −48.064 | 1.00 | 31.90 | O |
| ATOM | 860 | N | PHE | 125 | 3.419 | 51.128 | −46.615 | 1.00 | 31.96 | N |
| ATOM | 861 | CA | PHE | 125 | 2.576 | 51.868 | −45.683 | 1.00 | 32.00 | C |
| ATOM | 862 | CB | PHE | 125 | 1.660 | 50.907 | −44.905 | 1.00 | 32.14 | C |
| ATOM | 863 | CG | PHE | 125 | 2.394 | 49.832 | −44.132 | 1.00 | 32.47 | C |
| ATOM | 864 | CD1 | PHE | 125 | 3.038 | 50.126 | −42.932 | 1.00 | 32.28 | C |
| ATOM | 865 | CD2 | PHE | 125 | 2.421 | 48.519 | −44.601 | 1.00 | 32.76 | C |
| ATOM | 866 | CE1 | PHE | 125 | 3.697 | 49.131 | −42.206 | 1.00 | 31.38 | C |
| ATOM | 867 | CE2 | PHE | 125 | 3.077 | 47.514 | −43.885 | 1.00 | 32.65 | C |
| ATOM | 868 | CZ | PHE | 125 | 3.717 | 47.822 | −42.681 | 1.00 | 30.91 | C |
| ATOM | 869 | C | PHE | 125 | 3.406 | 52.709 | −44.726 | 1.00 | 31.21 | C |
| ATOM | 870 | O | PHE | 125 | 4.608 | 52.498 | −44.578 | 1.00 | 32.99 | O |
| ATOM | 871 | N | SER | 126 | 2.765 | 53.677 | −44.087 | 1.00 | 32.12 | N |
| ATOM | 872 | CA | SER | 126 | 3.460 | 54.540 | −43.145 | 1.00 | 33.01 | C |
| ATOM | 873 | CB | SER | 126 | 3.303 | 56.008 | −43.551 | 1.00 | 34.29 | C |
| ATOM | 874 | OG | SER | 126 | 1.941 | 56.376 | −43.561 | 1.00 | 35.69 | O |
| ATOM | 875 | C | SER | 126 | 2.885 | 54.315 | −41.755 | 1.00 | 31.94 | C |
| ATOM | 876 | O | SER | 126 | 1.956 | 53.524 | −41.579 | 1.00 | 31.18 | O |
| ATOM | 877 | N | ARG | 127 | 3.439 | 55.003 | −40.766 | 1.00 | 33.72 | N |
| ATOM | 878 | CA | ARG | 127 | 2.957 | 54.855 | −39.404 | 1.00 | 34.85 | C |
| ATOM | 879 | CB | ARC | 127 | 3.920 | 55.528 | −38.424 | 1.00 | 34.40 | C |
| ATOM | 880 | CG | ARG | 127 | 5.199 | 54.736 | −38.160 | 1.00 | 33.60 | C |
| ATOM | 881 | CD | ARC | 127 | 5.993 | 55.353 | −37.018 | 1.00 | 32.21 | C |
| ATOM | 882 | NE | ARC | 127 | 7.195 | 54.598 | −36.658 | 1.00 | 31.62 | N |
| ATOM | 883 | CZ | ARG | 127 | 7.211 | 53.507 | −35.893 | 1.00 | 30.76 | C |
| ATOM | 884 | NH1 | ARC | 127 | 6.085 | 53.018 | −35.396 | 1.00 | 27.78 | N |
| ATOM | 885 | NH2 | ARG | 127 | 8.364 | 52.916 | −35.604 | 1.00 | 30.85 | N |
| ATOM | 886 | C | ARC | 127 | 1.553 | 55.428 | −39.238 | 1.00 | 37.37 | C |
| ATOM | 887 | O | ARC | 127 | 1.233 | 56.485 | −39.787 | 1.00 | 37.57 | O |
| ATOM | 888 | N | GLY | 128 | 0.716 | 54.713 | −38.494 | 1.00 | 3878 | N |
| ATOM | 889 | CA | GLY | 128 | −0.641 | 55.166 | −38.253 | 1.00 | 40.45 | C |
| ATOM | 890 | C | GLY | 128 | −1.716 | 54.449 | −39.047 | 1.00 | 40.51 | C |
| ATOM | 891 | O | GLY | 128 | −2.903 | 54.663 | −38.811 | 1.00 | 42.58 | O |
| ATOM | 892 | N | GLN | 129 | −1.313 | 53.592 | −39.979 | 1.00 | 38.75 | N |
| ATOM | 893 | CA | GLN | 129 | −2.272 | 52.871 | −40.807 | 1.00 | 37.32 | C |
| ATOM | 894 | CB | GLN | 129 | −1.742 | 52.754 | −42.238 | 1.00 | 39.24 | C |
| ATOM | 895 | CG | GLN | 129 | −1.226 | 54.057 | −42.833 | 1.00 | 41.99 | C |
| ATOM | 896 | CD | GLN | 129 | −0.708 | 53.885 | −44.256 | 1.00 | 43.29 | C |
| ATOM | 897 | OE1 | GLN | 129 | −0.117 | 54.799 | −44.826 | 1.00 | 44.89 | O |
| ATOM | 898 | NE2 | GLN | 129 | −0.934 | 52.711 | −44.832 | 1.00 | 45.91 | N |
| ATOM | 899 | C | GLN | 129 | −2.583 | 51.474 | −40.278 | 1.00 | 36.07 | C |
| ATOM | 900 | O | GLN | 129 | −3.736 | 51.044 | −40.269 | 1.00 | 36.17 | O |
| ATOM | 901 | N | MET | 130 | −1.552 | 50.761 | −39.839 | 1.00 | 33.33 | N |
| ATOM | 902 | CA | MET | 130 | −1.741 | 49.407 | −39.340 | 1.00 | 32.52 | C |
| ATOM | 903 | CB | MET | 130 | −0.554 | 48.532 | −39.760 | 1.00 | 31.71 | C |
| ATOM | 904 | CG | MET | 130 | −0.244 | 48.572 | −41.249 | 1.00 | 33.63 | C |
| ATOM | 905 | SD | MET | 130 | −1.533 | 47.832 | −42.264 | 1.00 | 36.73 | S |
| ATOM | 906 | CE | MET | 130 | −0.783 | 47.898 | −43.882 | 1.00 | 36.14 | C |
| ATOM | 907 | C | MET | 130 | −1.871 | 49.390 | −37.823 | 1.00 | 31.76 | C |
| ATOM | 908 | O | MET | 130 | −1.551 | 50.373 | −37.157 | 1.00 | 31.56 | O |
| ATOM | 909 | N | GLN | 131 | −2.348 | 48.274 | −37.279 | 1.00 | 31.61 | N |
| ATOM | 910 | CA | GLN | 131 | −2.474 | 48.154 | −35.834 | 1.00 | 32.12 | C |
| ATOM | 911 | CB | GLN | 131 | −3.005 | 46.777 | −35.471 | 1.00 | 34.43 | C |
| ATOM | 912 | CG | GLN | 131 | −4.400 | 46.572 | −36.018 | 1.00 | 37.18 | C |
| ATOM | 913 | CD | GLN | 131 | −4.967 | 45.226 | −35.680 | 1.00 | 38.83 | C |
| ATOM | 914 | OE1 | GLN | 131 | −5.108 | 44.877 | −34.510 | 1.00 | 43.14 | O |
| ATOM | 915 | NE2 | GLN | 131 | −5.298 | 44.455 | −36.703 | 1.00 | 39.26 | N |
| ATOM | 916 | C | GLN | 131 | −1.093 | 48.402 | −35.249 | 1.00 | 31.69 | C |
| ATOM | 917 | O | GLN | 131 | −0.080 | 47.971 | −35.807 | 1.00 | 31.12 | O |
| ATOM | 918 | N | LYS | 132 | −1.061 | 49.113 | −34.130 | 1.00 | 28.74 | N |
| ATOM | 919 | CA | LYS | 132 | 0.183 | 49.499 | −33.492 | 1.00 | 26.91 | C |
| ATOM | 920 | CB | LYS | 132 | −0.123 | 50.136 | −32.133 | 1.00 | 27.76 | C |
| ATOM | 921 | CG | LYS | 132 | 1.037 | 50.907 | −31.535 | 1.00 | 27.78 | C |
| ATOM | 922 | CD | LYS | 132 | 1.653 | 51.862 | −32.532 | 1.00 | 26.57 | C |
| ATOM | 923 | CE | LYS | 132 | 2.829 | 52.600 | −31.910 | 1.00 | 27.44 | C |
| ATOM | 924 | NZ | LYS | 132 | 3.657 | 53.293 | −32.928 | 1.00 | 28.79 | N |
| ATOM | 925 | C | LYS | 132 | 1.273 | 48.434 | −33.342 | 1.00 | 26.22 | C |
| ATOM | 926 | O | LYS | 132 | 2.423 | 48.679 | −33.707 | 1.00 | 25.72 | O |
| ATOM | 927 | N | PRO | 133 | 0.936 | 47.254 | −32.797 | 1.00 | 24.78 | N |
| ATOM | 928 | CD | PRO | 133 | −0.361 | 46.831 | −32.242 | 1.00 | 26.39 | C |
| ATOM | 929 | CA | PRO | 133 | 1.944 | 46.198 | −32.628 | 1.00 | 26.25 | C |
| ATOM | 930 | CB | PRO | 133 | 1.148 | 45.060 | −31.990 | 1.00 | 27.61 | C |
| ATOM | 931 | CG | PRO | 133 | 0.055 | 45.778 | −31.247 | 1.00 | 27.91 | C |
| ATOM | 932 | C | PRO | 133 | 2.573 | 45.782 | −33.959 | 1.00 | 25.91 | C |
| ATOM | 933 | O | PRO | 133 | 3.787 | 45.566 | −34.048 | 1.00 | 25.34 | O |
| ATOM | 934 | N | PHE | 134 | 1.733 | 45.676 | −34.984 | 1.00 | 25.36 | N |
| ATOM | 935 | CA | PHE | 134 | 2.172 | 45.288 | −36.326 | 1.00 | 23.87 | C |
| ATOM | 936 | CB | PEE | 134 | 0.950 | 45.094 | −37.225 | 1.00 | 22.16 | C |
| ATOM | 937 | CG | PHE | 134 | 1.269 | 44.499 | −38.570 | 1.00 | 20.35 | C |
| ATOM | 938 | CD1 | PHE | 134 | 1.143 | 43.131 | −38.783 | 1.00 | 18.13 | C |
| ATOM | 939 | CD2 | PHE | 134 | 1.691 | 45.307 | −39.618 | 1.00 | 19.91 | C |
| ATOM | 940 | CE1 | PHE | 134 | 1.433 | 42.567 | −40.030 | 1.00 | 19.24 | C |
| ATOM | 941 | CE2 | PHE | 134 | 1.985 | 44.758 | −40.870 | 1.00 | 18.90 | C |
| ATOM | 942 | CZ | PHE | 134 | 1.855 | 43.383 | −41.075 | 1.00 | 19.12 | C |
| ATOM | 943 | C | PHE | 134 | 3.058 | 46.389 | −36.898 | 1.00 | 23.25 | C |
| ATOM | 944 | O | PHE | 134 | 4.102 | 46.128 | −37.502 | 1.00 | 22.16 | O |
| ATOM | 945 | N | GLU | 135 | 2.636 | 47.634 | −36.698 | 1.00 | 23.54 | N |
| ATOM | 946 | CA | GLU | 135 | 3.391 | 48.778 | −37.188 | 1.00 | 22.32 | C |
| ATOM | 947 | CB | GLU | 135 | 2.626 | 50.067 | −36.890 | 1.00 | 23.65 | C |
| ATOM | 948 | CG | GLU | 135 | 3.431 | 51.324 | −37.128 | 1.00 | 25.84 | C |
| ATOM | 949 | CD | GLU | 135 | 2.699 | 52.563 | −36.657 | 1.00 | 28.66 | C |
| ATOM | 950 | OE1 | GLU | 135 | 1.533 | 52.743 | −37.064 | 1.00 | 28.63 | O |
| ATOM | 951 | OE2 | GLU | 135 | 3.290 | 53.346 | −35.883 | 1.00 | 30.94 | O |
| ATOM | 952 | C | GLU | 135 | 4.794 | 48.859 | −36.585 | 1.00 | 22.06 | C |
| ATOM | 953 | O | GLU | 135 | 5.792 | 48.958 | −37.313 | 1.00 | 19.26 | O |
| ATOM | 954 | N | ASP | 136 | 4.877 | 48.829 | −35.257 | 1.00 | 21.97 | N |
| ATOM | 955 | CA | ASP | 136 | 6.176 | 48.913 | −34.590 | 1.00 | 21.65 | C |
| ATOM | 956 | CB | ASP | 136 | 6.016 | 48.909 | −33.064 | 1.00 | 24.59 | C |
| ATOM | 957 | CG | ASP | 136 | 5.397 | 50.196 | −32.538 | 1.00 | 24.60 | C |
| ATOM | 958 | OD1 | ASP | 136 | 5.452 | 51.217 | −33.251 | 1.00 | 24.55 | O |
| ATOM | 959 | OD2 | ASP | 136 | 4.866 | 50.188 | −31.411 | 1.00 | 25.54 | O |
| ATOM | 960 | C | ASP | 136 | 7.094 | 47.781 | −35.010 | 1.00 | 20.28 | C |
| ATOM | 961 | O | ASP | 136 | 8.282 | 47.997 | −35.243 | 1.00 | 20.40 | O |
| ATOM | 962 | N | ALA | 137 | 6.547 | 46.576 | −35.114 | 1.00 | 20.56 | N |
| ATOM | 963 | CA | ALA | 137 | 7.347 | 45.430 | −35.534 | 1.00 | 20.52 | C |
| ATOM | 964 | CB | ALA | 137 | 6.503 | 44.150 | −35.480 | 1.00 | 20.41 | C |
| ATOM | 965 | C | ALA | 137 | 7.855 | 45.675 | −36.961 | 1.00 | 20.52 | C |
| ATOM | 966 | O | ALA | 137 | 9.034 | 45.483 | −37.254 | 1.00 | 19.91 | O |
| ATOM | 967 | N | SER | 138 | 6.966 | 46.132 | −37.838 | 1.00 | 20.50 | N |
| ATOM | 968 | CA | SER | 138 | 7.332 | 46.392 | −39.229 | 1.00 | 21.18 | C |
| ATOM | 969 | CB | SER | 138 | 6.121 | 46.885 | −40.028 | 1.00 | 20.16 | C |
| ATOM | 970 | OG | SER | 138 | 5.124 | 45.886 | −40.118 | 1.00 | 18.18 | O |
| ATOM | 971 | C | SER | 138 | 8.459 | 47.396 | −39.381 | 1.00 | 22.52 | C |
| ATOM | 972 | O | SER | 138 | 9.391 | 47.164 | −40.152 | 1.00 | 21.15 | O |
| ATOM | 973 | N | PHE | 139 | 8.371 | 48.511 | −38.654 | 1.00 | 22.86 | N |
| ATOM | 974 | CA | PHE | 139 | 9.386 | 49.559 | −38.726 | 1.00 | 24.53 | C |
| ATOM | 975 | CB | PHE | 139 | 8.806 | 50.906 | −38.272 | 1.00 | 24.41 | C |
| ATOM | 976 | CG | PHE | 139 | 7.967 | 51.582 | −39.320 | 1.00 | 24.60 | C |
| ATOM | 977 | CD1 | PHE | 139 | 6.624 | 51.264 | −39.471 | 1.00 | 24.06 | C |
| ATOM | 978 | CD2 | PHE | 139 | 8.541 | 52.503 | −40.195 | 1.00 | 26.09 | C |
| ATOM | 979 | CE1 | PHE | 139 | 5.861 | 51.852 | −40.483 | 1.00 | 25.14 | C |
| ATOM | 980 | CE2 | PHE | 139 | 7.786 | 53.096 | −41.211 | 1.00 | 25.07 | C |
| ATOM | 981 | CZ | PHE | 139 | 6.446 | 52.768 | −41.353 | 1.00 | 24.16 | C |
| ATOM | 982 | C | PHE | 139 | 10.661 | 49.279 | −37.942 | 1.00 | 25.70 | C |
| ATOM | 983 | O | PHE | 139 | 11.638 | 50.027 | −38.052 | 1.00 | 26.40 | O |
| ATOM | 984 | N | ALA | 140 | 10.653 | 48.209 | −37.155 | 1.00 | 24.62 | N |
| ATOM | 985 | CA | ALA | 140 | 11.820 | 47.841 | −36.363 | 1.00 | 24.11 | C |
| ATOM | 986 | CB | ALA | 140 | 11.377 | 47.346 | −34.993 | 1.00 | 22.14 | C |
| ATOM | 987 | C | ALA | 140 | 12.617 | 46.758 | −37.096 | 1.00 | 24.35 | C |
| ATOM | 988 | O | ALA | 140 | 13.802 | 46.543 | −36.824 | 1.00 | 23.36 | O |
| ATOM | 989 | N | LEU | 141 | 11.958 | 46.077 | −38.028 | 1.00 | 25.20 | N |
| ATOM | 990 | CA | LEU | 141 | 12.615 | 45.040 | −38.815 | 1.00 | 25.89 | C |
| ATOM | 991 | CB | LEU | 141 | 11.590 | 44.234 | −39.625 | 1.00 | 25.61 | C |
| ATOM | 992 | CG | LEU | 141 | 10.696 | 43.197 | −38.942 | 1.00 | 25.20 | C |
| ATOM | 993 | CD1 | LEU | 141 | 9.626 | 42.727 | −39.923 | 1.00 | 22.19 | C |
| ATOM | 994 | CD2 | LEU | 141 | 11.536 | 42.015 | −38.466 | 1.00 | 24.10 | C |
| ATOM | 995 | C | LEU | 141 | 13.579 | 45.695 | −39.790 | 1.00 | 27.21 | C |
| ATOM | 996 | O | LEU | 141 | 13.336 | 46.811 | −40.254 | 1.00 | 27.62 | O |
| ATOM | 997 | N | ARG | 142 | 14.681 | 45.013 | −40.084 | 1.00 | 26.14 | N |
| ATOM | 998 | CA | ARG | 142 | 15.638 | 45.516 | −41.054 | 1.00 | 26.46 | C |
| ATOM | 999 | CB | ARG | 142 | 17.029 | 44.932 | −40.789 | 1.00 | 27.29 | C |
| ATOM | 1000 | CG | ARG | 142 | 17.614 | 45.279 | −39.416 | 1.00 | 27.36 | C |
| ATOM | 1001 | CD | ARG | 142 | 18.966 | 44.615 | −39.227 | 1.00 | 30.93 | C |
| ATOM | 1002 | NE | ARG | 142 | 18.847 | 43.157 | −39.178 | 1.00 | 33.42 | N |
| ATOM | 1003 | CZ | ARG | 142 | 18.768 | 42.448 | −38.057 | 1.00 | 32.99 | C |
| ATOM | 1004 | NH1 | ARG | 142 | 18.805 | 43.062 | −36.882 | 1.00 | 32.06 | N |
| ATOM | 1005 | NH2 | ARG | 142 | 18.638 | 41.127 | −38.111 | 1.00 | 31.91 | N |
| ATOM | 1006 | C | ARG | 142 | 15.096 | 45.009 | −42.388 | 1.00 | 27.13 | C |
| ATOM | 1007 | O | ARG | 142 | 14.310 | 44.062 | −42.414 | 1.00 | 25.87 | O |
| ATOM | 1008 | N | THR | 143 | 15.496 | 45.626 | −43.493 | 1.00 | 27.88 | N |
| ATOM | 1009 | CA | THR | 143 | 15.017 | 45.190 | −44.800 | 1.00 | 27.91 | C |
| ATOM | 1010 | CB | THR | 143 | 15.606 | 46.054 | −45.926 | 1.00 | 30.84 | C |
| ATOM | 1011 | OG1 | THR | 143 | 15.344 | 47.432 | −45.647 | 1.00 | 34.68 | O |
| ATOM | 1012 | CG2 | THR | 143 | 14.976 | 45.691 | −47.266 | 1.00 | 30.42 | C |
| ATOM | 1013 | C | THR | 143 | 15.413 | 43.737 | −45.039 | 1.00 | 27.33 | C |
| ATOM | 1014 | O | THR | 143 | 16.585 | 43.370 | −44.895 | 1.00 | 24.82 | O |
| ATOM | 1015 | N | GLY | 144 | 14.428 | 42.916 | −45.394 | 1.00 | 24.57 | N |
| ATOM | 1016 | CA | GLY | 144 | 14.681 | 41.505 | −45.636 | 1.00 | 25.36 | C |
| ATOM | 1017 | C | GLY | 144 | 14.530 | 40.618 | −44.406 | 1.00 | 24.34 | C |
| ATOM | 1018 | O | GLY | 144 | 14.527 | 39.386 | −44.518 | 1.00 | 24.89 | O |
| ATOM | 1019 | N | GLU | 145 | 14.399 | 41.235 | −43.234 | 1.00 | 22.32 | N |
| ATOM | 1020 | CA | GLU | 145 | 14.257 | 40.498 | −41.976 | 1.00 | 21.64 | C |
| ATOM | 1021 | CB | GLU | 145 | 14.792 | 41.341 | −40.810 | 1.00 | 20.75 | C |
| ATOM | 1022 | CG | GLU | 145 | 14.707 | 40.651 | −39.452 | 1.00 | 24.18 | C |
| ATOM | 1023 | CD | GLU | 145 | 15.227 | 41.512 | −38.314 | 1.00 | 26.80 | C |
| ATOM | 1024 | OE1 | GLU | 145 | 15.339 | 42.746 | −38.498 | 1.00 | 26.95 | O |
| ATOM | 1025 | OE2 | GLU | 145 | 15.509 | 40.958 | −37.229 | 1.00 | 25.61 | O |
| ATOM | 1026 | C | GLU | 145 | 12.820 | 40.056 | −41.678 | 1.00 | 20.47 | C |
| ATOM | 1027 | O | GLU | 145 | 11.848 | 40.691 | −42.098 | 1.00 | 20.76 | O |
| ATOM | 1028 | N | MET | 146 | 12.699 | 38.957 | −40.943 | 1.00 | 19.00 | N |
| ATOM | 1029 | CA | MET | 146 | 11.397 | 38.407 | −40.580 | 1.00 | 19.12 | C |
| ATOM | 1030 | CB | MET | 146 | 11.311 | 36.940 | −41.020 | 1.00 | 20.03 | C |
| ATOM | 1031 | CG | MET | 146 | 9.944 | 36.296 | −40.835 | 1.00 | 18.34 | C |
| ATOM | 1032 | SD | MET | 146 | 9.907 | 34.578 | −41.401 | 1.00 | 18.12 | S |
| ATOM | 1033 | CE | MET | 146 | 10.702 | 33.776 | −40.020 | 1.00 | 17.76 | C |
| ATOM | 1034 | C | MET | 146 | 11.196 | 38.520 | −39.072 | 1.00 | 18.91 | C |
| ATOM | 1035 | O | MET | 146 | 12.146 | 38.407 | −38.289 | 1.00 | 15.75 | O |
| ATOM | 1036 | N | SER | 147 | 9.955 | 38.738 | −38.661 | 1.00 | 17.79 | N |
| ATOM | 1037 | CA | SER | 147 | 9.651 | 38.873 | −37.247 | 1.00 | 17.17 | C |
| ATOM | 1038 | CB | SER | 147 | 8.412 | 39.752 | −37.060 | 1.00 | 16.31 | C |
| ATOM | 1039 | OG | SER | 147 | 7.258 | 39.013 | −37.452 | 1.00 | 16.54 | O |
| ATOM | 1040 | C | SER | 147 | 9.351 | 37.524 | −36.617 | 1.00 | 16.94 | C |
| ATOM | 1041 | O | SER | 147 | 9.291 | 36.498 | −37.296 | 1.00 | 15.50 | O |
| ATOM | 1042 | N | GLY | 148 | 9.184 | 37.552 | −35.299 | 1.00 | 18.15 | N |
| ATOM | 1043 | CA | GLY | 148 | 8.802 | 36.365 | −34.566 | 1.00 | 20.27 | C |
| ATOM | 1044 | C | GLY | 148 | 7.299 | 36.540 | −34.455 | 1.00 | 20.89 | C |
| ATOM | 1045 | O | GLY | 148 | 6.734 | 37.338 | −35.202 | 1.00 | 20.31 | O |
| ATOM | 1046 | N | PRO | 149 | 6.616 | 35.830 | −33.552 | 1.00 | 22.08 | N |
| ATOM | 1047 | CD | PRO | 149 | 7.077 | 34.695 | −32.736 | 1.00 | 21.15 | C |
| ATOM | 1048 | CA | PRO | 149 | 5.163 | 36.003 | −33.446 | 1.00 | 21.27 | C |
| ATOM | 1049 | CB | PRO | 149 | 4.760 | 34.946 | −32.416 | 1.00 | 22.62 | C |
| ATOM | 1050 | CG | PRO | 149 | 5.807 | 33.894 | −32.568 | 1.00 | 23.25 | C |
| ATOM | 1051 | C | PRO | 149 | 4.794 | 37.413 | −32.979 | 1.00 | 21.78 | C |
| ATOM | 1052 | O | PRO | 149 | 5.242 | 37.852 | −31.920 | 1.00 | 19.81 | O |
| ATOM | 1053 | N | VAL | 150 | 3.984 | 38.116 | −33.771 | 1.00 | 19.75 | N |
| ATOM | 1054 | CA | VAL | 150 | 3.545 | 39.467 | −33.410 | 1.00 | 19.92 | C |
| ATOM | 1055 | CB | VAL | 150 | 3.870 | 40.485 | −34.515 | 1.00 | 19.06 | C |
| ATOM | 1056 | CG1 | VAL | 150 | 3.323 | 41.859 | −34.137 | 1.00 | 17.37 | C |
| ATOM | 1057 | CG2 | VAL | 150 | 5.389 | 40.563 | −34.725 | 1.00 | 20.06 | C |
| ATOM | 1058 | C | VAL | 150 | 2.033 | 39.439 | −33.170 | 1.00 | 20.76 | C |
| ATOM | 1059 | O | VAL | 150 | 1.268 | 39.011 | −34.037 | 1.00 | 20.27 | O |
| ATOM | 1060 | N | PHE | 151 | 1.605 | 39.900 | −31.996 | 1.00 | 20.33 | N |
| ATOM | 1061 | CA | PHE | 151 | 0.185 | 39.880 | −31.651 | 1.00 | 22.41 | C |
| ATOM | 1062 | CB | PHE | 151 | 0.009 | 39.471 | −30.176 | 1.00 | 22.53 | C |
| ATOM | 1063 | CG | PHE | 151 | 0.625 | 38.140 | −29.831 | 1.00 | 23.14 | C |
| ATOM | 1064 | CD1 | PHE | 151 | 2.000 | 38.012 | −29.678 | 1.00 | 21.63 | C |
| ATOM | 1065 | CD2 | PHE | 151 | −0.172 | 37.012 | −29.668 | 1.00 | 22.87 | C |
| ATOM | 1066 | CE1 | PHE | 151 | 2.575 | 36.776 | −29.365 | 1.00 | 23.30 | C |
| ATOM | 1067 | CE2 | PHE | 151 | 0.393 | 35.771 | −29.354 | 1.00 | 22.95 | C |
| ATOM | 1068 | CZ | PHE | 151 | 1.769 | 35.655 | −29.203 | 1.00 | 21.34 | C |
| ATOM | 1069 | C | PHE | 151 | −0.555 | 41.202 | −31.893 | 1.00 | 23.09 | C |
| ATOM | 1070 | O | PHE | 151 | −0.017 | 42.285 | −31.652 | 1.00 | 24.09 | O |
| ATOM | 1071 | N | THR | 152 | −1.786 | 41.097 | −32.390 | 1.00 | 23.44 | N |
| ATOM | 1072 | CA | THR | 152 | −2.648 | 42.259 | −32.634 | 1.00 | 24.04 | C |
| ATOM | 1073 | CB | THR | 152 | −2.596 | 42.755 | −34.091 | 1.00 | 23.18 | C |
| ATOM | 1074 | OG1 | THR | 152 | −3.228 | 41.781 | −34.935 | 1.00 | 23.10 | O |
| ATOM | 1075 | CG2 | THR | 152 | −1.159 | 42.995 | −34.537 | 1.00 | 23.92 | C |
| ATOM | 1076 | C | THR | 152 | −4.072 | 41.778 | −32.398 | 1.00 | 23.73 | C |
| ATOM | 1077 | O | THR | 152 | −4.289 | 40.611 | −32.092 | 1.00 | 23.51 | O |
| ATOM | 1078 | N | ASP | 153 | −5.048 | 42.665 | −32.561 | 1.00 | 27.44 | N |
| ATOM | 1079 | CA | ASP | 153 | −6.437 | 42.268 | −32.370 | 1.00 | 29.62 | C |
| ATOM | 1080 | CB | ASP | 153 | −7.341 | 43.503 | −32.292 | 1.00 | 33.87 | C |
| ATOM | 1081 | CG | ASP | 153 | −7.091 | 44.324 | −31.045 | 1.00 | 37.22 | C |
| ATOM | 1082 | OD1 | ASP | 153 | −7.003 | 43.724 | −29.956 | 1.00 | 38.69 | O |
| ATOM | 1083 | OD2 | ASP | 153 | −6.988 | 45.565 | −31.147 | 1.00 | 40.78 | O |
| ATOM | 1084 | C | ASP | 153 | −6.929 | 41.335 | −33.473 | 1.00 | 29.34 | C |
| ATOM | 1085 | O | ASP | 153 | −7.957 | 40.682 | −33.316 | 1.00 | 30.86 | O |
| ATOM | 1086 | N | SER | 154 | −6.200 | 41.276 | −34.588 | 1.00 | 27.23 | N |
| ATOM | 1087 | CA | SER | 154 | −6.575 | 40.406 | −35.702 | 1.00 | 25.69 | C |
| ATOM | 1088 | CB | SER | 154 | −5.897 | 40.854 | −37.005 | 1.00 | 26.20 | C |
| ATOM | 1089 | OG | SER | 154 | −6.144 | 42.210 | −37.294 | 1.00 | 28.56 | O |
| ATOM | 1090 | C | SER | 154 | −6.171 | 38.960 | −35.437 | 1.00 | 24.84 | C |
| ATOM | 1091 | O | SER | 154 | −6.858 | 38.028 | −35.854 | 1.00 | 22.64 | O |
| ATOM | 1092 | N | GLY | 155 | −5.043 | 38.784 | −34.750 | 1.00 | 23.43 | N |
| ATOM | 1093 | CA | GLY | 155 | −4.549 | 37.449 | −34.462 | 1.00 | 22.29 | C |
| ATOM | 1094 | C | GLY | 155 | −3.046 | 37.480 | −34.240 | 1.00 | 21.28 | C |
| ATOM | 1095 | O | GLY | 155 | −2.523 | 38.419 | −33.639 | 1.00 | 22.08 | O |
| ATOM | 1096 | N | ILE | 156 | −2.350 | 36.458 | −34.728 | 1.00 | 20.17 | N |
| ATOM | 1097 | CA | ILE | 156 | −0.902 | 36.373 | −34.575 | 1.00 | 18.79 | C |
| ATOM | 1098 | CB | ILE | 156 | −0.507 | 35.048 | −33.896 | 1.00 | 19.07 | C |
| ATOM | 1099 | CG2 | ILE | 156 | 1.001 | 35.030 | −33.602 | 1.00 | 20.72 | C |
| ATOM | 1100 | CG1 | ILE | 156 | −1.304 | 34.881 | −32.594 | 1.00 | 18.77 | C |
| ATOM | 1101 | CD1 | ILE | 156 | −1.065 | 33.557 | −31.900 | 1.00 | 17.80 | C |
| ATOM | 1102 | C | ILE | 156 | −0.289 | 36.438 | −35.967 | 1.00 | 19.38 | C |
| ATOM | 1103 | O | ILE | 156 | −0.727 | 35.722 | 36.875 | 1.00 | 18.34 | O |
| ATOM | 1104 | N | HIS | 157 | 0.722 | 37.286 | −36.135 | 1.00 | 18.01 | N |
| ATOM | 1105 | CA | HIS | 157 | 1.356 | 37.451 | −37.438 | 1.00 | 19.24 | C |
| ATOM | 1106 | CB | HIS | 157 | 1.283 | 38.910 | −37.916 | 1.00 | 19.95 | C |
| ATOM | 1107 | CG | HIS | 157 | −0.031 | 39.587 | −37.688 | 1.00 | 21.88 | C |
| ATOM | 1108 | CD2 | HIS | 157 | −0.720 | 39.848 | −36.551 | 1.00 | 21.84 | C |
| ATOM | 1109 | ND1 | HIS | 157 | −0.733 | 40.190 | −38.708 | 1.00 | 20.39 | N |
| ATOM | 1110 | CE1 | HIS | 157 | −1.795 | 40.799 | −38.211 | 1.00 | 23.64 | C |
| ATOM | 1111 | NE2 | HIS | 157 | −1.811 | 40.607 | −36.904 | 1.00 | 21.95 | N |
| ATOM | 1112 | C | HIS | 157 | 2.839 | 37.102 | −37.484 | 1.00 | 19.21 | C |
| ATOM | 1113 | O | HIS | 157 | 3.541 | 37.156 | −36.475 | 1.00 | 19.34 | O |
| ATOM | 1114 | N | ILE | 158 | 3.288 | 36.751 | −38.686 | 1.00 | 18.54 | N |
| ATOM | 1115 | CA | ILE | 158 | 4.688 | 36.511 | −38.975 | 1.00 | 17.73 | C |
| ATOM | 1116 | CB | ILE | 158 | 4.960 | 35.163 | −39.644 | 1.00 | 19.32 | C |
| ATOM | 1117 | CG2 | ILE | 158 | 6.413 | 35.120 | −40.090 | 1.00 | 20.23 | C |
| ATOM | 1118 | CG1 | ILE | 158 | 4.689 | 34.016 | −38.668 | 1.00 | 19.48 | C |
| ATOM | 1119 | CD1 | ILE | 158 | 4.783 | 32.633 | −39.314 | 1.00 | 20.27 | C |
| ATOM | 1120 | C | ILE | 158 | 4.852 | 37.615 | −40.016 | 1.00 | 18.12 | C |
| ATOM | 1121 | O | ILE | 158 | 4.118 | 37.649 | −41.013 | 1.00 | 18.44 | O |
| ATOM | 1122 | N | ILE | 159 | 5.800 | 38.513 | −39.784 | 1.00 | 17.05 | N |
| ATOM | 1123 | CA | ILE | 159 | 6.003 | 39.651 | −40.670 | 1.00 | 18.79 | C |
| ATOM | 1124 | CB | ILE | 159 | 5.913 | 40.975 | −39.870 | 1.00 | 18.39 | C |
| ATOM | 1125 | CG2 | ILE | 159 | 6.015 | 42.170 | −40.815 | 1.00 | 19.94 | C |
| ATOM | 1126 | CG1 | ILE | 159 | 4.610 | 41.001 | −39.062 | 1.00 | 20.68 | C |
| ATOM | 1127 | CD1 | ILE | 159 | 4.461 | 42.216 | −38.129 | 1.00 | 17.02 | C |
| ATOM | 1128 | C | ILE | 159 | 7.328 | 39.642 | −41.417 | 1.00 | 18.45 | C |
| ATOM | 1129 | O | ILE | 159 | 8.383 | 39.418 | −40.831 | 1.00 | 20.19 | O |
| ATOM | 1130 | N | LEU | 160 | 7.263 | 39.886 | −42.720 | 1.00 | 18.39 | N |
| ATOM | 1131 | CA | LEU | 160 | 8.476 | 39.947 | −43.528 | 1.00 | 18.91 | C |
| ATOM | 1132 | CB | LEU | 160 | 8.453 | 38.901 | −44.650 | 1.00 | 16.90 | C |
| ATOM | 1133 | CG | LEU | 160 | 9.663 | 38.977 | −45.592 | 1.00 | 18.75 | C |
| ATOM | 1134 | CD1 | LEU | 160 | 10.915 | 38.508 | −44.852 | 1.00 | 19.35 | C |
| ATOM | 1135 | CD2 | LEU | 160 | 9.436 | 38.117 | −46.829 | 1.00 | 17.69 | C |
| ATOM | 1136 | C | LEU | 160 | 8.566 | 41.333 | −44.140 | 1.00 | 19.09 | C |
| ATOM | 1137 | O | LEU | 160 | 7.689 | 41.736 | −44.901 | 1.00 | 20.34 | O |
| ATOM | 1138 | N | ARG | 161 | 9.616 | 42.068 | −43.792 | 1.00 | 21.02 | N |
| ATOM | 1139 | CA | ARG | 161 | 9.808 | 43.397 | −44.346 | 1.00 | 22.02 | C |
| ATOM | 1140 | CB | ARG | 161 | 10.633 | 44.282 | −43.412 | 1.00 | 21.72 | C |
| ATOM | 1141 | CG | ARG | 161 | 10.864 | 45.646 | −44.032 | 1.00 | 24.85 | C |
| ATOM | 1142 | CD | ARG | 161 | 11.712 | 46.578 | −43.197 | 1.00 | 27.39 | C |
| ATOM | 1143 | NE | ARG | 161 | 12.036 | 47.761 | −43.992 | 1.00 | 27.33 | N |
| ATOM | 1144 | CZ | ARG | 161 | 12.907 | 48.695 | −43.634 | 1.00 | 29.68 | C |
| ATOM | 1145 | NH1 | ARG | 161 | 13.551 | 48.596 | −42.478 | 1.00 | 28.60 | N |
| ATOM | 1146 | NH2 | ARG | 161 | 13.144 | 49.719 | −44.443 | 1.00 | 27.90 | N |
| ATOM | 1147 | C | ARG | 161 | 10.538 | 43.236 | −45.673 | 1.00 | 21.63 | C |
| ATOM | 1148 | O | ARG | 161 | 11.654 | 42.729 | −45.714 | 1.00 | 21.71 | O |
| ATOM | 1149 | N | THR | 162 | 9.905 | 43.676 | −46.751 | 1.00 | 20.78 | N |
| ATOM | 1150 | CA | THR | 162 | 10.487 | 43.541 | −48.077 | 1.00 | 22.60 | C |
| ATOM | 1151 | CB | THR | 162 | 9.421 | 43.066 | −49.085 | 1.00 | 22.27 | C |
| ATOM | 1152 | OG1 | THR | 162 | 8.274 | 43.921 | −49.013 | 1.00 | 22.96 | O |
| ATOM | 1153 | CG2 | THR | 162 | 8.992 | 41.636 | −48.764 | 1.00 | 22.61 | C |
| ATOM | 1154 | C | THR | 162 | 11.146 | 44.814 | −48.591 | 1.00 | 23.00 | C |
| ATOM | 1155 | O | THR | 162 | 12.029 | 44.753 | −49.441 | 1.00 | 22.65 | O |
| ATOM | 1156 | N | GLU | 163 | 10.718 | 45.961 | −48.078 | 1.00 | 24.32 | N |
| ATOM | 1157 | CA | GLU | 163 | 11.284 | 47.242 | −48.490 | 1.00 | 26.46 | C |
| ATOM | 1158 | CB | GLU | 163 | 10.445 | 47.850 | −49.620 | 1.00 | 28.32 | C |
| ATOM | 1159 | CG | GLU | 163 | 10.507 | 47.101 | −50.943 | 1.00 | 30.56 | C |
| ATOM | 1160 | CD | GLU | 163 | 9.525 | 47.652 | −51.969 | 1.00 | 32.59 | C |
| ATOM | 1161 | OE1 | GLU | 163 | 9.172 | 48.850 | −51.881 | 1.00 | 33.11 | O |
| ATOM | 1162 | OE2 | GLU | 163 | 9.115 | 46.891 | −52.870 | 1.00 | 33.50 | O |
| ATOM | 1163 | C | GLU | 163 | 11.304 | 48.214 | −47.311 | 1.00 | 27.33 | C |
| ATOM | 1164 | O | GLU | 163 | 10.445 | 48.066 | −46.416 | 1.00 | 28.13 | O |
| ATOM | 1165 | OXT | GLU | 163 | 12.156 | 49.128 | −47.312 | 1.00 | 27.53 | O |
| ATOM | 1166 | OH2 | WAT S | 1 | 7.893 | 41.331 | −27.148 | 1.00 | 20.05 | O |
| ATOM | 1167 | OH2 | WAT S | 2 | 17.346 | 30.491 | −41.153 | 1.00 | 19.42 | O |
| ATOM | 1168 | OH2 | WAT S | 3 | −4.284 | 32.127 | −51.291 | 1.00 | 20.44 | O |
| ATOM | 1169 | OH2 | WAT S | 4 | −1.352 | 36.952 | −45.720 | 1.00 | 20.58 | O |
| ATOM | 1170 | OH2 | WAT S | 5 | 2.568 | 25.068 | −38.010 | 1.00 | 22.22 | O |
| ATOM | 1171 | OH2 | WAT S | 6 | 15.546 | 44.418 | −36.331 | 1.00 | 22.22 | O |
| ATOM | 1172 | OH2 | WAT S | 7 | 6.032 | 42.545 | −49.458 | 1.00 | 21.44 | O |
| ATOM | 1173 | OH2 | WAT S | 8 | 13.801 | 42.245 | −35.291 | 1.00 | 26.16 | O |
| ATOM | 1174 | OH2 | WAT S | 9 | −0.917 | 36.386 | −56.614 | 1.00 | 22.75 | O |
| ATOM | 1175 | OH2 | WAT S | 10 | 9.461 | 29.022 | −34.080 | 1.00 | 23.06 | O |
| ATOM | 1176 | OH2 | WAT S | 11 | −0.374 | 33.895 | −53.631 | 1.00 | 22.72 | O |
| ATOM | 1177 | OH2 | WAT S | 12 | 1.353 | 54.881 | −34.353 | 1.00 | 29.58 | O |
| ATOM | 1178 | OH2 | WAT S | 13 | −10.315 | 27.273 | −36.239 | 1.00 | 27.61 | O |
| ATOM | 1179 | OH2 | WAT S | 14 | −3.068 | 27.728 | −39.878 | 1.00 | 22.17 | O |
| ATOM | 1180 | OH2 | WAT S | 15 | 20.327 | 33.128 | −36.148 | 1.00 | 23.99 | O |
| ATOM | 1181 | OH2 | WAT S | 16 | 14.045 | 43.523 | −21.728 | 1.00 | 29.71 | O |
| ATOM | 1182 | OH2 | WAT S | 17 | −3.857 | 45.355 | −32.255 | 1.00 | 29.16 | O |
| ATOM | 1183 | OH2 | WAT S | 18 | 1.002 | 51.896 | −39.707 | 1.00 | 38.48 | O |
| ATOM | 1184 | OH2 | WAT S | 19 | 12.908 | 30.336 | −46.148 | 1.00 | 22.55 | O |
| ATOM | 1185 | OH2 | WAT S | 20 | 16.527 | 38.495 | −36.902 | 1.00 | 27.58 | O |
| ATOM | 1186 | OH2 | WAT S | 21 | −2.331 | 24.038 | −48.848 | 1.00 | 32.35 | O |
| ATOM | 1187 | OH2 | WAT S | 22 | 0.917 | 34.371 | −56.164 | 1.00 | 27.90 | O |
| ATOM | 1188 | OH2 | WAT S | 23 | 4.931 | 38.281 | −50.188 | 1.00 | 28.56 | O |
| ATOM | 1189 | OH2 | WAT S | 24 | 13.874 | 36.942 | −43.530 | 1.00 | 26.39 | O |
| ATOM | 1190 | OH2 | WAT S | 25 | 17.618 | 32.933 | −36.603 | 1.00 | 30.83 | O |
| ATOM | 1191 | OH2 | WAT S | 26 | −8.129 | 29.096 | −40.568 | 1.00 | 27.75 | O |
| ATOM | 1192 | OH2 | WAT S | 27 | −3.679 | 37.727 | −30.843 | 1.00 | 31.15 | O |
| ATOM | 1193 | OH2 | WAT S | 28 | 9.282 | 50.419 | −34.337 | 1.00 | 30.75 | O |
| ATOM | 1194 | OH2 | WAT S | 29 | 27.012 | 23.613 | −24.655 | 1.00 | 29.62 | O |
| ATOM | 1195 | OH2 | WAT S | 30 | −4.557 | 29.710 | −40.751 | 1.00 | 28.97 | O |
| ATOM | 1196 | OH2 | WAT S | 31 | −0.997 | 24.226 | −41.494 | 1.00 | 33.22 | O |
| ATOM | 1197 | OH2 | WAT S | 32 | 16.353 | 29.577 | −24.662 | 1.00 | 31.58 | O |
| ATOM | 1198 | OH2 | WAT S | 33 | 25.743 | 33.873 | −30.490 | 1.00 | 25.85 | O |
| ATOM | 1199 | OH2 | WAT S | 34 | 5.332 | 45.143 | −31.658 | 1.00 | 30.16 | O |
| ATOM | 1200 | OH2 | WAT S | 35 | 14.920 | 35.643 | −24.808 | 1.00 | 33.85 | O |
| ATOM | 1201 | OH2 | WAT S | 36 | 15.290 | 31.392 | −31.500 | 1.00 | 27.63 | O |
| ATOM | 1202 | OH2 | WAT S | 37 | −6.894 | 26.860 | −47.045 | 1.00 | 33.52 | O |
| ATOM | 1203 | OH2 | WAT S | 38 | −7.398 | 46.846 | −28.941 | 1.00 | 51.90 | O |
| ATOM | 1204 | OH2 | WAT S | 39 | 10.653 | 22.662 | −39.314 | 1.00 | 45.55 | O |
| ATOM | 1205 | OH2 | WAT S | 40 | 18.244 | 24.600 | −19.261 | 1.00 | 38.82 | O |
| ATOM | 1206 | OH2 | WAT S | 41 | −15.687 | 27.400 | −43.454 | 1.00 | 34.30 | O |
| ATOM | 1207 | OH2 | WAT S | 42 | −18.597 | 27.618 | −44.427 | 1.00 | 32.89 | O |
| ATOM | 1208 | OH2 | WAT S | 43 | 6.049 | 37.358 | −53.977 | 1.00 | 36.68 | O |
| ATOM | 1209 | OH2 | WAT S | 44 | 19.052 | 43.889 | −43.831 | 1.00 | 34.72 | O |
| ATOM | 1210 | OH2 | WAT S | 45 | 15.461 | 47.913 | −34.992 | 1.00 | 31.23 | O |
| ATOM | 1211 | OH2 | WAT S | 46 | 20.837 | 37.225 | −38.207 | 1.00 | 28.28 | O |
| ATOM | 1212 | OH2 | WAT S | 47 | 19.507 | 45.526 | −23.866 | 1.00 | 47.05 | O |
| ATOM | 1213 | OH2 | WAT S | 48 | 17.325 | 48.028 | −43.296 | 1.00 | 48.61 | O |
| ATOM | 1214 | OH2 | WAT S | 49 | 17.907 | 34.411 | −21.643 | 1.00 | 38.49 | O |
| ATOM | 1215 | OH2 | WAT S | 50 | 5.070 | 47.615 | −29.795 | 1.00 | 38.43 | O |
| ATOM | 1216 | OH2 | WAT S | 51 | −8.466 | 32.182 | −28.123 | 1.00 | 37.74 | O |
| ATOM | 1217 | OH2 | WAT S | 52 | 11.537 | 34.750 | −30.892 | 1.00 | 38.98 | O |
| ATOM | 1218 | 01 | PGA B | 180 | 16.565 | 30.648 | −29.156 | 1.00 | 34.95 | O |
| ATOM | 1219 | C2 | PGA B | 180 | 17.916 | 30.499 | −29.151 | 1.00 | 30.97 | C |
| ATOM | 1220 | C3 | PGA B | 180 | 18.549 | 31.257 | −30.326 | 1.00 | 29.19 | C |
| ATOM | 1221 | O4 | PGA B | 180 | 18.179 | 30.717 | −31.480 | 1.00 | 27.98 | O |
| ATOM | 1222 | C5 | PGA B | 180 | 18.543 | 31.454 | −32.527 | 1.00 | 24.65 | C |
| ATOM | 1223 | C6 | PGA B | 180 | 18.061 | 30.765 | −33.815 | 1.00 | 27.19 | C |
| ATOM | 1224 | O7 | PGA B | 180 | 16.757 | 30.428 | −33.686 | 1.00 | 28.41 | O |
| ATOM | 1225 | C8 | PGA B | 180 | 16.320 | 29.679 | −34.708 | 1.00 | 26.90 | C |
| ATOM | 1226 | C9 | PGA B | 180 | 14.835 | 29.337 | −34.488 | 1.00 | 27.50 | C |
| ATOM | 1227 | O10 | PGA B | 180 | 14.108 | 30.481 | −34.359 | 1.00 | 28.64 | O |
| ATOM | 1228 | C11 | PGA B | 180 | 12.816 | 30.227 | −34.105 | 1.00 | 27.73 | C |
| ATOM | 1229 | C12 | PGA B | 180 | 12.129 | 31.509 | −33.596 | 1.00 | 29.25 | C |
| ATOM | 1230 | O13 | PGA B | 180 | 12.280 | 32.504 | −34.479 | 1.00 | 27.90 | O |
| ATOM | 1231 | C14 | PGA B | 180 | 11.635 | 33.590 | −34.048 | 1.00 | 30.91 | C |
| ATOM | 1232 | C15 | PGA B | 180 | 11.739 | 34.725 | −35.087 | 1.00 | 30.86 | C |
| ATOM | 1233 | O16 | PGA B | 180 | 13.005 | 35.098 | −35.301 | 1.00 | 28.94 | O |
| ATOM | 1234 | C17 | PGA B | 180 | 13.004 | 35.725 | −36.434 | 1.00 | 35.98 | C |
| ATOM | 1235 | C18 | PGA B | 180 | 14.387 | 36.187 | −36.862 | 1.00 | 36.33 | C |
| ATOM | 1236 | O19 | PGA B | 180 | 14.291 | 36.621 | −38.093 | 1.00 | 34.83 | O |
| ATOM | 1237 | O1 | PGB C | 181 | −2.961 | 45.735 | −38.887 | 1.00 | 35.85 | O |
| ATOM | 1238 | C2 | PGB C | 181 | −2.391 | 44.535 | −38.920 | 1.00 | 38.14 | C |
| ATOM | 1239 | C3 | PGB C | 181 | −2.092 | 44.102 | −40.376 | 1.00 | 37.17 | C |
| ATOM | 1240 | O4 | PGB C | 181 | −3.218 | 43.989 | −41.089 | 1.00 | 39.98 | O |
| ATOM | 1241 | C5 | PGB C | 181 | −2.951 | 43.671 | −42.381 | 1.00 | 41.36 | C |
| ATOM | 1242 | C6 | PGB C | 181 | −4.245 | 43.666 | −43.231 | 1.00 | 43.82 | C |
| ATOM | 1243 | O7 | PGB C | 181 | −4.709 | 44.927 | −43.388 | 1.00 | 45.09 | O |
| ATOM | 1244 | S | SO4 | 190 | −9.615 | 41.396 | −38.442 | 1.00 | 58.70 | S |
| ATOM | 1245 | O1 | SO4 | 190 | −9.696 | 40.211 | −37.557 | 1.00 | 58.17 | O |
| ATOM | 1246 | O2 | SO4 | 190 | −8.575 | 42.323 | −37.939 | 1.00 | 57.55 | O |
| ATOM | 1247 | O3 | SO4 | 190 | −10.923 | 42.101 | −38.448 | 1.00 | 58.85 | O |
| ATOM | 1248 | O4 | SO4 | 190 | −9.294 | 40.994 | −39.836 | 1.00 | 57.04 | O |
| END | | | | | | | | | | |

### Example 2

### Crystallization and Structural Determination of pPin1(71) R14A

Pin1 R14A was made as described above. 100 uM Pin1 R14A was treated with 0.27 uM PKA in 50 mM Trip-HCl (pH 7.5), 10 mM MgCl₂, 5 mM DTT, and 5 mM ATP, overnight at 30° C. Crystals were grown by the hanging drop method screening 1.8 to 2.4 M ammonium sulfate and pH 7.3-8.0 in standard Linbro style plates. The resulting crystals diffracted to ˜2.0 angstroms. Analysis of the crystal structure indicated that Serine 71 was phosphorylated. The crystal structure coordinates for pPin1(71) R14A are presented in Table II.

TABLE II
Set of Atomic Coordinates for pPin1(71) R14A determined by X-ray Crystallography

| | | | | | | | | | | |
|---|---|---|---|---|---|---|---|---|---|---|
| ATOM | 1 | CB | LEU | 7 | 50.539 | 4.121 | −82.663 | 1.00 | 43.21 | 6 |
| ATOM | 2 | CG | LEU | 7 | 50.110 | 2.747 | −82.130 | 1.00 | 43.37 | 6 |
| ATOM | 3 | CD1 | LEU | 7 | 49.099 | 2.912 | −81.009 | 1.00 | 43.37 | 6 |
| ATOM | 4 | CD2 | LEU | 7 | 49.521 | 1.924 | −83.262 | 1.00 | 43.97 | 6 |
| ATOM | 5 | C | LEU | 7 | 51.846 | 5.572 | −84.226 | 1.00 | 42.29 | 6 |
| ATOM | 6 | O | LEU | 7 | 52.360 | 6.477 | −83.569 | 1.00 | 42.39 | 8 |
| ATOM | 7 | N | LEU | 7 | 52.973 | 3.747 | −82.956 | 1.00 | 43.33 | 7 |
| ATOM | 8 | CA | LEU | 7 | 51.717 | 4.164 | −83.646 | 1.00 | 42.84 | 6 |
| ATOM | 9 | N | PRO | 8 | 51.377 | 5.772 | −85.469 | 1.00 | 42.26 | 7 |
| ATOM | 10 | CD | PRO | 8 | 50.760 | 4.777 | −86.363 | 1.00 | 42.45 | 6 |
| ATOM | 11 | CA | PRO | 8 | 51.446 | 7.082 | −86.126 | 1.00 | 41.19 | 6 |
| ATOM | 12 | CB | PRO | 8 | 50.606 | 6.878 | −87.383 | 1.00 | 42.95 | 6 |
| ATOM | 13 | CG | PRO | 8 | 50.875 | 5.448 | −87.717 | 1.00 | 43.48 | 6 |
| ATOM | 14 | C | PRO | 8 | 50.901 | 8.196 | −85.234 | 1.00 | 39.64 | 6 |
| ATOM | 15 | O | PRO | 8 | 50.181 | 7.936 | −84.266 | 1.00 | 39.10 | 8 |
| ATOM | 16 | N | PRO | 9 | 51.234 | 9.454 | −85.556 | 1.00 | 38.35 | 7 |
| ATOM | 17 | CD | PRO | 9 | 52.004 | 9.890 | −86.735 | 1.00 | 37.71 | 6 |
| ATOM | 18 | CA | PRO | 9 | 50.782 | 10.610 | −84.779 | 1.00 | 37.19 | 6 |
| ATOM | 19 | CB | PRO | 9 | 51.139 | 11.788 | −85.681 | 1.00 | 37.46 | 6 |
| ATOM | 20 | CG | PRO | 9 | 52.376 | 11.305 | −86.369 | 1.00 | 37.68 | 6 |
| ATOM | 21 | C | PRO | 9 | 49.299 | 10.599 | −84.409 | 1.00 | 36.11 | 6 |
| ATOM | 22 | O | PRO | 9 | 48.427 | 10.563 | −85.282 | 1.00 | 34.50 | 8 |
| ATOM | 23 | N | GLY | 10 | 49.027 | 10.625 | −83.106 | 1.00 | 34.58 | 7 |
| ATOM | 24 | CA | GLY | 10 | 47.655 | 10.654 | −82.630 | 1.00 | 32.34 | 6 |
| ATOM | 25 | C | GLY | 10 | 46.967 | 9.338 | −82.308 | 1.00 | 30.19 | 6 |
| ATOM | 26 | O | GLY | 10 | 45.894 | 9.347 | −81.710 | 1.00 | 26.83 | 8 |
| ATOM | 27 | N | TRP | 11 | 47.566 | 8.211 | −82.686 | 1.00 | 30.16 | 7 |
| ATOM | 28 | CA | TRP | 11 | 46.948 | 6.908 | −82.431 | 1.00 | 30.58 | 6 |
| ATOM | 29 | CB | TRP | 11 | 47.403 | 5.886 | −83.476 | 1.00 | 27.72 | 6 |
| ATOM | 30 | CG | TRP | 11 | 46.890 | 6.143 | −84.853 | 1.00 | 24.71 | 6 |
| ATOM | 31 | CD2 | TRP | 11 | 45.605 | 5.777 | −85.371 | 1.00 | 24.26 | 6 |
| ATOM | 32 | CE2 | TRP | 11 | 45.566 | 6.191 | −86.722 | 1.00 | 23.81 | 6 |
| ATOM | 33 | CE3 | TRP | 11 | 44.483 | 5.140 | −84.825 | 1.00 | 21.69 | 6 |
| ATOM | 34 | CD1 | TRP | 11 | 47.555 | 6.751 | −85.876 | 1.00 | 25.23 | 6 |
| ATOM | 35 | NE1 | TRP | 11 | 46.768 | 6.782 | −87.003 | 1.00 | 23.94 | 7 |
| ATOM | 36 | CZ2 | TRP | 11 | 44.446 | 5.987 | −87.540 | 1.00 | 21.25 | 6 |
| ATOM | 37 | CZ3 | TRP | 11 | 43.368 | 4.938 | −85.637 | 1.00 | 21.42 | 6 |
| ATOM | 38 | CH2 | TRP | 11 | 43.361 | 5.361 | −86.981 | 1.00 | 21.06 | 6 |
| ATOM | 39 | C | TRP | 11 | 47.197 | 6.332 | −81.039 | 1.00 | 32.22 | 6 |
| ATOM | 40 | O | TRP | 11 | 48.250 | 6.543 | −80.440 | 1.00 | 33.25 | 8 |
| ATOM | 41 | N | GLU | 12 | 46.213 | 5.591 | −80.538 | 1.00 | 33.50 | 7 |
| ATOM | 42 | CA | GLU | 12 | 46.297 | 4.961 | −79.223 | 1.00 | 34.46 | 6 |
| ATOM | 43 | CB | GLU | 12 | 45.748 | 5.906 | −78.151 | 1.00 | 37.21 | 6 |
| ATOM | 44 | CG | GLU | 12 | 44.248 | 6.170 | −78.279 | 1.00 | 41.16 | 6 |
| ATOM | 45 | CD | GLU | 12 | 43.723 | 7.164 | −77.254 | 1.00 | 43.50 | 6 |
| ATOM | 46 | OE1 | GLU | 12 | 44.178 | 8.329 | −77.260 | 1.00 | 45.03 | 8 |
| ATOM | 47 | OE2 | GLU | 12 | 42.852 | 6.780 | −76.444 | 1.00 | 43.96 | 8 |
| ATOM | 48 | C | GLU | 12 | 45.468 | 3.674 | −79.229 | 1.00 | 34.51 | 6 |
| ATOM | 49 | O | GLU | 12 | 44.568 | 3.515 | −80.053 | 1.00 | 33.38 | 8 |
| ATOM | 50 | N | LYS | 13 | 45.771 | 2.759 | −78.313 | 1.00 | 34.64 | 7 |
| ATOM | 51 | CA | LYS | 13 | 45.022 | 1.513 | −78.223 | 1.00 | 35.73 | 6 |
| ATOM | 52 | CB | LYS | 13 | 45.885 | 0.386 | −77.649 | 1.00 | 38.68 | 6 |
| ATOM | 53 | CG | LYS | 13 | 46.835 | −0.255 | −78.643 | 1.00 | 42.64 | 6 |
| ATOM | 54 | CD | LYS | 13 | 47.533 | −1.455 | −78.012 | 1.00 | 46.05 | 6 |
| ATOM | 55 | CE | LYS | 13 | 48.483 | −2.133 | −78.987 | 1.00 | 48.15 | 6 |
| ATOM | 56 | NZ | LYS | 13 | 49.133 | −3.332 | −78.375 | 1.00 | 51.37 | 7 |
| ATOM | 57 | C | LYS | 13 | 43.815 | 1.722 | −77.328 | 1.00 | 34.11 | 6 |
| ATOM | 58 | O | LYS | 13 | 43.879 | 2.462 | −76.347 | 1.00 | 33.34 | 8 |
| ATOM | 59 | N | ALA | 14 | 42.710 | 1.075 | −77.674 | 1.00 | 32.41 | 7 |
| ATOM | 60 | CA | ALA | 14 | 41.494 | 1.191 | −76.887 | 1.00 | 31.63 | 6 |
| ATOM | 61 | CE | ALA | 14 | 40.525 | 2.157 | −77.560 | 1.00 | 30.83 | 6 |
| ATOM | 62 | C | ALA | 14 | 40.846 | −0.181 | −76.711 | 1.00 | 31.60 | 6 |
| ATOM | 63 | O | ALA | 14 | 41.230 | −1.154 | −77.365 | 1.00 | 29.64 | 8 |
| ATOM | 64 | N | MET | 15 | 39.860 | −0.246 | −75.825 | 1.00 | 32.04 | 7 |
| ATOM | 65 | CA | MET | 15 | 39.154 | −1.491 | −75.544 | 1.00 | 32.58 | 6 |
| ATOM | 66 | CE | MET | 15 | 39.290 | −1.837 | −74.056 | 1.00 | 34.01 | 6 |
| ATOM | 67 | CG | MET | 15 | 38.477 | −3.041 | −73.602 | 1.00 | 36.32 | 6 |
| ATOM | 68 | SD | MET | 15 | 38.881 | −4.514 | −74.547 | 1.00 | 37.45 | 16 |
| ATOM | 69 | CE | MET | 15 | 40.498 | −4.913 | −73.856 | 1.00 | 35.82 | 6 |
| ATOM | 70 | C | MET | 15 | 37.681 | −1.382 | −75.913 | 1.00 | 31.32 | 6 |
| ATOM | 71 | O | MET | 15 | 36.994 | −0.458 | −75.486 | 1.00 | 29.65 | 8 |
| ATOM | 72 | N | SER | 16 | 37.198 | −2.327 | −76.711 | 1.00 | 30.69 | 7 |
| ATOM | 73 | CA | SER | 16 | 35.797 | −2.329 | −77.110 | 1.00 | 31.21 | 6 |
| ATOM | 74 | CB | SER | 16 | 35.591 | −3.271 | −78.296 | 1.00 | 31.40 | 6 |
| ATOM | 75 | OG | SER | 16 | 34.215 | −3.387 | −78.604 | 1.00 | 33.42 | 8 |
| ATOM | 76 | C | SER | 16 | 34.917 | −2.782 | −75.945 | 1.00 | 30.62 | 6 |
| ATOM | 77 | O | SER | 16 | 35.158 | −3.837 | −75.361 | 1.00 | 29.54 | 8 |
| ATOM | 78 | N | ARG | 17 | 33.900 | −1.989 | −75.614 | 1.00 | 30.19 | 7 |
| ATOM | 79 | CA | ARG | 17 | 32.991 | −2.327 | −74.517 | 1.00 | 31.67 | 6 |
| ATOM | 80 | CE | ARG | 17 | 32.121 | −1.130 | −74.126 | 1.00 | 30.99 | 6 |
| ATOM | 81 | CG | ARG | 17 | 32.776 | −0.079 | −73.260 | 1.00 | 32.57 | 6 |
| ATOM | 82 | CD | ARG | 17 | 31.691 | 0.734 | −72.561 | 1.00 | 32.49 | 6 |
| ATOM | 83 | NE | ARG | 17 | 32.221 | 1.908 | −71.881 | 1.00 | 32.24 | 7 |
| ATOM | 84 | CZ | ARG | 17 | 31.509 | 2.677 | −71.063 | 1.00 | 34.01 | 6 |
| ATOM | 85 | NH1 | ARG | 17 | 30.235 | 2.391 | −70.821 | 1.00 | 32.33 | 7 |
| ATOM | 86 | NH2 | ARG | 17 | 32.071 | 3.733 | −70.488 | 1.00 | 33.01 | 7 |
| ATOM | 87 | C | ARG | 17 | 32.046 | −3.482 | −74.834 | 1.00 | 32.23 | 6 |
| ATOM | 88 | O | ARG | 17 | 31.614 | −4.200 | −73.935 | 1.00 | 31.89 | 8 |
| ATOM | 89 | N | SER | 18 | 31.708 | −3.653 | −76.106 | 1.00 | 33.57 | 7 |
| ATOM | 90 | CA | SER | 18 | 30.780 | −4.707 | −76.483 | 1.00 | 35.93 | 6 |
| ATOM | 91 | CE | SER | 18 | 29.882 | −4.227 | −77.630 | 1.00 | 36.87 | 6 |
| ATOM | 92 | OG | SER | 18 | 30.645 | −3.824 | −78.752 | 1.00 | 38.15 | 8 |
| ATOM | 93 | C | SER | 18 | 31.423 | −6.037 | −76.851 | 1.00 | 36.41 | 6 |
| ATOM | 94 | O | SER | 18 | 30.806 | −7.086 | −76.673 | 1.00 | 37.63 | 8 |
| ATOM | 95 | N | SER | 19 | 32.659 | −6.001 | −77.344 | 1.00 | 36.71 | 7 |
| ATOM | 96 | CA | SER | 19 | 33.344 | −7.228 | −77.742 | 1.00 | 36.38 | 6 |
| ATOM | 97 | CB | SER | 19 | 33.773 | −7.145 | −79.211 | 1.00 | 38.09 | 6 |
| ATOM | 98 | OG | SER | 19 | 34.797 | −6.183 | −79.397 | 1.00 | 39.13 | 8 |
| ATOM | 99 | C | SER | 19 | 34.559 | −7.569 | −76.890 | 1.00 | 35.34 | 6 |
| ATOM | 100 | O | SER | 19 | 34.951 | −8.732 | −76.813 | 1.00 | 35.33 | 8 |
| ATOM | 101 | N | GLY | 20 | 35.153 | −6.563 | −76.253 | 1.00 | 34.55 | 7 |
| ATOM | 102 | CA | GLY | 20 | 36.324 | −6.806 | −75.427 | 1.00 | 32.06 | 6 |
| ATOM | 103 | C | GLY | 20 | 37.593 | −6.922 | −76.256 | 1.00 | 31.88 | 6 |
| ATOM | 104 | O | GLY | 20 | 38.667 | −7.233 | −75.745 | 1.00 | 31.58 | 8 |
| ATOM | 105 | N | ARG | 21 | 37.468 | −6.673 | −77.551 | 1.00 | 31.76 | 7 |
| ATOM | 106 | CA | ARG | 21 | 38.608 | −6.746 | −78.451 | 1.00 | 30.08 | 6 |
| ATOM | 107 | CB | ARG | 21 | 38.137 | −7.149 | −79.847 | 1.00 | 33.63 | 6 |
| ATOM | 108 | CG | ARG | 21 | 38.935 | −8.267 | −80.495 | 1.00 | 36.32 | 6 |
| ATOM | 109 | CD | ARG | 21 | 38.707 | −8.251 | −81.993 | 1.00 | 37.88 | 6 |
| ATOM | 110 | NE | ARG | 21 | 37.291 | −8.117 | −82.314 | 1.00 | 37.90 | 7 |
| ATOM | 111 | CZ | ARG | 21 | 36.827 | −7.580 | −83.436 | 1.00 | 38.76 | 6 |
| ATOM | 112 | NH1 | ARG | 21 | 37.666 | −7.120 | −84.357 | 1.00 | 37.32 | 7 |
| ATOM | 113 | NH2 | ARG | 21 | 35.519 | −7.496 | −83.633 | 1.00 | 40.68 | 7 |
| ATOM | 114 | C | ARG | 21 | 39.275 | −5.375 | −78.514 | 1.00 | 28.02 | 6 |
| ATOM | 115 | O | ARG | 21 | 38.605 | −4.346 | −78.432 | 1.00 | 26.19 | 8 |
| ATOM | 116 | N | VAL | 22 | 40.594 | −5.357 | −78.651 | 1.00 | 26.54 | 7 |
| ATOM | 117 | CA | VAL | 22 | 41.304 | −4.091 | −78.731 | 1.00 | 25.89 | 6 |
| ATOM | 118 | CB | VAL | 22 | 42.821 | −4.273 | −78.489 | 1.00 | 27.26 | 6 |
| ATOM | 119 | CG1 | VAL | 22 | 43.060 | −4.888 | −77.118 | 1.00 | 29.47 | 6 |
| ATOM | 120 | CG2 | VAL | 22 | 43.426 | −5.144 | −79.584 | 1.00 | 27.95 | 6 |
| ATOM | 121 | C | VAL | 22 | 41.111 | −3.463 | −80.111 | 1.00 | 24.77 | 6 |
| ATOM | 122 | O | VAL | 22 | 40.875 | −4.159 | −81.102 | 1.00 | 23.17 | 8 |
| ATOM | 123 | N | TYR | 23 | 41.186 | −2.139 | −80.162 | 1.00 | 23.75 | 7 |
| ATOM | 124 | CA | TYR | 23 | 41.068 | −1.421 | −81.420 | 1.00 | 23.01 | 6 |
| ATOM | 125 | CB | TYR | 23 | 39.609 | −1.070 | −81.720 | 1.00 | 23.14 | 6 |
| ATOM | 126 | CG | TYR | 23 | 38.981 | −0.007 | −80.846 | 1.00 | 25.25 | 6 |
| ATOM | 127 | CD1 | TYR | 23 | 38.919 | 1.322 | −81.267 | 1.00 | 25.38 | 6 |
| ATOM | 128 | CE1 | TYR | 23 | 38.268 | 2.293 | −80.505 | 1.00 | 26.94 | 6 |
| ATOM | 129 | CD2 | TYR | 23 | 38.38.5 | −0.342 | −79.631 | 1.00 | 26.41 | 6 |
| ATOM | 130 | CE2 | TYR | 23 | 37.734 | 0.617 | −78.859 | 1.00 | 27.89 | 6 |
| ATOM | 131 | CZ | TYR | 23 | 37.676 | 1.931 | −79.303 | 1.00 | 29.48 | 6 |
| ATOM | 132 | OH | TYR | 23 | 37.019 | 2.874 | −78.549 | 1.00 | 28.95 | 8 |
| ATOM | 133 | C | TYR | 23 | 41.922 | −0.173 | −81.304 | 1.00 | 23.33 | 6 |
| ATOM | 134 | O | TYR | 23 | 42.532 | 0.064 | −80.263 | 1.00 | 23.71 | 8 |
| ATOM | 135 | N | TYR | 24 | 41.978 | 0.618 | −82.369 | 1.00 | 22.97 | 7 |
| ATOM | 136 | CA | TYR | 24 | 42.794 | 1.816 | −82.359 | 1.00 | 22.90 | 6 |
| ATOM | 137 | CB | TYR | 24 | 43.935 | 1.660 | −83.366 | 1.00 | 25.52 | 6 |
| ATOM | 138 | CG | TYR | 24 | 44.764 | 0.416 | −83.109 | 1.00 | 28.80 | 6 |
| ATOM | 139 | CD1 | TYR | 24 | 44.315 | −0.844 | −83.514 | 1.00 | 31.92 | 6 |
| ATOM | 140 | CE1 | TYR | 24 | 45.044 | −1.999 | −83.222 | 1.00 | 34.13 | 6 |
| ATOM | 141 | CD2 | TYR | 24 | 45.965 | 0.491 | −82.405 | 1.00 | 30.94 | 6 |
| ATOM | 142 | CE2 | TYR | 24 | 46.700 | −0.655 | −82.106 | 1.00 | 34.30 | 6 |
| ATOM | 143 | CZ | TYR | 24 | 46.235 | −1.895 | −82.517 | 1.00 | 36.19 | 6 |
| ATOM | 144 | OH | TYR | 24 | 46.966 | −3.023 | −82.228 | 1.00 | 38.51 | 8 |
| ATOM | 145 | GC | TYR | 24 | 41.988 | 3.077 | −82.634 | 1.00 | 22.54 | 6 |
| ATOM | 146 | O | TYR | 24 | 41.127 | 3.106 | −83.513 | 1.00 | 21.12 | 8 |
| ATOM | 147 | N | PHE | 25 | 42.291 | 4.112 | −81.860 | 1.00 | 22.40 | 7 |
| ATOM | 148 | CA | PHE | 25 | 41.626 | 5.407 | −81.933 | 1.00 | 22.80 | 6 |
| ATOM | 149 | CB | PHE | 25 | 40.848 | 5.637 | −80.629 | 1.00 | 23.24 | 6 |
| ATOM | 150 | CG | PHE | 25 | 40.233 | 7.004 | −80.510 | 1.00 | 25.53 | 6 |
| ATOM | 151 | CD1 | PHE | 25 | 39.186 | 7.390 | −81.347 | 1.00 | 25.99 | 6 |
| ATOM | 152 | CD2 | PHE | 25 | 40.704 | 7.912 | −79.562 | 1.00 | 24.54 | 6 |
| ATOM | 153 | CE1 | PHE | 25 | 38.617 | 8.667 | −81.242 | 1.00 | 25.92 | 6 |
| ATOM | 154 | CE2 | PHE | 25 | 40.143 | 9.189 | −79.450 | 1.00 | 24.42 | 6 |
| ATOM | 155 | CZ | PHE | 25 | 39.098 | 9.565 | −80.293 | 1.00 | 25.62 | 6 |
| ATOM | 156 | C | PHE | 25 | 42.668 | 6.512 | −82.108 | 1.00 | 22.98 | 6 |
| ATOM | 157 | O | PHE | 25 | 43.728 | 6.475 | −81.488 | 1.00 | 23.70 | 8 |
| ATOM | 158 | N | ASN | 26 | 42.367 | 7.488 | −82.958 | 1.00 | 22.33 | 7 |
| ATOM | 159 | CA | ASN | 26 | 43.271 | 8.613 | −83.176 | 1.00 | 21.39 | 6 |
| ATOM | 160 | CE | ASN | 26 | 43.511 | 8.841 | −84.674 | 1.00 | 20.74 | 6 |
| ATOM | 161 | CG | ASN | 26 | 44.568 | 9.904 | −84.938 | 1.00 | 19.42 | 6 |
| ATOM | 162 | OD1 | ASN | 26 | 44.434 | 11.044 | −84.504 | 1.00 | 17.56 | 8 |
| ATOM | 163 | ND2 | ASN | 26 | 45.625 | 9.529 | −85.646 | 1.00 | 20.21 | 7 |
| ATOM | 164 | C | ASN | 26 | 42.576 | 9.822 | −82.558 | 1.00 | 19.94 | 6 |
| ATOM | 165 | O | ASN | 26 | 41.483 | 10.196 | −82.981 | 1.00 | 20.96 | 8 |
| ATOM | 166 | N | HIS | 27 | 43.198 | 10.426 | −81.551 | 1.00 | 18.97 | 7 |
| ATOM | 167 | CA | HIS | 27 | 42.586 | 11.568 | −80.878 | 1.00 | 19.60 | 6 |
| ATOM | 168 | CB | HIS | 27 | 43.140 | 11.705 | −79.452 | 1.00 | 20.60 | 6 |
| ATOM | 169 | CG | HIS | 27 | 44.618 | 11.932 | −79.390 | 1.00 | 22.29 | 6 |
| ATOM | 170 | CD2 | HIS | 27 | 45.633 | 11.099 | −79.059 | 1.00 | 23.22 | 6 |
| ATOM | 171 | ND1 | HIS | 27 | 45.202 | 13.142 | −79.699 | 1.00 | 26.40 | 7 |
| ATOM | 172 | CE1 | HIS | 27 | 46.513 | 13.045 | −79.561 | 1.00 | 24.70 | 6 |
| ATOM | 173 | NE2 | HIS | 27 | 46.800 | 11.815 | −79.175 | 1.00 | 24.97 | 7 |
| ATOM | 174 | C | HIS | 27 | 42.725 | 12.889 | −81.626 | 1.00 | 18.91 | 6 |
| ATOM | 175 | O | HIS | 27 | 42.282 | 13.928 | −81.144 | 1.00 | 18.14 | 8 |
| ATOM | 176 | N | ILE | 28 | 43.334 | 12.849 | −82.805 | 1.00 | 19.29 | 7 |
| ATOM | 177 | CA | ILE | 28 | 43.488 | 14.058 | −83.604 | 1.00 | 21.59 | 6 |
| ATOM | 178 | CE | ILE | 28 | 44.905 | 14.164 | −84.207 | 1.00 | 20.91 | 6 |
| ATOM | 179 | CG2 | ILE | 28 | 45.011 | 15.423 | −85.061 | 1.00 | 21.78 | 6 |
| ATOM | 180 | CG1 | ILE | 28 | 45.948 | 14.206 | −83.088 | 1.00 | 22.35 | 6 |
| ATOM | 181 | CD1 | ILE | 28 | 47.388 | 14.248 | −83.582 | 1.00 | 22.58 | 6 |
| ATOM | 182 | C | ILE | 28 | 42.462 | 14.081 | −84.739 | 1.00 | 22.13 | 6 |
| ATOM | 183 | O | ILE | 28 | 41.878 | 15.121 | −85.034 | 1.00 | 23.98 | 8 |
| ATOM | 184 | N | THR | 29 | 42.237 | 12.927 | −85.361 | 1.00 | 21.97 | 7 |
| ATOM | 185 | CA | THR | 29 | 41.288 | 12.816 | −86.470 | 1.00 | 19.30 | 6 |
| ATOM | 186 | CE | THR | 29 | 41.847 | 11.917 | −87.574 | 1.00 | 17.41 | 6 |
| ATOM | 187 | OG1 | THR | 29 | 41.941 | 10.578 | −87.077 | 1.00 | 16.71 | 8 |
| ATOM | 188 | CG2 | THR | 29 | 43.239 | 12.385 | −88.003 | 1.00 | 17.68 | 6 |
| ATOM | 189 | C | THR | 29 | 39.951 | 12.213 | −86.035 | 1.00 | 18.81 | 6 |
| ATOM | 190 | O | THR | 29 | 38.954 | 12.312 | −86.750 | 1.00 | 17.98 | 8 |
| ATOM | 191 | N | ASN | 30 | 39.953 | 11.591 | −84.858 | 1.00 | 19.49 | 7 |
| ATOM | 192 | CA | ASN | 30 | 38.786 | 10.918 | −84.295 | 1.00 | 18.49 | 6 |
| ATOM | 193 | CE | ASN | 30 | 37.582 | 11.870 | −84.195 | 1.00 | 17.67 | 6 |
| ATOM | 194 | CG | ASN | 30 | 37.699 | 12.833 | −83.006 | 1.00 | 20.97 | 6 |
| ATOM | 195 | OD1 | ASN | 30 | 38.253 | 12.478 | −81.967 | 1.00 | 17.98 | 8 |
| ATOM | 196 | ND2 | ASN | 30 | 37.168 | 14.043 | −83.156 | 1.00 | 15.85 | 7 |
| ATOM | 197 | C | ASN | 30 | 38.423 | 9.653 | −85.081 | 1.00 | 17.99 | 6 |
| ATOM | 198 | O | ASN | 30 | 37.294 | 9.160 | −85.027 | 1.00 | 17.07 | 8 |
| ATOM | 199 | N | ALA | 31 | 39.396 | 9.128 | −85.813 | 1.00 | 17.33 | 7 |
| ATOM | 200 | CA | ALA | 31 | 39.187 | 7.905 | −86.567 | 1.00 | 16.71 | 6 |
| ATOM | 201 | CB | ALA | 31 | 40.221 | 7.783 | −87.686 | 1.00 | 15.30 | 6 |
| ATOM | 202 | C | ALA | 31 | 39.341 | 6.742 | −85.597 | 1.00 | 17.98 | 6 |
| ATOM | 203 | O | ALA | 31 | 40.080 | 6.837 | −84.612 | 1.00 | 15.51 | 8 |
| ATOM | 204 | N | SER | 32 | 38.630 | 5.653 | −85.871 | 1.00 | 16.31 | 7 |
| ATOM | 205 | CA | SER | 32 | 38.716 | 4.453 | −85.047 | 1.00 | 18.49 | 6 |
| ATOM | 206 | CB | SER | 32 | 37.627 | 4.448 | −83.966 | 1.00 | 19.12 | 6 |
| ATOM | 207 | OG | SER | 32 | 36.342 | 4.653 | −84.524 | 1.00 | 25.15 | 8 |
| ATOM | 208 | C | SER | 32 | 38.577 | 3.243 | −85.965 | 1.00 | 20.30 | 6 |
| ATOM | 209 | O | SER | 32 | 37.735 | 3.226 | −86.867 | 1.00 | 18.31 | 8 |
| ATOM | 210 | N | GLN | 33 | 39.419 | 2.241 | −85.740 | 1.00 | 20.77 | 7 |
| ATOM | 211 | CA | GLN | 33 | 39.412 | 1.037 | −86.555 | 1.00 | 22.60 | 6 |
| ATOM | 212 | CB | GLN | 33 | 40.253 | 1.269 | −87.813 | 1.00 | 22.79 | 6 |
| ATOM | 213 | CG | GLN | 33 | 41.671 | 1.711 | −87.513 | 1.00 | 24.56 | 6 |
| ATOM | 214 | CD | GLN | 33 | 42.396 | 2.210 | −88.744 | 1.00 | 28.27 | 6 |
| ATOM | 215 | OE1 | GLN | 33 | 41.888 | 3.066 | −89.466 | 1.00 | 31.64 | 8 |
| ATOM | 216 | NE2 | GLN | 33 | 43.591 | 1.686 | −88.986 | 1.00 | 27.26 | 7 |
| ATOM | 217 | C | GLN | 33 | 39.982 | −0.136 | −85.771 | 1.00 | 23.15 | 6 |
| ATOM | 218 | O | GLN | 33 | 40.689 | 0.057 | −84.780 | 1.00 | 21.93 | 8 |
| ATOM | 219 | N | TRP | 34 | 39.669 | −1.347 | −86.220 | 1.00 | 22.75 | 7 |
| ATOM | 220 | CA | TRP | 34 | 40.163 | −2.561 | −85.574 | 1.00 | 24.17 | 6 |
| ATOM | 221 | CB | TRP | 34 | 39.369 | −3.785 | −86.046 | 1.00 | 20.24 | 6 |
| ATOM | 222 | CG | TRP | 34 | 37.953 | −3.775 | −85.598 | 1.00 | 19.54 | 6 |
| ATOM | 223 | CD2 | TRP | 34 | 37.487 | −3.836 | −84.247 | 1.00 | 19.93 | 6 |
| ATOM | 224 | CE2 | TRP | 34 | 36.079 | −3.778 | −84.289 | 1.00 | 20.50 | 6 |
| ATOM | 225 | CE3 | TRP | 34 | 38.125 | −3.936 | −83.002 | 1.00 | 19.92 | 6 |
| ATOM | 226 | CD1 | TRP | 34 | 36.845 | −3.686 | −86.384 | 1.00 | 18.77 | 6 |
| ATOM | 227 | NE1 | TRP | 34 | 35.713 | −3.687 | −85.607 | 1.00 | 18.87 | 7 |
| ATOM | 228 | CZ2 | TRP | 34 | 35.291 | −3.815 | −83.133 | 1.00 | 21.58 | 6 |
| ATOM | 229 | CZ3 | TRP | 34 | 37.345 | −3.972 | −81.853 | 1.00 | 20.76 | 6 |
| ATOM | 230 | CH2 | TRP | 34 | 35.942 | −3.911 | −81.926 | 1.00 | 22.43 | 6 |
| ATOM | 231 | C | TRP | 34 | 41.623 | −2.778 | −85.919 | 1.00 | 25.81 | 6 |
| ATOM | 232 | O | TRP | 34 | 42.443 | −3.093 | −85.056 | 1.00 | 28.16 | 8 |
| ATOM | 233 | N | GLU | 35 | 41.932 | −2.614 | −87.198 | 1.00 | 29.50 | 7 |
| ATOM | 234 | CA | GLU | 35 | 43.280 | −2.811 | −87.708 | 1.00 | 32.42 | 6 |
| ATOM | 235 | CB | GLU | 35 | 43.294 | −2.667 | −89.235 | 1.00 | 33.35 | 6 |
| ATOM | 236 | CG | GLU | 35 | 42.307 | −3.558 | −90.001 | 1.00 | 34.92 | 6 |
| ATOM | 237 | CD | GLU | 35 | 40.851 | −3.154 | −89.804 | 1.00 | 36.66 | 6 |
| ATOM | 238 | OE1 | GLU | 35 | 40.593 | −1.965 | −89.505 | 1.00 | 34.91 | 8 |
| ATOM | 239 | OE2 | GLU | 35 | 39.962 | −4.025 | −89.964 | 1.00 | 36.89 | 8 |
| ATOM | 240 | C | GLU | 35 | 44.262 | −1.819 | −87.109 | 1.00 | 35.15 | 6 |
| ATOM | 241 | O | GLU | 35 | 43.937 | −0.643 | −86.924 | 1.00 | 34.58 | 8 |
| ATOM | 242 | N | ARG | 36 | 45.464 | −2.296 | −86.805 | 1.00 | 38.04 | 7 |
| ATOM | 243 | CA | ARG | 36 | 46.490 | −1.428 | −86.251 | 1.00 | 43.02 | 6 |
| ATOM | 244 | CB | ARG | 36 | 47.682 | −2.253 | −85.762 | 1.00 | 45.56 | 6 |
| ATOM | 245 | CG | ARG | 36 | 48.689 | −1.450 | −84.954 | 1.00 | 49.44 | 6 |
| ATOM | 246 | CD | ARG | 36 | 49.538. | −2.349 | −84.066 | 1.00 | 53.30 | 6 |
| ATOM | 247 | NE | ARG | 36 | 50.330 | −1.579 | −83.108 | 1.00 | 55.68 | 7 |
| ATOM | 248 | CZ | ARG | 36 | 50.937 | −2.099 | −82.046 | 1.00 | 57.14 | 6 |
| ATOM | 249 | NH1 | ARG | 36 | 50.846 | −3.400 | −81.798 | 1.00 | 58.93 | 7 |
| ATOM | 250 | NH2 | ARG | 36 | 51.626 | −1.318 | −81.225 | 1.00 | 57.97 | 7 |
| ATOM | 251 | C | ARG | 36 | 46.911 | −0.490 | −87.380 | 1.00 | 44.48 | 6 |
| ATOM | 252 | O | ARG | 36 | 47.230 | −0.938 | −88.482 | 1.00 | 44.85 | 8 |
| ATOM | 253 | N | PRO | 37 | 46.906 | 0.827 | −87.122 | 1.00 | 45.69 | 7 |
| ATOM | 254 | CD | PRO | 37 | 46.716 | 1.458 | −85.804 | 1.00 | 45.71 | 6 |
| ATOM | 255 | CA | PRO | 37 | 47.280 | 1.831 | −88.124 | 1.00 | 47.17 | 6 |
| ATOM | 256 | CB | PRO | 37 | 47.122 | 3.146 | −87.364 | 1.00 | 46.51 | 6 |
| ATOM | 257 | CG | PRO | 37 | 47.462 | 2.759 | −85.966 | 1.00 | 45.30 | 6 |
| ATOM | 258 | C | PRO | 37 | 48.675 | 1.664 | −88.721 | 1.00 | 48.54 | 6 |
| ATOM | 259 | O | PRO | 37 | 49.634 | 1.348 | −88.015 | 1.00 | 48.56 | 8 |
| ATOM | 260 | N | SER | 38. | 48.774 | 1.881 | −90.031 | 1.00 | 50.83 | 7 |
| ATOM | 261 | CA | SER | 38. | 50.041 | 1.766 | −90.748 | 1.00 | 52.90 | 6 |
| ATOM | 262 | CB | SER | 38. | 49.871 | 2.223 | −92.202 | 1.00 | 53.37 | 6 |
| ATOM | 263 | OG | SER | 38. | 48.922 | 1.426 | −92.891 | 1.00 | 54.31 | 8 |
| ATOM | 264 | C | SER | 38. | 51.124 | 2.604 | −90.076 | 1.00 | 53.30 | 6 |
| ATOM | 265 | O | SER | 38. | 51.142 | 3.829 | −90.202 | 1.00 | 53.52 | 8 |
| ATOM | 266 | N | GLU | 51 | 56.421 | 16.026 | −92.161 | 1.00 | 51.12 | 7 |
| ATOM | 267 | CA | GLU | 51 | 55.092 | 15.803 | −92.715 | 1.00 | 50.77 | 6 |
| ATOM | 268 | CB | GLU | 51 | 54.020 | 16.235 | −91.709 | 1.00 | 50.71 | 6 |
| ATOM | 269 | CG | GLU | 51 | 53.902 | 17.741 | −91.554 | 1.00 | 49.20 | 6 |
| ATOM | 270 | CD | GLU | 51 | 52.777 | 18.152 | −90.627 | 1.00 | 48.48 | 6 |
| ATOM | 271 | OE1 | GLU | 51 | 52.517 | 19.366 | −90.523 | 1.00 | 47.32 | 8 |
| ATOM | 272 | OE2 | GLU | 51 | 52.157 | 17.267 | −90.001 | 1.00 | 48.68 | 8 |
| ATOM | 273 | C | GLU | 51 | 54.936 | 16.610 | −94.003 | 1.00 | 51.06 | 6 |
| ATOM | 274 | O | GLU | 51 | 55.752 | 17.487 | −94.297 | 1.00 | 51.05 | 8 |
| ATOM | 275 | N | PRO | 52 | 53.881 | 16.325 | −94.789 | 1.00 | 50.14 | 7 |
| ATOM | 276 | CD | PRO | 52 | 52.893 | 15.248 | −94.600 | 1.00 | 49.79 | 6 |
| ATOM | 277 | CA | PRO | 52 | 53.633 | 17.038 | −96.047 | 1.00 | 48.26 | 6 |
| ATOM | 278 | CB | PRO | 52 | 52.327 | 16.420 | −96.541 | 1.00 | 48.69 | 6 |
| ATOM | 279 | CG | PRO | 52 | 52.394 | 15.027 | −96.006 | 1.00 | 49.76 | 6 |
| ATOM | 280 | C | PRO | 52 | 53.505 | 18.546 | −95.837 | 1.00 | 46.35 | 6 |
| ATOM | 281 | O | PRO | 52 | 53.023 | 18.994 | −94.799 | 1.00 | 46.08 | 8 |
| ATOM | 282 | N | ALA | 53 | 53.939 | 19.320 | −96.825 | 1.00 | 43.92 | 7 |
| ATOM | 283 | CA | ALA | 53 | 53.855 | 20.773 | −96.744 | 1.00 | 42.02 | 6 |
| ATOM | 284 | CB | ALA | 53 | 54.795 | 21.411 | −97.760 | 1.00 | 42.35 | 6 |
| ATOM | 285 | C | ALA | 53 | 52.418 | 21.173 | −97.036 | 1.00 | 40.63 | 6 |
| ATOM | 286 | O | ALA | 53 | 51.916 | 22.183 | −96.535 | 1.00 | 38.30 | 8 |
| ATOM | 287 | N | ARG | 54 | 51.763 | 20.365 | −97.862 | 1.00 | 38.64 | 7 |
| ATOM | 288 | CA | ARG | 54 | 50.380 | 20.600 | −98.238 | 1.00 | 36.17 | 6 |
| ATOM | 289 | CB | ARG | 54 | 50.302 | 21.385 | −99.550 | 1.00 | 37.92 | 6 |
| ATOM | 290 | CG | ARG | 54 | 51.028 | 22.715 | −99.555 | 1.00 | 41.37 | 6 |
| ATOM | 291 | CD | ARG | 54 | 51.050 | 23.283 | −100.959 | 1.00 | 43.96 | 6 |
| ATOM | 292 | NE | ARG | 54 | 49.712 | 23.630 | −101.426 | 1.00 | 47.32 | 7 |
| ATOM | 293 | CZ | ARG | 54 | 49.332 | 23.593 | −102.700 | 1.00 | 49.93 | 6 |
| ATOM | 294 | NH1 | ARG | 54 | 50.188 | 23.215 | −103.641 | 1.00 | 50.90 | 7 |
| ATOM | 295 | NH2 | ARG | 54 | 48.100 | 23.947 | −103.038 | 1.00 | 51.66 | 7 |
| ATOM | 296 | C | ARG | 54 | 49.681 | 19.263 | −98.428 | 1.00 | 33.80 | 6 |
| ATOM | 297 | O | ARG | 54 | 50.316 | 18.254 | −98.737 | 1.00 | 33.78 | 8 |
| ATOM | 298 | N | VAL | 55 | 48.369 | 19.264 | −98.234 | 1.00 | 30.02 | 7 |
| ATOM | 299 | CA | VAL | 55 | 47.566 | 18.067 | −98.420 | 1.00 | 27.00 | 6 |
| ATOM | 300 | CB | VAL | 55 | 47.176 | 17.406 | −97.070 | 1.00 | 27.76 | 6 |
| ATOM | 301 | CG1 | VAL | 55 | 48.428 | 17.033 | −96.288 | 1.00 | 25.03 | 6 |
| ATOM | 302 | CG2 | VAL | 55 | 46.291 | 18.347 | −96.262 | 1.00 | 27.29 | 6 |
| ATOM | 303 | C | VAL | 55 | 46.291 | 18.485 | −99.137 | 1.00 | 24.58 | 6 |
| ATOM | 304 | O | VAL | 55 | 45.927 | 19.665 | −99.150 | 1.00 | 24.66 | 8 |
| ATOM | 305 | N | ARG | 56 | 45.624 | 17.520 | −99.751 | 1.00 | 22.14 | 7 |
| ATOM | 306 | CA | ARG | 56 | 44.378 | 17.802 | −100.438 | 1.00 | 21.36 | 6 |
| ATOM | 307 | CB | ARG | 56 | 44.469 | 17.402 | −101.908 | 1.00 | 20.17 | 6 |
| ATOM | 308 | CG | ARG | 56 | 43.244 | 17.801 | −102.703 | 1.00 | 21.59 | 6 |
| ATOM | 309 | CD | ARG | 56 | 43.318 | 17.297 | −104.128 | 1.00 | 22.02 | 6 |
| ATOM | 310 | NE | ARG | 56 | 42.165 | 17.734 | −104.909 | 1.00 | 22.77 | 7 |
| ATOM | 311 | CZ | ARG | 56 | 42.021 | 17.503 | −106.209 | 1.00 | 22.75 | 6 |
| ATOM | 312 | NH1 | ARG | 56 | 42.961 | 16.836 | −106.869 | 1.00 | 20.56 | 7 |
| ATOM | 313 | NH2 | ARG | 56 | 40.944 | 17.946 | −106.850 | 1.00 | 19.46 | 7 |
| ATOM | 314 | C | ARG | 56 | 43.301 | 16.984 | −99.737 | 1.00 | 19.85 | 6 |
| ATOM | 315 | O | ARG | 56 | 43.486 | 15.796 | −99.491 | 1.00 | 19.97 | 8 |
| ATOM | 316 | N | CYS | 57 | 42.185 | 17.624 | −99.409 | 1.00 | 19.88 | 7 |
| ATOM | 317 | CA | CYS | 57 | 41.094 | 16.945 | −98.722 | 1.00 | 19.75 | 6 |
| ATOM | 318 | CB | CYS | 57 | 41.111 | 17.268 | −97.220 | 1.00 | 18.97 | 6 |
| ATOM | 319 | SC | CYS | 57 | 42.574 | 16.773 | −96.304 | 1.00 | 17.79 | 16 |
| ATOM | 320 | C | CYS | 57 | 39.730 | 17.353 | −99.241 | 1.00 | 19.44 | 6 |
| ATOM | 321 | O | CYS | 57 | 39.570 | 18.397 | −99.875 | 1.00 | 18.17 | 8 |
| ATOM | 322 | N | SER | 58 | 38.749 | 16.502 | −98.959 | 1.00 | 17.90 | 7 |
| ATOM | 323 | CA | SER | 58 | 37.358 | 16.780 | −99.279 | 1.00 | 16.15 | 6 |
| ATOM | 324 | CB | SER | 58 | 36.764 | 15.714 | −100.199 | 1.00 | 15.67 | 6 |
| ATOM | 325 | OG | SER | 58 | 37.380 | 15.732 | −101.472 | 1.00 | 17.18 | 8 |
| ATOM | 326 | C | SER | 58 | 36.709 | 16.671 | −97.904 | 1.00 | 15.92 | 6 |
| ATOM | 327 | O | SER | 58 | 37.301 | 16.101 | −96.982 | 1.00 | 15.39 | 8 |
| ATOM | 328 | N | HIS | 59 | 35.519 | 17.229 | −97.740 | 1.00 | 14.62 | 7 |
| ATOM | 329 | CA | HIS | 59 | 34.850 | 17.105 | −96.460 | 1.00 | 15.50 | 6 |
| ATOM | 330 | CB | HIS | 59 | 35.301 | 18.193 | −95.467 | 1.00 | 16.50 | 6 |
| ATOM | 331 | CG | HIS | 59 | 34.657 | 19.532 | −95.685 | 1.00 | 17.47 | 6 |
| ATOM | 332 | CD2 | HIS | 59 | 34.307 | 20.184 | −96.819 | 1.00 | 18.53 | 6 |
| ATOM | 333 | NO1 | HIS | 59 | 34.323 | 20.371 | −94.645 | 1.00 | 17.77 | 7 |
| ATOM | 334 | GE1 | HIS | 59 | 33.794 | 21.482 | −95.127 | 1.00 | 17.91 | 6 |
| ATOM | 335 | NE2 | HIS | 59 | 33.772 | 21.394 | −96.445 | 1.00 | 17.02 | 7 |
| ATOM | 336 | C | HIS | 59 | 33.352 | 17.180 | −96.647 | 1.00 | 15.30 | 6 |
| ATOM | 337 | O | HIS | 59 | 32.854 | 17.614 | −97.686 | 1.00 | 14.99 | 8 |
| ATOM | 338 | N | LEU | 60 | 32.645 | 16.722 | −95.628 | 1.00 | 15.86 | 7 |
| ATOM | 339 | CA | LEU | 60 | 31.202 | 16.752 | −95.611 | 1.00 | 15.21 | 6 |
| ATOM | 340 | CB | LED | 60 | 30.644 | 15.333 | −95.553 | 1.00 | 12.57 | 6 |
| ATOM | 341 | CG | LED | 60 | 29.128 | 15.188 | −95.710 | 1.00 | 13.68 | 6 |
| ATOM | 342 | CD1 | LED | 60 | 28.801 | 13.743 | −96.026 | 1.00 | 14.71 | 6 |
| ATOM | 343 | CD2 | LED | 60 | 28.420 | 15.631 | −94.441 | 1.00 | 11.80 | 6 |
| ATOM | 344 | C | LED | 60 | 30.930 | 17.499 | −94.314 | 1.00 | 16.58 | 6 |
| ATOM | 345 | O | LED | 60 | 31.327 | 17.050 | −93.239 | 1.00 | 16.19 | 8 |
| ATOM | 346 | N | LED | 61 | 30.279 | 18.650 | −94.428 | 1.00 | 17.07 | 7 |
| ATOM | 347 | CA | LED | 61 | 29.983 | 19.486 | −93.272 | 1.00 | 16.63 | 6 |
| ATOM | 348 | CB | LED | 61 | 30.407 | 20.935 | −93.553 | 1.00 | 17.57 | 6 |
| ATOM | 349 | CG | LED | 61 | 29.955 | 22.025 | −92.571 | 1.00 | 17.50 | 6 |
| ATOM | 350 | CD1 | LED | 61 | 30.645 | 21.832 | −91.221 | 1.00 | 16.44 | 6 |
| ATOM | 351 | CD2 | LED | 61 | 30.292 | 23.404 | −93.141 | 1.00 | 16.98 | 6 |
| ATOM | 352 | C | LED | 61 | 28.512 | 19.481 | −92.902 | 1.00 | 16.11 | 6 |
| ATOM | 353 | O | LED | 61 | 27.639 | 19.623 | −93.757 | 1.00 | 16.16 | 8 |
| ATOM | 354 | N | VAL | 62 | 28.241 | 19.305 | −91.617 | 1.00 | 17.29 | 7 |
| ATOM | 355 | CA | VAL | 62 | 26.875 | 19.351 | −91.135 | 1.00 | 17.50 | 6 |
| ATOM | 356 | CB | VAL | 62 | 26.433 | 18.028 | −90.498 | 1.00 | 18.92 | 6 |
| ATOM | 357 | CG1 | VAL | 62 | 25.021 | 18.172 | −89.935 | 1.00 | 17.42 | 6 |
| ATOM | 358 | CG2 | VAL | 62 | 26.467 | 16.923 | −91.539 | 1.00 | 18.64 | 6 |
| ATOM | 359 | C | VAL | 62 | 26.878 | 20.459 | −90.095 | 1.00 | 19.39 | 6 |
| ATOM | 360 | O | VAL | 62 | 27.543 | 20.363 | −89.056 | 1.00 | 16.40 | 8 |
| ATOM | 361 | N | LYS | 63 | 26.164 | 21.533 | −90.406 | 1.00 | 19.25 | 7 |
| ATOM | 362 | CA | LYS | 63 | 26.090 | 22.674 | −89.511 | 1.00 | 22.52 | 6 |
| ATOM | 363 | CB | LYS | 63 | 25.840 | 23.958 | −90.316 | 1.00 | 20.93 | 6 |
| ATOM | 364 | CG | LYS | 63 | 27.050 | 24.434 | −91.116 | 1.00 | 22.13 | 6 |
| ATOM | 365 | CD | LYS | 63 | 26.768 | 25.769 | −91.803 | 1.00 | 23.27 | 6 |
| ATOM | 366 | CE | LYS | 63 | 28.049 | 26.407 | −92.330 | 1.00 | 25.69 | 6 |
| ATOM | 367 | NZ | LYS | 63 | 27.781 | 27.745 | −92.942 | 1.00 | 26.16 | 7 |
| ATOM | 368 | C | LYS | 63 | 24.986 | 22.486 | −88.488 | 1.00 | 22.36 | 6 |
| ATOM | 369 | O | LYS | 63 | 24.154 | 21.586 | −88.611 | 1.00 | 22.63 | 8 |
| ATOM | 370 | N | HIS | 64 | 24.995 | 23.334 | −87.467 | 1.00 | 23.99 | 7 |
| ATOM | 371 | CA | HIS | 64 | 23.975 | 23.294 | −86.430 | 1.00 | 25.02 | 6 |
| ATOM | 372 | CB | HIS | 64 | 24.414 | 22.38.2 | −85.276 | 1.00 | 23.71 | 6 |
| ATOM | 373 | CG | HIS | 64 | 25.780 | 22.684 | −84.750 | 1.00 | 23.62 | 6 |
| ATOM | 374 | CD2 | HIS | 64 | 26.941 | 21.992 | −84.837 | 1.00 | 24.92 | 6 |
| ATOM | 375 | ND1 | HIS | 64 | 26.072 | 23.836 | −84.055 | 1.00 | 24.22 | 7 |
| ATOM | 376 | CE1 | HIS | 64 | 27.354 | 23.844 | −83.737 | 1.00 | 24.50 | 6 |
| ATOM | 377 | NE2 | HIS | 64 | 27.905 | 22.736 | −84.200 | 1.00 | 25.28 | 7 |
| ATOM | 378 | C | HIS | 64 | 23.725 | 24.723 | −85.950 | 1.00 | 26.23 | 6 |
| ATOM | 379 | O | HIS | 64 | 24.436 | 25.648 | −86.351 | 1.00 | 24.11 | 8 |
| ATOM | 380 | N | SER | 65 | 22.713 | 24.900 | −85.106 | 1.00 | 26.80 | 7 |
| ATOM | 381 | CA | SER | 65 | 22.353 | 26.224 | −84.602 | 1.00 | 28.69 | 6 |
| ATOM | 382 | CB | SER | 65 | 21.256 | 26.100 | −83.542 | 1.00 | 29.10 | 6 |
| ATOM | 383 | OG | SER | 65 | 21.695 | 25.318 | −82.447 | 1.00 | 31.05 | 8 |
| ATOM | 384 | C | SER | 65 | 23.510 | 27.054 | −84.046 | 1.00 | 29.02 | 6 |
| ATOM | 385 | O | SER | 65 | 23.475 | 28.280 | −84.110 | 1.00 | 30.47 | 8 |
| ATOM | 386 | N | GLN | 66 | 24.536 | 26.408 | −83.504 | 1.00 | 28.37 | 7 |
| ATOM | 387 | CA | GLN | 66 | 25.660 | 27.168 | −82.968 | 1.00 | 27.57 | 6 |
| ATOM | 388 | CB | GLN | 66 | 26.214 | 26.498 | −81.705 | 1.00 | 29.33 | 6 |
| ATOM | 389 | CG | GLN | 66 | 25.287 | 26.575 | −80.499 | 1.00 | 30.77 | 6 |
| ATOM | 390 | CD | GLN | 66 | 25.956 | 26.099 | −79.214 | 1.00 | 35.08 | 6 |
| ATOM | 391 | OE1 | GLN | 66 | 26.885 | 26.736 | −78.705 | 1.00 | 34.17 | 8 |
| ATOM | 392 | NE2 | GLN | 66 | 25.488 | 24.971 | −78.687 | 1.00 | 34.25 | 7 |
| ATOM | 393 | C | GLN | 66 | 26.799 | 27.411 | −83.963 | 1.00 | 27.37 | 6 |
| ATOM | 394 | O | GLN | 66 | 27.829 | 27.975 | −83.593 | 1.00 | 27.54 | 8 |
| ATOM | 395 | N | SER | 67 | 26.631 | 26.995 | −85.217 | 1.00 | 26.32 | 7 |
| ATOM | 396 | CA | SER | 67 | 27.679 | 27.218 | −86.216 | 1.00 | 28.55 | 6 |
| ATOM | 397 | CB | SER | 67 | 27.300 | 26.580 | −87.557 | 1.00 | 27.37 | 6 |
| ATOM | 398 | OG | SER | 67 | 27.239 | 25.169 | −87.459 | 1.00 | 26.17 | 8 |
| ATOM | 399 | C | SER | 67 | 27.876 | 28.722 | −86.408 | 1.00 | 30.70 | 6 |
| ATOM | 400 | O | SER | 67 | 26.908 | 29.482 | −86.401 | 1.00 | 29.47 | 8 |
| ATOM | 401 | N | ARG | 68 | 29.125 | 29.146 | −86.584 | 1.00 | 33.48 | 7 |
| ATOM | 402 | CA | ARG | 68 | 29.429 | 30.566 | −86.768 | 1.00 | 36.47 | 6 |
| ATOM | 403 | CB | ARG | 68 | 30.836 | 30.737 | −87.340 | 1.00 | 39.41 | 6 |
| ATOM | 404 | CG | ARG | 68 | 31.483 | 32.088 | −87.036 | 1.00 | 45.56 | 6 |
| ATOM | 405 | CD | ARG | 68 | 32.158 | 32.127 | −85.655 | 1.00 | 49.30 | 6 |
| ATOM | 406 | NE | ARG | 68 | 31.215 | 32.147 | −84.536 | 1.00 | 52.73 | 7 |
| ATOM | 407 | CZ | ARG | 68 | 31.575 | 32.130 | −83.253 | 1.00 | 53.77 | 6 |
| ATOM | 408 | NH1 | ARG | 68 | 32.860 | 32.091 | −82.920 | 1.00 | 53.46 | 7 |
| ATOM | 409 | NH2 | ARG | 68 | 30.652 | 32.155 | −82.299 | 1.00 | 53.75 | 7 |
| ATOM | 410 | C | ARG | 68 | 28.398 | 31.186 | −87.710 | 1.00 | 37.90 | 6 |
| ATOM | 411 | O | ARG | 68 | 27.892 | 32.283 | −87.460 | 1.00 | 38.63 | 8 |
| ATOM | 412 | N | ARG | 69 | 28.087 | 30.476 | −88.790 | 1.00 | 37.17 | 7 |
| ATOM | 413 | CA | ARG | 69 | 27.089 | 30.935 | −89.753 | 1.00 | 36.32 | 6 |
| ATOM | 414 | CB | ARG | 69 | 27.737 | 31.272 | −91.093 | 1.00 | 40.13 | 6 |
| ATOM | 415 | CG | ARG | 69 | 28.044 | 32.743 | −91.261 | 1.00 | 45.68 | 6 |
| ATOM | 416 | CD | ARG | 69 | 28.243 | 33.090 | −92.720 | 1.00 | 50.75 | 6 |
| ATOM | 417 | NE | ARG | 69 | 27.956 | 34.500 | −92.975 | 1.00 | 56.20 | 7 |
| ATOM | 418 | CZ | ARG | 69 | 26.777 | 35.077 | −92.752 | 1.00 | 57.63 | 6 |
| ATOM | 419 | NH1 | ARG | 69 | 25.764 | 34.370 | −92.266 | 1.00 | 58.33 | 7 |
| ATOM | 420 | NH2 | ARG | 69 | 26.611 | 36.365 | −93.016 | 1.00 | 59.70 | 7 |
| ATOM | 421 | C | ARG | 69 | 26.039 | 29.850 | −89.958 | 1.00 | 34.38 | 6 |
| ATOM | 422 | O | ARG | 69 | 26.194 | 28.980 | −90.815 | 1.00 | 34.43 | 8 |
| ATOM | 423 | N | PRO | 70 | 24.951 | 29.892 | −89.172 | 1.00 | 32.51 | 7 |
| ATOM | 424 | CD | PRO | 70 | 24.675 | 30.863 | −88.096 | 1.00 | 32.43 | 6 |
| ATOM | 425 | CA | PRO | 70 | 23.876 | 28.902 | −89.271 | 1.00 | 31.26 | 6 |
| ATOM | 426 | CB | PRO | 70 | 23.055 | 29.164 | −88.011 | 1.00 | 31.77 | 6 |
| ATOM | 427 | CG | PRO | 70 | 23.204 | 30.638. | −87.825 | 1.00 | 32.80 | 6 |
| ATOM | 428 | C | PRO | 70 | 23.041 | 28.984 | −90.549 | 1.00 | 30.06 | 6 |
| ATOM | 429 | O | PRO | 70 | 21.822 | 29.139 | −90.497 | 1.00 | 29.37 | 8 |
| ATOM | 430 | N | SEP | 71 | 23.711 | 28.858 | −91.692 | 1.00 | 28.90 | 7 |
| ATOM | 431 | CA | SEP | 71 | 23.052 | 28.906 | −92.993 | 1.00 | 28.76 | 6 |
| ATOM | 432 | CB | SEP | 71 | 22.786 | 30.358 | −93.410 | 1.00 | 29.36 | 6 |
| ATOM | 433 | OG | SEP | 71 | 23.992 | 31.104 | −93.569 | 1.00 | 33.73 | 8 |
| ATOM | 434 | C | SEP | 71 | 23.924 | 28.231 | −94.047 | 1.00 | 28.45 | 6 |
| ATOM | 435 | O | SEP | 71 | 25.143 | 28.148 | −93.891 | 1.00 | 27.63 | 8 |
| ATOM | 436 | P | SEP | 71 | 24.361 | 31.758 | −94.918 | 1.00 | 38.66 | 15 |
| ATOM | 437 | O1P | SEP | 71 | 24.644 | 31.405 | −96.249 | 1.00 | 39.66 | 8 |
| ATOM | 438 | O2P | SEP | 71 | 25.851 | 32.159 | −94.489 | 1.00 | 39.21 | 8 |
| ATOM | 439 | O3P | SEP | 71 | 23.563 | 32.797 | −95.138. | 1.00 | 36.04 | 8 |
| ATOM | 440 | N | SER | 72 | 23.295 | 27.747 | −95.114 | 1.00 | 27.08 | 7 |
| ATOM | 441 | CA | SER | 72 | 24.021 | 27.105 | −96.206 | 1.00 | 27.40 | 6 |
| ATOM | 442 | CB | SER | 72 | 24.462 | 25.687 | −95.814 | 1.00 | 25.69 | 6 |
| ATOM | 443 | OG | SER | 72 | 23.366 | 24.788 | −95.845 | 1.00 | 24.00 | 8 |
| ATOM | 444 | C | SER | 72 | 23.124 | 27.027 | −97.432 | 1.00 | 26.69 | 6 |
| ATOM | 445 | O | SER | 72 | 21.955 | 27.410 | −97.379 | 1.00 | 27.76 | 8 |
| ATOM | 446 | N | TRP | 73 | 23.670 | 26.521 | −98.533 | 1.00 | 26.98 | 7 |
| ATOM | 447 | CA | TRP | 73 | 22.901 | 26.38.4 | −99.761 | 1.00 | 25.71 | 6 |
| ATOM | 448 | CB | TRP | 73 | 23.792 | 25.873 | −100.901 | 1.00 | 26.37 | 6 |
| ATOM | 449 | CG | TRP | 73 | 24.328 | 24.482 | −100.671 | 1.00 | 26.28 | 6 |
| ATOM | 450 | CD2 | TRP | 73 | 23.716 | 23.249 | −101.077 | 1.00 | 24.43 | 6 |
| ATOM | 451 | CE2 | TRP | 73 | 24.538 | 22.201 | −100.610 | 1.00 | 23.09 | 6 |
| ATOM | 452 | CE3 | TRP | 73 | 22.550 | 22.930 | −101.790 | 1.00 | 25.71 | 6 |
| ATOM | 453 | CD1 | TRP | 73 | 25.465 | 24.140 | −99.993 | 1.00 | 23.83 | 6 |
| ATOM | 454 | NE1 | TRP | 73 | 25.597 | 22.771 | −99.953 | 1.00 | 24.60 | 7 |
| ATOM | 455 | CZ2 | TRP | 73 | 24.234 | 20.855 | −100.832 | 1.00 | 22.99 | 6 |
| ATOM | 456 | CZ3 | TRP | 73 | 22.247 | 21.586 | −102.010 | 1.00 | 27.04 | 6 |
| ATOM | 457 | CH2 | TRP | 73 | 23.090 | 20.566 | −101.530 | 1.00 | 24.93 | 6 |
| ATOM | 458 | C | TRP | 73 | 21.740 | 25.413 | −99.556 | 1.00 | 25.89 | 6 |
| ATOM | 459 | O | TRP | 73 | 20.774 | 25.425 | −100.316 | 1.00 | 26.99 | 8 |
| ATOM | 460 | N | ARG | 74 | 21.832 | 24.571 | −98.529 | 1.00 | 25.72 | 7 |
| ATOM | 461 | CA | ARG | 74 | 20.778 | 23.597 | −98.259 | 1.00 | 25.29 | 6 |
| ATOM | 462 | CB | ARG | 74 | 21.306 | 22.457 | −97.38.4 | 1.00 | 22.97 | 6 |
| ATOM | 463 | CG | ARG | 74 | 22.537 | 21.753 | −97.943 | 1.00 | 22.82 | 6 |
| ATOM | 464 | CD | ARG | 74 | 22.699 | 20.393 | −97.285 | 1.00 | 22.27 | 6 |
| ATOM | 465 | NE | ARG | 74 | 21.599 | 19.513 | −97.661 | 1.00 | 20.86 | 7 |
| ATOM | 466 | CZ | ARG | 74 | 20.905 | 18.766 | −96.810 | 1.00 | 22.44 | 6 |
| ATOM | 467 | NH1 | ARG | 74 | 21.191 | 18.782 | −95.514 | 1.00 | 23.69 | 7 |
| ATOM | 468 | NH2 | ARG | 74 | 19.921 | 18.000 | −97.261 | 1.00 | 23.46 | 7 |
| ATOM | 469 | C | ARG | 74 | 19.566 | 24.235 | −97.584 | 1.00 | 26.70 | 6 |
| ATOM | 470 | O | ARG | 74 | 18.431 | 23.825 | −97.820 | 1.00 | 25.54 | 8 |
| ATOM | 471 | N | GLN | 75 | 19.808 | 25.223 | −96.729 | 1.00 | 27.15 | 7 |
| ATOM | 472 | CA | GLN | 75 | 18.720 | 25.920 | −96.059 | 1.00 | 29.48 | 6 |
| ATOM | 473 | CB | GLN | 75 | 18.110 | 25.054 | −94.950 | 1.00 | 31.63 | 6 |
| ATOM | 474 | CG | GLN | 75 | 19.095 | 24.543 | −93.929 | 1.00 | 34.67 | 6 |
| ATOM | 475 | CD | GLN | 75 | 18.437 | 23.689 | −92.857 | 1.00 | 35.54 | 6 |
| ATOM | 476 | OE1 | GLN | 75 | 19.113 | 23.141 | −91.990 | 1.00 | 36.47 | 8 |
| ATOM | 477 | NE2 | GLN | 75 | 17.114 | 23.574 | −92.912 | 1.00 | 36.87 | 7 |
| ATOM | 478 | C | GLN | 75 | 19.176 | 27.261 | −95.502 | 1.00 | 28.91 | 6 |
| ATOM | 479 | O | GLN | 75 | 20.263 | 27.38.3 | −94.930 | 1.00 | 27.50 | 8 |
| ATOM | 480 | N | GLU | 76 | 18.326 | 28.266 | −95.686 | 1.00 | 28.12 | 7 |
| ATOM | 481 | CA | GLU | 76 | 18.613 | 29.620 | −95.242 | 1.00 | 27.71 | 6 |
| ATOM | 482 | CB | GLU | 76 | 17.452 | 30.546 | −95.613 | 1.00 | 26.82 | 6 |
| ATOM | 483 | CG | GLU | 76 | 17.782 | 32.023 | −95.477 | 1.00 | 27.06 | 6 |
| ATOM | 484 | CD | GLU | 76 | 16.761 | 32.913 | −96.160 | 1.00 | 26.28 | 6 |
| ATOM | 485 | OE1 | GLU | 76 | 16.992 | 34.140 | −96.224 | 1.00 | 27.07 | 8 |
| ATOM | 486 | OE2 | GLU | 76 | 15.732 | 32.38.5 | −96.632 | 1.00 | 22.88 | 8 |
| ATOM | 487 | C | GLU | 76 | 18.890 | 29.698 | −93.747 | 1.00 | 27.03 | 6 |
| ATOM | 488 | O | GLU | 76 | 19.749 | 30.462 | −93.310 | 1.00 | 26.10 | 8 |
| ATOM | 489 | N | LYS | 77 | 18.167 | 28.906 | −92.964 | 1.00 | 28.85 | 7 |
| ATOM | 490 | CA | LYS | 77 | 18.363 | 28.899 | −91.519 | 1.00 | 30.52 | 6 |
| ATOM | 491 | CB | LYS | 77 | 17.167 | 29.549 | −90.818 | 1.00 | 32.71 | 6 |
| ATOM | 492 | CG | LYS | 77 | 17.512 | 30.838 | −90.097 | 1.00 | 37.07 | 6 |
| ATOM | 493 | CD | LYS | 77 | 18.056 | 31.872 | −91.064 | 1.00 | 38.40 | 6 |
| ATOM | 494 | CE | LYS | 77 | 18.427 | 33.158 | −90.350 | 1.00 | 39.24 | 6 |
| ATOM | 495 | NZ | LYS | 77 | 18.918 | 34.181 | −91.315 | 1.00 | 40.36 | 7 |
| ATOM | 496 | C | LYS | 77 | 18.573 | 27.498 | −90.954 | 1.00 | 29.15 | 6 |
| ATOM | 497 | O | LYS | 77 | 17.651 | 26.687 | −90.936 | 1.00 | 30.23 | 8 |
| ATOM | 498 | N | ILE | 78 | 19.788 | 27.220 | −90.491 | 1.00 | 29.41 | 7 |
| ATOM | 499 | CA | ILE | 78 | 20.100 | 25.921 | −89.900 | 1.00 | 28.17 | 6 |
| ATOM | 500 | CB | ILE | 78 | 21.615 | 25.645 | −89.883 | 1.00 | 28.60 | 6 |
| ATOM | 501 | CG2 | ILE | 78 | 21.878 | 24.276 | −89.250 | 1.00 | 27.62 | 6 |
| ATOM | 502 | CG1 | ILE | 78 | 22.187 | 25.718 | −91.302 | 1.00 | 29.06 | 6 |
| ATOM | 503 | CD1 | ILE | 78 | 21.733 | 24.611 | −92.206 | 1.00 | 29.16 | 6 |
| ATOM | 504 | C | ILE | 78 | 19.616 | 25.949 | −88.451 | 1.00 | 28.15 | 6 |
| ATOM | 505 | O | ILE | 78 | 20.106 | 26.741 | −87.650 | 1.00 | 26.44 | 8 |
| ATOM | 506 | N | THR | 79 | 18.679 | 25.070 | −88.112 | 1.00 | 28.27 | 7 |
| ATOM | 507 | CA | THR | 79 | 18.133 | 25.037 | −86.761 | 1.00 | 29.78 | 6 |
| ATOM | 508 | CB | THR | 79 | 16.613 | 25.223 | −86.808 | 1.00 | 28.98 | 6 |
| ATOM | 509 | OG1 | THR | 79 | 16.024 | 24.139 | −87.533 | 1.00 | 30.59 | 8 |
| ATOM | 510 | CG2 | THR | 79 | 16.268 | 26.524 | −87.510 | 1.00 | 31.57 | 6 |
| ATOM | 511 | C | THR | 79 | 18.441 | 23.781 | −85.937 | 1.00 | 30.12 | 6 |
| ATOM | 512 | O | THR | 79 | 18.159 | 23.742 | −84.739 | 1.00 | 29.37 | 8 |
| ATOM | 513 | N | ARG | 80 | 19.015 | 22.757 | −86.563 | 1.00 | 28.78 | 7 |
| ATOM | 514 | CA | ARG | 80 | 19.329 | 21.528 | −85.836 | 1.00 | 26.16 | 6 |
| ATOM | 515 | CB | ARG | 80 | 19.841 | 20.448 | −86.803 | 1.00 | 25.90 | 6 |
| ATOM | 516 | CG | ARG | 80 | 21.174 | 20.760 | −87.483 | 1.00 | 22.66 | 6 |
| ATOM | 517 | CD | ARG | 80 | 21.694 | 19.537 | −88.249 | 1.00 | 21.37 | 6 |
| ATOM | 518 | NE | ARG | 80 | 21.011 | 19.306 | −89.525 | 1.00 | 19.80 | 7 |
| ATOM | 519 | CZ | ARG | 80 | 21.304 | 19.941 | −90.660 | 1.00 | 19.87 | 6 |
| ATOM | 520 | NH1 | ARG | 80 | 22.269 | 20.852 | −90.684 | 1.00 | 19.92 | 7 |
| ATOM | 521 | NH2 | ARG | 80 | 20.646 | 19.658 | −91.778 | 1.00 | 16.25 | 7 |
| ATOM | 522 | C | ARG | 80 | 20.358 | 21.772 | −84.731 | 1.00 | 24.44 | 6 |
| ATOM | 523 | O | ARG | 80 | 21.242 | 22.615 | −84.871 | 1.00 | 26.37 | 8 |
| ATOM | 524 | N | THR | 81 | 20.234 | 21.037 | −83.629 | 1.00 | 23.35 | 7 |
| ATOM | 525 | CA | THR | 81 | 21.163 | 21.174 | −82.512 | 1.00 | 24.30 | 6 |
| ATOM | 526 | CB | THR | 81 | 20.605 | 20.539 | −81.224 | 1.00 | 24.96 | 6 |
| ATOM | 527 | OG1 | THR | 81 | 20.480 | 19.121 | −81.404 | 1.00 | 26.91 | 8 |
| ATOM | 528 | CG2 | THR | 81 | 19.240 | 21.124 | −80.888 | 1.00 | 26.50 | 6 |
| ATOM | 529 | C | THR | 81 | 22.472 | 20.473 | −82.837 | 1.00 | 24.72 | 6 |
| ATOM | 530 | O | THR | 81 | 22.555 | 19.716 | −83.801 | 1.00 | 23.85 | 8 |
| ATOM | 531 | N | LYS | 82 | 23.493 | 20.731 | −82.027 | 1.00 | 25.59 | 7 |
| ATOM | 532 | CA | LYS | 82 | 24.796 | 20.112 | −82.216 | 1.00 | 27.19 | 6 |
| ATOM | 533 | CB | LYS | 82 | 25.777 | 20.590 | −81.143 | 1.00 | 30.27 | 6 |
| ATOM | 534 | CG | LYS | 82 | 26.041 | 22.082 | −81.137 | 1.00 | 34.64 | 6 |
| ATOM | 535 | CD | LYS | 82 | 27.012 | 22.458 | −80.019 | 1.00 | 38.89 | 6 |
| ATOM | 536 | CE | LYS | 82 | 28.376 | 21.801 | −80.215 | 1.00 | 39.40 | 6 |
| ATOM | 537 | NZ | LYS | 82 | 29.323 | 22.138 | −79.119 | 1.00 | 40.16 | 7 |
| ATOM | 538 | C | LYS | 82 | 24.666 | 18.596 | −82.121 | 1.00 | 26.80 | 6 |
| ATOM | 539 | O | LYS | 82 | 25.295 | 17.862 | −82.879 | 1.00 | 25.16 | 8 |
| ATOM | 540 | N | GLU | 83 | 23.847 | 18.134 | −81.181 | 1.00 | 26.38 | 7 |
| ATOM | 541 | CA | GLU | 83 | 23.646 | 16.705 | −80.976 | 1.00 | 26.57 | 6 |
| ATOM | 542 | CB | GLU | 83 | 22.779 | 16.462 | −79.736 | 1.00 | 28.25 | 6 |
| ATOM | 543 | CG | GLU | 83 | 23.415 | 16.923 | −78.430 | 1.00 | 31.95 | 6 |
| ATOM | 544 | CD | GLU | 83 | 23.404 | 18.436 | −78.256 | 1.00 | 35.64 | 6 |
| ATOM | 545 | OE1 | GLU | 83 | 24.137 | 18.938 | −77.375 | 1.00 | 38.69 | 8 |
| ATOM | 546 | OE2 | GLU | 83 | 22.660 | 19.124 | −78.986 | 1.00 | 35.86 | 8 |
| ATOM | 547 | C | GLU | 83 | 23.008 | 16.041 | −82.188 | 1.00 | 26.05 | 6 |
| ATOM | 548 | O | GLU | 83 | 23.370 | 14.925 | −82.561 | 1.00 | 24.96 | 8 |
| ATOM | 549 | N | GLU | 84 | 22.050 | 16.727 | −82.798 | 1.00 | 24.96 | 7 |
| ATOM | 550 | CA | GLU | 84 | 21.371 | 16.197 | −83.971 | 1.00 | 25.80 | 6 |
| ATOM | 551 | CB | GLU | 84 | 20.168 | 17.071 | −84.316 | 1.00 | 26.09 | 6 |
| ATOM | 552 | CG | GLU | 84 | 19.146 | 17.157 | −83.197 | 1.00 | 29.16 | 6 |
| ATOM | 553 | CD | GLU | 84 | 17.991 | 18.080 | −83.535 | 1.00 | 28.92 | 6 |
| ATOM | 554 | OE1 | GLU | 84 | 18.256 | 19.242 | −83.912 | 1.00 | 29.10 | 8 |
| ATOM | 555 | OE2 | GLU | 84 | 16.825 | 17.647 | −83.418 | 1.00 | 29.33 | 8 |
| ATOM | 556 | C | GLU | 84 | 22.337 | 16.145 | −85.151 | 1.00 | 24.73 | 6 |
| ATOM | 557 | O | GLU | 84 | 22.293 | 15.225 | −85.967 | 1.00 | 23.64 | 8 |
| ATOM | 558 | N | ALA | 85 | 23.211 | 17.140 | −85.227 | 1.00 | 23.91 | 7 |
| ATOM | 559 | CA | ALA | 85 | 24.204 | 17.212 | −86.292 | 1.00 | 24.23 | 6 |
| ATOM | 560 | CB | ALA | 85 | 24.932 | 18.554 | −86.232 | 1.00 | 23.35 | 6 |
| ATOM | 561 | C | ALA | 85 | 25.203 | 16.064 | −86.149 | 1.00 | 22.92 | 6 |
| ATOM | 562 | O | ALA | 85 | 25.638 | 15.476 | −87.142 | 1.00 | 20.56 | 8 |
| ATOM | 563 | N | LED | 86 | 25.570 | 15.749 | −84.911 | 1.00 | 23.09 | 7 |
| ATOM | 564 | CA | LED | 86 | 26.517 | 14.667 | −84.670 | 1.00 | 21.91 | 6 |
| ATOM | 565 | CB | LED | 86 | 26.956 | 14.657 | −83.201 | 1.00 | 21.73 | 6 |
| ATOM | 566 | CG | LEU | 86 | 28.002 | 13.607 | −82.799 | 1.00 | 23.14 | 6 |
| ATOM | 567 | CD1 | LED | 86 | 29.193 | 13.661 | −83.746 | 1.00 | 22.49 | 6 |
| ATOM | 568 | CD2 | LEU | 86 | 28.454 | 13.857 | −81.364 | 1.00 | 24.13 | 6 |
| ATOM | 569 | C | LED | 86 | 25.906 | 13.318 | −85.057 | 1.00 | 22.20 | 6 |
| ATOM | 570 | O | LED | 86 | 26.607 | 12.432 | −85.545 | 1.00 | 21.69 | 8 |
| ATOM | 571 | N | GLU | 87 | 24.601 | 13.163 | −84.850 | 1.00 | 21.03 | 7 |
| ATOM | 572 | CA | GLU | 87 | 23.934 | 11.913 | −85.207 | 1.00 | 23.57 | 6 |
| ATOM | 573 | CB | GLU | 87 | 22.518 | 11.868 | −84.624 | 1.00 | 26.65 | 6 |
| ATOM | 574 | CG | GLU | 87 | 22.497 | 11.322 | −83.209 | 1.00 | 35.29 | 6 |
| ATOM | 575 | CD | GLU | 87 | 23.088 | 9.920 | −83.137 | 1.00 | 39.32 | 6 |
| ATOM | 576 | OE1 | GLU | 87 | 22.473 | 8.988 | −83.699 | 1.00 | 42.90 | 8 |
| ATOM | 577 | OE2 | GLU | 87 | 24.172 | 9.752 | −82.534 | 1.00 | 42.88 | 8 |
| ATOM | 578 | C | GLU | 87 | 23.890 | 11.712 | −86.716 | 1.00 | 21.70 | 6 |
| ATOM | 579 | O | GLU | 87 | 24.030 | 10.588 | −87.204 | 1.00 | 21.57 | 8 |
| ATOM | 580 | N | LED | 88 | 23.694 | 12.802 | −87.451 | 1.00 | 19.60 | 7 |
| ATOM | 581 | CA | LED | 88 | 23.667 | 12.738 | −88.907 | 1.00 | 18.85 | 6 |
| ATOM | 582 | CB | LED | 88 | 23.270 | 14.098 | −89.488 | 1.00 | 18.76 | 6 |
| ATOM | 583 | CG | LED | 88 | 21.792 | 14.451 | −89.285 | 1.00 | 21.31 | 6 |
| ATOM | 584 | CD1 | LED | 88 | 21.545 | 15.925 | −89.600 | 1.00 | 21.59 | 6 |
| ATOM | 585 | CD2 | LED | 88 | 20.938 | 13.551 | −90.178 | 1.00 | 20.92 | 6 |
| ATOM | 586 | C | LED | 88 | 25.058 | 12.341 | −89.391 | 1.00 | 17.19 | 6 |
| ATOM | 587 | O | LED | 88 | 25.204 | 11.479 | −90.262 | 1.00 | 17.13 | 8 |
| ATOM | 588 | N | ILE | 89 | 26.075 | 12.974 | −88.810 | 1.00 | 15.89 | 7 |
| ATOM | 589 | CA | ILE | 89 | 27.467 | 12.691 | −89.147 | 1.00 | 16.17 | 6 |
| ATOM | 590 | CB | ILE | 89 | 28.429 | 13.582 | −88.315 | 1.00 | 15.65 | 6 |
| ATOM | 591 | CG2 | ILE | 89 | 29.851 | 13.017 | −88.350 | 1.00 | 15.23 | 6 |
| ATOM | 592 | CG1 | ILE | 89 | 28.414 | 15.018 | −88.861 | 1.00 | 14.89 | 6 |
| ATOM | 593 | CD1 | ILE | 89 | 29.252 | 15.218 | −90.129 | 1.00 | 13.85 | 6 |
| ATOM | 594 | C | ILE | 89 | 27.772 | 11.215 | −88.875 | 1.00 | 15.28 | 6 |
| ATOM | 595 | O | ILE | 89 | 28.318 | 10.525 | −89.727 | 1.00 | 17.26 | 8 |
| ATOM | 596 | N | ASN | 90 | 27.408 | 10.737 | −87.690 | 1.00 | 15.04 | 7 |
| ATOM | 597 | CA | ASN | 90 | 27.648 | 9.346 | −87.332 | 1.00 | 16.28 | 6 |
| ATOM | 598 | CB | ASN | 90 | 27.131 | 9.056 | −85.921 | 1.00 | 17.98 | 6 |
| ATOM | 599 | CG | ASN | 90 | 27.995 | 9.687 | −84.849 | 1.00 | 20.64 | 6 |
| ATOM | 600 | OD1 | ASN | 90 | 29.193 | 9.890 | −85.045 | 1.00 | 22.18 | 8 |
| ATOM | 601 | ND2 | ASN | 90 | 27.397 | 9.981 | −83.701 | 1.00 | 21.98 | 7 |
| ATOM | 602 | C | ASN | 90 | 26.987 | 8.400 | −88.325 | 1.00 | 16.72 | 6 |
| ATOM | 603 | O | ASN | 90 | 27.567 | 7.377 | −88.696 | 1.00 | 16.77 | 8 |
| ATOM | 604 | N | GLY | 91 | 25.776 | 8.751 | −88.752 | 1.00 | 16.18 | 7 |
| ATOM | 605 | CA | GLY | 91 | 25.053 | 7.935 | −89.710 | 1.00 | 17.14 | 6 |
| ATOM | 606 | C | GLY | 91 | 25.738 | 7.907 | −91.067 | 1.00 | 17.92 | 6 |
| ATOM | 607 | O | GLY | 91 | 25.813 | 6.861 | −91.716 | 1.00 | 16.83 | 8 |
| ATOM | 608 | N | TYR | 92 | 26.243 | 9.057 | −91.501 | 1.00 | 17.07 | 7 |
| ATOM | 609 | CA | TYR | 92 | 26.929 | 9.144 | −92.786 | 1.00 | 16.37 | 6 |
| ATOM | 610 | CB | TYR | 92 | 27.280 | 10.597 | −93.114 | 1.00 | 16.45 | 6 |
| ATOM | 611 | CG | TYR | 92 | 26.079 | 11.495 | −93.326 | 1.00 | 17.64 | 6 |
| ATOM | 612 | CD1 | TYR | 92 | 26.158 | 12.862 | −93.058 | 1.00 | 17.64 | 6 |
| ATOM | 613 | CE1 | TYR | 92 | 25.062 | 13.703 | −93.252 | 1.00 | 19.28 | 6 |
| ATOM | 614 | CD2 | TYR | 92 | 24.869 | 10.985 | −93.801 | 1.00 | 19.39 | 6 |
| ATOM | 615 | CE2 | TYR | 92 | 23.760 | 11.823 | −94.003 | 1.00 | 20.95 | 6 |
| ATOM | 616 | CZ | TYR | 92 | 23.872 | 13.181 | −93.720 | 1.00 | 19.49 | 6 |
| ATOM | 617 | OH | TYR | 92 | 22.795 | 14.020 | −93.891 | 1.00 | 22.48 | 8 |
| ATOM | 618 | C | TYR | 92 | 28.202 | 8.320 | −92.739 | 1.00 | 13.76 | 6 |
| ATOM | 619 | O | TYR | 92 | 28.556 | 7.662 | −93.712 | 1.00 | 13.20 | 8 |
| ATOM | 620 | N | ILE | 93 | 28.896 | 8.370 | −91.608 | 1.00 | 14.49 | 7 |
| ATOM | 621 | CA | ILE | 93 | 30.126 | 7.605 | −91.454 | 1.00 | 14.72 | 6 |
| ATOM | 622 | CB | ILE | 93 | 30.825 | 7.941 | −90.119 | 1.00 | 15.28 | 6 |
| ATOM | 623 | CG2 | ILE | 93 | 31.896 | 6.896 | −89.792 | 1.00 | 14.16 | 6 |
| ATOM | 624 | CG1 | ILE | 93 | 31.458 | 9.333 | −90.212 | 1.00 | 14.26 | 6 |
| ATOM | 625 | CD1 | ILE | 93 | 32.089 | 9.795 | −88.916 | 1.00 | 12.97 | 6 |
| ATOM | 626 | C | ILE | 93 | 29.811 | 6.112 | −91.519 | 1.00 | 16.84 | 6 |
| ATOM | 627 | O | ILE | 93 | 30.543 | 5.344 | −92.146 | 1.00 | 14.19 | 8 |
| ATOM | 628 | N | GLN | 94 | 28.716 | 5.699 | −90.884 | 1.00 | 16.66 | 7 |
| ATOM | 629 | CA | GLN | 94 | 28.348 | 4.290 | −90.911 | 1.00 | 19.54 | 6 |
| ATOM | 630 | CB | GLN | 94 | 27.100 | 4.031 | −90.061 | 1.00 | 20.46 | 6 |
| ATOM | 631 | CG | GLN | 94 | 27.346 | 4.092 | −88.571 | 1.00 | 21.94 | 6 |
| ATOM | 632 | CD | GLN | 94 | 26.120 | 3.703 | −87.773 | 1.00 | 29.30 | 6 |
| ATOM | 633 | OE1 | GLN | 94 | 25.090 | 4.372 | −87.829 | 1.00 | 31.60 | 8 |
| ATOM | 634 | NE2 | GLN | 94 | 26.222 | 2.606 | −87.029 | 1.00 | 31.65 | 7 |
| ATOM | 635 | C | GLN | 94 | 28.095 | 3.832 | −92.342 | 1.00 | 19.80 | 6 |
| ATOM | 636 | O | GLN | 94 | 28.584 | 2.783 | −92.756 | 1.00 | 19.84 | 8 |
| ATOM | 637 | N | LYS | 95 | 27.340 | 4.625 | −93.098 | 1.00 | 19.72 | 7 |
| ATOM | 638 | CA | LYS | 95 | 27.032 | 4.281 | −94.479 | 1.00 | 20.21 | 6 |
| ATOM | 639 | CB | LYS | 95 | 25.962 | 5.220 | −95.037 | 1.00 | 24.26 | 6 |
| ATOM | 640 | CG | LYS | 95 | 24.583 | 5.006 | −94.430 | 1.00 | 29.32 | 6 |
| ATOM | 641 | CD | LYS | 95 | 23.532 | 5.813 | −95.177 | 1.00 | 35.38 | 6 |
| ATOM | 642 | CE | LYS | 95 | 22.136 | 5.575 | −94.609 | 1.00 | 37.03 | 6 |
| ATOM | 643 | NZ | LYS | 95 | 22.029 | 6.031 | −93.189 | 1.00 | 41.01 | 7 |
| ATOM | 644 | C | LYS | 95 | 28.250 | 4.297 | −95.392 | 1.00 | 18.24 | 6 |
| ATOM | 645 | O | LYS | 95 | 28.305 | 3.559 | −96.372 | 1.00 | 17.20 | 8 |
| ATOM | 646 | N | ILE | 96 | 29.219 | 5.151 | −95.084 | 1.00 | 16.21 | 7 |
| ATOM | 647 | CA | ILE | 96 | 30.435 | 5.226 | −95.891 | 1.00 | 14.67 | 6 |
| ATOM | 648 | CE | ILE | 96 | 31.247 | 6.513 | −95.563 | 1.00 | 14.05 | 6 |
| ATOM | 649 | CG2 | ILE | 96 | 32.621 | 6.450 | −96.216 | 1.00 | 14.17 | 6 |
| ATOM | 650 | CG1 | ILE | 96 | 30.482 | 7.750 | −96.049 | 1.00 | 14.83 | 6 |
| ATOM | 651 | CD1 | ILE | 96 | 31.084 | 9.081 | −95.592 | 1.00 | 15.94 | 6 |
| ATOM | 652 | C | ILE | 96 | 31.303 | 3.997 | −95.601 | 1.00 | 14.90 | 6 |
| ATOM | 653 | O | ILE | 96 | 31.846 | 3.372 | −96.515 | 1.00 | 15.07 | 8 |
| ATOM | 654 | N | LYS | 97 | 31.419 | 3.648 | −94.324 | 1.00 | 13.73 | 7 |
| ATOM | 655 | CA | LYS | 97 | 32.228 | 2.501 | −93.914 | 1.00 | 16.38 | 6 |
| ATOM | 656 | CB | LYS | 97 | 32.423 | 2.502 | −92.395 | 1.00 | 16.19 | 6 |
| ATOM | 657 | CG | LYS | 97 | 33.452 | 3.503 | −91.897 | 1.00 | 18.15 | 6 |
| ATOM | 658 | CD | LYS | 97 | 33.845 | 3.203 | −90.455 | 1.00 | 18.42 | 6 |
| ATOM | 659 | CE | LYS | 97 | 35.071 | 4.007 | −90.048 | 1.00 | 20.15 | 6 |
| ATOM | 660 | NZ | LYS | 97 | 35.614 | 3.608 | −88.714 | 1.00 | 18.16 | 7 |
| ATOM | 661 | C | LYS | 97 | 31.657 | 1.153 | −94.350 | 1.00 | 16.98 | 6 |
| ATOM | 662 | O | LYS | 97 | 32.409 | 0.214 | −94.626 | 1.00 | 17.24 | 8 |
| ATOM | 663 | N | SER | 98 | 30.332 | 1.057 | −94.406 | 1.00 | 17.03 | 7 |
| ATOM | 664 | CA | SER | 98 | 29.670 | 0.180 | −94.811 | 1.00 | 18.11 | 6 |
| ATOM | 665 | CB | SER | 98 | 28.218 | 0.182 | −94.336 | 1.00 | 18.68 | 6 |
| ATOM | 666 | OG | SER | 98 | 27.446 | 0.723 | −95.113 | 1.00 | 19.16 | 8 |
| ATOM | 667 | C | SER | 98 | 29.687 | 0.308 | −96.333 | 1.00 | 18.78 | 6 |
| ATOM | 668 | O | SER | 98 | 29.472 | 1.389 | −96.880 | 1.00 | 17.71 | 8 |
| ATOM | 669 | N | GLY | 99 | 29.936 | 0.805 | −97.014 | 1.00 | 19.40 | 7 |
| ATOM | 670 | CA | GLY | 99 | 29.958 | 0.781 | −98.464 | 1.00 | 18.77 | 6 |
| ATOM | 671 | C | GLY | 99 | 28.585 | 1.010 | −99.070 | 1.00 | 21.80 | 6 |
| ATOM | 672 | O | GLY | 99 | 28.437 | 1.000 | −100.292 | 1.00 | 22.17 | 8 |
| ATOM | 673 | N | GLU | 100 | 27.578 | 1.213 | −98.223 | 1.00 | 23.11 | 7 |
| ATOM | 674 | CA | GLU | 100 | 26.217 | 1.457 | −98.693 | 1.00 | 25.94 | 6 |
| ATOM | 675 | CB | GLU | 100 | 25.260 | 1.543 | −97.502 | 1.00 | 28.92 | 6 |
| ATOM | 676 | CG | GLU | 100 | 25.030 | 0.214 | −96.806 | 1.00 | 37.13 | 6 |
| ATOM | 677 | CD | GLU | 100 | 24.270 | 0.358 | −95.502 | 1.00 | 40.36 | 6 |
| ATOM | 678 | OE1 | GLU | 100 | 24.832 | 0.925 | −94.540 | 1.00 | 43.10 | 8 |
| ATOM | 679 | OE2 | GLU | 100 | 23.108 | 0.095 | −95.441 | 1.00 | 44.50 | 8 |
| ATOM | 680 | C | GLU | 100 | 26.137 | 2.748 | −99.509 | 1.00 | 26.73 | 6 |
| ATOM | 681 | O | GLU | 100 | 25.38.9 | 2.839 | −100.481 | 1.00 | 26.36 | 8 |
| ATOM | 682 | N | GLU | 101 | 26.911 | 3.747 | −99.102 | 1.00 | 26.63 | 7 |
| ATOM | 683 | CA | GLU | 101 | 26.943 | 5.040 | −99.785 | 1.00 | 29.01 | 6 |
| ATOM | 684 | CB | GLU | 101 | 26.068 | 6.052 | −99.032 | 1.00 | 31.60 | 6 |
| ATOM | 685 | CG | GLU | 101 | 24.582 | 5.888 | −99.262 | 1.00 | 37.18 | 6 |
| ATOM | 686 | CD | GLU | 101 | 24.125 | 6.597 | −100.514 | 1.00 | 38.07 | 6 |
| ATOM | 687 | OE1 | GLU | 101 | 24.751 | 6.395 | −101.576 | 1.00 | 42.16 | 8 |
| ATOM | 688 | OE2 | GLU | 101 | 23.141 | 7.359 | −100.437 | 1.00 | 41.69 | 8 |
| ATOM | 689 | C | GLU | 101 | 28.38.2 | 5.528 | −99.780 | 1.00 | 28.05 | 6 |
| ATOM | 690 | O | GLU | 101 | 29.082 | 5.351 | −98.788 | 1.00 | 29.11 | 8 |
| ATOM | 691 | N | ASP | 102 | 28.849 | 6.134 | −100.867 | 1.00 | 26.56 | 7 |
| ATOM | 692 | CA | ASP | 102 | 30.222 | 6.619 | −100.833 | 1.00 | 27.19 | 6 |
| ATOM | 693 | CB | ASP | 102 | 30.943 | 6.367 | −102.165 | 1.00 | 32.95 | 6 |
| ATOM | 694 | CG | ASP | 102 | 30.237 | 6.975 | −103.349 | 1.00 | 35.72 | 6 |
| ATOM | 695 | OD1 | ASP | 102 | 30.569 | 6.576 | −104.488 | 1.00 | 39.04 | 8 |
| ATOM | 696 | OD2 | ASP | 102 | 29.366 | 7.845 | −103.149 | 1.00 | 39.37 | 8 |
| ATOM | 697 | C | ASP | 102 | 30.298 | 8.087 | −100.427 | 1.00 | 23.53 | 6 |
| ATOM | 698 | O | ASP | 102 | 29.345 | 8.850 | −100.593 | 1.00 | 20.78 | 8 |
| ATOM | 699 | N | PHE | 103 | 31.440 | 8.454 | −99.863 | 1.00 | 20.71 | 7 |
| ATOM | 700 | CA | PHE | 103 | 31.695 | 9.807 | −99.388 | 1.00 | 20.03 | 6 |
| ATOM | 701 | CE | PHE | 103 | 33.191 | 9.966 | −99.081 | 1.00 | 20.10 | 6 |
| ATOM | 702 | CG | PHE | 103 | 33.574 | 11.347 | −98.629 | 1.00 | 19.54 | 6 |
| ATOM | 703 | CD1 | PHE | 103 | 33.547 | 11.685 | −97.281 | 1.00 | 18.73 | 6 |
| ATOM | 704 | CD2 | PHE | 103 | 33.913 | 12.326 | −99.560 | 1.00 | 20.10 | 6 |
| ATOM | 705 | CE1 | PHE | 103 | 33.849 | 12.980 | −96.864 | 1.00 | 18.45 | 6 |
| ATOM | 706 | CE2 | PHE | 103 | 34.214 | 13.622 | −99.154 | 1.00 | 20.26 | 6 |
| ATOM | 707 | CZ | PHE | 103 | 34.181 | 13.949 | −97.805 | 1.00 | 19.18 | 6 |
| ATOM | 708 | C | PHE | 103 | 31.251 | 10.913 | −100.351 | 1.00 | 17.90 | |
| ATOM | 709 | O | PHE | 103 | 30.539 | 11.833 | −99.957 | 1.00 | 16.50 | 8 |
| ATOM | 710 | N | GLU | 104 | 31.663 | 10.812 | −101.612 | 1.00 | 18.97 | 7 |
| ATOM | 711 | CA | GLU | 104 | 31.341 | 11.831 | −102.612 | 1.00 | 20.50 | 6 |
| ATOM | 712 | CB | GLU | 104 | 31.992 | 11.491 | −103.962 | 1.00 | 20.41 | 6 |
| ATOM | 713 | CG | GLU | 104 | 33.508 | 11.436 | −103.922 | 1.00 | 22.85 | 6 |
| ATOM | 714 | CD | GLU | 104 | 34.036 | 10.077 | −103.498 | 1.00 | 24.56 | 6 |
| ATOM | 715 | OE1 | GLU | 104 | 33.287 | 9.319 | −102.844 | 1.00 | 23.41 | 8 |
| ATOM | 716 | OE2 | GLU | 104 | 35.207 | 9.771 | −103.815 | 1.00 | 26.00 | 8 |
| ATOM | 717 | C | GLU | 104 | 29.853 | 12.064 | −102.822 | 1.00 | 20.34 | 6 |
| ATOM | 718 | OC | GLU | 104 | 29.415 | 13.204 | −103.001 | 1.00 | 19.81 | 8 |
| ATOM | 719 | N | SER | 105 | 29.086 | 10.980 | −102.819 | 1.00 | 20.50 | 7 |
| ATOM | 720 | CA | SER | 105 | 27.643 | 11.059 | −103.009 | 1.00 | 21.22 | 6 |
| ATOM | 721 | CB | SER | 105 | 27.060 | 9.649 | −103.139 | 1.00 | 22.53 | 6 |
| ATOM | 722 | OG | SER | 105 | 25.649 | 9.674 | −103.221 | 1.00 | 26.65 | 8 |
| ATOM | 723 | C | SER | 105 | 26.980 | 11.797 | −101.843 | 1.00 | 19.51 | 6 |
| ATOM | 724 | O | SER | 105 | 26.184 | 12.716 | −102.048 | 1.00 | 19.50 | 8 |
| ATOM | 725 | N | LEU | 106 | 27.317 | 11.399 | −100.621 | 1.00 | 18.06 | 7 |
| ATOM | 726 | CA | LEU | 106 | 26.739 | 12.022 | −99.430 | 1.00 | 16.09 | 6 |
| ATOM | 727 | CB | LEU | 106 | 27.125 | 11.229 | −98.173 | 1.00 | 14.33 | 6 |
| ATOM | 728 | CG | LEU | 106 | 26.715 | 9.750 | −98.141 | 1.00 | 15.03 | 6 |
| ATOM | 729 | CD1 | LEU | 106 | 27.150 | 9.130 | −96.811 | 1.00 | 10.96 | 6 |
| ATOM | 730 | CD2 | LEU | 106 | 25.199 | 9.617 | −98.308 | 1.00 | 12.25 | 6 |
| ATOM | 731 | C | LEU | 106 | 27.159 | 13.481 | −99.270 | 1.00 | 15.52 | 6 |
| ATOM | 732 | O | LEU | 106 | 26.381 | 14.302 | −98.788 | 1.00 | 16.27 | 8 |
| ATOM | 733 | N | ALA | 107 | 28.386 | 13.808 | −99.663 | 1.00 | 15.14 | 7 |
| ATOM | 734 | CA | ALA | 107 | 28.853 | 15.187 | −99.557 | 1.00 | 16.06 | 6 |
| ATOM | 735 | CB | ALA | 107 | 30.347 | 15.261 | −99.844 | 1.00 | 13.47 | 6 |
| ATOM | 736 | C | ALA | 107 | 28.083 | 16.068 | −100.542 | 1.00 | 16.83 | 6 |
| ATOM | 737 | O | ALA | 107 | 27.643 | 17.167 | −100.200 | 1.00 | 15.43 | 8 |
| ATOM | 738 | N | SER | 108 | 27.928 | 15.574 | −101.767 | 1.00 | 16.86 | 7 |
| ATOM | 739 | CA | SER | 108 | 27.214 | 16.300 | −102.808 | 1.00 | 17.01 | 6 |
| ATOM | 740 | CB | SER | 108 | 27.214 | 15.493 | −104.112 | 1.00 | 15.11 | 6 |
| ATOM | 741 | OG | SER | 108 | 28.525 | 15.380 | −104.636 | 1.00 | 22.09 | 8 |
| ATOM | 742 | C | SER | 108 | 25.776 | 16.559 | −102.392 | 1.00 | 18.40 | 6 |
| ATOM | 743 | O | SER | 108 | 25.211 | 17.613 | −102.683 | 1.00 | 17.83 | 8 |
| ATOM | 744 | N | GLN | 109 | 25.193 | 15.589 | −101.701 | 1.00 | 18.13 | 7 |
| ATOM | 745 | CA | GLN | 109 | 23.808 | 15.675 | −101.257 | 1.00 | 20.27 | 6 |
| ATOM | 746 | CB | GLN | 109 | 23.222 | 14.273 | −101.117 | 1.00 | 21.15 | 6 |
| ATOM | 747 | CG | GLN | 109 | 22.931 | 13.538. | −102.401 | 1.00 | 22.47 | 6 |
| ATOM | 748 | CD | GLN | 109 | 22.469 | 12.127 | −102.121 | 1.00 | 23.94 | 6 |
| ATOM | 749 | OE1 | GLN | 109 | 23.281 | 11.214 | −101.945 | 1.00 | 24.29 | 8 |
| ATOM | 750 | NE2 | GLN | 109 | 21.162 | 11.942 | −102.050 | 1.00 | 22.04 | 7 |
| ATOM | 751 | C | GLN | 109 | 23.541 | 16.387 | −99.939 | 1.00 | 19.89 | 6 |
| ATOM | 752 | O | GLN | 109 | 22.561 | 17.114 | −99.812 | 1.00 | 19.48 | 8 |
| ATOM | 753 | N | PHE | 110 | 24.402 | 16.177 | −98.954 | 1.00 | 19.10 | 7 |
| ATOM | 754 | CA | PHE | 110 | 24.128 | 16.739 | −97.644 | 1.00 | 19.98 | 6 |
| ATOM | 755 | CB | PHE | 110 | 23.830 | 15.583 | −96.676 | 1.00 | 19.30 | 6 |
| ATOM | 756 | CG | PHE | 110 | 22.837 | 14.576 | −97.210 | 1.00 | 19.59 | 6 |
| ATOM | 757 | CD1 | PHE | 110 | 23.239 | 13.278 | −97.519 | 1.00 | 19.06 | 6 |
| ATOM | 758 | CD2 | PHE | 110 | 21.499 | 14.922 | −97.395 | 1.00 | 20.48 | 6 |
| ATOM | 759 | GE1 | PHE | 110 | 22.328 | 12.334 | −98.003 | 1.00 | 20.49 | 6 |
| ATOM | 760 | CE2 | PHE | 110 | 20.575 | 13.987 | −97.880 | 1.00 | 20.94 | 6 |
| ATOM | 761 | CZ | PHE | 110 | 20.992 | 12.686 | −98.185 | 1.00 | 20.51 | 6 |
| ATOM | 762 | C | PHE | 110 | 25.133 | 17.688 | −96.991 | 1.00 | 19.44 | 6 |
| ATOM | 763 | O | PHE | 110 | 24.884 | 18.153 | −95.884 | 1.00 | 20.18 | 8 |
| ATOM | 764 | N | SER | 111 | 26.250 | 17.990 | −97.644 | 1.00 | 17.53 | 7 |
| ATOM | 765 | CA | SER | 111 | 27.221 | 18.893 | −97.023 | 1.00 | 18.25 | 6 |
| ATOM | 766 | CB | SER | 111 | 28.558 | 18.834 | −97.748 | 1.00 | 15.50 | 6 |
| ATOM | 767 | OG | SER | 111 | 29.495 | 19.676 | −97.099 | 1.00 | 15.03 | 8 |
| ATOM | 768 | C | SER | 111 | 26.738 | 20.342 | −96.998 | 1.00 | 19.06 | 6 |
| ATOM | 769 | O | SER | 111 | 26.319 | 20.877 | −98.024 | 1.00 | 18.43 | 8 |
| ATOM | 770 | N | ASP | 112 | 26.803 | 20.973 | −95.826 | 1.00 | 18.80 | 7 |
| ATOM | 771 | CA | ASP | 112 | 26.369 | 22.361 | −95.689 | 1.00 | 19.60 | 6 |
| ATOM | 772 | CB | ASP | 112 | 25.957 | 22.665 | −94.244 | 1.00 | 16.06 | 6 |
| ATOM | 773 | CG | ASP | 112 | 24.576 | 22.139 | −93.911 | 1.00 | 18.54 | 6 |
| ATOM | 774 | OD1 | ASP | 112 | 23.635 | 22.436 | −94.675 | 1.00 | 17.83 | 8 |
| ATOM | 775 | OD2 | ASP | 112 | 24.424 | 21.434 | −92.888 | 1.00 | 17.41 | 8 |
| ATOM | 776 | C | ASP | 112 | 27.453 | 23.335 | −96.125 | 1.00 | 20.09 | 6 |
| ATOM | 777 | O | ASP | 112 | 27.482 | 24.481 | −95.691 | 1.00 | 21.88 | 8 |
| ATOM | 778 | N | CYS | 113 | 28.345 | 22.870 | −96.987 | 1.00 | 21.26 | 7 |
| ATOM | 779 | CA | CYS | 113 | 29.421 | 23.703 | −97.494 | 1.00 | 20.34 | 6 |
| ATOM | 780 | CB | CYS | 113 | 30.767 | 23.033 | −97.229 | 1.00 | 20.07 | 6 |
| ATOM | 781 | SG | CYS | 113 | 32.208 | 23.999 | −97.733 | 1.00 | 20.52 | 16 |
| ATOM | 782 | C | CYS | 113 | 29.217 | 23.881 | −98.990 | 1.00 | 20.53 | 6 |
| ATOM | 783 | O | CYS | 113 | 28.627 | 23.021 | −99.644 | 1.00 | 18.54 | 8 |
| ATOM | 784 | N | SER | 114 | 29.690 | 25.002 | −99.529 | 1.00 | 20.03 | 7 |
| ATOM | 785 | CA | SER | 114 | 29.560 | 25.257 | −100.958 | 1.00 | 21.77 | 6 |
| ATOM | 786 | CB | SER | 114 | 29.956 | 26.704 | −101.284 | 1.00 | 23.37 | 6 |
| ATOM | 787 | OG | SER | 114 | 31.209 | 27.036 | −100.709 | 1.00 | 28.15 | 8 |
| ATOM | 788 | C | SER | 114 | 30.443 | 24.286 | −101.740 | 1.00 | 21.75 | 6 |
| ATOM | 789 | O | SER | 114 | 30.282 | 24.124 | −102.954 | 1.00 | 21.05 | 8 |
| ATOM | 790 | N | SER | 115 | 31.371 | 23.636 | −101.039 | 1.00 | 20.37 | 7 |
| ATOM | 791 | CA | SER | 115 | 32.263 | 22.670 | −101.672 | 1.00 | 19.40 | 6 |
| ATOM | 792 | CB | SER | 115 | 33.414 | 22.297 | −100.730 | 1.00 | 19.04 | 6 |
| ATOM | 793 | OG | SER | 115 | 32.926 | 21.666 | −99.558 | 1.00 | 19.75 | 8 |
| ATOM | 794 | C | SER | 115 | 31.466 | 21.420 | −102.034 | 1.00 | 18.82 | 6 |
| ATOM | 795 | O | SER | 115 | 31.953 | 20.544 | −102.746 | 1.00 | 18.69 | 8 |
| ATOM | 796 | N | ALA | 116 | 30.236 | 21.340 | −101.536 | 1.00 | 18.27 | 7 |
| ATOM | 797 | CA | ALA | 116 | 29.370 | 20.202 | −101.825 | 1.00 | 15.63 | 6 |
| ATOM | 798 | CB | ALA | 116 | 27.988 | 20.435 | −101.231 | 1.00 | 16.73 | 6 |
| ATOM | 799 | C | ALA | 116 | 29.262 | 20.017 | −103.339 | 1.00 | 18.76 | 6 |
| ATOM | 800 | O | ALA | 116 | 29.242 | 18.886 | −103.845 | 1.00 | 15.11 | 8 |
| ATOM | 801 | N | LYS | 117 | 29.188 | 21.141 | −104.051 | 1.00 | 16.75 | 7 |
| ATOM | 802 | CA | LYS | 117 | 29.072 | 21.147 | −105.509 | 1.00 | 18.04 | 6 |
| ATOM | 803 | CE | LYS | 117 | 28.944 | 22.586 | −106.022 | 1.00 | 20.56 | 6 |
| ATOM | 804 | CG | LYS | 117 | 27.791 | 23.354 | −105.410 | 1.00 | 26.14 | 6 |
| ATOM | 805 | CD | LYS | 117 | 26.461 | 22.813 | −105.875 | 1.00 | 30.18 | 6 |
| ATOM | 806 | CE | LYS | 117 | 25.344 | 23.192 | −104.907 | 1.00 | 32.81 | 6 |
| ATOM | 807 | NZ | LYS | 117 | 25.379 | 22.328 | −103.691 | 1.00 | 30.54 | 7 |
| ATOM | 808 | C | LYS | 117 | 30.264 | 20.480 | −106.186 | 1.00 | 16.32 | 6 |
| ATOM | 809 | O | LYS | 117 | 30.187 | 20.094 | −107.352 | 1.00 | 14.87 | 8 |
| ATOM | 810 | N | ALA | 118 | 31.371 | 20.372 | −105.461 | 1.00 | 15.62 | 7 |
| ATOM | 811 | CA | ALA | 118 | 32.570 | 19.731 | −105.987 | 1.00 | 17.05 | 6 |
| ATOM | 812 | CB | ALA | 118 | 33.777 | 20.643 | −105.813 | 1.00 | 17.60 | 6 |
| ATOM | 813 | C | ALA | 118 | 32.798 | 18.413 | −105.245 | 1.00 | 16.59 | 6 |
| ATOM | 814 | O | ALA | 118 | 33.934 | 17.988 | −105.047 | 1.00 | 15.92 | 8 |
| ATOM | 815 | N | ARG | 119 | 31.706 | 17.781 | −104.834 | 1.00 | 18.14 | 7 |
| ATOM | 816 | CA | ARG | 119 | 31.764 | 16.515 | −104.108 | 1.00 | 19.34 | 6 |
| ATOM | 817 | CB | ARG | 119 | 32.380 | 15.423 | −104.990 | 1.00 | 23.02 | 6 |
| ATOM | 818 | CG | ARG | 119 | 31.719 | 15.297 | −106.355 | 1.00 | 29.34 | 6 |
| ATOM | 819 | CD | ARG | 119 | 32.647 | 14.617 | −107.357 | 1.00 | 34.72 | 6 |
| ATOM | 820 | NE | ARG | 119 | 32.409 | 13.183 | −107.483 | 1.00 | 40.32 | 7 |
| ATOM | 821 | CZ | ARG | 119 | 31.491 | 12.642 | −108.280 | 1.00 | 44.43 | 6 |
| ATOM | 822 | NH1 | ARG | 119 | 30.717 | 13.419 | −109.024 | 1.00 | 45.83 | 7 |
| ATOM | 823 | NH2 | ARG | 119 | 31.358 | 11.322 | −108.349 | 1.00 | 44.84 | 7 |
| ATOM | 824 | C | ARG | 119 | 32.577 | 16.671 | −102.829 | 1.00 | 19.60 | 6 |
| ATOM | 825 | O | ARG | 119 | 33.205 | 15.721 | −102.365 | 1.00 | 17.58 | 8 |
| ATOM | 826 | N | GLY | 120 | 32.574 | 17.882 | −102.275 | 1.00 | 17.95 | 7 |
| ATOM | 827 | CA | GLY | 120 | 33.292 | 18.138 | −101.039 | 1.00 | 16.72 | 6 |
| ATOM | 828 | C | GLY | 120 | 34.758 | 18.510 | −101.164 | 1.00 | 17.94 | 6 |
| ATOM | 829 | O | GLY | 120 | 35.392 | 18.869 | −100.174 | 1.00 | 16.29 | 8 |
| ATOM | 830 | N | ASP | 121 | 35.309 | 18.430 | −102.369 | 1.00 | 17.64 | 7 |
| ATOM | 831 | CA | ASP | 121 | 36.715 | 18.762 | −102.568 | 1.00 | 17.43 | 6 |
| ATOM | 832 | CB | ASP | 121 | 37.132 | 18.456 | −104.011 | 1.00 | 18.27 | 6 |
| ATOM | 833 | CG | ASP | 121 | 38.560 | 18.877 | −104.306 | 1.00 | 20.49 | 6 |
| ATOM | 834 | OD1 | ASP | 121 | 39.451 | 18.599 | −103.475 | 1.00 | 20.46 | 8 |
| ATOM | 835 | OD2 | ASP | 121 | 38.796 | 19.476 | −105.373 | 1.00 | 19.97 | 8 |
| ATOM | 836 | C | ASP | 121 | 37.028 | 20.223 | −102.239 | 1.00 | 17.81 | 6 |
| ATOM | 837 | O | ASP | 121 | 36.330 | 21.139 | −102.677 | 1.00 | 15.80 | 8 |
| ATOM | 838 | N | LED | 122 | 38.089 | 20.424 | −101.462 | 1.00 | 17.98 | 7 |
| ATOM | 839 | CA | LED | 122 | 38.518 | 21.755 | −101.062 | 1.00 | 19.96 | 6 |
| ATOM | 840 | CB | LED | 122 | 38.702 | 21.813 | −99.541 | 1.00 | 18.98 | 6 |
| ATOM | 841 | CG | LED | 122 | 37.493 | 21.491 | −98.657 | 1.00 | 16.15 | 6 |
| ATOM | 842 | CD1 | LED | 122 | 37.951 | 21.338 | −97.208 | 1.00 | 16.60 | 6 |
| ATOM | 843 | CD2 | LED | 122 | 36.454 | 22.585 | −98.783 | 1.00 | 15.56 | 6 |
| ATOM | 844 | C | LED | 122 | 39.834 | 22.139 | −101.732 | 1.00 | 20.80 | 6 |
| ATOM | 845 | O | LED | 122 | 40.308 | 23.257 | −101.572 | 1.00 | 23.22 | 8 |
| ATOM | 846 | N | GLY | 123 | 40.429 | 21.212 | −102.476 | 1.00 | 23.28 | 7 |
| ATOM | 847 | CA | GLY | 123 | 41.697 | 21.503 | −103.119 | 1.00 | 21.01 | 6 |
| ATOM | 848 | C | GLY | 123 | 42.816 | 21.348 | −102.107 | 1.00 | 22.64 | 6 |
| ATOM | 849 | O | GLY | 123 | 42.566 | 20.993 | −100.952 | 1.00 | 23.80 | 8 |
| ATOM | 850 | N | ALA | 124 | 44.047 | 21.617 | −102.522 | 1.00 | 21.86 | 7 |
| ATOM | 851 | CA | ALA | 124 | 45.198 | 21.484 | −101.636 | 1.00 | 23.48 | 6 |
| ATOM | 852 | CB | ALA | 124 | 46.462 | 21.243 | −102.460 | 1.00 | 23.90 | 6 |
| ATOM | 853 | C | ALA | 124 | 45.376 | 22.716 | −100.753 | 1.00 | 24.83 | 6 |
| ATOM | 854 | O | ALA | 124 | 44.994 | 23.821 | −101.131 | 1.00 | 25.51 | 8 |
| ATOM | 855 | N | PHE | 125 | 45.952 | 22.518 | −99.572 | 1.00 | 24.98 | 7 |
| ATOM | 856 | CA | PHE | 125 | 46.186 | 23.620 | −98.647 | 1.00 | 26.31 | 6 |
| ATOM | 857 | CB | PHE | 125 | 44.900 | 23.944 | −97.864 | 1.00 | 25.69 | 6 |
| ATOM | 858 | CG | PHE | 125 | 44.317 | 22.766 | −97.111 | 1.00 | 26.95 | 6 |
| ATOM | 859 | CD1 | PHE | 125 | 44.874 | 22.342 | −95.906 | 1.00 | 26.54 | 6 |
| ATOM | 860 | CD2 | PHE | 125 | 43.209 | 22.083 | −97.611 | 1.00 | 25.95 | 6 |
| ATOM | 861 | CE1 | PHE | 125 | 44.338 | 21.256 | −95.207 | 1.00 | 26.42 | 6 |
| ATOM | 862 | CE2 | PHE | 125 | 42.664 | 20.993 | −96.919 | 1.00 | 28.09 | 6 |
| ATOM | 863 | CZ | PHE | 125 | 43.232 | 20.580 | −95.713 | 1.00 | 24.17 | 6 |
| ATOM | 864 | C | PHE | 125 | 47.332 | 23.308 | −97.694 | 1.00 | 26.19 | 6 |
| ATOM | 865 | O | PHE | 125 | 47.760 | 22.162 | −97.580 | 1.00 | 28.53 | 8 |
| ATOM | 866 | N | SER | 126 | 47.846 | 24.336 | −97.028 | 1.00 | 27.90 | 7 |
| ATOM | 867 | CA | SER | 126 | 48.936 | 24.153 | −96.078 | 1.00 | 28.63 | 6 |
| ATOM | 868 | CB | SER | 126 | 50.149 | 24.999 | −96.481 | 1.00 | 31.22 | 6 |
| ATOM | 869 | OG | SER | 126 | 49.848 | 26.381 | −96.415 | 1.00 | 31.69 | 8 |
| ATOM | 870 | C | SER | 126 | 48.448 | 24.577 | −94.701 | 1.00 | 28.14 | 6 |
| ATOM | 871 | O | SER | 126 | 47.327 | 25.067 | −94.558 | 1.00 | 27.13 | 8 |
| ATOM | 872 | N | ARG | 127 | 49.280 | 24.382 | −93.686 | 1.00 | 29.19 | 7 |
| ATOM | 873 | CA | ARG | 127 | 48.900 | 24.772 | −92.338 | 1.00 | 31.19 | 6 |
| ATOM | 874 | CB | ARG | 127 | 49.953 | 24.308 | −91.329 | 1.00 | 30.85 | 6 |
| ATOM | 875 | CG | ARG | 127 | 49.885 | 22.821 | −91.006 | 1.00 | 29.53 | 6 |
| ATOM | 876 | CD | ARG | 127 | 50.870 | 22.456 | −89.913 | 1.00 | 29.23 | 6 |
| ATOM | 877 | NE | ARG | 127 | 50.840 | 21.036 | −89.566 | 1.00 | 27.18 | 7 |
| ATOM | 878 | CZ | ARG | 127 | 49.891 | 20.452 | −88.837 | 1.00 | 25.14 | 6 |
| ATOM | 879 | NH1 | ARG | 127 | 48.875 | 21.159 | −88.367 | 1.00 | 22.98 | 7 |
| ATOM | 880 | NH2 | ARG | 127 | 49.970 | 19.156 | −88.566 | 1.00 | 25.04 | 7 |
| ATOM | 881 | C | ARG | 127 | 48.719 | 26.284 | −92.255 | 1.00 | 33.31 | 6 |
| ATOM | 882 | O | ARG | 127 | 49.320 | 27.036 | −93.024 | 1.00 | 35.02 | 8 |
| ATOM | 883 | N | GLY | 128 | 47.871 | 26.721 | −91.331 | 1.00 | 34.55 | 7 |
| ATOM | 884 | CA | GLY | 128 | 47.623 | 28.139 | −91.158 | 1.00 | 35.59 | 6 |
| ATOM | 885 | C | GLY | 128 | 46.494 | 28.692 | −92.009 | 1.00 | 35.87 | 6 |
| ATOM | 886 | O | GLY | 128 | 46.143 | 29.865 | −91.878 | 1.00 | 36.97 | 8 |
| ATOM | 887 | N | GLN | 129 | 45.906 | 27.861 | −92.867 | 1.00 | 33.00 | 7 |
| ATOM | 888 | CA | GLN | 129 | 44.828 | 28.325 | −93.737 | 1.00 | 32.73 | 6 |
| ATOM | 889 | CS | GLN | 129 | 45.083 | 27.849 | −95.168 | 1.00 | 33.54 | 6 |
| ATOM | 890 | CG | GLN | 129 | 46.407 | 28.344 | −95.733 | 1.00 | 35.45 | 6 |
| ATOM | 891 | CD | GLN | 129 | 46.580 | 28.019 | −97.203 | 1.00 | 38.37 | 6 |
| ATOM | 892 | OE1 | GLN | 129 | 46.642 | 26.853 | −97.592 | 1.00 | 38.18 | 8 |
| ATOM | 893 | NE2 | GLN | 129 | 46.659 | 29.055 | −98.029 | 1.00 | 40.47 | 7 |
| ATOM | 894 | C | GLN | 129 | 43.415 | 27.933 | −93.307 | 1.00 | 31.98 | 6 |
| ATOM | 895 | O | GLN | 129 | 42.477 | 28.717 | −93.453 | 1.00 | 31.06 | 8 |
| ATOM | 896 | N | MET | 130 | 43.262 | 26.726 | −92.778 | 1.00 | 30.66 | 7 |
| ATOM | 897 | CA | MET | 130 | 41.951 | 26.252 | −92.344 | 1.00 | 30.15 | 6 |
| ATOM | 898 | CB | MET | 130 | 41.767 | 24.785 | −92.742 | 1.00 | 30.87 | 6 |
| ATOM | 899 | CG | MET | 130 | 42.069 | 24.473 | −94.199 | 1.00 | 31.09 | 6 |
| ATOM | 900 | SD | MET | 130 | 40.927 | 25.246 | −95.345 | 1.00 | 34.31 | 16 |
| ATOM | 901 | CE | MET | 130 | 39.412 | 24.418 | −94.924 | 1.00 | 36.22 | 6 |
| ATOM | 902 | C | MET | 130 | 41.832 | 26.366 | −90.831 | 1.00 | 29.32 | 6 |
| ATOM | 903 | O | MET | 130 | 42.824 | 26.593 | −90.144 | 1.00 | 29.87 | 8 |
| ATOM | 904 | N | GLN | 131 | 40.620 | 26.214 | −90.310 | 1.00 | 28.47 | 7 |
| ATOM | 905 | CA | GLN | 131 | 40.436 | 26.261 | −88.869 | 1.00 | 28.52 | 6 |
| ATOM | 906 | CB | GLN | 131 | 38.971 | 26.056 | −88.516 | 1.00 | 30.93 | 6 |
| ATOM | 907 | CG | GLN | 131 | 38.092 | 27.145 | −89.083 | 1.00 | 32.06 | 6 |
| ATOM | 908 | CD | GLN | 131 | 36.656 | 27.012 | −88.657 | 1.00 | 33.27 | 6 |
| ATOM | 909 | OE1 | GLN | 131 | 36.337 | 27.099 | −87.473 | 1.00 | 38.30 | 8 |
| ATOM | 910 | NE2 | GLN | 131 | 35.775 | 26.797 | −89.620 | 1.00 | 35.11 | 7 |
| ATOM | 911 | C | GLN | 131 | 41.299 | 25.151 | −88.282 | 1.00 | 27.72 | 6 |
| ATOM | 912 | O | GLN | 131 | 41.353 | 24.039 | −88.814 | 1.00 | 26.02 | 8 |
| ATOM | 913 | N | LYS | 132 | 41.978 | 25.476 | −87.188 | 1.00 | 26.17 | 7 |
| ATOM | 914 | CA | LYS | 132 | 42.900 | 24.571 | −86.516 | 1.00 | 24.31 | 6 |
| ATOM | 915 | CB | LYS | 132 | 43.285 | 25.171 | −85.157 | 1.00 | 24.74 | 6 |
| ATOM | 916 | CG | LYS | 132 | 44.463 | 24.490 | −84.496 | 1.00 | 23.27 | 6 |
| ATOM | 917 | CD | LYS | 132 | 45.664 | 24.460 | −85.419 | 1.00 | 21.69 | 6 |
| ATOM | 918 | CE | LYS | 132 | 46.837 | 23.775 | −84.747 | 1.00 | 25.05 | 6 |
| ATOM | 919 | NZ | LYS | 132 | 47.958 | 23.528 | −85.687 | 1.00 | 25.90 | 7 |
| ATOM | 920 | C | LYS | 132 | 42.499 | 23.099 | −86.338. | 1.00 | 22.74 | 6 |
| ATOM | 921 | O | LYS | 132 | 43.285 | 22.205 | −86.650 | 1.00 | 23.41 | 8 |
| ATOM | 922 | N | PRO | 133 | 41.292 | 22.822 | −85.817 | 1.00 | 20.23 | 7 |
| ATOM | 923 | CD | PRO | 133 | 40.286 | 23.715 | −85.220 | 1.00 | 20.68 | 6 |
| ATOM | 924 | CA | PRO | 133 | 40.911 | 21.412 | −85.645 | 1.00 | 20.72 | 6 |
| ATOM | 925 | CB | PRO | 133 | 39.539 | 21.501 | −84.973 | 1.00 | 20.88 | 6 |
| ATOM | 926 | CG | PRO | 133 | 39.615 | 22.803 | −84.212 | 1.00 | 20.58 | 6 |
| ATOM | 927 | C | PRO | 133 | 40.857 | 20.657 | −86.976 | 1.00 | 19.31 | 6 |
| ATOM | 928 | O | PRO | 133 | 41.264 | 19.499 | −87.065 | 1.00 | 19.30 | 8 |
| ATOM | 929 | N | PHE | 134 | 40.348 | 21.328 | −88.004 | 1.00 | 20.16 | 7 |
| ATOM | 930 | CA | PHE | 134 | 40.235 | 20.753 | −89.342 | 1.00 | 17.39 | 6 |
| ATOM | 931 | CB | PHE | 134 | 39.504 | 21.734 | −90.261 | 1.00 | 18.57 | 6 |
| ATOM | 932 | CG | PHE | 134 | 39.145 | 21.162 | −91.603 | 1.00 | 16.72 | 6 |
| ATOM | 933 | CD1 | PHE | 134 | 37.923 | 20.534 | −91.799 | 1.00 | 16.56 | 6 |
| ATOM | 934 | CD2 | PHE | 134 | 40.041 | 21.229 | −92.662 | 1.00 | 17.33 | 6 |
| ATOM | 935 | CE1 | PHE | 134 | 37.592 | 19.977 | −93.035 | 1.00 | 16.65 | 6 |
| ATOM | 936 | CE2 | PHE | 134 | 39.721 | 20.673 | −93.905 | 1.00 | 17.05 | 6 |
| ATOM | 937 | CZ | PHE | 134 | 38.495 | 20.047 | −94.087 | 1.00 | 16.14 | 6 |
| ATOM | 938 | C | PHE | 134 | 41.637 | 20.495 | −89.882 | 1.00 | 18.38 | 6 |
| ATOM | 939 | O | PHE | 134 | 41.923 | 19.440 | −90.453 | 1.00 | 15.83 | 8 |
| ATOM | 940 | N | GLU | 135 | 42.514 | 21.475 | −89.687 | 1.00 | 18.89 | 7 |
| ATOM | 941 | CA | GLU | 135 | 43.895 | 21.387 | −90.142 | 1.00 | 18.62 | 6 |
| ATOM | 942 | CB | GLU | 135 | 44.631 | 22.687 | −89.816 | 1.00 | 20.34 | 6 |
| ATOM | 943 | CG | GLU | 135 | 46.116 | 22.640 | −90.103 | 1.00 | 23.27 | 6 |
| ATOM | 944 | CD | GLU | 135 | 46.826 | 23.896 | −89.631 | 1.00 | 25.96 | 6 |
| ATOM | 945 | OE1 | GLU | 135 | 46.390 | 24.999 | −90.020 | 1.00 | 25.71 | 8 |
| ATOM | 946 | OE2 | GLU | 135 | 47.814 | 23.773 | −88.875 | 1.00 | 25.79 | 8 |
| ATOM | 947 | C | GLU | 135 | 44.665 | 20.217 | −89.532 | 1.00 | 18.46 | 6 |
| ATOM | 948 | O | GLU | 135 | 45.269 | 19.413 | −90.252 | 1.00 | 16.37 | 8 |
| ATOM | 949 | N | ASP | 136 | 44.655 | 20.126 | −88.207 | 1.00 | 18.15 | 7 |
| ATOM | 950 | CA | ASP | 136 | 45.373 | 19.048 | −87.535 | 1.00 | 18.37 | 6 |
| ATOM | 951 | CB | ASP | 136 | 45.283 | 19.201 | −86.009 | 1.00 | 20.71 | 6 |
| ATOM | 952 | CG | ASP | 136 | 46.074 | 20.397 | −85.496 | 1.00 | 24.12 | 6 |
| ATOM | 953 | OD1 | ASP | 136 | 47.112 | 20.725 | −86.112 | 1.00 | 23.48 | 8 |
| ATOM | 954 | OD2 | ASP | 136 | 45.670 | 21.001 | −84.478 | 1.00 | 25.14 | 8 |
| ATOM | 955 | C | ASP | 136 | 44.852 | 17.683 | −87.961 | 1.00 | 15.88 | 6 |
| ATOM | 956 | O | ASP | 136 | 45.628 | 16.756 | −88.177 | 1.00 | 14.90 | 8 |
| ATOM | 957 | N | ALA | 137 | 43.536 | 17.561 | −88.088 | 1.00 | 16.60 | 7 |
| ATOM | 958 | CA | ALA | 137 | 42.948 | 16.294 | −88.508 | 1.00 | 15.53 | 6 |
| ATOM | 959 | CB | ALA | 137 | 41.420 | 16.391 | −88.478 | 1.00 | 14.06 | 6 |
| ATOM | 960 | C | ALA | 137 | 43.439 | 15.974 | −89.923 | 1.00 | 17.03 | 6 |
| ATOM | 961 | O | ALA | 137 | 43.914 | 14.871 | −90.198 | 1.00 | 18.29 | 8 |
| ATOM | 962 | N | SER | 138 | 43.348 | 16.959 | −90.811 | 1.00 | 18.95 | 7 |
| ATOM | 963 | CA | SER | 138 | 43.770 | 16.791 | −92.198 | 1.00 | 18.92 | 6 |
| ATOM | 964 | CB | SER | 138 | 43.568 | 18.097 | −92.973 | 1.00 | 18.00 | 6 |
| ATOM | 965 | OG | SER | 138 | 42.196 | 18.450 | −93.033 | 1.00 | 16.03 | 8 |
| ATOM | 966 | C | SER | 138 | 45.215 | 16.337 | −92.351 | 1.00 | 20.64 | 6 |
| ATOM | 967 | O | SER | 138 | 45.509 | 15.457 | −93.161 | 1.00 | 21.57 | 8 |
| ATOM | 968 | N | PHE | 139 | 46.118 | 16.934 | −91.581 | 1.00 | 21.16 | 7 |
| ATOM | 969 | CA | PHE | 139 | 47.524 | 16.574 | −91.667 | 1.00 | 22.95 | 6 |
| ATOM | 970 | CB | PHE | 139 | 48.408 | 17.733 | −91.181 | 1.00 | 24.16 | 6 |
| ATOM | 971 | CG | PHE | 139 | 48.624 | 18.802 | −92.221 | 1.00 | 24.18 | 6 |
| ATOM | 972 | CD1 | PHE | 139 | 47.655 | 19.773 | −92.456 | 1.00 | 23.62 | 6 |
| ATOM | 973 | CD2 | PHE | 139 | 49.768 | 18.793 | −93.015 | 1.00 | 25.16 | 6 |
| ATOM | 974 | CE1 | PHE | 139 | 47.816 | 20.719 | −93.470 | 1.00 | 22.81 | 6 |
| ATOM | 975 | CE2 | PHE | 139 | 49.941 | 19.734 | −94.034 | 1.00 | 24.56 | 6 |
| ATOM | 976 | CZ | PHE | 139 | 48.962 | 20.698 | −94.263 | 1.00 | 24.40 | 6 |
| ATOM | 977 | C | PHE | 139 | 47.878 | 15.291 | −90.919 | 1.00 | 22.86 | 6 |
| ATOM | 978 | O | PHE | 139 | 48.940 | 14.720 | −91.141 | 1.00 | 24.27 | 8 |
| ATOM | 979 | N | ALA | 140 | 46.988 | 14.836 | −90.044 | 1.00 | 21.99 | 7 |
| ATOM | 980 | CA | ALA | 140 | 47.236 | 13.616 | −89.286 | 1.00 | 21.24 | 6 |
| ATOM | 981 | CS | ALA | 140 | 46.547 | 13.693 | −87.936 | 1.00 | 19.93 | 6 |
| ATOM | 982 | C | ALA | 140 | 46.751 | 12.393 | −90.067 | 1.00 | 21.13 | 6 |
| ATOM | 983 | O | ALA | 140 | 47.157 | 11.265 | −89.793 | 1.00 | 22.85 | 8 |
| ATOM | 984 | N | LEU | 141 | 45.875 | 12.620 | −91.038. | 1.00 | 21.30 | 7 |
| ATOM | 985 | CA | LEU | 141 | 45.366 | 11.535 | −91.875 | 1.00 | 21.71 | 6 |
| ATOM | 986 | CS | LEU | 141 | 44.101 | 11.966 | −92.618 | 1.00 | 18.40 | 6 |
| ATOM | 987 | CG | LEU | 141 | 42.784 | 12.148 | −91.872 | 1.00 | 17.68 | 6 |
| ATOM | 988 | CD1 | LED | 141 | 41.798 | 12.884 | −92.784 | 1.00 | 15.42 | 6 |
| ATOM | 989 | CD2 | LED | 141 | 42.242 | 10.784 | −91.446 | 1.00 | 16.17 | 6 |
| ATOM | 990 | C | LED | 141 | 46.408 | 11.182 | −92.924 | 1.00 | 23.64 | 6 |
| ATOM | 991 | O | LED | 141 | 47.212 | 12.027 | −93.324 | 1.00 | 23.58 | 8 |
| ATOM | 992 | N | ARG | 142 | 46.398 | 9.934 | −93.368 | 1.00 | 24.74 | 7 |
| ATOM | 993 | CA | ARG | 142 | 47.313 | 9.520 | −94.419 | 1.00 | 26.64 | 6 |
| ATOM | 994 | CB | ARG | 142 | 47.853 | 8.123 | −94.130 | 1.00 | 31.37 | 6 |
| ATOM | 995 | CG | ARG | 142 | 49.002 | 8.143 | −93.131 | 1.00 | 36.54 | 6 |
| ATOM | 996 | CD | ARG | 142 | 49.104 | 6.842 | −92.369 | 1.00 | 42.72 | 6 |
| ATOM | 997 | NE | ARG | 142 | 47.988 | 6.670 | −91.447 | 1.00 | 45.09 | 7 |
| ATOM | 998 | CZ | ARG | 142 | 47.849 | 5.626 | −90.638 | 1.00 | 47.78 | 6 |
| ATOM | 999 | NH1 | ARG | 142 | 48.758 | 4.663 | −90.643 | 1.00 | 49.09 | 7 |
| ATOM | 1000 | NH2 | ARG | 142 | 46.806 | 5.544 | −89.822 | 1.00 | 49.62 | 7 |
| ATOM | 1001 | C | ARG | 142 | 46.493 | 9.547 | −95.701 | 1.00 | 25.60 | 6 |
| ATOM | 1002 | O | ARG | 142 | 45.263 | 9.513 | −95.648 | 1.00 | 24.09 | 8 |
| ATOM | 1003 | N | THR | 143 | 47.158 | 9.641 | −96.847 | 1.00 | 24.75 | 7 |
| ATOM | 1004 | CA | THR | 143 | 46.442 | 9.682 | −98.115 | 1.00 | 25.11 | 6 |
| ATOM | 1005 | CS | THR | 143 | 47.406 | 9.588 | −99.306 | 1.00 | 27.77 | 6 |
| ATOM | 1006 | OG1 | THR | 143 | 48.316 | 10.692 | −99.261 | 1.00 | 31.88 | 8 |
| ATOM | 1007 | CG2 | THR | 143 | 46.641 | 9.629 | −100.624 | 1.00 | 28.55 | 6 |
| ATOM | 1008 | C | THR | 143 | 45.452 | 8.532 | −98.188 | 1.00 | 23.59 | 6 |
| ATOM | 1009 | O | THR | 143 | 45.820 | 7.375 | −97.997 | 1.00 | 22.62 | 8 |
| ATOM | 1010 | N | GLY | 144 | 44.191 | 8.862 | −98.445 | 1.00 | 22.24 | 7 |
| ATOM | 1011 | CA | GLY | 144 | 43.161 | 7.845 | −98.543 | 1.00 | 21.11 | 6 |
| ATOM | 1012 | C | GLY | 144 | 42.329 | 7.642 | −97.288 | 1.00 | 20.32 | 6 |
| ATOM | 1013 | O | GLY | 144 | 41.196 | 7.167 | −97.372 | 1.00 | 21.72 | 8 |
| ATOM | 1014 | N | GLU | 145 | 42.876 | 8.001 | −96.129 | 1.00 | 19.79 | 7 |
| ATOM | 1015 | CA | GLU | 145 | 42.164 | 7.826 | −94.863 | 1.00 | 20.11 | 6 |
| ATOM | 1016 | CB | GLU | 145 | 43.141 | 7.850 | −93.685 | 1.00 | 20.11 | 6 |
| ATOM | 1017 | CG | GLU | 145 | 44.217 | 6.780 | −93.729 | 1.00 | 25.11 | 6 |
| ATOM | 1018 | CD | GLU | 145 | 45.062 | 6.773 | −92.466 | 1.00 | 27.35 | 6 |
| ATOM | 1019 | OE1 | GLU | 145 | 45.480 | 7.865 | −92.019 | 1.00 | 28.41 | 8 |
| ATOM | 1020 | OE2 | GLU | 145 | 45.311 | 5.677 | −91.922 | 1.00 | 29.76 | 8 |
| ATOM | 1021 | C | GLU | 145 | 41.085 | 8.872 | −94.619 | 1.00 | 18.58 | 6 |
| ATOM | 1022 | O | GLU | 145 | 41.160 | 9.994 | −95.121 | 1.00 | 19.31 | 8 |
| ATOM | 1023 | N | MET | 146 | 40.090 | 8.491 | −93.825 | 1.00 | 16.82 | 7 |
| ATOM | 1024 | CA | MET | 146 | 38.979 | 9.366 | −93.493 | 1.00 | 15.28 | 6 |
| ATOM | 1025 | CB | MET | 146 | 37.671 | 8.754 | −93.988 | 1.00 | 16.30 | 6 |
| ATOM | 1026 | CG | MET | 146 | 36.441 | 9.609 | −93.758 | 1.00 | 16.10 | 6 |
| ATOM | 1027 | SD | MET | 146 | 34.956 | 8.785 | −94.388 | 1.00 | 17.23 | 16 |
| ATOM | 1028 | CE | MET | 146 | 34.621 | 7.624 | −93.034 | 1.00 | 16.25 | 6 |
| ATOM | 1029 | C | MET | 146 | 38.927 | 9.567 | −91.986 | 1.00 | 15.52 | 6 |
| ATOM | 1030 | O | MET | 146 | 39.263 | 8.669 | −91.220 | 1.00 | 13.19 | 8 |
| ATOM | 1031 | N | SER | 147 | 38.509 | 10.757 | −91.570 | 1.00 | 13.30 | 7 |
| ATOM | 1032 | CA | SER | 147 | 38.425 | 11.085 | −90.156 | 1.00 | 12.46 | 6 |
| ATOM | 1033 | CB | SER | 147 | 38.538 | 12.594 | −89.964 | 1.00 | 11.46 | 6 |
| ATOM | 1034 | OG | SER | 147 | 37.305 | 13.209 | −90.323 | 1.00 | 14.08 | 8 |
| ATOM | 1035 | C | SER | 147 | 37.092 | 10.656 | −89.570 | 1.00 | 14.26 | 6 |
| ATOM | 1036 | O | SER | 147 | 36.217 | 10.150 | −90.273 | 1.00 | 13.67 | 8 |
| ATOM | 1037 | N | GLY | 148 | 36.965 | 10.860 | −88.264 | 1.00 | 14.51 | 7 |
| ATOM | 1038 | CA | GLY | 148 | 35.725 | 10.586 | −87.571 | 1.00 | 15.49 | 6 |
| ATOM | 1039 | C | GLY | 148 | 35.150 | 11.990 | −87.441 | 1.00 | 18.18 | 6 |
| ATOM | 1040 | O | GLY | 148 | 35.591 | 12.884 | −88.164 | 1.00 | 17.36 | 8 |
| ATOM | 1041 | N | PRO | 149 | 34.179 | 12.229 | −86.553 | 1.00 | 18.15 | 7 |
| ATOM | 1042 | CD | PRO | 149 | 33.382 | 11.270 | −85.770 | 1.00 | 18.03 | 6 |
| ATOM | 1043 | CA | PRO | 149 | 33.637 | 13.589 | −86.436 | 1.00 | 19.44 | 6 |
| ATOM | 1044 | CB | PRO | 149 | 32.519 | 13.430 | −85.409 | 1.00 | 17.92 | 6 |
| ATOM | 1045 | CG | PRO | 149 | 32.070 | 12.006 | −85.607 | 1.00 | 20.63 | 6 |
| ATOM | 1046 | C | PRO | 149 | 34.695 | 14.599 | −85.973 | 1.00 | 19.40 | 6 |
| ATOM | 1047 | O | PRO | 149 | 35.341 | 14.393 | −84.945 | 1.00 | 18.89 | 8 |
| ATOM | 1048 | N | VAL | 150 | 34.865 | 15.680 | −86.734 | 1.00 | 18.59 | 7 |
| ATOM | 1049 | CA | VAL | 150 | 35.826 | 16.740 | −86.398 | 1.00 | 17.12 | 6 |
| ATOM | 1050 | CB | VAL | 150 | 36.863 | 16.929 | −87.531 | 1.00 | 16.74 | 6 |
| ATOM | 1051 | CD1 | VAL | 150 | 37.791 | 18.100 | −87.212 | 1.00 | 17.38 | 6 |
| ATOM | 1052 | CG2 | VAL | 150 | 37.684 | 15.642 | −87.700 | 1.00 | 14.98 | 6 |
| ATOM | 1053 | C | VAL | 150 | 35.045 | 18.047 | −86.173 | 1.00 | 18.98 | 6 |
| ATOM | 1054 | O | VAL | 150 | 34.306 | 18.497 | −87.049 | 1.00 | 19.25 | 8 |
| ATOM | 1055 | N | PHE | 151 | 35.220 | 18.652 | −84.999 | 1.00 | 17.99 | 7 |
| ATOM | 1056 | CA | PHE | 151 | 34.491 | 19.865 | −84.641 | 1.00 | 18.84 | 6 |
| ATOM | 1057 | CB | PHE | 151 | 34.035 | 19.794 | −83.170 | 1.00 | 16.95 | 6 |
| ATOM | 1058 | CG | PHE | 151 | 33.254 | 18.554 | −82.826 | 1.00 | 18.22 | 6 |
| ATOM | 1059 | CD1 | PHE | 151 | 33.893 | 17.321 | −82.714 | 1.00 | 16.96 | 6 |
| ATOM | 1060 | CD2 | PHE | 151 | 31.879 | 18.616 | −82.619 | 1.00 | 19.10 | 6 |
| ATOM | 1061 | CE1 | PHE | 151 | 33.171 | 16.162 | −82.401 | 1.00 | 18.65 | 6 |
| ATOM | 1062 | CE2 | PHE | 151 | 31.146 | 17.459 | −82.307 | 1.00 | 17.82 | 6 |
| ATOM | 1063 | CZ | PHE | 151 | 31.798 | 16.232 | −82.198 | 1.00 | 18.31 | 6 |
| ATOM | 1064 | C | PHE | 151 | 35.247 | 21.180 | −84.844 | 1.00 | 18.22 | 6 |
| ATOM | 1065 | O | PHE | 151 | 36.415 | 21.299 | −84.480 | 1.00 | 18.24 | 8 |
| ATOM | 1066 | N | THR | 152 | 34.561 | 22.162 | −85.420 | 1.00 | 18.91 | 7 |
| ATOM | 1067 | CA | THR | 152 | 35.130 | 23.494 | −85.645 | 1.00 | 19.81 | 6 |
| ATOM | 1068 | CB | THR | 152 | 35.657 | 23.676 | −87.078 | 1.00 | 20.00 | 6 |
| ATOM | 1069 | OG1 | THR | 152 | 34.538 | 23.757 | −87.972 | 1.00 | 18.21 | 8 |
| ATOM | 1070 | CG2 | THR | 152 | 36.564 | 22.513 | −87.487 | 1.00 | 18.48 | 6 |
| ATOM | 1071 | C | THR | 152 | 33.984 | 24.487 | −85.478 | 1.00 | 20.32 | 6 |
| ATOM | 1072 | O | THR | 152 | 32.850 | 24.091 | −85.222 | 1.00 | 19.16 | 8 |
| ATOM | 1073 | N | ASP | 153 | 34.276 | 25.774 | −85.644 | 1.00 | 23.58 | 7 |
| ATOM | 1074 | CA | ASP | 153 | 33.244 | 26.800 | −85.539 | 1.00 | 25.55 | 6 |
| ATOM | 1075 | CB | ASP | 153 | 33.886 | 28.186 | −85.510 | 1.00 | 29.19 | 6 |
| ATOM | 1076 | CG | ASP | 153 | 34.248 | 28.629 | −84.109 | 1.00 | 34.57 | 6 |
| ATOM | 1077 | OD1 | ASP | 153 | 34.795 | 27.808 | −83.343 | 1.00 | 36.82 | 8 |
| ATOM | 1078 | OD2 | ASP | 153 | 33.987 | 29.805 | −83.779 | 1.00 | 38.88 | 8 |
| ATOM | 1079 | C | ASP | 153 | 32.242 | 26.722 | −86.694 | 1.00 | 25.57 | 6 |
| ATOM | 1080 | O | ASP | 153 | 31.165 | 27.316 | −86.628 | 1.00 | 26.92 | 8 |
| ATOM | 1081 | N | SER | 154 | 32.595 | 26.002 | −87.756 | 1.00 | 23.39 | 7 |
| ATOM | 1082 | CA | SER | 154 | 31.692 | 25.866 | −88.896 | 1.00 | 22.31 | 6 |
| ATOM | 1083 | CB | SER | 154 | 32.455 | 25.457 | −90.153 | 1.00 | 21.12 | 6 |
| ATOM | 1084 | OG | SER | 154 | 33.419 | 26.424 | −90.498 | 1.00 | 26.31 | 8 |
| ATOM | 1085 | C | SER | 154 | 30.625 | 24.821 | −88.615 | 1.00 | 21.14 | 6 |
| ATOM | 1086 | O | SER | 154 | 29.491 | 24.947 | −89.070 | 1.00 | 22.35 | 8 |
| ATOM | 1087 | N | GLY | 155 | 31.000 | 23.787 | −87.867 | 1.00 | 19.79 | 7 |
| ATOM | 1088 | CA | GLY | 155 | 30.073 | 22.714 | −87.548 | 1.00 | 18.57 | 6 |
| ATOM | 1089 | C | GLY | 155 | 30.831 | 21.417 | −87.318 | 1.00 | 17.77 | 6 |
| ATOM | 1090 | O | GLY | 155 | 31.900 | 21.429 | −86.714 | 1.00 | 19.15 | 8 |
| ATOM | 1091 | N | ILE | 156 | 30.288 | 20.297 | −87.791 | 1.00 | 18.10 | 7 |
| ATOM | 1092 | CA | ILE | 156 | 30.952 | 19.003 | −87.623 | 1.00 | 16.42 | 6 |
| ATOM | 1093 | CB | ILE | 156 | 30.020 | 17.974 | −86.925 | 1.00 | 18.62 | 6 |
| ATOM | 1094 | CG2 | ILE | 156 | 30.831 | 16.752 | −86.469 | 1.00 | 15.12 | 6 |
| ATOM | 1095 | CG1 | ILE | 156 | 29.357 | 18.614 | −85.698 | 1.00 | 17.05 | 6 |
| ATOM | 1096 | CD1 | ILE | 156 | 28.384 | 17.704 | −84.968 | 1.00 | 17.30 | 6 |
| ATOM | 1097 | C | ILE | 156 | 31.348 | 18.494 | −89.009 | 1.00 | 16.14 | 6 |
| ATOM | 1098 | O | ILE | 156 | 30.533 | 18.486 | −89.930 | 1.00 | 14.90 | 8 |
| ATOM | 1099 | N | HIS | 157 | 32.602 | 18.073 | −89.150 | 1.00 | 16.46 | 7 |
| ATOM | 1100 | CA | HIS | 157 | 33.114 | 17.597 | −90.436 | 1.00 | 16.20 | 6 |
| ATOM | 1101 | CB | HIS | 157 | 34.396 | 18.352 | −90.825 | 1.00 | 17.32 | 6 |
| ATOM | 1102 | CG | HIS | 157 | 34.330 | 19.839 | −90.652 | 1.00 | 19.54 | 6 |
| ATOM | 1103 | CD2 | HIS | 157 | 34.359 | 20.612 | −89.539 | 1.00 | 18.95 | 6 |
| ATOM | 1104 | ND1 | HIS | 157 | 34.318 | 20.708 | −91.721 | 1.00 | 19.36 | 7 |
| ATOM | 1105 | GE1 | HIS | 157 | 34.349 | 21.952 | −91.275 | 1.00 | 21.59 | 6 |
| ATOM | 1106 | NE2 | HIS | 157 | 34.374 | 21.922 | −89.954 | 1.00 | 19.45 | 7 |
| ATOM | 1107 | C | HIS | 157 | 33.501 | 16.120 | −90.473 | 1.00 | 15.17 | 6 |
| ATOM | 1108 | O | HIS | 157 | 33.849 | 15.528 | −89.454 | 1.00 | 14.78 | 8 |
| ATOM | 1109 | N | ILE | 158 | 33.437 | 15.549 | −91.672 | 1.00 | 13.54 | 7 |
| ATOM | 1110 | CA | ILE | 158 | 33.915 | 14.197 | −91.924 | 1.00 | 13.65 | 6 |
| ATOM | 1111 | CB | ILE | 158 | 32.875 | 13.272 | −92.581 | 1.00 | 14.47 | 6 |
| ATOM | 1112 | CG2 | ILE | 158 | 33.545 | 11.945 | −92.971 | 1.00 | 14.66 | 6 |
| ATOM | 1113 | CG1 | ILE | 158 | 31.741 | 12.971 | −91.601 | 1.00 | 13.46 | 6 |
| ATOM | 1114 | CD1 | ILE | 158 | 30.597 | 12.199 | −92.229 | 1.00 | 14.08 | 6 |
| ATOM | 1115 | C | ILE | 158 | 34.967 | 14.575 | −92.960 | 1.00 | 13.80 | 6 |
| ATOM | 1116 | O | ILE | 158 | 34.656 | 15.249 | −93.944 | 1.00 | 12.48 | 8 |
| ATOM | 1117 | N | ILE | 159 | 36.211 | 14.173 | −92.737 | 1.00 | 14.98 | 7 |
| ATOM | 1118 | CA | ILE | 159 | 37.276 | 14.546 | −93.654 | 1.00 | 16.11 | 6 |
| ATOM | 1119 | CB | ILE | 159 | 38.382 | 15.337 | −92.910 | 1.00 | 15.43 | 6 |
| ATOM | 1120 | CG2 | ILE | 159 | 39.377 | 15.910 | −93.910 | 1.00 | 14.11 | 6 |
| ATOM | 1121 | CG1 | ILE | 159 | 37.755 | 16.463 | −92.084 | 1.00 | 15.83 | 6 |
| ATOM | 1122 | CD1 | ILE | 159 | 38.749 | 17.226 | −91.217 | 1.00 | 13.84 | 6 |
| ATOM | 1123 | C | ILE | 159 | 37.931 | 13.362 | −94.348 | 1.00 | 16.10 | 6 |
| ATOM | 1124 | O | ILE | 159 | 38.242 | 12.354 | −93.717 | 1.00 | 17.29 | 8 |
| ATOM | 1125 | N | LEU | 160 | 38.137 | 13.498 | −95.654 | 1.00 | 16.76 | 7 |
| ATOM | 1126 | CA | LEU | 160 | 38.790 | 12.460 | −96.440 | 1.00 | 17.32 | 6 |
| ATOM | 1127 | CB | LEU | 160 | 37.870 | 11.946 | −97.547 | 1.00 | 16.89 | 6 |
| ATOM | 1128 | CG | LEU | 160 | 38.553 | 10.920 | −98.460 | 1.00 | 16.81 | 6 |
| ATOM | 1129 | CD1 | LED | 160 | 38.670 | 9.593 | −97.720 | 1.00 | 15.48 | 6 |
| ATOM | 1130 | CD2 | LED | 160 | 37.765 | 10.737 | −99.747 | 1.00 | 15.66 | 6 |
| ATOM | 1131 | C | LED | 160 | 40.029 | 13.057 | −97.089 | 1.00 | 17.29 | 6 |
| ATOM | 1132 | O | LED | 160 | 39.924 | 13.963 | −97.912 | 1.00 | 17.67 | 8 |
| ATOM | 1133 | N | ARG | 161 | 41.206 | 12.563 | −96.729 | 1.00 | 18.95 | 7 |
| ATOM | 1134 | CA | ARG | 161 | 42.417 | 13.084 | −97.348 | 1.00 | 19.69 | 6 |
| ATOM | 1135 | CB | ARG | 161 | 43.637 | 12.898 | −96.442 | 1.00 | 19.29 | 6 |
| ATOM | 1136 | CG | ARG | 161 | 44.899 | 13.399 | −97.127 | 1.00 | 21.27 | 6 |
| ATOM | 1137 | CD | ARG | 161 | 46.155 | 13.245 | −96.300 | 1.00 | 23.14 | 6 |
| ATOM | 1138 | NE | ARG | 161 | 47.327 | 13.504 | −97.131 | 1.00 | 23.12 | 7 |
| ATOM | 1139 | CZ | ARG | 161 | 48.582 | 13.282 | −96.754 | 1.00 | 26.89 | 6 |
| ATOM | 1140 | NH1 | ARG | 161 | 48.843 | 12.798 | −95.546 | 1.00 | 25.03 | 7 |
| ATOM | 1141 | NH2 | ARG | 161 | 49.578 | 13.523 | −97.597 | 1.00 | 25.94 | 7 |
| ATOM | 1142 | C | ARG | 161 | 42.646 | 12.358 | −98.675 | 1.00 | 18.11 | 6 |
| ATOM | 1143 | O | ARG | 161 | 42.806 | 11.140 | −98.702 | 1.00 | 15.71 | 8 |
| ATOM | 1144 | N | THR | 162 | 42.662 | 13.114 | −99.770 | 1.00 | 17.76 | 7 |
| ATOM | 1145 | CA | THR | 162 | 42.852 | 12.536 | −101.095 | 1.00 | 19.44 | 6 |
| ATOM | 1146 | CB | THR | 162 | 41.875 | 13.161 | −102.111 | 1.00 | 21.16 | 6 |
| ATOM | 1147 | OG1 | THR | 162 | 42.086 | 14.577 | −102.169 | 1.00 | 19.24 | 8 |
| ATOM | 1148 | CD2 | THR | 162 | 40.432 | 12.885 | −101.699 | 1.00 | 21.75 | 6 |
| ATOM | 1149 | C | THR | 162 | 44.276 | 12.663 | −101.649 | 1.00 | 20.55 | 6 |
| ATOM | 1150 | O | THR | 162 | 44.653 | 11.930 | −102.553 | 1.00 | 18.75 | 8 |
| ATOM | 1151 | N | GLU | 163 | 45.057 | 13.600 | −101.123 | 1.00 | 21.92 | 7 |
| ATOM | 1152 | CA | GLU | 163 | 46.439 | 13.779 | −101.576 | 1.00 | 23.87 | 6 |
| ATOM | 1153 | CB | GLU | 163 | 46.511 | 14.812 | −102.708 | 1.00 | 25.95 | 6 |
| ATOM | 1154 | CG | GLU | 163 | 46.002 | 14.325 | −104.063 | 1.00 | 26.27 | 6 |
| ATOM | 1155 | CD | GLU | 163 | 46.064 | 15.405 | −105.136 | 1.00 | 26.96 | 6 |
| ATOM | 1156 | OE1 | GLU | 163 | 46.944 | 16.285 | −105.051 | 1.00 | 29.39 | 8 |
| ATOM | 1157 | OE2 | GLU | 163 | 45.244 | 15.367 | −106.074 | 1.00 | 28.48 | 8 |
| ATOM | 1158 | C | GLU | 163 | 47.312 | 14.243 | −100.419 | 1.00 | 24.65 | 6 |
| ATOM | 1159 | OT1 | GLU | 163 | 46.744 | 14.737 | −99.425 | 1.00 | 25.34 | 8 |
| ATOM | 1160 | OT2 | GLU | 163 | 48.551 | 14.126 | −100.523 | 1.00 | 25.26 | 8 |
| ATOM | 1161 | OH2 | TIP S | 1 | 37.338 | 26.056 | −85.330 | 1.00 | 27.95 | 8 |
| ATOM | 1162 | OH2 | TIP S | 2 | 39.617 | 13.884 | −80.173 | 1.00 | 16.89 | 8 |
| ATOM | 1163 | OH2 | TIP S | 3 | 25.624 | 19.643 | −104.315 | 1.00 | 14.37 | 8 |
| ATOM | 1164 | OH2 | TIP S | 4 | 39.784 | 16.056 | −102.424 | 1.00 | 17.92 | 8 |
| ATOM | 1165 | OH2 | TIP S | 5 | 34.891 | 0.375 | −93.811 | 1.00 | 16.77 | 8 |
| ATOM | 1166 | OH2 | TIP S | 6 | 31.365 | 19.592 | −98.724 | 1.00 | 18.40 | 8 |
| ATOM | 1167 | OH2 | TIP S | 7 | 45.901 | 8.713 | −89.501 | 1.00 | 22.03 | 8 |
| ATOM | 1168 | OH2 | TIP S | 8 | 43.358 | 8.896 | −88.404 | 1.00 | 21.31 | 8 |
| ATOM | 1169 | OH2 | TIP S | 9 | 31.214 | 18.715 | −109.535 | 1.00 | 12.89 | 8 |
| ATOM | 1170 | OH2 | TIP S | 10 | 35.587 | 14.674 | −102.935 | 1.00 | 20.80 | 8 |
| ATOM | 1171 | OH2 | TIP S | 11 | 32.596 | 3.975 | −98.954 | 1.00 | 16.66 | 8 |
| ATOM | 1172 | OH2 | TIP S | 12 | 22.455 | 16.448 | −92.996 | 1.00 | 24.48 | 8 |
| ATOM | 1173 | OH2 | TIP S | 13 | 29.690 | 6.336 | −87.127 | 1.00 | 17.32 | 8 |
| ATOM | 1174 | OH2 | TIP S | 14 | 45.372 | 24.821 | −92.715 | 1.00 | 30.69 | 8 |
| ATOM | 1175 | OH2 | TIP S | 15 | 22.806 | 10.233 | −91.149 | 1.00 | 20.72 | 8 |
| ATOM | 1176 | OH2 | TIP S | 16 | 29.184 | 17.213 | −106.571 | 1.00 | 19.79 | 8 |
| ATOM | 1177 | OH2 | TIP S | 17 | 36.789 | 1.149 | −89.550 | 1.00 | 20.60 | 8 |
| ATOM | 1178 | OH2 | TIP S | 18 | 21.186 | 21.710 | −93.890 | 1.00 | 27.61 | 8 |
| ATOM | 1179 | OH2 | TIP S | 19 | 39.828 | 18.175 | −82.775 | 1.00 | 25.97 | 8 |
| ATOM | 1180 | OH2 | TIP S | 20 | 34.643 | 2.750 | −84.166 | 1.00 | 20.25 | 8 |
| ATOM | 1181 | OH2 | TIP S | 21 | 19.367 | 13.795 | −101.649 | 1.00 | 36.62 | 8 |
| ATOM | 1182 | OH2 | TIP S | 22 | 41.937 | −5.267 | −83.224 | 1.00 | 24.08 | 8 |
| ATOM | 1183 | OH2 | TIP S | 23 | 35.650 | 7.436 | −83.805 | 1.00 | 28.41 | 8 |
| ATOM | 1184 | OH2 | TIP S | 24 | 31.245 | 8.288 | −85.945 | 1.00 | 28.61 | 8 |
| ATOM | 1185 | OH2 | TIP S | 25 | 48.061 | 17.344 | −87.341 | 1.00 | 22.36 | 8 |
| ATOM | 1186 | OH2 | TIP S | 26 | 33.735 | 0.438 | −77.119 | 1.00 | 37.26 | 8 |
| ATOM | 1187 | OH2 | TIP S | 27 | 23.570 | 18.960 | −93.663 | 1.00 | 22.24 | 8 |
| ATOM | 1188 | OH2 | TIP S | 28 | 20.448 | 12.834 | −94.558 | 1.00 | 29.22 | 8 |
| ATOM | 1189 | OH2 | TIP S | 29 | 42.315 | 4.962 | −91.201 | 1.00 | 25.54 | 8 |
| ATOM | 1190 | OH2 | TIP S | 30 | 50.396 | 16.057 | −100.098 | 1.00 | 33.77 | 8 |
| ATOM | 1191 | OH2 | TIP S | 31 | 19.187 | 34.818 | −95.069 | 1.00 | 36.44 | 8 |
| ATOM | 1192 | OH2 | TIP S | 32 | 43.279 | 16.685 | −81.044 | 1.00 | 35.73 | 8 |
| ATOM | 1193 | OH2 | TIP S | 33 | 16.494 | 35.879 | −98.232 | 1.00 | 35.02 | 8 |
| ATOM | 1194 | OH2 | TIP S | 34 | 51.646 | 23.529 | −94.359 | 1.00 | 35.08 | 8 |
| ATOM | 1195 | OH2 | TIP S | 35 | 48.414 | 18.236 | −102.717 | 1.00 | 41.86 | 8 |
| ATOM | 1196 | OH2 | TIP S | 36 | 19.859 | 19.515 | −99.848 | 1.00 | 37.41 | 8 |
| ATOM | 1197 | OH2 | TIP S | 37 | 15.321 | 27.901 | −93.602 | 1.00 | 30.54 | 8 |
| ATOM | 1198 | OH2 | TIP S | 38 | 19.119 | 17.852 | −79.702 | 1.00 | 37.11 | 8 |
| ATOM | 1199 | OH2 | TIP S | 39 | 22.984 | 8.025 | −86.159 | 1.00 | 42.41 | 8 |
| ATOM | 1200 | OH2 | TIP S | 40 | 49.106 | 10.191 | −88.064 | 1.00 | 31.04 | 8 |
| ATOM | 1201 | OH2 | TIP S | 41 | 46.216 | 4.197 | −94.867 | 1.00 | 37.88 | 8 |
| ATOM | 1202 | OH2 | TIP S | 42 | 20.059 | 13.882 | −86.118 | 1.00 | 29.92 | 8 |
| ATOM | 1203 | OH2 | TIP S | 43 | 38.046 | −1.333 | −88.694 | 1.00 | 22.37 | 8 |
| ATOM | 1204 | OH2 | TIP S | 44 | 41.865 | 7.761 | −90.567 | 1.00 | 33.22 | 8 |
| ATOM | 1205 | OH2 | TIP S | 45 | 18.610 | 17.951 | −89.419 | 1.00 | 28.19 | 8 |
| ATOM | 1206 | OH2 | TIP S | 46 | 41.189 | −9.190 | −83.564 | 1.00 | 32.62 | 8 |
| ATOM | 1207 | OH2 | TIP S | 47 | 42.162 | −8.018 | −78.830 | 1.00 | 37.32 | 8 |
| ATOM | 1208 | OH2 | TIP S | 48 | 37.438 | 20.091 | −107.613 | 1.00 | 43.11 | 8 |
| ATOM | 1209 | OH2 | TIP S | 49 | 39.376 | 11.272 | −104.791 | 1.00 | 46.80 | 8 |
| ATOM | 1210 | OH2 | TIP S | 50 | 40.491 | 27.132 | −83.333 | 1.00 | 35.90 | 8 |
| ATOM | 1211 | OH2 | TIP S | 51 | 19.738 | 16.609 | −93.049 | 1.00 | 35.94 | 8 |
| ATOM | 1212 | OH2 | TIP S | 52 | 49.688 | 11.865 | −92.333 | 1.00 | 33.33 | 8 |
| ATOM | 1213 | OH2 | TIP S | 53 | 38.712 | 30.099 | −82.836 | 1.00 | 53.20 | 8 |
| ATOM | 1214 | OH2 | TIP S | 54 | 44.233 | 18.598 | −82.720 | 1.00 | 45.71 | 8 |
| ATOM | 1215 | OH2 | TIP S | 55 | 30.389 | 10.613 | −82.121 | 1.00 | 31.56 | 8 |
| ATOM | 1216 | OH2 | TIP S | 56 | 36.221 | −2.932 | −89.966 | 1.00 | 35.38 | 8 |
| ATOM | 1217 | OH2 | TIP S | 57 | 20.096 | 11.403 | −87.330 | 1.00 | 37.13 | 8 |
| ATOM | 1218 | OH2 | TIP S | 58 | 24.615 | −2.035 | −92.742 | 1.00 | 39.32 | 8 |
| ATOM | 1219 | OH2 | TIP S | 59 | 13.735 | 33.858 | −97.626 | 1.00 | 31.41 | 8 |
| ATOM | 1220 | OH2 | TIP S | 60 | 32.535 | −0.853 | −83.722 | 1.00 | 35.88 | 8 |
| ATOM | 1221 | OH2 | TIP S | 61 | 33.591 | 6.442 | −99.706 | 1.00 | 23.44 | 8 |
| ATOM | 1222 | OH2 | TIP S | 62 | 38.824 | 6.183 | −96.354 | 1.00 | 25.18 | 8 |
| ATOM | 1223 | OH2 | TIP S | 63 | 49.393 | 21.558 | −84.495 | 1.00 | 35.78 | 8 |
| ATOM | 1224 | OH2 | TIP S | 64 | 46.226 | −5.488 | −82.907 | 1.00 | 45.72 | 8 |
| ATOM | 1225 | OH2 | TIP S | 65 | 36.171 | 6.587 | −96.681 | 1.00 | 17.76 | 8 |
| ATOM | 1226 | OH2 | TIP S | 66 | 37.727 | 20.373 | −82.453 | 1.00 | 23.04 | 8 |
| ATOM | 1227 | OH2 | TIP S | 67 | 29.016 | 2.626 | −85.560 | 1.00 | 36.61 | 8 |
| ATOM | 1228 | OH2 | TIP S | 68 | 18.559 | 22.472 | −89.651 | 1.00 | 21.39 | 8 |
| ATOM | 1229 | OH2 | TIP S | 69 | 30.801 | 22.033 | −83.997 | 1.00 | 22.38 | 8 |
| ATOM | 1230 | OH2 | TIP S | 70 | 40.407 | −6.938 | −84.715 | 1.00 | 24.62 | 8 |
| ATOM | 1231 | OH2 | TIP S | 71 | 45.283 | 28.967 | −86.800 | 1.00 | 33.00 | 8 |
| ATOM | 1232 | OH2 | TIP S | 72 | 42.523 | 24.569 | −102.132 | 1.00 | 40.29 | 8 |
| ATOM | 1233 | OH2 | TIP S | 73 | 29.179 | 14.579 | −111.086 | 1.00 | 24.64 | 8 |
| ATOM | 1234 | OH2 | TIP S | 74 | 31.349 | 5.617 | −83.271 | 1.00 | 46.15 | 8 |
| ATOM | 1235 | OH2 | TIP S | 75 | 51.245 | 11.596 | −95.336 | 1.00 | 59.44 | 8 |
| ATOM | 1236 | OH2 | TIP S | 76 | 36.691 | 11.751 | −104.728 | 1.00 | 53.18 | 8 |
| ATOM | 1237 | OH2 | TIP S | 77 | 20.270 | 16.286 | −101.042 | 1.00 | 32.12 | 8 |
| ATOM | 1238 | OH2 | TIP S | 78 | 30.414 | 17.273 | −78.231 | 1.00 | 43.35 | 8 |
| ATOM | 1239 | OH2 | TIP S | 79 | 29.893 | 28.475 | −90.089 | 1.00 | 36.38 | 8 |
| ATOM | 1240 | OH2 | TIP S | 80 | 38.339 | 25.344 | −91.906 | 1.00 | 25.65 | 8 |
| ATOM | 1241 | OH2 | TIP S | 81 | 26.506 | 26.686 | −98.658 | 1.00 | 26.25 | 8 |
| ATOM | 1242 | OH2 | TIP S | 82 | 20.931 | 33.253 | −93.898 | 1.00 | 32.56 | 8 |
| ATOM | 1243 | OH2 | TIP S | 83 | 50.439 | 9.512 | −96.860 | 1.00 | 41.66 | 8 |
| ATOM | 1244 | OH2 | TIP S | 84 | 28.721 | 5.120 | −85.086 | 1.00 | 35.65 | 8 |
| ATOM | 1245 | OH2 | TIP S | 85 | 39.831 | 14.906 | −104.823 | 1.00 | 23.60 | 8 |
| ATOM | 1246 | OH2 | TIP S | 86 | 15.917 | 27.693 | −96.993 | 1.00 | 32.07 | 8 |
| ATOM | 1247 | OH2 | TIP S | 87 | 34.165 | 11.498 | −107.831 | 1.00 | 34.90 | 8 |
| ATOM | 1248 | OH2 | TIP S | 88 | 43.593 | 18.774 | −79.631 | 1.00 | 37.19 | 8 |
| ATOM | 1249 | OH2 | TIP S | 89 | 32.931 | 8.287 | −84.037 | 1.00 | 33.65 | 8 |
| ATOM | 1250 | OH2 | TIP S | 90 | 34.606 | 2.900 | −72.366 | 1.00 | 32.24 | 8 |
| ATOM | 1251 | OH2 | TIP S | 91 | 30.378 | 27.216 | −97.432 | 1.00 | 36.95 | 8 |
| ATOM | 1252 | OH2 | TIP S | 92 | 24.961 | 30.248 | −84.641 | 1.00 | 32.21 | 8 |
| ATOM | 1253 | OH2 | TIP S | 93 | 24.512 | 13.305 | −80.729 | 1.00 | 34.08 | 8 |
| ATOM | 1254 | OH2 | TIP S | 94 | 38.742 | −7.661 | −73.209 | 1.00 | 40.23 | 8 |
| ATOM | 1255 | OH2 | TIP S | 95 | 21.270 | 9.709 | −89.196 | 1.00 | 36.25 | 8 |
| ATOM | 1256 | OH2 | TIP S | 96 | 30.231 | 18.973 | −76.291 | 1.00 | 44.08 | 8 |
| ATOM | 1257 | OH2 | TIP S | 97 | 27.628 | 6.903 | −106.292 | 1.00 | 55.19 | 8 |
| ATOM | 1258 | OH2 | TIP S | 98 | 53.788 | 8.697 | −83.986 | 1.00 | 43.46 | 8 |
| ATOM | 1259 | OH2 | TIP S | 99 | 33.952 | 24.807 | −93.205 | 1.00 | 28.65 | 8 |
| ATOM | 1260 | OH2 | TIP S | 100 | 29.387 | 27.385 | −79.448 | 1.00 | 47.70 | 8 |
| ATOM | 1261 | OH2 | TIP S | 101 | 47.199 | 27.727 | −85.440 | 1.00 | 37.59 | 8 |
| ATOM | 1262 | OH2 | TIP S | 102 | 15.585 | 23.814 | −98.144 | 1.00 | 45.38 | 8 |
| ATOM | 1263 | OH2 | TIP S | 103 | 38.292 | 5.342 | −78.013 | 1.00 | 47.52 | 8 |
| ATOM | 1264 | OH2 | TIP S | 104 | 18.225 | 15.410 | −87.461 | 1.00 | 55.24 | 8 |
| ATOM | 1265 | OH2 | TIP S | 105 | 36.715 | 24.433 | −93.775 | 1.00 | 33.51 | 8 |
| ATOM | 1266 | OH2 | TIP S | 106 | 40.406 | −5.755 | −91.932 | 1.00 | 28.37 | 8 |
| ATOM | 1267 | OH2 | TIP S | 107 | 45.814 | 20.389 | −79.951 | 1.00 | 35.21 | 8 |
| ATOM | 1268 | OH2 | TIP S | 108 | 37.198 | −4.638 | −91.702 | 1.00 | 30.55 | 8 |
| ATOM | 1269 | OH2 | TIP S | 109 | 28.111 | 10.358 | −80.677 | 1.00 | 33.61 | 8 |
| ATOM | 1270 | OH2 | TIP S | 110 | 24.401 | 10.341 | −106.003 | 1.00 | 45.21 | 8 |
| ATOM | 1271 | OH2 | TIP S | 111 | 14.499 | 25.620 | −96.558 | 1.00 | 41.27 | 8 |
| ATOM | 1272 | OH2 | TIP S | 112 | 32.043 | 25.827 | −95.145 | 1.00 | 41.03 | 8 |
| ATOM | 1273 | OH2 | TIP S | 113 | 14.508 | 30.154 | −97.273 | 1.00 | 37.09 | 8 |
| ATOM | 1274 | OH2 | TIP S | 114 | 37.028 | 22.310 | −81.091 | 1.00 | 41.65 | 8 |
| ATOM | 1275 | OH2 | TIP S | 115 | 37.729 | 27.272 | −93.503 | 1.00 | 39.90 | 8 |
| ATOM | 1276 | OH2 | TIP S | 116 | 40.992 | 27.749 | −85.957 | 1.00 | 45.33 | 8 |
| ATOM | 1277 | O1 | PGB C | 180 | 33.586 | 3.402 | −81.087 | 1.00 | 39.42 | 8 |
| ATOM | 1278 | C2 | PGB C | 180 | 34.200 | 2.512 | −80.258 | 1.00 | 39.57 | 6 |
| ATOM | 1279 | C3 | PGB C | 180 | 34.104 | 1.072 | −80.801 | 1.00 | 37.05 | 6 |
| ATOM | 1280 | O4 | PGB C | 180 | 34.844 | 0.919 | −81.904 | 1.00 | 34.88 | 8 |
| ATOM | 1281 | C5 | PGB C | 180 | 35.514 | −0.236 | −81.922 | 1.00 | 30.68 | 6 |
| ATOM | 1282 | C6 | PGB C | 180 | 36.343 | −0.339 | −83.226 | 1.00 | 28.92 | 6 |
| ATOM | 1283 | O7 | PGB C | 180 | 35.501 | −0.263 | −84.291 | 1.00 | 28.32 | 8 |
| ATOM | 1284 | C8 | PGB C | 180 | 36.135 | −0.344 | −85.475 | 1.00 | 23.58 | 6 |
| ATOM | 1285 | C9 | PGB C | 180 | 35.093 | −0.249 | −86.614 | 1.00 | 22.87 | 6 |
| ATOM | 1286 | O10 | PGB C | 180 | 34.242 | 0.791 | −86.381 | 1.00 | 23.64 | 8 |
| ATOM | 1287 | C11 | PGB C | 180 | 33.307 | 0.921 | −87.340 | 1.00 | 22.61 | 6 |
| ATOM | 1288 | C12 | PGB C | 180 | 32.436 | 2.164 | −87.037 | 1.00 | 24.50 | 6 |
| ATOM | 1289 | O13 | PGB C | 180 | 33.155 | 3.293 | −87.183 | 1.00 | 24.23 | 8 |
| ATOM | 1290 | C14 | PGB C | 180 | 32.469 | 4.345 | −86.725 | 1.00 | 23.36 | 6 |
| ATOM | 1291 | C15 | PGB C | 180 | 33.414 | 5.557 | −86.488 | 1.00 | 24.16 | 6 |
| ATOM | 1292 | O16 | PGB C | 180 | 34.236 | 5.784 | −87.533 | 1.00 | 26.10 | 8 |
| ATOM | 1293 | C17 | PGB C | 180 | 34.987 | 6.807 | −87.243 | 1.00 | 27.20 | 6 |
| ATOM | 1294 | C18 | PGB C | 180 | 35.964 | 7.158 | −88.364 | 1.00 | 27.22 | 6 |
| ATOM | 1295 | O19 | PGB C | 180 | 36.864 | 6.208 | −88.420 | 1.00 | 28.71 | 8 |
| ATOM | 1296 | C20 | PGB C | 180 | 37.541 | 6.353 | −89.519 | 1.00 | 34.05 | 6 |
| ATOM | 1297 | C21 | PGB C | 180 | 38.693 | 5.665 | −90.239 | 1.00 | 34.56 | 6 |
| ATOM | 1298 | O22 | PGB C | 180 | 38.422 | 4.382 | −90.275 | 1.00 | 39.66 | 8 |
| END | | | | | | | | | | |

### Example 3

### Activity of pPin1(71) R14A

To determine if the phosphorylation of Pin1 at position 71 resulted in the loss of catalytic activity, PKA treated Pin1 R1 4A was tested using an assay that measures the rate constant of isomerization of Pin1 substrate as described below.

The proline isomerase activity assay is based on the method described by Fisher et al. (Biomed Biochim. Acta, (1984) 43: 1101-1111). Specifically, the enzyme (112 ng) was preincubated with 72 mM substrate at 4° C. for 30 minutes in an 80 μL reaction volume containing 0.02 mg/μL BSA, 0.8 mM DTT, and 35 mM HEPES (pH 7.8). Proteolysis of the substrate was initiated by the addition of 80 μL of trypsin at 0.4 mg/mL in 35 mM HEPES (pH 7.8) and the release of p-nitroaniline was monitored every 10 seconds at 390 nm using a microplate reader (MRD/8V/DIAS, Dynex Technologies).

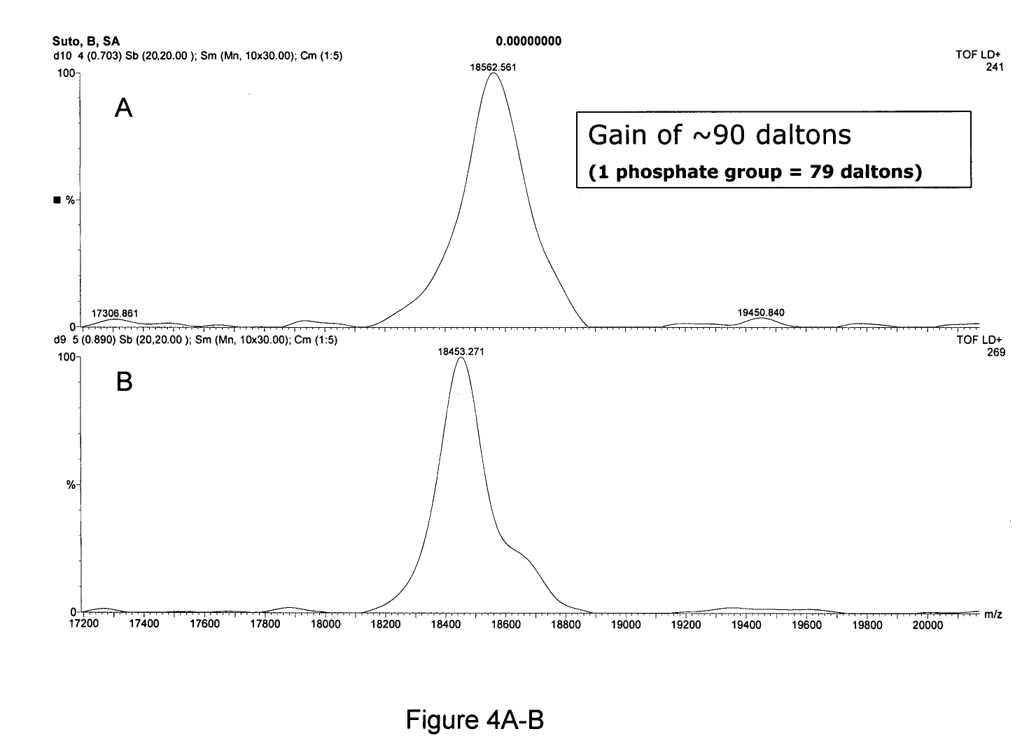

The data indicate that phosphorylation of Pin1 R14A at position 71 by PKA inhibits the ability of the enzyme to isomerizes substrate (FIG. 3).

### Example 4

### Phosphorylation of Pin1 by PKA

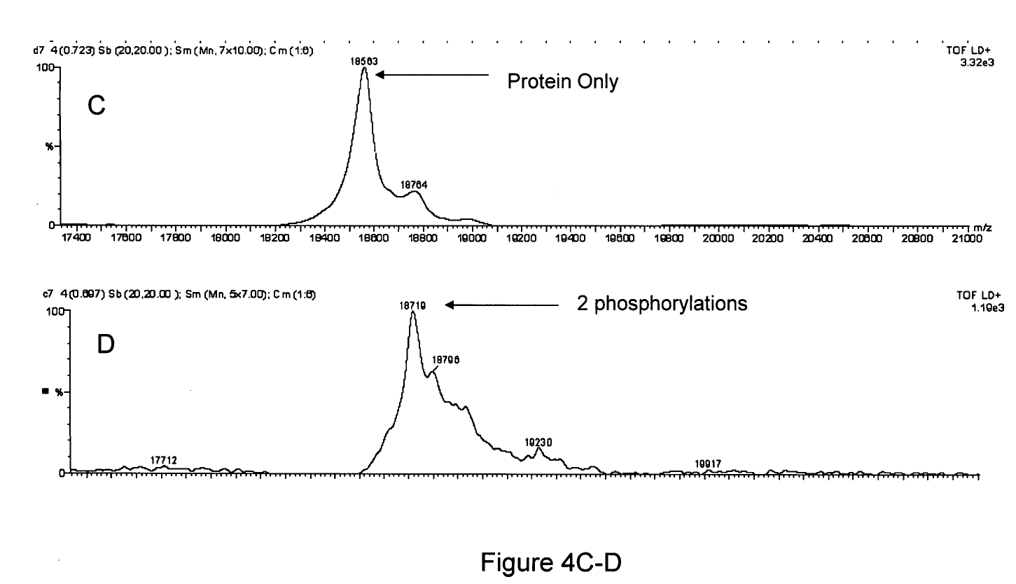

To investigate the phosphorylation of Pin1 by PKA, MALDI-TOF was used. Native Pin1 (FIG. 4A) treated with PKA (FIG. 4B) resulting in a mass increase of ˜160 Daltons. This is characteristic of the addition of two phosphate groups to Pin1 by PKA. Pin1 R14A was treated with PKA (FIG. 4C) resulting in an increase in mass of 90 Daltons as compared to non-PKA treated Pin1 R14A (FIG. 4D).

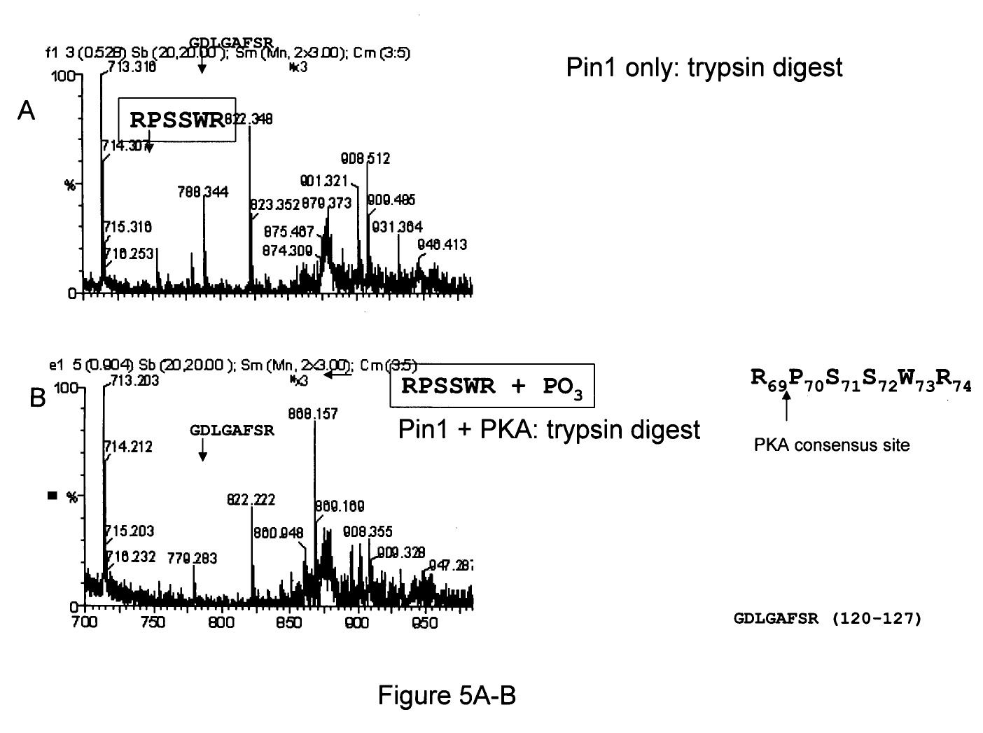

To map the location of the phosphorylation of Pin1 R14A, partial tryptic digests were used in conjunction with MALDI-TOF spectroscopy. Pin1 R14A was treated with PKA and then subjected to partial digest by trypsin. Analysis of the MALDI TOF spectra indicate that the peak corresponding to residues 69-74 of Pin1 gains ˜80 Daltons after treatment with PKA indicating that the phosphorylation event is on this peptide (see FIGS. 5A-B).

### Example 5

### Pin1 Phosphorylated at Position 16 and 71

Wild-type Pin1 was phosphorylated using PKA as described above which resulted in a Pin1 molecule that is phosphorylated at position 16 and 71. Crystals of this polypeptide were grown and subjected to X-ray diffraction. The coordinates of the solved structure are presented below in Table 3.

TABLE III
Set of Atomic Coordinates for pPin1(71) (16) determined by X-ray
Crystallography

| | | | | | | | | | | | |
|---|---|---|---|---|---|---|---|---|---|---|---|
| ATOM | 1 | CB | GLU | | 5 | 49.841 | 61.163 | −87.680 | 1.00 | 69.33 | C |
| ATOM | 2 | CG | GLU | | 5 | 49.566 | 59.671 | −87.455 | 1.00 | 68.63 | C |
| ATOM | 3 | CD | GLU | | 5 | 48.882 | 58.994 | −88.650 | 1.00 | 68.09 | C |
| ATOM | 4 | OE1 | GLU | | 5 | 49.425 | 59.041 | −89.779 | 1.00 | 63.92 | O |
| ATOM | 5 | OE2 | GLU | | 5 | 47.794 | 58.408 | −88.450 | 1.00 | 57.45 | O |
| ATOM | 6 | C | GLU | | 5 | 48.735 | 62.100 | −85.619 | 1.00 | 65.34 | C |
| ATOM | 7 | O | GLU | | 5 | 48.126 | 61.077 | −85.292 | 1.00 | 65.91 | O |
| ATOM | 8 | N | GLU | | 5 | 51.159 | 61.438 | −85.591 | 1.00 | 68.37 | N |
| ATOM | 9 | CA | GLU | | 5 | 50.048 | 62.007 | −86.405 | 1.00 | 66.75 | C |
| ATOM | 10 | N | LYS | | 6 | 48.291 | 63.321 | −85.303 | 1.00 | 61.56 | N |
| ATOM | 11 | CA | LYS | | 6 | 47.039 | 63.539 | −84.569 | 1.00 | 57.35 | C |
| ATOM | 12 | CB | LYS | | 6 | 47.187 | 64.616 | −83.499 | 1.00 | 57.91 | C |
| ATOM | 13 | CG | LYS | | 6 | 45.871 | 64.842 | −82.767 | 1.00 | 59.37 | C |
| ATOM | 14 | CD | LYS | | 6 | 45.789 | 66.181 | −82.062 | 1.00 | 62.00 | C |
| ATOM | 15 | CE | LYS | | 6 | 44.412 | 66.348 | −81.427 | 1.00 | 63.10 | C |
| ATOM | 16 | NZ | LYS | | 6 | 44.228 | 67.675 | −80.788 | 1.00 | 62.34 | N |
| ATOM | 17 | C | LYS | | 6 | 45.892 | 63.955 | −85.488 | 1.00 | 52.18 | C |
| ATOM | 18 | O | LYS | | 6 | 45.790 | 65.110 | −85.899 | 1.00 | 52.50 | O |
| ATOM | 19 | N | LEU | | 7 | 45.000 | 63.023 | −85.786 | 1.00 | 43.58 | N |
| ATOM | 20 | CA | LEU | | 7 | 43.850 | 63.176 | −86.656 | 1.00 | 34.18 | C |
| ATOM | 21 | CB | LEU | | 7 | 43.432 | 61.791 | −87.159 | 1.00 | 28.33 | C |
| ATOM | 22 | CG | LEU | | 7 | 44.564 | 61.008 | −87.829 | 1.00 | 24.55 | C |
| ATOM | 23 | CD1 | LEU | | 7 | 44.083 | 59.618 | −88.179 | 1.00 | 27.82 | C |
| ATOM | 24 | CD2 | LEU | | 7 | 45.008 | 61.724 | −89.089 | 1.00 | 27.08 | C |
| ATOM | 25 | C | LEU | | 7 | 42.672 | 63.869 | −85.978 | 1.00 | 35.03 | C |
| ATOM | 26 | O | LEU | | 7 | 42.581 | 63.898 | −84.751 | 1.00 | 35.89 | O |
| ATOM | 27 | N | PRO | | 8 | 41.752 | 64.443 | −86.775 | 1.00 | 30.43 | N |
| ATOM | 28 | CD | PRO | | 8 | 41.780 | 64.572 | −88.242 | 1.00 | 24.69 | C |
| ATOM | 29 | CA | PRO | | 8 | 40.581 | 65.130 | −86.228 | 1.00 | 26.58 | C |
| ATOM | 30 | CB | PRO | | 8 | 39.889 | 65.675 | −87.476 | 1.00 | 23.65 | C |
| ATOM | 31 | CG | PRO | | 8 | 41.016 | 65.839 | −88.443 | 1.00 | 28.93 | C |
| ATOM | 32 | C | PRO | | 8 | 39.711 | 64.133 | −85.473 | 1.00 | 29.94 | C |
| ATOM | 33 | O | PRO | | 8 | 39.893 | 62.929 | −85.603 | 1.00 | 30.76 | O |
| ATOM | 34 | N | PRO | | 9 | 38.740 | 64.624 | −84.691 | 1.00 | 35.82 | N |
| ATOM | 35 | CD | PRO | | 9 | 38.493 | 66.046 | −84.398 | 1.00 | 38.08 | C |
| ATOM | 36 | CA | PRO | | 9 | 37.837 | 63.784 | −83.905 | 1.00 | 35.02 | C |
| ATOM | 37 | CB | PRO | | 9 | 36.855 | 64.795 | −83.322 | 1.00 | 40.95 | C |
| ATOM | 38 | CG | PRO | | 9 | 37.714 | 65.972 | −83.094 | 1.00 | 37.57 | C |
| ATOM | 39 | C | PRO | | 9 | 37.133 | 62.650 | −84.637 | 1.00 | 34.81 | C |
| ATOM | 40 | O | PRO | | 9 | 36.492 | 62.851 | −85.672 | 1.00 | 33.33 | O |
| ATOM | 41 | N | GLY | | 10 | 37.236 | 61.444 | −84.090 | 1.00 | 25.95 | N |
| ATOM | 42 | CA | GLY | | 10 | 36.594 | 60.298 | −84.696 | 1.00 | 26.35 | C |
| ATOM | 43 | C | GLY | | 10 | 37.443 | 59.579 | −85.723 | 1.00 | 17.01 | C |
| ATOM | 44 | O | GLY | | 10 | 37.167 | 58.429 | −86.058 | 1.00 | 21.21 | O |
| ATOM | 45 | N | TRP | | 11 | 38.495 | 60.228 | −86.213 | 1.00 | 20.56 | N |
| ATOM | 46 | CA | TRP | | 11 | 39.335 | 59.605 | −87.238 | 1.00 | 21.81 | C |
| ATOM | 47 | CB | TRP | | 11 | 39.958 | 60.657 | −88.159 | 1.00 | 18.68 | C |
| ATOM | 48 | CG | TRP | | 11 | 38.985 | 61.365 | −89.047 | 1.00 | 15.37 | C |
| ATOM | 49 | CD2 | TRP | | 11 | 38.462 | 60.891 | −90.290 | 1.00 | 12.22 | C |
| ATOM | 50 | CE2 | TRP | | 11 | 37.592 | 61.882 | −90.783 | 1.00 | 13.38 | C |
| ATOM | 51 | CE3 | TRP | | 11 | 38.648 | 59.723 | −91.039 | 1.00 | 14.68 | C |
| ATOM | 52 | CD1 | TRP | | 11 | 38.425 | 62.590 | −88.835 | 1.00 | 17.31 | C |
| ATOM | 53 | NE1 | TRP | | 11 | 37.585 | 62.911 | −89.876 | 1.00 | 16.42 | N |
| ATOM | 54 | CZ2 | TRP | | 11 | 36.907 | 61.743 | −91.996 | 1.00 | 12.18 | C |
| ATOM | 55 | CZ3 | TRP | | 11 | 37.962 | 59.585 | −92.253 | 1.00 | 15.92 | C |
| ATOM | 56 | CH2 | TRP | | 11 | 37.103 | 60.593 | −92.713 | 1.00 | 15.47 | C |
| ATOM | 57 | C | TRP | | 11 | 40.476 | 58.748 | −86.727 | 1.00 | 21.53 | C |
| ATOM | 58 | O | TRP | | 11 | 41.179 | 59.116 | −85.788 | 1.00 | 23.68 | O |
| ATOM | 59 | N | GLU | | 12 | 40.693 | 57.610 | −87.369 | 1.00 | 20.31 | N |
| ATOM | 60 | CA | GLU | | 12 | 41.781 | 56.688 | −87.065 | 1.00 | 17.75 | C |
| ATOM | 61 | CB | GLU | | 12 | 41.325 | 55.571 | −86.116 | 1.00 | 22.15 | C |
| ATOM | 62 | CG | GLU | | 12 | 42.398 | 54.499 | −85.889 | 1.00 | 26.68 | C |
| ATOM | 63 | CD | GLU | | 12 | 41.911 | 53.322 | −85.063 | 1.00 | 33.59 | C |
| ATOM | 64 | OE1 | GLU | | 12 | 40.713 | 53.293 | −84.714 | 1.00 | 41.67 | O |
| ATOM | 65 | OE2 | GLU | | 12 | 42.725 | 52.423 | −84.761 | 1.00 | 33.23 | O |
| ATOM | 66 | C | GLU | | 12 | 42.246 | 56.066 | −88.372 | 1.00 | 21.94 | C |
| ATOM | 67 | O | GLU | | 12 | 41.437 | 55.830 | −89.280 | 1.00 | 20.26 | O |
| ATOM | 68 | N | LYS | | 13 | 43.547 | 55.815 | −88.496 | 1.00 | 14.49 | N |
| ATOM | 69 | CA | LYS | | 13 | 44.138 | 55.179 | −89.671 | 1.00 | 16.28 | C |
| ATOM | 70 | CB | LYS | | 13 | 45.648 | 55.417 | −89.661 | 1.00 | 16.29 | C |
| ATOM | 71 | CG | LYS | | 13 | 46.403 | 54.826 | −90.829 | 1.00 | 21.62 | C |
| ATOM | 72 | CD | LYS | | 13 | 47.890 | 55.106 | −90.635 | 1.00 | 26.49 | C |
| ATOM | 73 | CE | LYS | | 13 | 48.694 | 54.869 | −91.887 | 1.00 | 30.03 | C |
| ATOM | 74 | NZ | LYS | | 13 | 50.109 | 55.277 | −91.628 | 1.00 | 29.08 | N |
| ATOM | 75 | C | LYS | | 13 | 43.854 | 53.680 | −89.575 | 1.00 | 20.28 | C |
| ATOM | 76 | O | LYS | | 13 | 44.127 | 53.056 | −88.545 | 1.00 | 22.25 | O |
| ATOM | 77 | N | ARG | | 14 | 43.291 | 53.088 | −90.622 | 1.00 | 20.73 | N |
| ATOM | 78 | CA | ARG | | 14 | 43.024 | 51.656 | −90.497 | 1.00 | 21.48 | C |
| ATOM | 79 | CB | ARG | | 14 | 41.508 | 51.384 | −90.411 | 1.00 | 16.45 | C |
| ATOM | 80 | CG | ARG | | 14 | 40.783 | 52.190 | −89.334 | 1.00 | 23.92 | C |
| ATOM | 81 | CD | ARG | | 14 | 41.002 | 51.633 | −87.950 | 1.00 | 23.23 | C |
| ATOM | 82 | NE | ARG | | 14 | 40.296 | 50.365 | −87.781 | 1.00 | 31.04 | N |
| ATOM | 83 | CZ | ARG | | 14 | 40.315 | 49.637 | −86.677 | 1.00 | 19.88 | C |
| ATOM | 84 | NH1 | ARG | | 14 | 41.005 | 50.044 | −85.620 | 1.00 | 30.60 | N |
| ATOM | 85 | NH2 | ARG | | 14 | 39.649 | 48.489 | −86.635 | 1.00 | 29.80 | N |
| ATOM | 86 | C | ARG | | 14 | 43.598 | 50.880 | −91.674 | 1.00 | 19.25 | C |
| ATOM | 87 | O | ARG | | 14 | 44.294 | 51.428 | −92.523 | 1.00 | 22.43 | O |
| ATOM | 88 | N | MET | | 15 | 43.282 | 49.598 | −91.749 | 1.00 | 19.10 | N |
| ATOM | 89 | CA | MET | | 15 | 43.795 | 48.801 | −92.839 | 1.00 | 23.42 | C |
| ATOM | 90 | CB | MET | | 15 | 45.095 | 48.147 | −92.375 | 1.00 | 30.63 | C |
| ATOM | 91 | CG | MET | | 15 | 45.733 | 47.134 | −93.291 | 1.00 | 38.63 | C |
| ATOM | 92 | SD | MET | | 15 | 47.456 | 46.964 | −92.733 | 1.00 | 51.66 | S |
| ATOM | 93 | CE | MET | | 15 | 47.225 | 46.424 | −90.940 | 1.00 | 31.26 | C |
| ATOM | 94 | C | MET | | 15 | 42.817 | 47.749 | −93.331 | 1.00 | 26.24 | C |
| ATOM | 95 | O | MET | | 15 | 42.104 | 47.128 | −92.540 | 1.00 | 29.04 | O |
| ATOM | 96 | N | SEP | | 16 | 42.767 | 47.557 | −94.646 | 1.00 | 27.66 | N |
| ATOM | 97 | CA | SEP | | 16 | 42.065 | 46.644 | −95.535 | 1.00 | 37.50 | C |
| ATOM | 98 | CB | SEP | | 16 | 40.607 | 46.974 | −95.800 | 1.00 | 44.79 | C |
| ATOM | 99 | OG | SEP | | 16 | 40.071 | 45.945 | −96.632 | 1.00 | 51.86 | O |
| ATOM | 100 | C | SEP | | 16 | 42.213 | 45.223 | −94.999 | 1.00 | 31.11 | C |
| ATOM | 101 | O | SEP | | 16 | 43.279 | 44.644 | −95.119 | 1.00 | 29.72 | O |
| ATOM | 102 | P | SEP | | 16 | 39.292 | 46.241 | −97.882 | 1.00 | 58.92 | P |
| ATOM | 103 | O1P | SEP | | 16 | 38.288 | 47.314 | −97.609 | 1.00 | 56.25 | O |
| ATOM | 104 | O2P | SEP | | 16 | 38.579 | 44.997 | −98.302 | 1.00 | 59.95 | O |
| ATOM | 105 | O3P | SEP | | 16 | 40.221 | 46.677 | −98.978 | 1.00 | 48.60 | O |
| ATOM | 106 | N | ARG | | 17 | 41.170 | 44.655 | −94.397 | 1.00 | 34.49 | N |
| ATOM | 107 | CA | ARG | | 17 | 41.481 | 43.293 | −93.959 | 1.00 | 39.81 | C |
| ATOM | 108 | CB | ARG | | 17 | 40.307 | 42.658 | −93.210 | 1.00 | 43.17 | C |
| ATOM | 109 | CG | ARG | | 17 | 40.032 | 43.205 | −91.829 | 1.00 | 51.47 | C |
| ATOM | 110 | CD | ARG | | 17 | 39.185 | 42.208 | −91.061 | 1.00 | 60.59 | C |
| ATOM | 111 | NE | ARG | | 17 | 39.928 | 40.971 | −90.818 | 1.00 | 65.81 | N |
| ATOM | 112 | CZ | ARG | | 17 | 39.389 | 39.754 | −90.827 | 1.00 | 67.42 | C |
| ATOM | 113 | NH1 | ARG | | 17 | 38.093 | 39.599 | −91.071 | 1.00 | 64.44 | N |
| ATOM | 114 | NH2 | ARG | | 17 | 40.149 | 38.689 | −90.595 | 1.00 | 64.51 | N |
| ATOM | 115 | C | ARG | | 17 | 41.820 | 42.397 | −95.149 | 1.00 | 43.85 | C |
| ATOM | 116 | O | ARG | | 17 | 42.747 | 41.585 | −95.086 | 1.00 | 39.33 | O |
| ATOM | 117 | N | SER | | 18 | 41.069 | 42.540 | −96.239 | 1.00 | 44.79 | N |
| ATOM | 118 | CA | SER | | 18 | 41.139 | 41.831 | −97.513 | 1.00 | 48.12 | C |
| ATOM | 119 | CB | SER | | 18 | 39.934 | 42.191 | −98.394 | 1.00 | 46.28 | C |
| ATOM | 120 | OG | SER | | 18 | 38.705 | 41.872 | −97.764 | 1.00 | 52.85 | O |
| ATOM | 121 | C | SER | | 18 | 42.406 | 42.132 | −98.294 | 1.00 | 48.86 | C |
| ATOM | 122 | O | SER | | 18 | 43.099 | 41.214 | −98.736 | 1.00 | 51.41 | O |
| ATOM | 123 | N | SER | | 19 | 42.725 | 43.415 | −98.464 | 1.00 | 47.45 | N |
| ATOM | 124 | CA | SER | | 19 | 43.876 | 43.897 | −99.231 | 1.00 | 43.61 | C |
| ATOM | 125 | CB | SER | | 19 | 43.429 | 45.033 | −100.149 | 1.00 | 48.10 | C |
| ATOM | 126 | OG | SER | | 19 | 42.882 | 46.105 | −99.397 | 1.00 | 45.08 | O |
| ATOM | 127 | C | SER | | 19 | 45.097 | 44.371 | −98.450 | 1.00 | 43.01 | C |
| ATOM | 128 | O | SER | | 19 | 46.224 | 44.305 | −98.943 | 1.00 | 44.51 | O |
| ATOM | 129 | N | GLY | | 20 | 44.897 | 44.860 | −97.237 | 1.00 | 37.27 | N |
| ATOM | 130 | CA | GLY | | 20 | 46.023 | 45.366 | −96.478 | 1.00 | 36.65 | C |
| ATOM | 131 | C | GLY | | 20 | 46.193 | 46.847 | −96.775 | 1.00 | 35.27 | C |
| ATOM | 132 | O | GLY | | 20 | 47.016 | 47.536 | −96.169 | 1.00 | 38.48 | O |
| ATOM | 133 | N | ARG | | 21 | 45.407 | 47.354 | −97.718 | 1.00 | 30.77 | N |
| ATOM | 134 | CA | ARG | | 21 | 45.395 | 48.750 | −98.144 | 1.00 | 32.73 | C |
| ATOM | 135 | CB | ARG | | 21 | 44.370 | 48.955 | −99.265 | 1.00 | 37.99 | C |
| ATOM | 136 | CG | ARG | | 21 | 44.940 | 49.008 | −100.675 | 1.00 | 50.22 | C |
| ATOM | 137 | CD | ARG | | 21 | 43.821 | 49.291 | −101.671 | 1.00 | 55.93 | C |
| ATOM | 138 | NE | ARG | | 21 | 44.309 | 49.801 | −102.950 | 1.00 | 63.15 | N |
| ATOM | 139 | CZ | ARG | | 21 | 43.516 | 50.191 | −103.947 | 1.00 | 67.94 | C |
| ATOM | 140 | NH1 | ARG | | 21 | 42.196 | 50.130 | −103.814 | 1.00 | 70.14 | N |
| ATOM | 141 | NH2 | ARG | | 21 | 44.039 | 50.642 | −105.081 | 1.00 | 70.99 | N |
| ATOM | 142 | C | ARG | | 21 | 45.038 | 49.663 | −96.981 | 1.00 | 24.20 | C |
| ATOM | 143 | O | ARG | | 21 | 43.999 | 49.491 | −96.335 | 1.00 | 24.68 | O |
| ATOM | 144 | N | VAL | | 22 | 45.879 | 50.652 | −96.712 | 1.00 | 24.31 | N |
| ATOM | 145 | CA | VAL | | 22 | 45.676 | 51.604 | −95.630 | 1.00 | 26.78 | C |
| ATOM | 146 | CB | VAL | | 22 | 47.003 | 52.322 | −95.280 | 1.00 | 29.97 | C |
| ATOM | 147 | CG1 | VAL | | 22 | 46.753 | 53.409 | −94.266 | 1.00 | 27.71 | C |
| ATOM | 148 | CG2 | VAL | | 22 | 48.002 | 51.325 | −94.734 | 1.00 | 31.41 | C |
| ATOM | 149 | C | VAL | | 22 | 44.621 | 52.663 | −95.964 | 1.00 | 22.85 | C |
| ATOM | 150 | O | VAL | | 22 | 44.532 | 53.131 | −97.096 | 1.00 | 21.90 | O |
| ATOM | 151 | N | TYR | | 23 | 43.804 | 53.037 | −94.985 | 1.00 | 18.43 | N |
| ATOM | 152 | CA | TYR | | 23 | 42.813 | 54.067 | −95.245 | 1.00 | 18.57 | C |
| ATOM | 153 | CB | TYR | | 23 | 41.573 | 53.467 | −95.919 | 1.00 | 17.18 | C |
| ATOM | 154 | CG | TYR | | 23 | 40.773 | 52.508 | −95.065 | 1.00 | 21.49 | C |
| ATOM | 155 | CD1 | TYR | | 23 | 39.620 | 52.934 | −94.399 | 1.00 | 19.06 | C |
| ATOM | 156 | CE1 | TYR | | 23 | 38.844 | 52.042 | −93.653 | 1.00 | 23.90 | C |
| ATOM | 157 | CD2 | TYR | | 23 | 41.138 | 51.164 | −94.961 | 1.00 | 20.91 | C |
| ATOM | 158 | CE2 | TYR | | 23 | 40.377 | 50.265 | −94.224 | 1.00 | 24.59 | C |
| ATOM | 159 | CZ | TYR | | 23 | 39.233 | 50.708 | −93.575 | 1.00 | 27.51 | C |
| ATOM | 160 | OH | TYR | | 23 | 38.481 | 49.824 | −92.857 | 1.00 | 26.65 | O |
| ATOM | 161 | C | TYR | | 23 | 42.439 | 54.779 | −93.956 | 1.00 | 24.02 | C |
| ATOM | 162 | O | TYR | | 23 | 42.951 | 54.451 | −92.879 | 1.00 | 22.51 | O |
| ATOM | 163 | N | TYR | | 24 | 41.576 | 55.780 | −94.050 | 1.00 | 14.89 | N |
| ATOM | 164 | CA | TYR | | 24 | 41.209 | 56.467 | −92.825 | 1.00 | 15.50 | C |
| ATOM | 165 | CB | TYR | | 24 | 41.614 | 57.945 | −92.915 | 1.00 | 11.81 | C |
| ATOM | 166 | CG | TYR | | 24 | 43.119 | 58.132 | −93.016 | 1.00 | 15.11 | C |
| ATOM | 167 | CD1 | TYR | | 24 | 43.773 | 58.027 | −94.241 | 1.00 | 18.03 | C |
| ATOM | 168 | CE1 | TYR | | 24 | 45.159 | 58.095 | −94.330 | 1.00 | 22.29 | C |
| ATOM | 169 | CD2 | TYR | | 24 | 43.898 | 58.325 | −91.870 | 1.00 | 15.46 | C |
| ATOM | 170 | CE2 | TYR | | 24 | 45.296 | 58.402 | −91.947 | 1.00 | 20.74 | C |
| ATOM | 171 | CZ | TYR | | 24 | 45.915 | 58.279 | −93.180 | 1.00 | 22.40 | C |
| ATOM | 172 | OH | TYR | | 24 | 47.287 | 58.324 | −93.270 | 1.00 | 26.32 | O |
| ATOM | 173 | C | TYR | | 24 | 39.706 | 56.298 | −92.573 | 1.00 | 19.38 | C |
| ATOM | 174 | O | TYR | | 24 | 38.885 | 56.477 | −93.472 | 1.00 | 16.49 | O |
| ATOM | 175 | N | PHE | | 25 | 39.337 | 55.927 | −91.353 | 1.00 | 15.04 | N |
| ATOM | 176 | CA | PHE | | 25 | 37.947 | 55.668 | −90.970 | 1.00 | 14.37 | C |
| ATOM | 177 | CB | PHE | | 25 | 37.813 | 54.216 | −90.521 | 1.00 | 18.52 | C |
| ATOM | 178 | CG | PHE | | 25 | 36.402 | 53.803 | −90.161 | 1.00 | 20.76 | C |
| ATOM | 179 | CD1 | PHE | | 25 | 35.450 | 53.591 | −91.154 | 1.00 | 24.01 | C |
| ATOM | 180 | CD2 | PHE | | 25 | 36.051 | 53.574 | −88.838 | 1.00 | 18.95 | C |
| ATOM | 181 | CE1 | PHE | | 25 | 34.162 | 53.149 | −90.835 | 1.00 | 16.10 | C |
| ATOM | 182 | CE2 | PHE | | 25 | 34.766 | 53.130 | −88.504 | 1.00 | 20.31 | C |
| ATOM | 183 | CZ | PHE | | 25 | 33.828 | 52.919 | −89.499 | 1.00 | 21.80 | C |
| ATOM | 184 | C | PHE | | 25 | 37.471 | 56.560 | −89.842 | 1.00 | 11.56 | C |
| ATOM | 185 | O | PHE | | 25 | 38.218 | 56.830 | −88.903 | 1.00 | 13.74 | O |
| ATOM | 186 | N | ASN | | 26 | 36.237 | 57.046 | −89.931 | 1.00 | 14.91 | N |
| ATOM | 187 | CA | ASN | | 26 | 35.740 | 57.857 | −88.826 | 1.00 | 18.22 | C |
| ATOM | 188 | CB | ASN | | 26 | 35.109 | 59.149 | −89.342 | 1.00 | 15.72 | C |
| ATOM | 189 | CG | ASN | | 26 | 34.693 | 60.069 | −88.215 | 1.00 | 19.71 | C |
| ATOM | 190 | OD1 | ASN | | 26 | 33.880 | 59.697 | −87.368 | 1.00 | 17.15 | O |
| ATOM | 191 | ND2 | ASN | | 26 | 35.254 | 61.268 | −88.192 | 1.00 | 17.11 | N |
| ATOM | 192 | C | ASN | | 26 | 34.702 | 57.018 | −88.073 | 1.00 | 18.01 | C |
| ATOM | 193 | O | ASN | | 26 | 33.688 | 56.623 | −88.639 | 1.00 | 17.23 | O |
| ATOM | 194 | N | HIS | | 27 | 34.957 | 56.731 | −86.799 | 1.00 | 18.86 | N |
| ATOM | 195 | CA | HIS | | 27 | 34.109 | 55.909 | −85.947 | 1.00 | 19.50 | C |
| ATOM | 196 | CB | HIS | | 27 | 34.842 | 55.542 | −84.642 | 1.00 | 24.19 | C |
| ATOM | 197 | CG | HIS | | 27 | 36.024 | 54.643 | −84.845 | 1.00 | 30.41 | C |
| ATOM | 198 | CD2 | HIS | | 27 | 36.117 | 53.292 | −84.904 | 1.00 | 32.97 | C |
| ATOM | 199 | ND1 | HIS | | 27 | 37.298 | 55.123 | −85.051 | 1.00 | 30.06 | N |
| ATOM | 200 | CE1 | HIS | | 27 | 38.127 | 54.108 | −85.226 | 1.00 | 33.59 | C |
| ATOM | 201 | NE2 | HIS | | 27 | 37.436 | 52.985 | −85.143 | 1.00 | 35.42 | N |
| ATOM | 202 | C | HIS | | 27 | 32.779 | 56.535 | −85.598 | 1.00 | 21.39 | C |
| ATOM | 203 | O | HIS | | 27 | 31.867 | 55.847 | −85.125 | 1.00 | 19.10 | O |
| ATOM | 204 | N | ILE | | 28 | 32.645 | 57.839 | −85.813 | 1.00 | 20.09 | N |
| ATOM | 205 | CA | ILE | | 28 | 31.392 | 58.524 | −85.480 | 1.00 | 18.15 | C |
| ATOM | 206 | CB | ILE | | 28 | 31.696 | 59.909 | −84.872 | 1.00 | 17.58 | C |
| ATOM | 207 | CG2 | ILE | | 28 | 30.396 | 60.619 | −84.493 | 1.00 | 15.87 | C |
| ATOM | 208 | CG1 | ILE | | 28 | 32.598 | 59.754 | −83.643 | 1.00 | 19.10 | C |
| ATOM | 209 | CD1 | ILE | | 28 | 33.157 | 61.081 | −83.141 | 1.00 | 23.70 | C |
| ATOM | 210 | C | ILE | | 28 | 30.482 | 58.706 | −86.696 | 1.00 | 20.68 | C |
| ATOM | 211 | O | ILE | | 28 | 29.254 | 58.565 | −86.611 | 1.00 | 15.92 | O |
| ATOM | 212 | N | THR | | 29 | 31.077 | 59.022 | −87.846 | 1.00 | 16.85 | N |
| ATOM | 213 | CA | THR | | 29 | 30.289 | 59.218 | −89.044 | 1.00 | 14.27 | C |
| ATOM | 214 | CB | THR | | 29 | 30.844 | 60.392 | −89.862 | 1.00 | 12.70 | C |
| ATOM | 215 | OG1 | THR | | 29 | 32.148 | 60.050 | −90.336 | 1.00 | 10.70 | O |
| ATOM | 216 | CG2 | THR | | 29 | 30.990 | 61.615 | −88.986 | 1.00 | 13.62 | C |
| ATOM | 217 | C | THR | | 29 | 30.323 | 57.980 | −89.926 | 1.00 | 17.71 | C |
| ATOM | 218 | O | THR | | 29 | 29.578 | 57.885 | −90.900 | 1.00 | 18.28 | O |
| ATOM | 219 | N | ASN | | 30 | 31.176 | 57.015 | −89.591 | 1.00 | 12.37 | N |
| ATOM | 220 | CA | ASN | | 30 | 31.357 | 55.793 | −90.367 | 1.00 | 12.68 | C |
| ATOM | 221 | CB | ASN | | 30 | 30.056 | 54.988 | −90.441 | 1.00 | 14.38 | C |
| ATOM | 222 | CG | ASN | | 30 | 29.627 | 54.499 | −89.087 | 1.00 | 20.16 | C |
| ATOM | 223 | OD1 | ASN | | 30 | 30.474 | 54.248 | −88.226 | 1.00 | 20.50 | O |
| ATOM | 224 | ND2 | ASN | | 30 | 28.323 | 54.358 | −88.881 | 1.00 | 18.60 | N |
| ATOM | 225 | C | ASN | | 30 | 31.893 | 56.054 | −91.773 | 1.00 | 12.32 | C |
| ATOM | 226 | O | ASN | | 30 | 31.782 | 55.207 | −92.661 | 1.00 | 16.97 | O |
| ATOM | 227 | N | ALA | | 31 | 32.496 | 57.217 | −91.983 | 1.00 | 15.08 | N |
| ATOM | 228 | CA | ALA | | 31 | 33.067 | 57.530 | −93.294 | 1.00 | 15.31 | C |
| ATOM | 229 | CB | ALA | | 31 | 33.204 | 59.062 | −93.446 | 1.00 | 13.76 | C |
| ATOM | 230 | C | ALA | | 31 | 34.445 | 56.882 | −93.444 | 1.00 | 15.85 | C |
| ATOM | 231 | O | ALA | | 31 | 35.171 | 56.720 | −92.464 | 1.00 | 15.93 | O |
| ATOM | 232 | N | SER | | 32 | 34.809 | 56.493 | −94.665 | 1.00 | 15.06 | N |
| ATOM | 233 | CA | SER | | 32 | 36.098 | 55.911 | −95.033 | 1.00 | 18.70 | C |
| ATOM | 234 | CB | SER | | 32 | 35.974 | 54.438 | −95.426 | 1.00 | 16.77 | C |
| ATOM | 235 | OG | SER | | 32 | 35.454 | 53.661 | −94.363 | 1.00 | 23.74 | O |
| ATOM | 236 | C | SER | | 32 | 36.639 | 56.669 | −96.232 | 1.00 | 18.95 | C |
| ATOM | 237 | O | SER | | 32 | 35.875 | 57.069 | −97.111 | 1.00 | 18.38 | O |
| ATOM | 238 | N | GLN | | 33 | 37.946 | 56.894 | −96.268 | 1.00 | 14.33 | N |
| ATOM | 239 | CA | GLN | | 33 | 38.578 | 57.565 | −97.396 | 1.00 | 15.22 | C |
| ATOM | 240 | CB | GLN | | 33 | 38.386 | 59.073 | −97.312 | 1.00 | 16.19 | C |
| ATOM | 241 | CG | GLN | | 33 | 39.052 | 59.712 | −96.111 | 1.00 | 14.18 | C |
| ATOM | 242 | CD | GLN | | 33 | 38.689 | 61.171 | −95.985 | 1.00 | 19.20 | C |
| ATOM | 243 | OE1 | GLN | | 33 | 37.522 | 61.542 | −96.139 | 1.00 | 16.81 | O |
| ATOM | 244 | NE2 | GLN | | 33 | 39.679 | 62.013 | −95.702 | 1.00 | 15.74 | N |
| ATOM | 245 | C | GLN | | 33 | 40.066 | 57.249 | −97.430 | 1.00 | 17.41 | C |
| ATOM | 246 | O | GLN | | 33 | 40.677 | 56.956 | −96.403 | 1.00 | 14.34 | O |
| ATOM | 247 | N | TRP | | 34 | 40.655 | 57.280 | −98.618 | 1.00 | 14.94 | N |
| ATOM | 248 | CA | TRP | | 34 | 42.069 | 57.010 | −98.783 | 1.00 | 16.95 | C |
| ATOM | 249 | CB | TRP | | 34 | 42.383 | 56.787 | −100.264 | 1.00 | 18.89 | C |
| ATOM | 250 | CG | TRP | | 34 | 41.639 | 55.658 | −100.879 | 1.00 | 19.70 | C |
| ATOM | 251 | CD2 | TRP | | 34 | 41.723 | 54.286 | −100.509 | 1.00 | 23.58 | C |
| ATOM | 252 | CE2 | TRP | | 34 | 40.850 | 53.568 | −101.358 | 1.00 | 28.79 | C |
| ATOM | 253 | CE3 | TRP | | 34 | 42.451 | 53.586 | −99.542 | 1.00 | 24.35 | C |
| ATOM | 254 | CD1 | TRP | | 34 | 40.745 | 55.724 | −101.914 | 1.00 | 29.43 | C |
| ATOM | 255 | NE1 | TRP | | 34 | 40.266 | 54.470 | −102.209 | 1.00 | 25.77 | N |
| ATOM | 256 | CZ2 | TRP | | 34 | 40.686 | 52.185 | −101.263 | 1.00 | 29.72 | C |
| ATOM | 257 | CZ3 | TRP | | 34 | 42.286 | 52.215 | −99.449 | 1.00 | 23.91 | C |
| ATOM | 258 | CH2 | TRP | | 34 | 41.410 | 51.529 | −100.304 | 1.00 | 30.70 | C |
| ATOM | 259 | C | TRP | | 34 | 42.901 | 58.177 | −98.277 | 1.00 | 21.07 | C |
| ATOM | 260 | O | TRP | | 34 | 43.994 | 57.995 | −97.729 | 1.00 | 20.27 | O |
| ATOM | 261 | N | GLU | | 35 | 42.394 | 59.394 | −98.457 | 1.00 | 17.09 | N |
| ATOM | 262 | CA | GLU | | 35 | 43.091 | 60.620 | −98.073 | 1.00 | 23.23 | C |
| ATOM | 263 | CB | GLU | | 35 | 42.451 | 61.816 | −98.779 | 1.00 | 21.52 | C |
| ATOM | 264 | CG | GLU | | 35 | 42.540 | 61.761 | −100.302 | 1.00 | 22.63 | C |
| ATOM | 265 | CD | GLU | | 35 | 41.449 | 60.912 | −100.975 | 1.00 | 30.20 | C |
| ATOM | 266 | OE1 | GLU | | 35 | 41.576 | 60.666 | −102.199 | 1.00 | 25.87 | O |
| ATOM | 267 | OE2 | GLU | | 35 | 40.473 | 60.499 | −100.303 | 1.00 | 17.07 | O |
| ATOM | 268 | C | GLU | | 35 | 43.158 | 60.910 | −96.578 | 1.00 | 24.84 | C |
| ATOM | 269 | O | GLU | | 35 | 42.142 | 60.866 | −95.880 | 1.00 | 24.06 | O |
| ATOM | 270 | N | ARG | | 36 | 44.354 | 61.223 | −96.075 | 1.00 | 19.86 | N |
| ATOM | 271 | CA | ARG | | 36 | 44.513 | 61.553 | −94.659 | 1.00 | 19.66 | C |
| ATOM | 272 | CB | ARG | | 36 | 45.957 | 61.979 | −94.376 | 1.00 | 26.03 | C |
| ATOM | 273 | CG | ARG | | 36 | 46.315 | 61.938 | −92.905 | 1.00 | 22.38 | C |
| ATOM | 274 | CD | ARG | | 36 | 47.809 | 62.127 | −92.662 | 1.00 | 29.42 | C |
| ATOM | 275 | NE | ARG | | 36 | 48.116 | 62.010 | −91.238 | 1.00 | 34.15 | N |
| ATOM | 276 | CZ | ARG | | 36 | 47.893 | 62.960 | −90.336 | 1.00 | 36.50 | C |
| ATOM | 277 | NH1 | ARG | | 36 | 47.369 | 64.121 | −90.696 | 1.00 | 42.46 | N |
| ATOM | 278 | NH2 | ARG | | 36 | 48.162 | 62.738 | −89.060 | 1.00 | 42.91 | N |
| ATOM | 279 | C | ARG | | 36 | 43.543 | 62.700 | −94.413 | 1.00 | 20.16 | C |
| ATOM | 280 | O | ARG | | 36 | 43.527 | 63.663 | −95.175 | 1.00 | 19.90 | O |
| ATOM | 281 | N | PRO | | 37 | 42.699 | 62.601 | −93.367 | 1.00 | 22.09 | N |
| ATOM | 282 | CD | PRO | | 37 | 42.622 | 61.481 | −92.411 | 1.00 | 16.30 | C |
| ATOM | 283 | CA | PRO | | 37 | 41.703 | 63.627 | −93.036 | 1.00 | 19.67 | C |
| ATOM | 284 | CB | PRO | | 37 | 40.835 | 62.933 | −91.973 | 1.00 | 17.49 | C |
| ATOM | 285 | CG | PRO | | 37 | 41.812 | 62.078 | −91.273 | 1.00 | 15.96 | C |
| ATOM | 286 | C | PRO | | 37 | 42.191 | 65.000 | −92.597 | 1.00 | 23.35 | C |
| ATOM | 287 | O | PRO | | 37 | 41.388 | 65.914 | −92.422 | 1.00 | 23.74 | O |
| ATOM | 288 | N | SER | | 38 | 43.498 | 65.162 | −92.414 | 1.00 | 21.26 | N |
| ATOM | 289 | CA | SER | | 38 | 44.062 | 66.457 | −92.034 | 1.00 | 27.89 | C |
| ATOM | 290 | CB | SER | | 38 | 43.894 | 66.706 | −90.534 | 1.00 | 36.29 | C |
| ATOM | 291 | OG | SER | | 38 | 44.529 | 65.696 | −89.761 | 1.00 | 36.61 | O |
| ATOM | 292 | C | SER | | 38 | 45.536 | 66.490 | −92.398 | 1.00 | 34.95 | C |
| ATOM | 293 | O | SER | | 38 | 46.172 | 65.446 | −92.536 | 1.00 | 33.52 | O |
| ATOM | 294 | N | GLY | | 39 | 46.093 | 67.679 | −92.581 | 1.00 | 43.46 | N |
| ATOM | 295 | CA | GLY | | 39 | 47.502 | 67.777 | −92.921 | 1.00 | 51.70 | C |
| ATOM | 296 | C | GLY | | 39 | 48.308 | 68.187 | −91.703 | 1.00 | 55.32 | C |
| ATOM | 297 | O | GLY | | 39 | 47.765 | 68.755 | −90.752 | 1.00 | 56.20 | O |
| ATOM | 298 | N | ASN | | 40 | 49.607 | 67.908 | −91.705 | 1.00 | 59.94 | N |
| ATOM | 299 | CA | ASN | | 40 | 50.331 | 68.331 | −90.507 | 1.00 | 63.30 | C |
| ATOM | 300 | CB | ASN | | 40 | 51.686 | 67.616 | −90.425 | 1.00 | 65.04 | C |
| ATOM | 301 | CG | ASN | | 40 | 51.543 | 66.101 | −90.298 | 1.00 | 68.78 | C |
| ATOM | 302 | OD1 | ASN | | 40 | 51.045 | 65.434 | −91.208 | 1.00 | 69.78 | O |
| ATOM | 303 | ND2 | ASN | | 40 | 51.975 | 65.555 | −89.163 | 1.00 | 68.06 | N |
| ATOM | 304 | C | ASN | | 40 | 50.524 | 69.847 | −90.483 | 1.00 | 62.48 | C |
| ATOM | 305 | O | ASN | | 40 | 50.620 | 70.484 | −91.531 | 1.00 | 58.64 | O |
| ATOM | 306 | N | SER | | 41 | 50.569 | 70.439 | −89.291 | 1.00 | 65.81 | N |
| ATOM | 307 | CA | SER | | 41 | 50.736 | 71.887 | −89.156 | 1.00 | 66.16 | C |
| ATOM | 308 | CB | SER | | 41 | 49.364 | 72.564 | −89.031 | 1.00 | 67.72 | C |
| ATOM | 309 | OG | SER | | 41 | 48.641 | 72.076 | −87.910 | 1.00 | 68.82 | O |
| ATOM | 310 | C | SER | | 41 | 51.608 | 72.285 | −87.964 | 1.00 | 65.99 | C |
| ATOM | 311 | O | SER | | 41 | 51.919 | 71.464 | −87.100 | 1.00 | 66.68 | O |
| ATOM | 312 | N | GLY | | 48 | 41.156 | 74.174 | −79.433 | 1.00 | 47.23 | N |
| ATOM | 313 | CA | GLY | | 48 | 40.070 | 74.433 | −78.506 | 1.00 | 45.79 | C |
| ATOM | 314 | C | GLY | | 48 | 38.783 | 74.711 | −79.257 | 1.00 | 41.88 | C |
| ATOM | 315 | O | GLY | | 48 | 37.811 | 75.220 | −78.703 | 1.00 | 44.68 | O |
| ATOM | 316 | N | GLN | | 49 | 38.773 | 74.377 | −80.540 | 1.00 | 37.67 | N |
| ATOM | 317 | CA | GLN | | 49 | 37.715 | 74.508 | −81.533 | 1.00 | 35.64 | C |
| ATOM | 318 | CB | GLN | | 49 | 38.237 | 73.995 | −82.874 | 1.00 | 44.40 | C |
| ATOM | 319 | CG | GLN | | 49 | 37.336 | 74.208 | −84.071 | 1.00 | 47.30 | C |
| ATOM | 320 | CD | GLN | | 49 | 37.936 | 73.593 | −85.324 | 1.00 | 54.28 | C |
| ATOM | 321 | OE1 | GLN | | 49 | 37.355 | 73.660 | −86.413 | 1.00 | 52.76 | O |
| ATOM | 322 | NE2 | GLN | | 49 | 39.112 | 72.984 | −85.174 | 1.00 | 51.95 | N |
| ATOM | 323 | C | GLN | | 49 | 36.431 | 73.756 | −81.152 | 1.00 | 32.56 | C |
| ATOM | 324 | O | GLN | | 49 | 35.322 | 74.227 | −81.430 | 1.00 | 29.51 | O |
| ATOM | 325 | N | GLY | | 50 | 36.562 | 72.587 | −80.528 | 1.00 | 26.84 | N |
| ATOM | 326 | CA | GLY | | 50 | 35.380 | 71.825 | −80.147 | 1.00 | 30.72 | C |
| ATOM | 327 | C | GLY | | 50 | 34.477 | 71.522 | −81.340 | 1.00 | 28.13 | C |
| ATOM | 328 | O | GLY | | 50 | 34.958 | 71.353 | −82.462 | 1.00 | 36.99 | O |
| ATOM | 329 | N | GLU | | 51 | 33.165 | 71.450 | −81.127 | 1.00 | 24.26 | N |
| ATOM | 330 | CA | GLU | | 51 | 32.302 | 71.167 | −82.274 | 1.00 | 24.47 | C |
| ATOM | 331 | CB | GLU | | 51 | 31.478 | 69.889 | −82.007 | 1.00 | 24.12 | C |
| ATOM | 332 | CG | GLU | | 51 | 30.407 | 70.031 | −80.925 | 1.00 | 25.59 | C |
| ATOM | 333 | CD | GLU | | 51 | 29.633 | 68.734 | −80.642 | 1.00 | 26.70 | C |
| ATOM | 334 | OE1 | GLU | | 51 | 28.646 | 68.781 | −79.882 | 1.00 | 32.49 | O |
| ATOM | 335 | OE2 | GLU | | 51 | 30.005 | 67.663 | −81.156 | 1.00 | 25.01 | O |
| ATOM | 336 | C | GLU | | 51 | 31.369 | 72.356 | −82.567 | 1.00 | 22.09 | C |
| ATOM | 337 | O | GLU | | 51 | 31.204 | 73.234 | −81.725 | 1.00 | 22.68 | O |
| ATOM | 338 | N | PRO | | 52 | 30.769 | 72.405 | −83.775 | 1.00 | 18.03 | N |
| ATOM | 339 | CD | PRO | | 52 | 30.812 | 71.357 | −84.813 | 1.00 | 16.98 | C |
| ATOM | 340 | CA | PRO | | 52 | 29.853 | 73.492 | −84.150 | 1.00 | 17.22 | C |
| ATOM | 341 | CB | PRO | | 52 | 29.359 | 73.098 | −85.547 | 1.00 | 18.52 | C |
| ATOM | 342 | CG | PRO | | 52 | 30.319 | 72.071 | −86.018 | 1.00 | 19.93 | C |
| ATOM | 343 | C | PRO | | 52 | 28.685 | 73.471 | −83.178 | 1.00 | 19.29 | C |
| ATOM | 344 | O | PRO | | 52 | 28.342 | 72.410 | −82.661 | 1.00 | 17.53 | O |
| ATOM | 345 | N | ALA | | 53 | 28.055 | 74.621 | −82.939 | 1.00 | 21.12 | N |
| ATOM | 346 | CA | ALA | | 53 | 26.901 | 74.660 | −82.034 | 1.00 | 23.87 | C |
| ATOM | 347 | CB | ALA | | 53 | 26.752 | 76.048 | −81.416 | 1.00 | 22.67 | C |
| ATOM | 348 | C | ALA | | 53 | 25.637 | 74.295 | −82.798 | 1.00 | 20.59 | C |
| ATOM | 349 | O | ALA | | 53 | 24.649 | 73.854 | −82.219 | 1.00 | 20.02 | O |
| ATOM | 350 | N | ARG | | 54 | 25.650 | 74.496 | −84.107 | 1.00 | 15.87 | N |
| ATOM | 351 | CA | ARG | | 54 | 24.578 | 74.199 | −85.050 | 1.00 | 20.86 | C |
| ATOM | 352 | CB | ARG | | 54 | 23.670 | 75.414 | −85.260 | 1.00 | 26.59 | C |
| ATOM | 353 | CG | ARG | | 54 | 22.739 | 75.725 | −84.101 | 1.00 | 34.05 | C |
| ATOM | 354 | CD | ARG | | 54 | 22.010 | 77.041 | −84.338 | 1.00 | 36.72 | C |
| ATOM | 355 | NE | ARG | | 54 | 21.056 | 76.992 | −85.446 | 1.00 | 40.92 | N |
| ATOM | 356 | CZ | ARG | | 54 | 19.912 | 76.315 | −85.417 | 1.00 | 36.61 | C |
| ATOM | 357 | NH1 | ARG | | 54 | 19.577 | 75.616 | −84.341 | 1.00 | 46.15 | N |
| ATOM | 358 | NH2 | ARG | | 54 | 19.079 | 76.373 | −86.442 | 1.00 | 40.16 | N |
| ATOM | 359 | C | ARG | | 54 | 25.158 | 73.812 | −86.405 | 1.00 | 20.69 | C |
| ATOM | 360 | O | ARG | | 54 | 26.193 | 74.336 | −86.824 | 1.00 | 21.34 | O |
| ATOM | 361 | N | VAL | | 55 | 24.489 | 72.905 | −87.106 | 1.00 | 17.73 | N |
| ATOM | 362 | CA | VAL | | 55 | 24.915 | 72.480 | −88.425 | 1.00 | 16.06 | C |
| ATOM | 363 | CB | VAL | | 55 | 25.587 | 71.082 | −88.414 | 1.00 | 14.14 | C |
| ATOM | 364 | CG1 | VAL | | 55 | 26.825 | 71.090 | −87.521 | 1.00 | 18.25 | C |
| ATOM | 365 | CG2 | VAL | | 55 | 24.600 | 70.029 | −87.961 | 1.00 | 17.35 | C |
| ATOM | 366 | C | VAL | | 55 | 23.710 | 72.383 | −89.342 | 1.00 | 23.63 | C |
| ATOM | 367 | O | VAL | | 55 | 22.579 | 72.312 | −88.886 | 1.00 | 21.69 | O |
| ATOM | 368 | N | ARG | | 56 | 23.945 | 72.410 | −90.646 | 1.00 | 16.32 | N |
| ATOM | 369 | CA | ARG | | 56 | 22.903 | 72.228 | −91.617 | 1.00 | 16.56 | C |
| ATOM | 370 | CB | ARG | | 56 | 22.815 | 73.394 | −92.598 | 1.00 | 20.58 | C |
| ATOM | 371 | CG | ARG | | 56 | 21.651 | 73.210 | −93.606 | 1.00 | 20.30 | C |
| ATOM | 372 | CD | ARG | | 56 | 21.469 | 74.444 | −94.469 | 1.00 | 23.99 | C |
| ATOM | 373 | NE | ARG | | 56 | 20.355 | 74.313 | −95.405 | 1.00 | 20.71 | N |
| ATOM | 374 | CZ | ARG | | 56 | 20.007 | 75.256 | −96.274 | 1.00 | 25.63 | C |
| ATOM | 375 | NH1 | ARG | | 56 | 20.681 | 76.400 | −96.318 | 1.00 | 27.93 | N |
| ATOM | 376 | NH2 | ARG | | 56 | 19.006 | 75.046 | −97.115 | 1.00 | 30.91 | N |
| ATOM | 377 | C | ARG | | 56 | 23.282 | 70.972 | −92.381 | 1.00 | 15.24 | C |
| ATOM | 378 | O | ARG | | 56 | 24.419 | 70.833 | −92.831 | 1.00 | 15.67 | O |
| ATOM | 379 | N | CYS | | 57 | 22.346 | 70.044 | −92.530 | 1.00 | 15.39 | N |
| ATOM | 380 | CA | CYS | | 57 | 22.676 | 68.825 | −93.249 | 1.00 | 14.42 | C |
| ATOM | 381 | CB | CYS | | 57 | 22.895 | 67.653 | −92.280 | 1.00 | 16.54 | C |
| ATOM | 382 | SG | CYS | | 57 | 24.321 | 67.804 | −91.188 | 1.00 | 18.14 | S |
| ATOM | 383 | C | CYS | | 57 | 21.580 | 68.396 | −94.185 | 1.00 | 16.29 | C |
| ATOM | 384 | O | CYS | | 57 | 20.417 | 68.764 | −94.008 | 1.00 | 16.29 | O |
| ATOM | 385 | N | SER | | 58 | 21.943 | 67.603 | −95.189 | 1.00 | 15.57 | N |
| ATOM | 386 | CA | SER | | 58 | 21.033 | 66.987 | −96.142 | 1.00 | 17.29 | C |
| ATOM | 387 | CB | SER | | 58 | 21.395 | 67.369 | −97.574 | 1.00 | 20.49 | C |
| ATOM | 388 | OG | SER | | 58 | 21.209 | 68.756 | −97.766 | 1.00 | 20.09 | O |
| ATOM | 389 | C | SER | | 58 | 21.232 | 65.490 | −95.942 | 1.00 | 18.58 | C |
| ATOM | 390 | O | SER | | 58 | 22.272 | 65.066 | −95.417 | 1.00 | 18.17 | O |
| ATOM | 391 | N | HIS | | 59 | 20.232 | 64.684 | −96.283 | 1.00 | 16.27 | N |
| ATOM | 392 | CA | HIS | | 59 | 20.500 | 63.273 | −96.110 | 1.00 | 15.04 | C |
| ATOM | 393 | CB | HIS | | 59 | 20.169 | 62.807 | −94.675 | 1.00 | 18.41 | C |
| ATOM | 394 | CG | HIS | | 59 | 18.705 | 62.595 | −94.406 | 1.00 | 19.46 | C |
| ATOM | 395 | CD2 | HIS | | 59 | 17.608 | 63.284 | −94.802 | 1.00 | 19.84 | C |
| ATOM | 396 | ND1 | HIS | | 59 | 18.246 | 61.590 | −93.582 | 1.00 | 19.43 | N |
| ATOM | 397 | CE1 | HIS | | 59 | 16.931 | 61.669 | −93.481 | 1.00 | 18.19 | C |
| ATOM | 398 | NE2 | HIS | | 59 | 16.519 | 62.687 | −94.212 | 1.00 | 17.42 | N |
| ATOM | 399 | C | HIS | | 59 | 19.739 | 62.424 | −97.112 | 1.00 | 15.74 | C |
| ATOM | 400 | O | HIS | | 59 | 18.771 | 62.879 | −97.731 | 1.00 | 20.23 | O |
| ATOM | 401 | N | LEU | | 60 | 20.196 | 61.195 | −97.314 | 1.00 | 14.60 | N |
| ATOM | 402 | CA | LEU | | 60 | 19.539 | 60.231 | −98.180 | 1.00 | 18.62 | C |
| ATOM | 403 | CB | LEU | | 60 | 20.451 | 59.737 | −99.314 | 1.00 | 18.48 | C |
| ATOM | 404 | CG | LEU | | 60 | 19.808 | 58.854 | −100.402 | 1.00 | 16.62 | C |
| ATOM | 405 | CD1 | LEU | | 60 | 20.727 | 58.835 | −101.619 | 1.00 | 15.75 | C |
| ATOM | 406 | CD2 | LEU | | 60 | 19.557 | 57.410 | −99.898 | 1.00 | 15.40 | C |
| ATOM | 407 | C | LEU | | 60 | 19.225 | 59.086 | −97.251 | 1.00 | 17.45 | C |
| ATOM | 408 | O | LEU | | 60 | 20.121 | 58.501 | −96.653 | 1.00 | 17.04 | O |
| ATOM | 409 | N | LEU | | 61 | 17.945 | 58.776 | −97.096 | 1.00 | 17.58 | N |
| ATOM | 410 | CA | LEU | | 61 | 17.509 | 57.709 | −96.217 | 1.00 | 15.18 | C |
| ATOM | 411 | CB | LEU | | 61 | 16.370 | 58.207 | −95.310 | 1.00 | 18.74 | C |
| ATOM | 412 | CG | LEU | | 61 | 15.672 | 57.129 | −94.467 | 1.00 | 18.02 | C |
| ATOM | 413 | CD1 | LEU | | 61 | 16.633 | 56.587 | −93.406 | 1.00 | 23.06 | C |
| ATOM | 414 | CD2 | LEU | | 61 | 14.422 | 57.711 | −93.826 | 1.00 | 17.15 | C |
| ATOM | 415 | C | LEU | | 61 | 17.025 | 56.489 | −96.973 | 1.00 | 18.37 | C |
| ATOM | 416 | O | LEU | | 61 | 16.266 | 56.607 | −97.936 | 1.00 | 17.24 | O |
| ATOM | 417 | N | VAL | | 62 | 17.489 | 55.312 | −96.569 | 1.00 | 17.00 | N |
| ATOM | 418 | CA | VAL | | 62 | 16.980 | 54.098 | −97.181 | 1.00 | 19.39 | C |
| ATOM | 419 | CB | VAL | | 62 | 18.060 | 53.251 | −97.887 | 1.00 | 12.97 | C |
| ATOM | 420 | CG1 | VAL | | 62 | 17.407 | 51.977 | −98.496 | 1.00 | 14.65 | C |
| ATOM | 421 | CG2 | VAL | | 62 | 18.717 | 54.052 | −98.989 | 1.00 | 14.75 | C |
| ATOM | 422 | C | VAL | | 62 | 16.388 | 53.289 | −96.033 | 1.00 | 22.52 | C |
| ATOM | 423 | O | VAL | | 62 | 17.100 | 52.829 | −95.133 | 1.00 | 16.99 | O |
| ATOM | 424 | N | LYS | | 63 | 15.072 | 53.149 | −96.030 | 1.00 | 19.20 | N |
| ATOM | 425 | CA | LYS | | 63 | 14.329 | 52.419 | −95.027 | 1.00 | 18.08 | C |
| ATOM | 426 | CB | LYS | | 63 | 12.881 | 52.924 | −94.994 | 1.00 | 19.50 | C |
| ATOM | 427 | CG | LYS | | 63 | 12.728 | 54.282 | −94.343 | 1.00 | 26.15 | C |
| ATOM | 428 | CD | LYS | | 63 | 11.286 | 54.532 | −93.932 | 1.00 | 22.92 | C |
| ATOM | 429 | CE | LYS | | 63 | 11.144 | 55.899 | −93.315 | 1.00 | 23.10 | C |
| ATOM | 430 | NZ | LYS | | 63 | 9.714 | 56.267 | −93.080 | 1.00 | 21.90 | N |
| ATOM | 431 | C | LYS | | 63 | 14.348 | 50.935 | −95.325 | 1.00 | 21.75 | C |
| ATOM | 432 | O | LYS | | 63 | 14.758 | 50.522 | −96.411 | 1.00 | 19.07 | O |
| ATOM | 433 | N | HIS | | 64 | 13.937 | 50.117 | −94.356 | 1.00 | 20.61 | N |
| ATOM | 434 | CA | HIS | | 64 | 13.834 | 48.668 | −94.476 | 1.00 | 23.12 | C |
| ATOM | 435 | CB | HIS | | 64 | 15.141 | 47.979 | −94.049 | 1.00 | 19.57 | C |
| ATOM | 436 | CG | HIS | | 64 | 15.647 | 48.403 | −92.708 | 1.00 | 25.71 | C |
| ATOM | 437 | CD2 | HIS | | 64 | 16.662 | 49.229 | −92.360 | 1.00 | 20.46 | C |
| ATOM | 438 | ND1 | HIS | | 64 | 15.078 | 47.978 | −91.527 | 1.00 | 22.18 | N |
| ATOM | 439 | CE1 | HIS | | 64 | 15.722 | 48.523 | −90.510 | 1.00 | 20.88 | C |
| ATOM | 440 | NE2 | HIS | | 64 | 16.688 | 49.286 | −90.989 | 1.00 | 25.47 | N |
| ATOM | 441 | C | HIS | | 64 | 12.627 | 48.154 | −93.670 | 1.00 | 23.94 | C |
| ATOM | 442 | O | HIS | | 64 | 11.965 | 48.921 | −92.965 | 1.00 | 19.08 | O |
| ATOM | 443 | N | SER | | 65 | 12.327 | 46.862 | −93.778 | 1.00 | 27.27 | N |
| ATOM | 444 | CA | SER | | 65 | 11.179 | 46.262 | −93.096 | 1.00 | 27.89 | C |
| ATOM | 445 | CB | SER | | 65 | 11.084 | 44.768 | −93.427 | 1.00 | 26.96 | C |
| ATOM | 446 | OG | SER | | 65 | 12.178 | 44.066 | −92.876 | 1.00 | 29.35 | O |
| ATOM | 447 | C | SER | | 65 | 11.173 | 46.460 | −91.586 | 1.00 | 27.90 | C |
| ATOM | 448 | O | SER | | 65 | 10.114 | 46.475 | −90.968 | 1.00 | 34.45 | O |
| ATOM | 449 | N | GLN | | 66 | 12.343 | 46.631 | −90.978 | 1.00 | 27.99 | N |
| ATOM | 450 | CA | GLN | | 66 | 12.395 | 46.846 | −89.530 | 1.00 | 25.91 | C |
| ATOM | 451 | CB | GLN | | 66 | 13.582 | 46.093 | −88.921 | 1.00 | 33.39 | C |
| ATOM | 452 | CG | GLN | | 66 | 13.502 | 44.580 | −89.061 | 1.00 | 37.71 | C |
| ATOM | 453 | CD | GLN | | 66 | 12.311 | 43.991 | −88.329 | 1.00 | 41.26 | C |
| ATOM | 454 | OE1 | GLN | | 66 | 12.083 | 44.291 | −87.156 | 1.00 | 41.31 | O |
| ATOM | 455 | NE2 | GLN | | 66 | 11.550 | 43.144 | −89.013 | 1.00 | 42.73 | N |
| ATOM | 456 | C | GLN | | 66 | 12.477 | 48.321 | −89.131 | 1.00 | 26.34 | C |
| ATOM | 457 | O | GLN | | 66 | 12.706 | 48.650 | −87.970 | 1.00 | 30.77 | O |
| ATOM | 458 | N | SER | | 67 | 12.287 | 49.225 | −90.085 | 1.00 | 28.06 | N |
| ATOM | 459 | CA | SER | | 67 | 12.331 | 50.653 | −89.777 | 1.00 | 26.34 | C |
| ATOM | 460 | CB | SER | | 67 | 12.235 | 51.484 | −91.062 | 1.00 | 28.49 | C |
| ATOM | 461 | OG | SER | | 67 | 13.384 | 51.337 | −91.870 | 1.00 | 25.35 | O |
| ATOM | 462 | C | SER | | 67 | 11.166 | 51.042 | −88.878 | 1.00 | 29.92 | C |
| ATOM | 463 | O | SER | | 67 | 10.097 | 50.442 | −88.950 | 1.00 | 30.12 | O |
| ATOM | 464 | N | ARG | | 68 | 11.352 | 52.064 | −88.052 | 1.00 | 27.24 | N |
| ATOM | 465 | CA | ARG | | 68 | 10.304 | 52.537 | −87.152 | 1.00 | 32.91 | C |
| ATOM | 466 | CB | ARG | | 68 | 10.706 | 53.877 | −86.540 | 1.00 | 32.93 | C |
| ATOM | 467 | CG | ARG | | 68 | 9.775 | 54.365 | −85.451 | 1.00 | 47.05 | C |
| ATOM | 468 | CD | ARG | | 68 | 10.482 | 55.338 | −84.526 | 1.00 | 52.46 | C |
| ATOM | 469 | NE | ARG | | 68 | 11.004 | 56.503 | −85.237 | 1.00 | 59.99 | N |
| ATOM | 470 | CZ | ARG | | 68 | 11.776 | 57.431 | −84.678 | 1.00 | 63.82 | C |
| ATOM | 471 | NH1 | ARG | | 68 | 12.119 | 57.328 | −83.398 | 1.00 | 61.92 | N |
| ATOM | 472 | NH2 | ARG | | 68 | 12.195 | 58.469 | −85.394 | 1.00 | 67.04 | N |
| ATOM | 473 | C | ARG | | 68 | 8.990 | 52.675 | −87.918 | 1.00 | 34.45 | C |
| ATOM | 474 | O | ARG | | 68 | 7.948 | 52.187 | −87.481 | 1.00 | 36.45 | O |
| ATOM | 475 | N | ARG | | 69 | 9.033 | 53.330 | −89.073 | 1.00 | 28.84 | N |
| ATOM | 476 | CA | ARG | | 69 | 7.890 | 53.524 | −89.955 | 1.00 | 33.21 | C |
| ATOM | 477 | CB | ARG | | 69 | 7.547 | 55.010 | −90.049 | 1.00 | 34.15 | C |
| ATOM | 478 | CG | ARG | | 69 | 6.918 | 55.609 | −88.791 | 1.00 | 45.92 | C |
| ATOM | 479 | CD | ARG | | 69 | 6.967 | 57.129 | −88.848 | 1.00 | 47.36 | C |
| ATOM | 480 | NE | ARG | | 69 | 6.710 | 57.608 | −90.204 | 1.00 | 60.07 | N |
| ATOM | 481 | CZ | ARG | | 69 | 6.896 | 58.862 | −90.610 | 1.00 | 65.85 | C |
| ATOM | 482 | NH1 | ARG | | 69 | 7.342 | 59.783 | −89.763 | 1.00 | 66.71 | N |
| ATOM | 483 | NH2 | ARG | | 69 | 6.649 | 59.194 | −91.871 | 1.00 | 69.74 | N |
| ATOM | 484 | C | ARG | | 69 | 8.231 | 52.986 | −91.348 | 1.00 | 31.71 | C |
| ATOM | 485 | O | ARG | | 69 | 8.732 | 53.724 | −92.195 | 1.00 | 29.53 | O |
| ATOM | 486 | N | PRO | | 70 | 7.963 | 51.693 | −91.598 | 1.00 | 32.65 | N |
| ATOM | 487 | CD | PRO | | 70 | 7.304 | 50.753 | −90.676 | 1.00 | 34.88 | C |
| ATOM | 488 | CA | PRO | | 70 | 8.241 | 51.046 | −92.890 | 1.00 | 33.80 | C |
| ATOM | 489 | CB | PRO | | 70 | 8.032 | 49.573 | −92.573 | 1.00 | 32.16 | C |
| ATOM | 490 | CG | PRO | | 70 | 6.902 | 49.620 | −91.601 | 1.00 | 35.61 | C |
| ATOM | 491 | C | PRO | | 70 | 7.301 | 51.565 | −93.979 | 1.00 | 35.17 | C |
| ATOM | 492 | O | PRO | | 70 | 6.767 | 50.808 | −94.791 | 1.00 | 37.54 | O |
| ATOM | 493 | N | SEP | | 71 | 7.087 | 52.875 | −93.975 | 1.00 | 40.94 | N |
| ATOM | 494 | CA | SEP | | 71 | 6.431 | 54.048 | −94.536 | 1.00 | 43.40 | C |
| ATOM | 495 | CB | SEP | | 71 | 5.138 | 53.702 | −95.256 | 1.00 | 42.68 | C |
| ATOM | 496 | OG | SEP | | 71 | 4.253 | 54.811 | −95.163 | 1.00 | 58.73 | O |
| ATOM | 497 | C | SEP | | 71 | 7.352 | 54.857 | −95.449 | 1.00 | 33.80 | C |
| ATOM | 498 | O | SEP | | 71 | 8.513 | 55.050 | −95.127 | 1.00 | 40.90 | O |
| ATOM | 499 | P | SEP | | 71 | 3.574 | 55.150 | −93.863 | 1.00 | 60.87 | P |
| ATOM | 500 | O1P | SEP | | 71 | 2.483 | 54.160 | −93.622 | 1.00 | 62.48 | O |
| ATOM | 501 | O2P | SEP | | 71 | 3.004 | 56.523 | −93.951 | 1.00 | 57.06 | O |
| ATOM | 502 | O3P | SEP | | 71 | 4.539 | 55.104 | −92.731 | 1.00 | 57.69 | O |
| ATOM | 503 | N | SER | | 72 | 6.831 | 55.370 | −96.561 | 1.00 | 30.92 | N |
| ATOM | 504 | CA | SER | | 72 | 7.600 | 56.185 | −97.496 | 1.00 | 36.01 | C |
| ATOM | 505 | CB | SER | | 72 | 8.847 | 55.399 | −97.940 | 1.00 | 38.35 | C |
| ATOM | 506 | OG | SER | | 72 | 9.370 | 55.864 | −99.163 | 1.00 | 33.11 | O |
| ATOM | 507 | C | SER | | 72 | 6.791 | 56.631 | −98.723 | 1.00 | 34.45 | C |
| ATOM | 508 | O | SER | | 72 | 5.957 | 55.880 | −99.228 | 1.00 | 29.56 | O |
| ATOM | 509 | N | TRP | | 73 | 7.033 | 57.851 | −99.210 | 1.00 | 30.75 | N |
| ATOM | 510 | CA | TRP | | 73 | 6.367 | 58.390 | −100.402 | 1.00 | 31.53 | C |
| ATOM | 511 | CB | TRP | | 73 | 6.794 | 59.832 | −100.678 | 1.00 | 29.62 | C |
| ATOM | 512 | CG | TRP | | 73 | 8.275 | 59.975 | −100.917 | 1.00 | 31.63 | C |
| ATOM | 513 | CD2 | TRP | | 73 | 8.953 | 59.963 | −102.184 | 1.00 | 29.70 | C |
| ATOM | 514 | CE2 | TRP | | 73 | 10.337 | 60.078 | −101.920 | 1.00 | 28.88 | C |
| ATOM | 515 | CE3 | TRP | | 73 | 8.526 | 59.864 | −103.514 | 1.00 | 30.48 | C |
| ATOM | 516 | CD1 | TRP | | 73 | 9.252 | 60.094 | −99.966 | 1.00 | 33.56 | C |
| ATOM | 517 | NE1 | TRP | | 73 | 10.492 | 60.159 | −100.562 | 1.00 | 31.67 | N |
| ATOM | 518 | CZ2 | TRP | | 73 | 11.295 | 60.098 | −102.936 | 1.00 | 25.94 | C |
| ATOM | 519 | CZ3 | TRP | | 73 | 9.485 | 59.885 | −104.527 | 1.00 | 33.42 | C |
| ATOM | 520 | CH2 | TRP | | 73 | 10.854 | 60.000 | −104.227 | 1.00 | 33.53 | C |
| ATOM | 521 | C | TRP | | 73 | 6.758 | 57.538 | −101.604 | 1.00 | 33.07 | C |
| ATOM | 522 | O | TRP | | 73 | 6.107 | 57.571 | −102.645 | 1.00 | 33.86 | O |
| ATOM | 523 | N | ARG | | 74 | 7.841 | 56.781 | −101.474 | 1.00 | 26.69 | N |
| ATOM | 524 | CA | ARG | | 74 | 8.332 | 55.895 | −102.520 | 1.00 | 33.68 | C |
| ATOM | 525 | CB | ARG | | 74 | 9.805 | 55.548 | −102.292 | 1.00 | 28.92 | C |
| ATOM | 526 | CG | ARG | | 74 | 10.735 | 56.745 | −102.331 | 1.00 | 30.68 | C |
| ATOM | 527 | CD | ARG | | 74 | 12.135 | 56.295 | −102.653 | 1.00 | 26.31 | C |
| ATOM | 528 | NE | ARG | | 74 | 12.190 | 55.781 | −104.014 | 1.00 | 26.27 | N |
| ATOM | 529 | CZ | ARG | | 74 | 12.857 | 54.691 | −104.376 | 1.00 | 28.59 | C |
| ATOM | 530 | NH1 | ARG | | 74 | 13.527 | 53.992 | −103.474 | 1.00 | 31.13 | N |
| ATOM | 531 | NH2 | ARG | | 74 | 12.862 | 54.306 | −105.642 | 1.00 | 26.54 | N |
| ATOM | 532 | C | ARG | | 74 | 7.512 | 54.617 | −102.527 | 1.00 | 32.12 | C |
| ATOM | 533 | O | ARG | | 74 | 7.230 | 54.050 | −103.582 | 1.00 | 34.62 | O |
| ATOM | 534 | N | GLN | | 75 | 7.147 | 54.134 | −101.346 | 1.00 | 32.51 | N |
| ATOM | 535 | CA | GLN | | 75 | 6.330 | 52.934 | −101.326 | 1.00 | 36.82 | C |
| ATOM | 536 | CB | GLN | | 75 | 7.161 | 51.687 | −101.660 | 1.00 | 39.32 | C |
| ATOM | 537 | CG | GLN | | 75 | 8.444 | 51.506 | −100.907 | 1.00 | 39.98 | C |
| ATOM | 538 | CD | GLN | | 75 | 9.187 | 50.265 | −101.362 | 1.00 | 37.30 | C |
| ATOM | 539 | OE1 | GLN | | 75 | 10.285 | 49.978 | −100.894 | 1.00 | 35.40 | O |
| ATOM | 540 | NE2 | GLN | | 75 | 8.587 | 49.520 | −102.280 | 1.00 | 38.16 | N |
| ATOM | 541 | C | GLN | | 75 | 5.583 | 52.757 | −100.020 | 1.00 | 34.50 | C |
| ATOM | 542 | O | GLN | | 75 | 6.146 | 52.896 | −98.931 | 1.00 | 30.87 | O |
| ATOM | 543 | N | GLU | | 76 | 4.289 | 52.470 | −100.121 | 1.00 | 35.11 | N |
| ATOM | 544 | CA | GLU | | 76 | 3.350 | 52.273 | −99.028 | 1.00 | 34.11 | C |
| ATOM | 545 | CB | GLU | | 76 | 1.985 | 51.873 | −99.591 | 1.00 | 37.48 | C |
| ATOM | 546 | CG | GLU | | 76 | 0.876 | 51.835 | −98.561 | 1.00 | 45.67 | C |
| ATOM | 547 | CD | GLU | | 76 | −0.494 | 51.654 | −99.190 | 1.00 | 48.00 | C |
| ATOM | 548 | OE1 | GLU | | 76 | −1.441 | 51.324 | −98.451 | 1.00 | 47.83 | O |
| ATOM | 549 | OE2 | GLU | | 76 | −0.622 | 51.842 | −100.422 | 1.00 | 48.75 | O |
| ATOM | 550 | C | GLU | | 76 | 3.843 | 51.218 | −98.052 | 1.00 | 30.35 | C |
| ATOM | 551 | O | GLU | | 76 | 3.583 | 51.294 | −96.856 | 1.00 | 34.22 | O |
| ATOM | 552 | N | LYS | | 77 | 4.573 | 50.228 | −98.556 | 1.00 | 33.60 | N |
| ATOM | 553 | CA | LYS | | 77 | 5.086 | 49.164 | −97.706 | 1.00 | 37.68 | C |
| ATOM | 554 | CB | LYS | | 77 | 4.205 | 47.920 | −97.842 | 1.00 | 44.79 | C |
| ATOM | 555 | CG | LYS | | 77 | 4.741 | 46.694 | −97.127 | 1.00 | 48.22 | C |
| ATOM | 556 | CD | LYS | | 77 | 4.785 | 46.894 | −95.619 | 1.00 | 55.63 | C |
| ATOM | 557 | CE | LYS | | 77 | 5.392 | 45.675 | −94.924 | 1.00 | 58.24 | C |
| ATOM | 558 | NZ | LYS | | 77 | 4.719 | 44.394 | −95.329 | 1.00 | 56.03 | N |
| ATOM | 559 | C | LYS | | 77 | 6.529 | 48.809 | −98.047 | 1.00 | 34.41 | C |
| ATOM | 560 | O | LYS | | 77 | 6.793 | 48.124 | −99.038 | 1.00 | 30.85 | O |
| ATOM | 561 | N | ILE | | 78 | 7.473 | 49.275 | −97.230 | 1.00 | 35.38 | N |
| ATOM | 562 | CA | ILE | | 78 | 8.910 | 49.041 | −97.374 | 1.00 | 27.02 | C |
| ATOM | 563 | CB | ILE | | 78 | 9.723 | 49.897 | −96.384 | 1.00 | 23.81 | C |
| ATOM | 564 | CG2 | ILE | | 78 | 11.214 | 49.667 | −96.614 | 1.00 | 23.62 | C |
| ATOM | 565 | CG1 | ILE | | 78 | 9.374 | 51.381 | −96.547 | 1.00 | 28.25 | C |
| ATOM | 566 | CD1 | ILE | | 78 | 9.860 | 51.988 | −97.844 | 1.00 | 37.59 | C |
| ATOM | 567 | C | ILE | | 78 | 9.181 | 47.581 | −97.057 | 1.00 | 26.47 | C |
| ATOM | 568 | O | ILE | | 78 | 9.048 | 47.158 | −95.915 | 1.00 | 27.67 | O |
| ATOM | 569 | N | THR | | 79 | 9.592 | 46.802 | −98.049 | 1.00 | 25.89 | N |
| ATOM | 570 | CA | THR | | 79 | 9.813 | 45.405 | −97.722 | 1.00 | 25.84 | C |
| ATOM | 571 | CB | THR | | 79 | 8.977 | 44.505 | −98.643 | 1.00 | 28.70 | C |
| ATOM | 572 | OG1 | THR | | 79 | 9.359 | 44.730 | −100.007 | 1.00 | 32.22 | O |
| ATOM | 573 | CG2 | THR | | 79 | 7.488 | 44.827 | −98.483 | 1.00 | 31.63 | C |
| ATOM | 574 | C | THR | | 79 | 11.264 | 44.935 | −97.786 | 1.00 | 28.19 | C |
| ATOM | 575 | O | THR | | 79 | 11.559 | 43.801 | −97.424 | 1.00 | 23.84 | O |
| ATOM | 576 | N | ARG | | 80 | 12.184 | 45.799 | −98.212 | 1.00 | 29.18 | N |
| ATOM | 577 | CA | ARG | | 80 | 13.558 | 45.301 | −98.288 | 1.00 | 28.31 | C |
| ATOM | 578 | CB | ARG | | 80 | 14.457 | 46.313 | −99.029 | 1.00 | 28.65 | C |
| ATOM | 579 | CG | ARG | | 80 | 14.708 | 47.610 | −98.270 | 1.00 | 24.55 | C |
| ATOM | 580 | CD | ARG | | 80 | 15.642 | 48.554 | −99.052 | 1.00 | 20.28 | C |
| ATOM | 581 | NE | ARG | | 80 | 14.976 | 49.223 | −100.173 | 1.00 | 22.03 | N |
| ATOM | 582 | CZ | ARG | | 80 | 14.234 | 50.326 | −100.070 | 1.00 | 19.12 | C |
| ATOM | 583 | NH1 | ARG | | 80 | 14.049 | 50.902 | −98.899 | 1.00 | 18.15 | N |
| ATOM | 584 | NH2 | ARG | | 80 | 13.691 | 50.875 | −101.153 | 1.00 | 23.42 | N |
| ATOM | 585 | C | ARG | | 80 | 14.137 | 45.008 | −96.911 | 1.00 | 20.13 | C |
| ATOM | 586 | O | ARG | | 80 | 13.712 | 45.576 | −95.917 | 1.00 | 24.28 | O |
| ATOM | 587 | N | THR | | 81 | 15.107 | 44.105 | −96.842 | 1.00 | 26.53 | N |
| ATOM | 588 | CA | THR | | 81 | 15.720 | 43.780 | −95.562 | 1.00 | 26.06 | C |
| ATOM | 589 | CB | THR | | 81 | 16.438 | 42.427 | −95.612 | 1.00 | 28.99 | C |
| ATOM | 590 | OG1 | THR | | 81 | 17.501 | 42.486 | −96.577 | 1.00 | 29.21 | O |
| ATOM | 591 | CG2 | THR | | 81 | 15.458 | 41.317 | −96.002 | 1.00 | 34.21 | C |
| ATOM | 592 | C | THR | | 81 | 16.758 | 44.821 | −95.189 | 1.00 | 26.70 | C |
| ATOM | 593 | O | THR | | 81 | 17.187 | 45.616 | −96.028 | 1.00 | 24.42 | O |
| ATOM | 594 | N | LYS | | 82 | 17.189 | 44.814 | −93.933 | 1.00 | 22.31 | N |
| ATOM | 595 | CA | LYS | | 82 | 18.198 | 45.776 | −93.531 | 1.00 | 27.81 | C |
| ATOM | 596 | CB | LYS | | 82 | 18.510 | 45.648 | −92.034 | 1.00 | 24.95 | C |
| ATOM | 597 | CG | LYS | | 82 | 19.359 | 46.815 | −91.508 | 1.00 | 39.52 | C |
| ATOM | 598 | CD | LYS | | 82 | 19.303 | 46.984 | −89.987 | 1.00 | 44.27 | C |
| ATOM | 599 | CE | LYS | | 82 | 20.288 | 46.086 | −89.262 | 1.00 | 49.49 | C |
| ATOM | 600 | NZ | LYS | | 82 | 20.018 | 44.641 | −89.496 | 1.00 | 53.28 | N |
| ATOM | 601 | C | LYS | | 82 | 19.464 | 45.560 | −94.350 | 1.00 | 26.79 | C |
| ATOM | 602 | O | LYS | | 82 | 20.145 | 46.508 | −94.699 | 1.00 | 21.05 | O |
| ATOM | 603 | N | GLU | | 83 | 19.779 | 44.309 | −94.672 | 1.00 | 23.21 | N |
| ATOM | 604 | CA | GLU | | 83 | 20.949 | 43.933 | −95.465 | 1.00 | 27.50 | C |
| ATOM | 605 | CB | GLU | | 83 | 21.032 | 42.400 | −95.562 | 1.00 | 34.32 | C |
| ATOM | 606 | CG | GLU | | 83 | 21.369 | 41.684 | −94.237 | 1.00 | 45.90 | C |
| ATOM | 607 | CD | GLU | | 83 | 20.418 | 42.016 | −93.068 | 1.00 | 53.02 | C |
| ATOM | 608 | OE1 | GLU | | 83 | 20.932 | 42.363 | −91.974 | 1.00 | 52.01 | O |
| ATOM | 609 | OE2 | GLU | | 83 | 19.174 | 41.917 | −93.230 | 1.00 | 40.00 | O |
| ATOM | 610 | C | GLU | | 83 | 20.842 | 44.551 | −96.862 | 1.00 | 23.06 | C |
| ATOM | 611 | O | GLU | | 83 | 21.799 | 45.108 | −97.396 | 1.00 | 19.79 | O |
| ATOM | 612 | N | GLU | | 84 | 19.667 | 44.463 | −97.466 | 1.00 | 23.89 | N |
| ATOM | 613 | CA | GLU | | 84 | 19.428 | 45.024 | −98.783 | 1.00 | 27.81 | C |
| ATOM | 614 | CB | GLU | | 84 | 18.052 | 44.584 | −99.291 | 1.00 | 30.90 | C |
| ATOM | 615 | CG | GLU | | 84 | 17.951 | 43.085 | −99.531 | 1.00 | 36.56 | C |
| ATOM | 616 | CD | GLU | | 84 | 16.556 | 42.650 | −99.952 | 1.00 | 39.46 | C |
| ATOM | 617 | OE1 | GLU | | 84 | 15.563 | 43.189 | −99.417 | 1.00 | 30.31 | O |
| ATOM | 618 | OE2 | GLU | | 84 | 16.451 | 41.752 | −100.808 | 1.00 | 47.07 | O |
| ATOM | 619 | C | GLU | | 84 | 19.510 | 46.548 | −98.740 | 1.00 | 25.49 | C |
| ATOM | 620 | O | GLU | | 84 | 19.997 | 47.182 | −99.685 | 1.00 | 24.58 | O |
| ATOM | 621 | N | ALA | | 85 | 19.024 | 47.151 | −97.656 | 1.00 | 22.63 | N |
| ATOM | 622 | CA | ALA | | 85 | 19.105 | 48.612 | −97.559 | 1.00 | 19.16 | C |
| ATOM | 623 | CB | ALA | | 85 | 18.385 | 49.117 | −96.317 | 1.00 | 17.68 | C |
| ATOM | 624 | C | ALA | | 85 | 20.550 | 49.062 | −97.509 | 1.00 | 19.92 | C |
| ATOM | 625 | O | ALA | | 85 | 20.917 | 50.053 | −98.153 | 1.00 | 26.19 | O |
| ATOM | 626 | N | LEU | | 86 | 21.382 | 48.355 | −96.741 | 1.00 | 20.33 | N |
| ATOM | 627 | CA | LEU | | 86 | 22.795 | 48.718 | −96.629 | 1.00 | 20.45 | C |
| ATOM | 628 | CB | LEU | | 86 | 23.495 | 47.850 | −95.579 | 1.00 | 21.14 | C |
| ATOM | 629 | CG | LEU | | 86 | 24.985 | 48.135 | −95.361 | 1.00 | 21.68 | C |
| ATOM | 630 | CD1 | LEU | | 86 | 25.175 | 49.560 | −94.859 | 1.00 | 14.14 | C |
| ATOM | 631 | CD2 | LEU | | 86 | 25.560 | 47.134 | −94.366 | 1.00 | 26.61 | C |
| ATOM | 632 | C | LEU | | 86 | 23.515 | 48.590 | −97.972 | 1.00 | 20.64 | C |
| ATOM | 633 | O | LEU | | 86 | 24.400 | 49.404 | −98.303 | 1.00 | 16.24 | O |
| ATOM | 634 | N | GLU | | 87 | 23.160 | 47.570 | −98.754 | 1.00 | 22.72 | N |
| ATOM | 635 | CA | GLU | | 87 | 23.777 | 47.383 | −100.063 | 1.00 | 21.14 | C |
| ATOM | 636 | CB | GLU | | 87 | 23.289 | 46.081 | −100.714 | 1.00 | 33.28 | C |
| ATOM | 637 | CG | GLU | | 87 | 23.980 | 44.823 | −100.173 | 1.00 | 48.64 | C |
| ATOM | 638 | CD | GLU | | 87 | 25.348 | 44.571 | −100.815 | 1.00 | 59.97 | C |
| ATOM | 639 | OE1 | GLU | | 87 | 25.434 | 43.710 | −101.721 | 1.00 | 59.16 | O |
| ATOM | 640 | OE2 | GLU | | 87 | 26.334 | 45.238 | −100.423 | 1.00 | 63.53 | O |
| ATOM | 641 | C | GLU | | 87 | 23.419 | 48.556 | −100.941 | 1.00 | 20.69 | C |
| ATOM | 642 | O | GLU | | 87 | 24.237 | 49.025 | −101.734 | 1.00 | 23.88 | O |
| ATOM | 643 | N | LEU | | 88 | 22.187 | 49.038 | −100.824 | 1.00 | 17.75 | N |
| ATOM | 644 | CA | LEU | | 88 | 21.721 | 50.191 | −101.604 | 1.00 | 21.40 | C |
| ATOM | 645 | CB | LEU | | 88 | 20.226 | 50.429 | −101.373 | 1.00 | 15.66 | C |
| ATOM | 646 | CG | LEU | | 88 | 19.276 | 49.509 | −102.154 | 1.00 | 17.76 | C |
| ATOM | 647 | CD1 | LEU | | 88 | 17.857 | 49.688 | −101.659 | 1.00 | 21.58 | C |
| ATOM | 648 | CD2 | LEU | | 88 | 19.365 | 49.821 | −103.643 | 1.00 | 24.99 | C |
| ATOM | 649 | C | LEU | | 88 | 22.500 | 51.428 | −101.185 | 1.00 | 21.48 | C |
| ATOM | 650 | O | LEU | | 88 | 22.969 | 52.200 | −102.025 | 1.00 | 18.15 | O |
| ATOM | 651 | N | ILE | | 89 | 22.635 | 51.633 | −99.880 | 1.00 | 19.61 | N |
| ATOM | 652 | CA | ILE | | 89 | 23.386 | 52.764 | −99.340 | 1.00 | 19.93 | C |
| ATOM | 653 | CB | ILE | | 89 | 23.362 | 52.759 | −97.786 | 1.00 | 19.27 | C |
| ATOM | 654 | CG2 | ILE | | 89 | 24.429 | 53.705 | −97.226 | 1.00 | 21.74 | C |
| ATOM | 655 | CG1 | ILE | | 89 | 21.974 | 53.166 | −97.282 | 1.00 | 18.37 | C |
| ATOM | 656 | CD1 | ILE | | 89 | 21.685 | 54.675 | −97.338 | 1.00 | 14.84 | C |
| ATOM | 657 | C | ILE | | 89 | 24.839 | 52.717 | −99.817 | 1.00 | 20.83 | C |
| ATOM | 658 | O | ILE | | 89 | 25.385 | 53.729 | −100.252 | 1.00 | 22.46 | O |
| ATOM | 659 | N | ASN | | 90 | 25.472 | 51.545 | −99.753 | 1.00 | 17.83 | N |
| ATOM | 660 | CA | ASN | | 90 | 26.870 | 51.455 | −100.173 | 1.00 | 19.16 | C |
| ATOM | 661 | CB | ASN | | 90 | 27.453 | 50.071 | −99.826 | 1.00 | 18.78 | C |
| ATOM | 662 | CG | ASN | | 90 | 27.708 | 49.902 | −98.323 | 1.00 | 23.96 | C |
| ATOM | 663 | OD1 | ASN | | 90 | 27.936 | 50.872 | −97.602 | 1.00 | 22.85 | O |
| ATOM | 664 | ND2 | ASN | | 90 | 27.684 | 48.670 | −97.857 | 1.00 | 23.17 | N |
| ATOM | 665 | C | ASN | | 90 | 27.011 | 51.748 | −101.659 | 1.00 | 16.89 | C |
| ATOM | 666 | O | ASN | | 90 | 28.008 | 52.321 | −102.105 | 1.00 | 20.76 | O |
| ATOM | 667 | N | GLY | | 91 | 26.008 | 51.365 | −102.440 | 1.00 | 17.23 | N |
| ATOM | 668 | CA | GLY | | 91 | 26.025 | 51.625 | −103.867 | 1.00 | 20.35 | C |
| ATOM | 669 | C | GLY | | 91 | 25.979 | 53.118 | −104.144 | 1.00 | 20.52 | C |
| ATOM | 670 | O | GLY | | 91 | 26.733 | 53.629 | −104.969 | 1.00 | 24.05 | O |
| ATOM | 671 | N | TYR | | 92 | 25.089 | 53.835 | −103.465 | 1.00 | 16.80 | N |
| ATOM | 672 | CA | TYR | | 92 | 24.965 | 55.289 | −103.631 | 1.00 | 16.07 | C |
| ATOM | 673 | CB | TYR | | 92 | 23.828 | 55.811 | −102.750 | 1.00 | 12.48 | C |
| ATOM | 674 | CG | TYR | | 92 | 22.448 | 55.323 | −103.143 | 1.00 | 17.86 | C |
| ATOM | 675 | CD1 | TYR | | 92 | 21.448 | 55.157 | −102.182 | 1.00 | 18.17 | C |
| ATOM | 676 | CE1 | TYR | | 92 | 20.181 | 54.731 | −102.536 | 1.00 | 17.84 | C |
| ATOM | 677 | CD2 | TYR | | 92 | 22.136 | 55.049 | −104.474 | 1.00 | 17.91 | C |
| ATOM | 678 | CE2 | TYR | | 92 | 20.872 | 54.619 | −104.839 | 1.00 | 23.80 | C |
| ATOM | 679 | CZ | TYR | | 92 | 19.901 | 54.461 | −103.869 | 1.00 | 22.36 | C |
| ATOM | 680 | OH | TYR | | 92 | 18.656 | 54.034 | −104.233 | 1.00 | 20.44 | O |
| ATOM | 681 | C | TYR | | 92 | 26.279 | 55.969 | −103.214 | 1.00 | 14.10 | C |
| ATOM | 682 | O | TYR | | 92 | 26.786 | 56.870 | −103.887 | 1.00 | 18.00 | O |
| ATOM | 683 | N | ILE | | 93 | 26.834 | 55.549 | −102.087 | 1.00 | 11.98 | N |
| ATOM | 684 | CA | ILE | | 93 | 28.082 | 56.149 | −101.652 | 1.00 | 13.75 | C |
| ATOM | 685 | CB | ILE | | 93 | 28.545 | 55.511 | −100.327 | 1.00 | 17.06 | C |
| ATOM | 686 | CG2 | ILE | | 93 | 29.981 | 55.918 | −100.018 | 1.00 | 13.56 | C |
| ATOM | 687 | CG1 | ILE | | 93 | 27.589 | 55.919 | −99.200 | 1.00 | 16.69 | C |
| ATOM | 688 | CD1 | ILE | | 93 | 27.847 | 55.191 | −97.892 | 1.00 | 15.68 | C |
| ATOM | 689 | C | ILE | | 93 | 29.137 | 55.922 | −102.737 | 1.00 | 17.64 | C |
| ATOM | 690 | O | ILE | | 93 | 29.897 | 56.830 | −103.074 | 1.00 | 14.83 | O |
| ATOM | 691 | N | GLN | | 94 | 29.171 | 54.719 | −103.309 | 1.00 | 13.47 | N |
| ATOM | 692 | CA | GLN | | 94 | 30.161 | 54.457 | −104.344 | 1.00 | 17.51 | C |
| ATOM | 693 | CB | GLN | | 94 | 30.134 | 52.985 | −104.758 | 1.00 | 19.49 | C |
| ATOM | 694 | CG | GLN | | 94 | 31.179 | 52.621 | −105.798 | 1.00 | 26.78 | C |
| ATOM | 695 | CD | GLN | | 94 | 31.500 | 51.124 | −105.832 | 1.00 | 44.17 | C |
| ATOM | 696 | OE1 | GLN | | 94 | 32.242 | 50.659 | −106.697 | 1.00 | 48.98 | O |
| ATOM | 697 | NE2 | GLN | | 94 | 30.949 | 50.370 | −104.878 | 1.00 | 50.61 | N |
| ATOM | 698 | C | GLN | | 94 | 29.930 | 55.335 | −105.568 | 1.00 | 16.33 | C |
| ATOM | 699 | O | GLN | | 94 | 30.878 | 55.845 | −106.152 | 1.00 | 14.84 | O |
| ATOM | 700 | N | LYS | | 95 | 28.673 | 55.510 | −105.969 | 1.00 | 13.64 | N |
| ATOM | 701 | CA | LYS | | 95 | 28.351 | 56.339 | −107.124 | 1.00 | 15.80 | C |
| ATOM | 702 | CB | LYS | | 95 | 26.859 | 56.199 | −107.469 | 1.00 | 19.52 | C |
| ATOM | 703 | CG | LYS | | 95 | 26.458 | 54.804 | −108.002 | 1.00 | 18.04 | C |
| ATOM | 704 | CD | LYS | | 95 | 26.734 | 54.676 | −109.484 | 1.00 | 37.15 | C |
| ATOM | 705 | CE | LYS | | 95 | 26.532 | 53.252 | −110.014 | 1.00 | 39.75 | C |
| ATOM | 706 | NZ | LYS | | 95 | 27.743 | 52.390 | −109.837 | 1.00 | 37.17 | N |
| ATOM | 707 | C | LYS | | 95 | 28.688 | 57.802 | −106.859 | 1.00 | 16.15 | C |
| ATOM | 708 | O | LYS | | 95 | 29.151 | 58.515 | −107.745 | 1.00 | 16.68 | O |
| ATOM | 709 | N | ILE | | 96 | 28.452 | 58.268 | −105.639 | 1.00 | 13.90 | N |
| ATOM | 710 | CA | ILE | | 96 | 28.760 | 59.668 | −105.347 | 1.00 | 16.88 | C |
| ATOM | 711 | CB | ILE | | 96 | 28.163 | 60.067 | −103.970 | 1.00 | 13.67 | C |
| ATOM | 712 | CG2 | ILE | | 96 | 28.657 | 61.475 | −103.561 | 1.00 | 12.39 | C |
| ATOM | 713 | CG1 | ILE | | 96 | 26.623 | 60.039 | −104.051 | 1.00 | 14.67 | C |
| ATOM | 714 | CD1 | ILE | | 96 | 25.945 | 59.867 | −102.713 | 1.00 | 16.06 | C |
| ATOM | 715 | C | ILE | | 96 | 30.275 | 59.905 | −105.368 | 1.00 | 16.78 | C |
| ATOM | 716 | O | ILE | | 96 | 30.771 | 60.876 | −105.963 | 1.00 | 17.46 | O |
| ATOM | 717 | N | LYS | | 97 | 31.029 | 59.008 | −104.748 | 1.00 | 15.68 | N |
| ATOM | 718 | CA | LYS | | 97 | 32.481 | 59.174 | −104.734 | 1.00 | 15.59 | C |
| ATOM | 719 | CB | LYS | | 97 | 33.123 | 58.159 | −103.781 | 1.00 | 15.41 | C |
| ATOM | 720 | CG | LYS | | 97 | 32.835 | 58.403 | −102.295 | 1.00 | 13.42 | C |
| ATOM | 721 | CD | LYS | | 97 | 33.582 | 57.355 | −101.453 | 1.00 | 10.14 | C |
| ATOM | 722 | CE | LYS | | 97 | 33.390 | 57.597 | −99.961 | 1.00 | 19.78 | C |
| ATOM | 723 | NZ | LYS | | 97 | 34.231 | 56.636 | −99.200 | 1.00 | 18.47 | N |
| ATOM | 724 | C | LYS | | 97 | 33.102 | 59.000 | −106.121 | 1.00 | 16.11 | C |
| ATOM | 725 | O | LYS | | 97 | 34.113 | 59.602 | −106.420 | 1.00 | 17.49 | O |
| ATOM | 726 | N | SER | | 98 | 32.503 | 58.170 | −106.972 | 1.00 | 14.18 | N |
| ATOM | 727 | CA | SER | | 98 | 33.135 | 58.008 | −108.282 | 1.00 | 19.20 | C |
| ATOM | 728 | CB | SER | | 98 | 32.569 | 56.789 | −108.994 | 1.00 | 15.50 | C |
| ATOM | 729 | OG | SER | | 98 | 31.215 | 57.025 | −109.328 | 1.00 | 16.49 | O |
| ATOM | 730 | C | SER | | 98 | 32.928 | 59.222 | −109.186 | 1.00 | 20.00 | C |
| ATOM | 731 | O | SER | | 98 | 33.726 | 59.474 | −110.087 | 1.00 | 18.32 | O |
| ATOM | 732 | N | GLY | | 99 | 31.844 | 59.961 | −108.963 | 1.00 | 17.90 | N |
| ATOM | 733 | CA | GLY | | 99 | 31.528 | 61.122 | −109.784 | 1.00 | 15.44 | C |
| ATOM | 734 | C | GLY | | 99 | 30.380 | 60.805 | −110.739 | 1.00 | 17.84 | C |
| ATOM | 735 | O | GLY | | 99 | 29.862 | 61.702 | −111.411 | 1.00 | 14.61 | O |
| ATOM | 736 | N | GLU | | 100 | 29.960 | 59.536 | −110.808 | 1.00 | 14.51 | N |
| ATOM | 737 | CA | GLU | | 100 | 28.875 | 59.241 | −111.746 | 1.00 | 19.40 | C |
| ATOM | 738 | CB | GLU | | 100 | 28.792 | 57.740 | −112.065 | 1.00 | 26.85 | C |
| ATOM | 739 | CG | GLU | | 100 | 28.880 | 56.834 | −110.886 | 1.00 | 34.32 | C |
| ATOM | 740 | CD | GLU | | 100 | 29.124 | 55.377 | −111.278 | 1.00 | 36.01 | C |
| ATOM | 741 | OE1 | GLU | | 100 | 29.835 | 54.691 | −110.516 | 1.00 | 28.68 | O |
| ATOM | 742 | OE2 | GLU | | 100 | 28.604 | 54.921 | −112.326 | 1.00 | 38.68 | O |
| ATOM | 743 | C | GLU | | 100 | 27.522 | 59.753 | −111.300 | 1.00 | 18.89 | C |
| ATOM | 744 | O | GLU | | 100 | 26.628 | 59.944 | −112.125 | 1.00 | 19.16 | O |
| ATOM | 745 | N | GLU | | 101 | 27.351 | 59.979 | −109.999 | 1.00 | 19.45 | N |
| ATOM | 746 | CA | GLU | | 101 | 26.086 | 60.520 | −109.492 | 1.00 | 20.99 | C |
| ATOM | 747 | CB | GLU | | 101 | 25.223 | 59.432 | −108.831 | 1.00 | 25.89 | C |
| ATOM | 748 | CG | GLU | | 101 | 24.876 | 58.221 | −109.716 | 1.00 | 17.91 | C |
| ATOM | 749 | CD | GLU | | 101 | 23.930 | 58.543 | −110.861 | 1.00 | 28.00 | C |
| ATOM | 750 | OE1 | GLU | | 101 | 23.285 | 59.609 | −110.836 | 1.00 | 23.75 | O |
| ATOM | 751 | OE2 | GLU | | 101 | 23.823 | 57.712 | −111.790 | 1.00 | 33.55 | O |
| ATOM | 752 | C | GLU | | 101 | 26.391 | 61.613 | −108.476 | 1.00 | 18.28 | C |
| ATOM | 753 | O | GLU | | 101 | 27.476 | 61.662 | −107.915 | 1.00 | 15.74 | O |
| ATOM | 754 | N | ASP | | 102 | 25.436 | 62.504 | −108.248 | 1.00 | 15.93 | N |
| ATOM | 755 | CA | ASP | | 102 | 25.454 | 63.658 | −107.359 | 1.00 | 18.64 | C |
| ATOM | 756 | CB | ASP | | 102 | 24.894 | 64.869 | −108.111 | 1.00 | 28.43 | C |
| ATOM | 757 | CG | ASP | | 102 | 24.757 | 66.086 | −107.235 | 1.00 | 33.93 | C |
| ATOM | 758 | OD1 | ASP | | 102 | 23.636 | 66.365 | −106.764 | 1.00 | 37.39 | O |
| ATOM | 759 | OD2 | ASP | | 102 | 25.774 | 66.759 | −107.004 | 1.00 | 42.34 | O |
| ATOM | 760 | C | ASP | | 102 | 24.613 | 63.341 | −106.127 | 1.00 | 22.26 | C |
| ATOM | 761 | O | ASP | | 102 | 23.540 | 62.766 | −106.234 | 1.00 | 22.92 | O |
| ATOM | 762 | N | PHE | | 103 | 25.099 | 63.704 | −104.949 | 1.00 | 20.77 | N |
| ATOM | 763 | CA | PHE | | 103 | 24.356 | 63.407 | −103.735 | 1.00 | 18.22 | C |
| ATOM | 764 | CB | PHE | | 103 | 25.100 | 63.972 | −102.528 | 1.00 | 14.61 | C |
| ATOM | 765 | CG | PHE | | 103 | 24.349 | 63.836 | −101.236 | 1.00 | 22.17 | C |
| ATOM | 766 | CD1 | PHE | | 103 | 24.536 | 62.725 | −100.424 | 1.00 | 20.09 | C |
| ATOM | 767 | CD2 | PHE | | 103 | 23.404 | 64.791 | −100.863 | 1.00 | 18.56 | C |
| ATOM | 768 | CE1 | PHE | | 103 | 23.791 | 62.560 | −99.270 | 1.00 | 24.84 | C |
| ATOM | 769 | CE2 | PHE | | 103 | 22.660 | 64.631 | −99.713 | 1.00 | 20.83 | C |
| ATOM | 770 | CZ | PHE | | 103 | 22.850 | 63.513 | −98.916 | 1.00 | 19.79 | C |
| ATOM | 771 | C | PHE | | 103 | 22.934 | 63.953 | −103.732 | 1.00 | 22.29 | C |
| ATOM | 772 | O | PHE | | 103 | 21.971 | 63.216 | −103.513 | 1.00 | 17.67 | O |
| ATOM | 773 | N | GLU | | 104 | 22.786 | 65.256 | −103.949 | 1.00 | 25.95 | N |
| ATOM | 774 | CA | GLU | | 104 | 21.437 | 65.818 | −103.920 | 1.00 | 22.77 | C |
| ATOM | 775 | CB | GLU | | 104 | 21.509 | 67.336 | −104.083 | 1.00 | 29.64 | C |
| ATOM | 776 | CG | GLU | | 104 | 22.390 | 67.982 | −103.027 | 1.00 | 40.73 | C |
| ATOM | 777 | CD | GLU | | 104 | 22.243 | 69.484 | −102.979 | 1.00 | 46.69 | C |
| ATOM | 778 | OE1 | GLU | | 104 | 22.255 | 70.116 | −104.059 | 1.00 | 47.14 | O |
| ATOM | 779 | OE2 | GLU | | 104 | 22.123 | 70.028 | −101.861 | 1.00 | 45.80 | O |
| ATOM | 780 | C | GLU | | 104 | 20.520 | 65.206 | −104.962 | 1.00 | 22.70 | C |
| ATOM | 781 | O | GLU | | 104 | 19.322 | 65.025 | −104.724 | 1.00 | 21.30 | O |
| ATOM | 782 | N | SER | | 105 | 21.067 | 64.880 | −106.128 | 1.00 | 21.75 | N |
| ATOM | 783 | CA | SER | | 105 | 20.314 | 64.246 | −107.187 | 1.00 | 22.04 | C |
| ATOM | 784 | CB | SER | | 105 | 21.186 | 64.073 | −108.426 | 1.00 | 25.70 | C |
| ATOM | 785 | OG | SER | | 105 | 20.540 | 63.228 | −109.353 | 1.00 | 31.44 | O |
| ATOM | 786 | C | SER | | 105 | 19.827 | 62.879 | −106.721 | 1.00 | 23.58 | C |
| ATOM | 787 | O | SER | | 105 | 18.655 | 62.528 | −106.892 | 1.00 | 20.99 | O |
| ATOM | 788 | N | LEU | | 106 | 20.715 | 62.093 | −106.119 | 1.00 | 17.50 | N |
| ATOM | 789 | CA | LEU | | 106 | 20.255 | 60.780 | −105.656 | 1.00 | 18.99 | C |
| ATOM | 790 | CB | LEU | | 106 | 21.428 | 59.926 | −105.172 | 1.00 | 16.26 | C |
| ATOM | 791 | CG | LEU | | 106 | 22.381 | 59.394 | −106.240 | 1.00 | 22.29 | C |
| ATOM | 792 | CD1 | LEU | | 106 | 23.449 | 58.541 | −105.604 | 1.00 | 20.00 | C |
| ATOM | 793 | CD2 | LEU | | 106 | 21.598 | 58.575 | −107.239 | 1.00 | 23.22 | C |
| ATOM | 794 | C | LEU | | 106 | 19.256 | 60.895 | −104.518 | 1.00 | 19.65 | C |
| ATOM | 795 | O | LEU | | 106 | 18.311 | 60.095 | −104.410 | 1.00 | 19.82 | O |
| ATOM | 796 | N | ALA | | 107 | 19.468 | 61.868 | −103.640 | 1.00 | 17.00 | N |
| ATOM | 797 | CA | ALA | | 107 | 18.560 | 62.024 | −102.519 | 1.00 | 15.73 | C |
| ATOM | 798 | CB | ALA | | 107 | 19.065 | 63.133 | −101.595 | 1.00 | 15.49 | C |
| ATOM | 799 | C | ALA | | 107 | 17.164 | 62.358 | −103.030 | 1.00 | 16.11 | C |
| ATOM | 800 | O | ALA | | 107 | 16.174 | 61.840 | −102.533 | 1.00 | 18.35 | O |
| ATOM | 801 | N | SER | | 108 | 17.073 | 63.220 | −104.036 | 1.00 | 18.72 | N |
| ATOM | 802 | CA | SER | | 108 | 15.723 | 63.534 | −104.492 | 1.00 | 20.30 | C |
| ATOM | 803 | CB | SER | | 108 | 15.744 | 64.750 | −105.425 | 1.00 | 24.33 | C |
| ATOM | 804 | OG | SER | | 108 | 16.442 | 64.443 | −106.611 | 1.00 | 34.31 | O |
| ATOM | 805 | C | SER | | 108 | 15.039 | 62.348 | −105.173 | 1.00 | 22.28 | C |
| ATOM | 806 | O | SER | | 108 | 13.822 | 62.206 | −105.107 | 1.00 | 21.67 | O |
| ATOM | 807 | N | GLN | | 109 | 15.809 | 61.464 | −105.797 | 1.00 | 23.78 | N |
| ATOM | 808 | CA | GLN | | 109 | 15.124 | 60.335 | −106.431 | 1.00 | 22.14 | C |
| ATOM | 809 | CB | GLN | | 109 | 15.923 | 59.798 | −107.621 | 1.00 | 27.25 | C |
| ATOM | 810 | CG | GLN | | 109 | 16.438 | 60.822 | −108.602 | 1.00 | 26.07 | C |
| ATOM | 811 | CD | GLN | | 109 | 17.447 | 60.211 | −109.545 | 1.00 | 30.67 | C |
| ATOM | 812 | OE1 | GLN | | 109 | 17.155 | 59.229 | −110.225 | 1.00 | 28.89 | O |
| ATOM | 813 | NE2 | GLN | | 109 | 18.654 | 60.781 | −109.585 | 1.00 | 30.61 | N |
| ATOM | 814 | C | GLN | | 109 | 14.899 | 59.135 | −105.521 | 1.00 | 26.49 | C |
| ATOM | 815 | O | GLN | | 109 | 13.890 | 58.426 | −105.650 | 1.00 | 22.02 | O |
| ATOM | 816 | N | PHE | | 110 | 15.824 | 58.890 | −104.598 | 1.00 | 19.97 | N |
| ATOM | 817 | CA | PHE | | 110 | 15.699 | 57.684 | −103.802 | 1.00 | 18.01 | C |
| ATOM | 818 | CB | PHE | | 110 | 16.890 | 56.795 | −104.120 | 1.00 | 18.95 | C |
| ATOM | 819 | CG | PHE | | 110 | 17.058 | 56.510 | −105.583 | 1.00 | 26.77 | C |
| ATOM | 820 | CD1 | PHE | | 110 | 18.152 | 57.012 | −106.281 | 1.00 | 21.83 | C |
| ATOM | 821 | CD2 | PHE | | 110 | 16.142 | 55.701 | −106.258 | 1.00 | 29.37 | C |
| ATOM | 822 | CE1 | PHE | | 110 | 18.338 | 56.711 | −107.620 | 1.00 | 22.29 | C |
| ATOM | 823 | CE2 | PHE | | 110 | 16.312 | 55.392 | −107.599 | 1.00 | 27.28 | C |
| ATOM | 824 | CZ | PHE | | 110 | 17.412 | 55.893 | −108.289 | 1.00 | 30.93 | C |
| ATOM | 825 | C | PHE | | 110 | 15.564 | 57.789 | −102.288 | 1.00 | 20.19 | C |
| ATOM | 826 | O | PHE | | 110 | 15.394 | 56.774 | −101.620 | 1.00 | 17.94 | O |
| ATOM | 827 | N | SER | | 111 | 15.656 | 58.992 | −101.730 | 1.00 | 20.34 | N |
| ATOM | 828 | CA | SER | | 111 | 15.533 | 58.983 | −100.279 | 1.00 | 17.14 | C |
| ATOM | 829 | CB | SER | | 111 | 15.910 | 60.341 | −99.699 | 1.00 | 12.51 | C |
| ATOM | 830 | OG | SER | | 111 | 15.856 | 60.348 | −98.277 | 1.00 | 16.39 | O |
| ATOM | 831 | C | SER | | 111 | 14.119 | 58.640 | −99.831 | 1.00 | 22.63 | C |
| ATOM | 832 | O | SER | | 111 | 13.142 | 59.196 | −100.344 | 1.00 | 18.74 | O |
| ATOM | 833 | N | ASP | | 112 | 14.000 | 57.717 | −98.878 | 1.00 | 21.12 | N |
| ATOM | 834 | CA | ASP | | 112 | 12.729 | 57.308 | −98.307 | 1.00 | 23.71 | C |
| ATOM | 835 | CB | ASP | | 112 | 12.845 | 55.957 | −97.587 | 1.00 | 18.39 | C |
| ATOM | 836 | CG | ASP | | 112 | 12.703 | 54.802 | −98.540 | 1.00 | 21.83 | C |
| ATOM | 837 | OD1 | ASP | | 112 | 11.750 | 54.831 | −99.348 | 1.00 | 25.39 | O |
| ATOM | 838 | OD2 | ASP | | 112 | 13.531 | 53.873 | −98.498 | 1.00 | 20.00 | O |
| ATOM | 839 | C | ASP | | 112 | 12.209 | 58.358 | −97.351 | 1.00 | 22.32 | C |
| ATOM | 840 | O | ASP | | 112 | 11.132 | 58.206 | −96.799 | 1.00 | 23.32 | O |
| ATOM | 841 | N | CYS | | 113 | 12.975 | 59.430 | −97.136 | 1.00 | 21.02 | N |
| ATOM | 842 | CA | CYS | | 113 | 12.514 | 60.506 | −96.255 | 1.00 | 20.91 | C |
| ATOM | 843 | CB | CYS | | 113 | 13.689 | 61.159 | −95.518 | 1.00 | 18.87 | C |
| ATOM | 844 | SG | CYS | | 113 | 13.263 | 62.623 | −94.524 | 1.00 | 21.79 | S |
| ATOM | 845 | C | CYS | | 113 | 11.814 | 61.552 | −97.099 | 1.00 | 22.77 | C |
| ATOM | 846 | O | CYS | | 113 | 12.226 | 61.817 | −98.220 | 1.00 | 18.80 | O |
| ATOM | 847 | N | SER | | 114 | 10.752 | 62.157 | −96.574 | 1.00 | 25.68 | N |
| ATOM | 848 | CA | SER | | 114 | 9.977 | 63.179 | −97.278 | 1.00 | 26.05 | C |
| ATOM | 849 | CB | SER | | 114 | 8.809 | 63.660 | −96.411 | 1.00 | 26.35 | C |
| ATOM | 850 | OG | SER | | 114 | 9.289 | 64.285 | −95.232 | 1.00 | 40.21 | O |
| ATOM | 851 | C | SER | | 114 | 10.831 | 64.377 | −97.672 | 1.00 | 27.38 | C |
| ATOM | 852 | O | SER | | 114 | 10.464 | 65.139 | −98.566 | 1.00 | 22.86 | O |
| ATOM | 853 | N | SER | | 115 | 11.977 | 64.551 | −97.016 | 1.00 | 22.03 | N |
| ATOM | 854 | CA | SER | | 115 | 12.881 | 65.657 | −97.320 | 1.00 | 22.23 | C |
| ATOM | 855 | CB | SER | | 115 | 14.013 | 65.740 | −96.278 | 1.00 | 20.87 | C |
| ATOM | 856 | OG | SER | | 115 | 14.855 | 64.606 | −96.343 | 1.00 | 20.23 | O |
| ATOM | 857 | C | SER | | 115 | 13.466 | 65.487 | −98.712 | 1.00 | 18.48 | C |
| ATOM | 858 | O | SER | | 115 | 14.180 | 66.354 | −99.206 | 1.00 | 20.02 | O |
| ATOM | 859 | N | ALA | | 116 | 13.186 | 64.355 | −99.351 | 1.00 | 19.80 | N |
| ATOM | 860 | CA | ALA | | 116 | 13.660 | 64.125 | −100.710 | 1.00 | 19.73 | C |
| ATOM | 861 | CB | ALA | | 116 | 13.159 | 62.780 | −101.212 | 1.00 | 19.33 | C |
| ATOM | 862 | C | ALA | | 116 | 13.137 | 65.246 | −101.610 | 1.00 | 23.70 | C |
| ATOM | 863 | O | ALA | | 116 | 13.746 | 65.579 | −102.621 | 1.00 | 21.46 | O |
| ATOM | 864 | N | LYS | | 117 | 12.004 | 65.846 | −101.240 | 1.00 | 22.22 | N |
| ATOM | 865 | CA | LYS | | 117 | 11.382 | 66.925 | −102.009 | 1.00 | 22.47 | C |
| ATOM | 866 | CB | LYS | | 117 | 10.007 | 67.272 | −101.422 | 1.00 | 32.54 | C |
| ATOM | 867 | CG | LYS | | 117 | 10.078 | 68.192 | −100.203 | 1.00 | 38.29 | C |
| ATOM | 868 | CD | LYS | | 117 | 9.043 | 67.850 | −99.121 | 1.00 | 49.03 | C |
| ATOM | 869 | CE | LYS | | 117 | 7.613 | 68.137 | −99.548 | 1.00 | 52.85 | C |
| ATOM | 870 | NZ | LYS | | 117 | 6.670 | 67.909 | −98.411 | 1.00 | 52.29 | N |
| ATOM | 871 | C | LYS | | 117 | 12.265 | 68.171 | −102.001 | 1.00 | 21.60 | C |
| ATOM | 872 | O | LYS | | 117 | 12.164 | 69.014 | −102.868 | 1.00 | 24.19 | O |
| ATOM | 873 | N | ALA | | 118 | 13.133 | 68.294 | −101.004 | 1.00 | 23.62 | N |
| ATOM | 874 | CA | ALA | | 118 | 14.041 | 69.423 | −100.889 | 1.00 | 22.70 | C |
| ATOM | 875 | CB | ALA | | 118 | 13.949 | 70.016 | −99.491 | 1.00 | 29.32 | C |
| ATOM | 876 | C | ALA | | 118 | 15.470 | 68.965 | −101.170 | 1.00 | 23.38 | C |
| ATOM | 877 | O | ALA | | 118 | 16.419 | 69.487 | −100.601 | 1.00 | 20.06 | O |
| ATOM | 878 | N | ARG | | 119 | 15.630 | 67.985 | −102.054 | 1.00 | 20.10 | N |
| ATOM | 879 | CA | ARG | | 119 | 16.900 | 67.386 | −102.450 | 1.00 | 26.59 | C |
| ATOM | 880 | CB | ARG | | 119 | 17.766 | 68.401 | −103.207 | 1.00 | 28.51 | C |
| ATOM | 881 | CG | ARG | | 119 | 17.010 | 69.180 | −104.288 | 1.00 | 34.77 | C |
| ATOM | 882 | CD | ARG | | 119 | 17.831 | 69.306 | −105.568 | 1.00 | 42.34 | C |
| ATOM | 883 | NE | ARG | | 119 | 17.391 | 68.322 | −106.554 | 1.00 | 45.62 | N |
| ATOM | 884 | CZ | ARG | | 119 | 18.160 | 67.787 | −107.498 | 1.00 | 43.53 | C |
| ATOM | 885 | NH1 | ARG | | 119 | 19.438 | 68.125 | −107.609 | 1.00 | 38.05 | N |
| ATOM | 886 | NH2 | ARG | | 119 | 17.642 | 66.903 | −108.335 | 1.00 | 51.45 | N |
| ATOM | 887 | C | ARG | | 119 | 17.649 | 66.866 | −101.221 | 1.00 | 24.45 | C |
| ATOM | 888 | O | ARG | | 119 | 18.878 | 66.861 | −101.180 | 1.00 | 26.67 | O |
| ATOM | 889 | N | GLY | | 120 | 16.904 | 66.430 | −100.210 | 1.00 | 22.99 | N |
| ATOM | 890 | CA | GLY | | 120 | 17.502 | 65.899 | −99.004 | 1.00 | 19.62 | C |
| ATOM | 891 | C | GLY | | 120 | 17.820 | 66.886 | −97.895 | 1.00 | 21.15 | C |
| ATOM | 892 | O | GLY | | 120 | 18.232 | 66.467 | −96.826 | 1.00 | 17.62 | O |
| ATOM | 893 | N | ASP | | 121 | 17.647 | 68.189 | −98.126 | 1.00 | 19.16 | N |
| ATOM | 894 | CA | ASP | | 121 | 17.941 | 69.213 | −97.130 | 1.00 | 18.19 | C |
| ATOM | 895 | CB | ASP | | 121 | 17.881 | 70.614 | −97.771 | 1.00 | 19.43 | C |
| ATOM | 896 | CG | ASP | | 121 | 18.091 | 71.747 | −96.758 | 1.00 | 25.60 | C |
| ATOM | 897 | OD1 | ASP | | 121 | 18.919 | 71.600 | −95.843 | 1.00 | 22.71 | O |
| ATOM | 898 | OD2 | ASP | | 121 | 17.436 | 72.804 | −96.882 | 1.00 | 24.12 | O |
| ATOM | 899 | C | ASP | | 121 | 16.990 | 69.137 | −95.938 | 1.00 | 17.82 | C |
| ATOM | 900 | O | ASP | | 121 | 15.790 | 69.019 | −96.110 | 1.00 | 17.89 | O |
| ATOM | 901 | N | LEU | | 122 | 17.531 | 69.176 | −94.725 | 1.00 | 18.47 | N |
| ATOM | 902 | CA | LEU | | 122 | 16.819 | 69.134 | −93.461 | 1.00 | 20.46 | C |
| ATOM | 903 | CB | LEU | | 122 | 17.433 | 68.089 | −92.533 | 1.00 | 22.72 | C |
| ATOM | 904 | CG | LEU | | 122 | 17.408 | 66.632 | −92.979 | 1.00 | 19.68 | C |
| ATOM | 905 | CD1 | LEU | | 122 | 18.071 | 65.778 | −91.911 | 1.00 | 19.58 | C |
| ATOM | 906 | CD2 | LEU | | 122 | 15.984 | 66.194 | −93.214 | 1.00 | 21.59 | C |
| ATOM | 907 | C | LEU | | 122 | 16.873 | 70.480 | −92.771 | 1.00 | 18.78 | C |
| ATOM | 908 | O | LEU | | 122 | 16.221 | 70.697 | −91.748 | 1.00 | 23.15 | O |
| ATOM | 909 | N | GLY | | 123 | 17.664 | 71.394 | −93.303 | 1.00 | 19.94 | N |
| ATOM | 910 | CA | GLY | | 123 | 17.776 | 72.694 | −92.672 | 1.00 | 24.12 | C |
| ATOM | 911 | C | GLY | | 123 | 18.753 | 72.601 | −91.515 | 1.00 | 26.59 | C |
| ATOM | 912 | O | GLY | | 123 | 19.330 | 71.547 | −91.265 | 1.00 | 23.63 | O |
| ATOM | 913 | N | ALA | | 124 | 18.940 | 73.694 | −90.785 | 1.00 | 23.42 | N |
| ATOM | 914 | CA | ALA | | 124 | 19.877 | 73.689 | −89.676 | 1.00 | 24.30 | C |
| ATOM | 915 | CB | ALA | | 124 | 20.376 | 75.091 | −89.419 | 1.00 | 28.07 | C |
| ATOM | 916 | C | ALA | | 124 | 19.253 | 73.129 | −88.416 | 1.00 | 25.18 | C |
| ATOM | 917 | O | ALA | | 124 | 18.041 | 73.157 | −88.247 | 1.00 | 26.44 | O |
| ATOM | 918 | N | PHE | | 125 | 20.077 | 72.605 | −87.524 | 1.00 | 19.55 | N |
| ATOM | 919 | CA | PHE | | 125 | 19.587 | 72.061 | −86.273 | 1.00 | 22.64 | C |
| ATOM | 920 | CB | PHE | | 125 | 18.982 | 70.676 | −86.497 | 1.00 | 23.99 | C |
| ATOM | 921 | CG | PHE | | 125 | 19.931 | 69.680 | −87.099 | 1.00 | 17.14 | C |
| ATOM | 922 | CD1 | PHE | | 125 | 20.745 | 68.891 | −86.286 | 1.00 | 22.57 | C |
| ATOM | 923 | CD2 | PHE | | 125 | 20.009 | 69.530 | −88.484 | 1.00 | 19.16 | C |
| ATOM | 924 | CE1 | PHE | | 125 | 21.627 | 67.963 | −86.839 | 1.00 | 20.21 | C |
| ATOM | 925 | CE2 | PHE | | 125 | 20.882 | 68.615 | −89.044 | 1.00 | 18.47 | C |
| ATOM | 926 | CZ | PHE | | 125 | 21.698 | 67.826 | −88.220 | 1.00 | 18.16 | C |
| ATOM | 927 | C | PHE | | 125 | 20.680 | 72.007 | −85.228 | 1.00 | 24.12 | C |
| ATOM | 928 | O | PHE | | 125 | 21.873 | 72.127 | −85.542 | 1.00 | 23.87 | O |
| ATOM | 929 | N | SER | | 126 | 20.288 | 71.850 | −83.967 | 1.00 | 23.66 | N |
| ATOM | 930 | CA | SER | | 126 | 21.115 | 71.772 | −82.775 | 1.00 | 22.99 | C |
| ATOM | 931 | CB | SER | | 126 | 20.613 | 72.762 | −81.709 | 1.00 | 26.31 | C |
| ATOM | 932 | OG | SER | | 126 | 20.539 | 74.074 | −82.224 | 1.00 | 36.73 | O |
| ATOM | 933 | C | SER | | 126 | 21.063 | 70.373 | −82.188 | 1.00 | 19.39 | C |
| ATOM | 934 | O | SER | | 126 | 20.234 | 69.547 | −82.587 | 1.00 | 22.74 | O |
| ATOM | 935 | N | ARG | | 127 | 21.947 | 70.091 | −81.239 | 1.00 | 20.63 | N |
| ATOM | 936 | CA | ARG | | 127 | 21.847 | 68.767 | −80.660 | 1.00 | 23.05 | C |
| ATOM | 937 | CB | ARG | | 127 | 22.991 | 68.513 | −79.681 | 1.00 | 26.37 | C |
| ATOM | 938 | CG | ARG | | 127 | 24.352 | 68.306 | −80.333 | 1.00 | 20.71 | C |
| ATOM | 939 | CD | ARG | | 127 | 25.279 | 67.631 | −79.334 | 1.00 | 19.42 | C |
| ATOM | 940 | NE | ARG | | 127 | 26.546 | 67.197 | −79.912 | 1.00 | 24.97 | N |
| ATOM | 941 | CZ | ARG | | 127 | 26.768 | 66.005 | −80.462 | 1.00 | 22.99 | C |
| ATOM | 942 | NH1 | ARG | | 127 | 25.805 | 65.093 | −80.528 | 1.00 | 27.22 | N |
| ATOM | 943 | NH2 | ARG | | 127 | 27.977 | 65.712 | −80.911 | 1.00 | 17.15 | N |
| ATOM | 944 | C | ARG | | 127 | 20.529 | 68.675 | −79.916 | 1.00 | 28.93 | C |
| ATOM | 945 | O | ARG | | 127 | 19.965 | 69.688 | −79.502 | 1.00 | 27.94 | O |
| ATOM | 946 | N | GLY | | 128 | 20.023 | 67.464 | −79.741 | 1.00 | 24.21 | N |
| ATOM | 947 | CA | GLY | | 128 | 18.772 | 67.289 | −79.034 | 1.00 | 30.86 | C |
| ATOM | 948 | C | GLY | | 128 | 17.572 | 67.258 | −79.953 | 1.00 | 30.55 | C |
| ATOM | 949 | O | GLY | | 128 | 16.458 | 67.005 | −79.502 | 1.00 | 29.19 | O |
| ATOM | 950 | N | GLN | | 129 | 17.782 | 67.475 | −81.253 | 1.00 | 24.47 | N |
| ATOM | 951 | CA | GLN | | 129 | 16.607 | 67.490 | −82.121 | 1.00 | 26.10 | C |
| ATOM | 952 | CB | GLN | | 129 | 16.575 | 68.812 | −82.907 | 1.00 | 27.10 | C |
| ATOM | 953 | CG | GLN | | 129 | 16.795 | 70.017 | −81.991 | 1.00 | 27.40 | C |
| ATOM | 954 | CD | GLN | | 129 | 16.599 | 71.358 | −82.676 | 1.00 | 32.90 | C |
| ATOM | 955 | OE1 | GLN | | 129 | 17.268 | 71.668 | −83.659 | 1.00 | 28.77 | O |
| ATOM | 956 | NE2 | GLN | | 129 | 15.683 | 72.172 | −82.143 | 1.00 | 29.11 | N |
| ATOM | 957 | C | GLN | | 129 | 16.449 | 66.293 | −83.057 | 1.00 | 29.05 | C |
| ATOM | 958 | O | GLN | | 129 | 15.327 | 65.923 | −83.397 | 1.00 | 31.80 | O |
| ATOM | 959 | N | MET | | 130 | 17.554 | 65.675 | −83.478 | 1.00 | 23.38 | N |
| ATOM | 960 | CA | MET | | 130 | 17.513 | 64.496 | −84.352 | 1.00 | 22.70 | C |
| ATOM | 961 | CB | MET | | 130 | 18.564 | 64.607 | −85.466 | 1.00 | 26.23 | C |
| ATOM | 962 | CG | MET | | 130 | 18.405 | 65.803 | −86.369 | 1.00 | 25.24 | C |
| ATOM | 963 | SD | MET | | 130 | 16.923 | 65.720 | −87.375 | 1.00 | 36.87 | S |
| ATOM | 964 | CE | MET | | 130 | 16.932 | 67.388 | −88.084 | 1.00 | 32.28 | C |
| ATOM | 965 | C | MET | | 130 | 17.820 | 63.238 | −83.545 | 1.00 | 22.14 | C |
| ATOM | 966 | O | MET | | 130 | 18.198 | 63.316 | −82.377 | 1.00 | 28.11 | O |
| ATOM | 967 | N | GLN | | 131 | 17.667 | 62.069 | −84.160 | 1.00 | 19.89 | N |
| ATOM | 968 | CA | GLN | | 131 | 18.001 | 60.868 | −83.418 | 1.00 | 22.02 | C |
| ATOM | 969 | CB | GLN | | 131 | 17.583 | 59.652 | −84.226 | 1.00 | 29.99 | C |
| ATOM | 970 | CG | GLN | | 131 | 16.094 | 59.653 | −84.465 | 1.00 | 33.31 | C |
| ATOM | 971 | CD | GLN | | 131 | 15.635 | 58.451 | −85.228 | 1.00 | 38.48 | C |
| ATOM | 972 | OE1 | GLN | | 131 | 15.950 | 57.321 | −84.864 | 1.00 | 43.53 | O |
| ATOM | 973 | NE2 | GLN | | 131 | 14.875 | 58.677 | −86.288 | 1.00 | 40.55 | N |
| ATOM | 974 | C | GLN | | 131 | 19.487 | 60.852 | −83.125 | 1.00 | 22.78 | C |
| ATOM | 975 | O | GLN | | 131 | 20.294 | 61.265 | −83.949 | 1.00 | 21.23 | O |
| ATOM | 976 | N | LYS | | 132 | 19.871 | 60.355 | −81.955 | 1.00 | 22.84 | N |
| ATOM | 977 | CA | LYS | | 132 | 21.271 | 60.374 | −81.536 | 1.00 | 22.66 | C |
| ATOM | 978 | CB | LYS | | 132 | 21.432 | 59.660 | −80.194 | 1.00 | 22.91 | C |
| ATOM | 979 | CG | LYS | | 132 | 22.728 | 60.026 | −79.463 | 1.00 | 34.09 | C |
| ATOM | 980 | CD | LYS | | 132 | 22.898 | 61.547 | −79.368 | 1.00 | 37.74 | C |
| ATOM | 981 | CE | LYS | | 132 | 23.915 | 61.952 | −78.302 | 1.00 | 37.08 | C |
| ATOM | 982 | NZ | LYS | | 132 | 25.203 | 61.222 | −78.416 | 1.00 | 39.19 | N |
| ATOM | 983 | C | LYS | | 132 | 22.334 | 59.880 | −82.520 | 1.00 | 21.24 | C |
| ATOM | 984 | O | LYS | | 132 | 23.374 | 60.509 | −82.651 | 1.00 | 20.90 | O |
| ATOM | 985 | N | PRO | | 133 | 22.115 | 58.743 | −83.197 | 1.00 | 23.53 | N |
| ATOM | 986 | CD | PRO | | 133 | 21.141 | 57.650 | −82.991 | 1.00 | 24.69 | C |
| ATOM | 987 | CA | PRO | | 133 | 23.179 | 58.336 | −84.128 | 1.00 | 21.53 | C |
| ATOM | 988 | CB | PRO | | 133 | 22.706 | 56.973 | −84.623 | 1.00 | 22.42 | C |
| ATOM | 989 | CG | PRO | | 133 | 21.936 | 56.431 | −83.424 | 1.00 | 22.46 | C |
| ATOM | 990 | C | PRO | | 133 | 23.344 | 59.347 | −85.270 | 1.00 | 23.95 | C |
| ATOM | 991 | O | PRO | | 133 | 24.460 | 59.647 | −85.695 | 1.00 | 17.72 | O |
| ATOM | 992 | N | PHE | | 134 | 22.232 | 59.875 | −85.771 | 1.00 | 18.40 | N |
| ATOM | 993 | CA | PHE | | 134 | 22.186 | 60.853 | −86.846 | 1.00 | 18.01 | C |
| ATOM | 994 | CB | PHE | | 134 | 20.732 | 61.200 | −87.186 | 1.00 | 17.39 | C |
| ATOM | 995 | CG | PHE | | 134 | 20.580 | 62.050 | −88.419 | 1.00 | 17.29 | C |
| ATOM | 996 | CD1 | PHE | | 134 | 20.416 | 61.463 | −89.664 | 1.00 | 14.55 | C |
| ATOM | 997 | CD2 | PHE | | 134 | 20.666 | 63.436 | −88.342 | 1.00 | 18.45 | C |
| ATOM | 998 | CE1 | PHE | | 134 | 20.348 | 62.243 | −90.825 | 1.00 | 14.24 | C |
| ATOM | 999 | CE2 | PHE | | 134 | 20.599 | 64.225 | −89.501 | 1.00 | 18.03 | C |
| ATOM | 1000 | CZ | PHE | | 134 | 20.442 | 63.619 | −90.742 | 1.00 | 13.82 | C |
| ATOM | 1001 | C | PHE | | 134 | 22.891 | 62.115 | −86.365 | 1.00 | 19.17 | C |
| ATOM | 1002 | O | PHE | | 134 | 23.715 | 62.689 | −87.067 | 1.00 | 17.27 | O |
| ATOM | 1003 | N | GLU | | 135 | 22.560 | 62.560 | −85.159 | 1.00 | 14.92 | N |
| ATOM | 1004 | CA | GLU | | 135 | 23.119 | 63.749 | −84.556 | 1.00 | 15.39 | C |
| ATOM | 1005 | CB | GLU | | 135 | 22.417 | 64.024 | −83.229 | 1.00 | 17.15 | C |
| ATOM | 1006 | CG | GLU | | 135 | 23.020 | 65.142 | −82.434 | 1.00 | 21.64 | C |
| ATOM | 1007 | CD | GLU | | 135 | 22.376 | 65.255 | −81.067 | 1.00 | 27.85 | C |
| ATOM | 1008 | OE1 | GLU | | 135 | 21.155 | 65.466 | −81.018 | 1.00 | 21.72 | O |
| ATOM | 1009 | OE2 | GLU | | 135 | 23.089 | 65.124 | −80.056 | 1.00 | 21.29 | O |
| ATOM | 1010 | C | GLU | | 135 | 24.619 | 63.644 | −84.335 | 1.00 | 14.81 | C |
| ATOM | 1011 | O | GLU | | 135 | 25.367 | 64.561 | −84.688 | 1.00 | 17.85 | O |
| ATOM | 1012 | N | ASP | | 136 | 25.076 | 62.541 | −83.747 | 1.00 | 14.16 | N |
| ATOM | 1013 | CA | ASP | | 136 | 26.513 | 62.379 | −83.509 | 1.00 | 13.51 | C |
| ATOM | 1014 | CB | ASP | | 136 | 26.800 | 61.040 | −82.827 | 1.00 | 19.16 | C |
| ATOM | 1015 | CG | ASP | | 136 | 26.396 | 61.030 | −81.365 | 1.00 | 22.91 | C |
| ATOM | 1016 | OD1 | ASP | | 136 | 26.183 | 62.124 | −80.802 | 1.00 | 18.61 | O |
| ATOM | 1017 | OD2 | ASP | | 136 | 26.313 | 59.923 | −80.788 | 1.00 | 22.85 | O |
| ATOM | 1018 | C | ASP | | 136 | 27.310 | 62.436 | −84.807 | 1.00 | 17.13 | C |
| ATOM | 1019 | O | ASP | | 136 | 28.389 | 63.023 | −84.862 | 1.00 | 13.87 | O |
| ATOM | 1020 | N | ALA | | 137 | 26.789 | 61.817 | −85.864 | 1.00 | 19.83 | N |
| ATOM | 1021 | CA | ALA | | 137 | 27.524 | 61.835 | −87.121 | 1.00 | 14.61 | C |
| ATOM | 1022 | CB | ALA | | 137 | 26.891 | 60.869 | −88.100 | 1.00 | 12.76 | C |
| ATOM | 1023 | C | ALA | | 137 | 27.548 | 63.236 | −87.722 | 1.00 | 19.38 | C |
| ATOM | 1024 | O | ALA | | 137 | 28.585 | 63.695 | −88.213 | 1.00 | 13.43 | O |
| ATOM | 1025 | N | SER | | 138 | 26.405 | 63.919 | −87.708 | 1.00 | 14.52 | N |
| ATOM | 1026 | CA | SER | | 138 | 26.234 | 65.266 | −88.240 | 1.00 | 15.13 | C |
| ATOM | 1027 | CB | SER | | 138 | 24.811 | 65.767 | −87.982 | 1.00 | 16.58 | C |
| ATOM | 1028 | OG | SER | | 138 | 23.857 | 65.028 | −88.713 | 1.00 | 15.35 | O |
| ATOM | 1029 | C | SER | | 138 | 27.203 | 66.242 | −87.599 | 1.00 | 14.39 | C |
| ATOM | 1030 | O | SER | | 138 | 27.815 | 67.075 | −88.279 | 1.00 | 17.79 | O |
| ATOM | 1031 | N | PHE | | 139 | 27.366 | 66.144 | −86.286 | 1.00 | 13.37 | N |
| ATOM | 1032 | CA | PHE | | 139 | 28.256 | 67.087 | −85.625 | 1.00 | 19.32 | C |
| ATOM | 1033 | CB | PHE | | 139 | 27.831 | 67.315 | −84.162 | 1.00 | 16.77 | C |
| ATOM | 1034 | CG | PHE | | 139 | 26.640 | 68.228 | −84.023 | 1.00 | 17.26 | C |
| ATOM | 1035 | CD1 | PHE | | 139 | 25.361 | 67.755 | −84.226 | 1.00 | 13.59 | C |
| ATOM | 1036 | CD2 | PHE | | 139 | 26.816 | 69.591 | −83.787 | 1.00 | 19.69 | C |
| ATOM | 1037 | CE1 | PHE | | 139 | 24.264 | 68.612 | −84.204 | 1.00 | 16.74 | C |
| ATOM | 1038 | CE2 | PHE | | 139 | 25.725 | 70.461 | −83.765 | 1.00 | 16.39 | C |
| ATOM | 1039 | CZ | PHE | | 139 | 24.439 | 69.964 | −83.976 | 1.00 | 16.53 | C |
| ATOM | 1040 | C | PHE | | 139 | 29.720 | 66.733 | −85.706 | 1.00 | 14.37 | C |
| ATOM | 1041 | O | PHE | | 139 | 30.565 | 67.572 | −85.420 | 1.00 | 18.41 | O |
| ATOM | 1042 | N | ALA | | 140 | 30.039 | 65.503 | −86.108 | 1.00 | 13.52 | N |
| ATOM | 1043 | CA | ALA | | 140 | 31.433 | 65.098 | −86.247 | 1.00 | 12.57 | C |
| ATOM | 1044 | CB | ALA | | 140 | 31.592 | 63.603 | −85.919 | 1.00 | 15.84 | C |
| ATOM | 1045 | C | ALA | | 140 | 31.902 | 65.368 | −87.679 | 1.00 | 17.87 | C |
| ATOM | 1046 | O | ALA | | 140 | 33.092 | 65.296 | −87.986 | 1.00 | 18.29 | O |
| ATOM | 1047 | N | LEU | | 141 | 30.964 | 65.651 | −88.576 | 1.00 | 14.95 | N |
| ATOM | 1048 | CA | LEU | | 141 | 31.320 | 65.962 | −89.959 | 1.00 | 16.19 | C |
| ATOM | 1049 | CB | LEU | | 141 | 30.085 | 65.840 | −90.869 | 1.00 | 15.82 | C |
| ATOM | 1050 | CG | LEU | | 141 | 29.553 | 64.452 | −91.230 | 1.00 | 15.53 | C |
| ATOM | 1051 | CD1 | LEU | | 141 | 28.195 | 64.582 | −91.949 | 1.00 | 14.87 | C |
| ATOM | 1052 | CD2 | LEU | | 141 | 30.582 | 63.734 | −92.133 | 1.00 | 11.09 | C |
| ATOM | 1053 | C | LEU | | 141 | 31.801 | 67.406 | −90.020 | 1.00 | 20.61 | C |
| ATOM | 1054 | O | LEU | | 141 | 31.300 | 68.264 | −89.279 | 1.00 | 14.18 | O |
| ATOM | 1055 | N | ARG | | 142 | 32.785 | 67.689 | −90.873 | 1.00 | 18.37 | N |
| ATOM | 1056 | CA | ARG | | 142 | 33.250 | 69.061 | −91.082 | 1.00 | 20.47 | C |
| ATOM | 1057 | CB | ARG | | 142 | 34.758 | 69.090 | −91.366 | 1.00 | 18.34 | C |
| ATOM | 1058 | CG | ARG | | 142 | 35.574 | 68.757 | −90.105 | 1.00 | 19.98 | C |
| ATOM | 1059 | CD | ARG | | 142 | 37.031 | 68.415 | −90.371 | 1.00 | 22.10 | C |
| ATOM | 1060 | NE | ARG | | 142 | 37.204 | 67.189 | −91.149 | 1.00 | 15.92 | N |
| ATOM | 1061 | CZ | ARG | | 142 | 38.387 | 66.697 | −91.496 | 1.00 | 19.46 | C |
| ATOM | 1062 | NH1 | ARG | | 142 | 39.492 | 67.322 | −91.124 | 1.00 | 15.52 | N |
| ATOM | 1063 | NH2 | ARG | | 142 | 38.471 | 65.602 | −92.239 | 1.00 | 16.96 | N |
| ATOM | 1064 | C | ARG | | 142 | 32.438 | 69.554 | −92.286 | 1.00 | 16.68 | C |
| ATOM | 1065 | O | ARG | | 142 | 31.841 | 68.747 | −93.000 | 1.00 | 14.83 | O |
| ATOM | 1066 | N | THR | | 143 | 32.390 | 70.859 | −92.519 | 1.00 | 12.63 | N |
| ATOM | 1067 | CA | THR | | 143 | 31.574 | 71.349 | −93.632 | 1.00 | 15.12 | C |
| ATOM | 1068 | CB | THR | | 143 | 31.618 | 72.899 | −93.715 | 1.00 | 24.29 | C |
| ATOM | 1069 | OG1 | THR | | 143 | 31.272 | 73.447 | −92.438 | 1.00 | 20.51 | O |
| ATOM | 1070 | CG2 | THR | | 143 | 30.602 | 73.419 | −94.735 | 1.00 | 17.77 | C |
| ATOM | 1071 | C | THR | | 143 | 31.963 | 70.753 | −94.981 | 1.00 | 15.98 | C |
| ATOM | 1072 | O | THR | | 143 | 33.141 | 70.666 | −95.331 | 1.00 | 16.43 | O |
| ATOM | 1073 | N | GLY | | 144 | 30.970 | 70.320 | −95.745 | 1.00 | 16.11 | N |
| ATOM | 1074 | CA | GLY | | 144 | 31.237 | 69.727 | −97.039 | 1.00 | 14.17 | C |
| ATOM | 1075 | C | GLY | | 144 | 31.526 | 68.233 | −96.942 | 1.00 | 16.38 | C |
| ATOM | 1076 | O | GLY | | 144 | 31.657 | 67.564 | −97.963 | 1.00 | 16.61 | O |
| ATOM | 1077 | N | GLU | | 145 | 31.633 | 67.691 | −95.729 | 1.00 | 12.77 | N |
| ATOM | 1078 | CA | GLU | | 145 | 31.902 | 66.246 | −95.682 | 1.00 | 12.81 | C |
| ATOM | 1079 | CB | GLU | | 145 | 32.718 | 65.871 | −94.440 | 1.00 | 10.46 | C |
| ATOM | 1080 | CG | GLU | | 145 | 34.177 | 66.361 | −94.451 | 1.00 | 11.69 | C |
| ATOM | 1081 | CD | GLU | | 145 | 34.961 | 65.765 | −93.311 | 1.00 | 17.18 | C |
| ATOM | 1082 | OE1 | GLU | | 145 | 34.590 | 66.014 | −92.134 | 1.00 | 17.64 | O |
| ATOM | 1083 | OE2 | GLU | | 145 | 35.941 | 65.041 | −93.587 | 1.00 | 16.41 | O |
| ATOM | 1084 | C | GLU | | 145 | 30.632 | 65.404 | −95.685 | 1.00 | 15.86 | C |
| ATOM | 1085 | O | GLU | | 145 | 29.559 | 65.855 | −95.271 | 1.00 | 14.91 | O |
| ATOM | 1086 | N | MET | | 146 | 30.758 | 64.157 | −96.121 | 1.00 | 13.61 | N |
| ATOM | 1087 | CA | MET | | 146 | 29.720 | 63.153 | −96.231 | 1.00 | 15.81 | C |
| ATOM | 1088 | CB | MET | | 146 | 29.598 | 62.696 | −97.692 | 1.00 | 15.56 | C |
| ATOM | 1089 | CG | MET | | 146 | 28.410 | 61.753 | −97.944 | 1.00 | 20.27 | C |
| ATOM | 1090 | SD | MET | | 146 | 28.151 | 61.322 | −99.696 | 1.00 | 17.35 | S |
| ATOM | 1091 | CE | MET | | 146 | 29.631 | 60.415 | −100.015 | 1.00 | 22.76 | C |
| ATOM | 1092 | C | MET | | 146 | 30.023 | 61.963 | −95.321 | 1.00 | 11.85 | C |
| ATOM | 1093 | O | MET | | 146 | 31.165 | 61.497 | −95.212 | 1.00 | 14.18 | O |
| ATOM | 1094 | N | SER | | 147 | 29.000 | 61.460 | −94.651 | 1.00 | 14.15 | N |
| ATOM | 1095 | CA | SER | | 147 | 29.197 | 60.337 | −93.753 | 1.00 | 15.11 | C |
| ATOM | 1096 | CB | SER | | 147 | 28.054 | 60.277 | −92.744 | 1.00 | 13.31 | C |
| ATOM | 1097 | OG | SER | | 147 | 26.903 | 59.718 | −93.369 | 1.00 | 13.47 | O |
| ATOM | 1098 | C | SER | | 147 | 29.207 | 59.007 | −94.488 | 1.00 | 13.31 | C |
| ATOM | 1099 | O | SER | | 147 | 29.001 | 58.942 | −95.693 | 1.00 | 12.58 | O |
| ATOM | 1100 | N | GLY | | 148 | 29.441 | 57.929 | −93.747 | 1.00 | 16.85 | N |
| ATOM | 1101 | CA | GLY | | 148 | 29.359 | 56.603 | −94.324 | 1.00 | 14.08 | C |
| ATOM | 1102 | C | GLY | | 148 | 27.953 | 56.165 | −93.905 | 1.00 | 17.77 | C |
| ATOM | 1103 | O | GLY | | 148 | 27.142 | 57.018 | −93.523 | 1.00 | 15.60 | O |
| ATOM | 1104 | N | PRO | | 149 | 27.627 | 54.862 | −93.946 | 1.00 | 17.52 | N |
| ATOM | 1105 | CD | PRO | | 149 | 28.412 | 53.720 | −94.456 | 1.00 | 13.23 | C |
| ATOM | 1106 | CA | PRO | | 149 | 26.284 | 54.435 | −93.541 | 1.00 | 18.15 | C |
| ATOM | 1107 | CB | PRO | | 149 | 26.291 | 52.923 | −93.817 | 1.00 | 17.17 | C |
| ATOM | 1108 | CG | PRO | | 149 | 27.324 | 52.771 | −94.906 | 1.00 | 23.29 | C |
| ATOM | 1109 | C | PRO | | 149 | 26.063 | 54.731 | −92.062 | 1.00 | 13.01 | C |
| ATOM | 1110 | O | PRO | | 149 | 26.878 | 54.374 | −91.214 | 1.00 | 20.09 | O |
| ATOM | 1111 | N | VAL | | 150 | 24.968 | 55.392 | −91.738 | 1.00 | 15.22 | N |
| ATOM | 1112 | CA | VAL | | 150 | 24.628 | 55.732 | −90.354 | 1.00 | 17.96 | C |
| ATOM | 1113 | CB | VAL | | 150 | 24.529 | 57.272 | −90.142 | 1.00 | 22.32 | C |
| ATOM | 1114 | CG1 | VAL | | 150 | 23.968 | 57.579 | −88.758 | 1.00 | 21.54 | C |
| ATOM | 1115 | CG2 | VAL | | 150 | 25.900 | 57.922 | −90.306 | 1.00 | 19.02 | C |
| ATOM | 1116 | C | VAL | | 150 | 23.278 | 55.107 | −90.045 | 1.00 | 19.34 | C |
| ATOM | 1117 | O | VAL | | 150 | 22.284 | 55.390 | −90.718 | 1.00 | 16.89 | O |
| ATOM | 1118 | N | PHE | | 151 | 23.227 | 54.253 | −89.025 | 1.00 | 24.57 | N |
| ATOM | 1119 | CA | PHE | | 151 | 22.005 | 53.545 | −88.632 | 1.00 | 24.78 | C |
| ATOM | 1120 | CB | PHE | | 151 | 22.326 | 52.121 | −88.156 | 1.00 | 23.35 | C |
| ATOM | 1121 | CG | PHE | | 151 | 22.970 | 51.249 | −89.195 | 1.00 | 25.89 | C |
| ATOM | 1122 | CD1 | PHE | | 151 | 24.307 | 51.405 | −89.524 | 1.00 | 31.26 | C |
| ATOM | 1123 | CD2 | PHE | | 151 | 22.232 | 50.272 | −89.845 | 1.00 | 27.93 | C |
| ATOM | 1124 | CE1 | PHE | | 151 | 24.899 | 50.599 | −90.484 | 1.00 | 32.61 | C |
| ATOM | 1125 | CE2 | PHE | | 151 | 22.813 | 49.463 | −90.807 | 1.00 | 32.62 | C |
| ATOM | 1126 | CZ | PHE | | 151 | 24.152 | 49.628 | −91.126 | 1.00 | 29.04 | C |
| ATOM | 1127 | C | PHE | | 151 | 21.221 | 54.215 | −87.520 | 1.00 | 18.24 | C |
| ATOM | 1128 | O | PHE | | 151 | 21.774 | 54.541 | −86.483 | 1.00 | 20.34 | O |
| ATOM | 1129 | N | THR | | 152 | 19.922 | 54.408 | −87.723 | 1.00 | 17.63 | N |
| ATOM | 1130 | CA | THR | | 152 | 19.041 | 54.972 | −86.707 | 1.00 | 21.51 | C |
| ATOM | 1131 | CB | THR | | 152 | 18.642 | 56.406 | −87.003 | 1.00 | 19.21 | C |
| ATOM | 1132 | OG1 | THR | | 152 | 17.660 | 56.394 | −88.050 | 1.00 | 14.84 | O |
| ATOM | 1133 | CG2 | THR | | 152 | 19.863 | 57.244 | −87.409 | 1.00 | 16.50 | C |
| ATOM | 1134 | C | THR | | 152 | 17.758 | 54.142 | −86.746 | 1.00 | 21.84 | C |
| ATOM | 1135 | O | THR | | 152 | 17.644 | 53.209 | −87.552 | 1.00 | 22.14 | O |
| ATOM | 1136 | N | ASP | | 153 | 16.781 | 54.473 | −85.903 | 1.00 | 21.41 | N |
| ATOM | 1137 | CA | ASP | | 153 | 15.571 | 53.664 | −85.985 | 1.00 | 25.25 | C |
| ATOM | 1138 | CB | ASP | | 153 | 14.621 | 53.971 | −84.815 | 1.00 | 27.79 | C |
| ATOM | 1139 | CG | ASP | | 153 | 15.212 | 53.601 | −83.472 | 1.00 | 34.22 | C |
| ATOM | 1140 | OD1 | ASP | | 153 | 15.964 | 52.600 | −83.400 | 1.00 | 32.70 | O |
| ATOM | 1141 | OD2 | ASP | | 153 | 14.914 | 54.303 | −82.486 | 1.00 | 35.40 | O |
| ATOM | 1142 | C | ASP | | 153 | 14.833 | 53.887 | −87.292 | 1.00 | 23.07 | C |
| ATOM | 1143 | O | ASP | | 153 | 14.038 | 53.045 | −87.722 | 1.00 | 30.01 | O |
| ATOM | 1144 | N | SER | | 154 | 15.080 | 55.014 | −87.944 | 1.00 | 22.07 | N |
| ATOM | 1145 | CA | SER | | 154 | 14.387 | 55.286 | −89.198 | 1.00 | 20.97 | C |
| ATOM | 1146 | CB | SER | | 154 | 14.585 | 56.744 | −89.597 | 1.00 | 29.80 | C |
| ATOM | 1147 | OG | SER | | 154 | 14.115 | 57.604 | −88.576 | 1.00 | 33.35 | O |
| ATOM | 1148 | C | SER | | 154 | 14.858 | 54.401 | −90.340 | 1.00 | 22.77 | C |
| ATOM | 1149 | O | SER | | 154 | 14.087 | 54.075 | −91.239 | 1.00 | 23.74 | O |
| ATOM | 1150 | N | GLY | | 155 | 16.127 | 54.009 | −90.313 | 1.00 | 18.60 | N |
| ATOM | 1151 | CA | GLY | | 155 | 16.695 | 53.186 | −91.368 | 1.00 | 19.61 | C |
| ATOM | 1152 | C | GLY | | 155 | 18.187 | 53.466 | −91.449 | 1.00 | 21.93 | C |
| ATOM | 1153 | O | GLY | | 155 | 18.847 | 53.651 | −90.422 | 1.00 | 21.38 | O |
| ATOM | 1154 | N | ILE | | 156 | 18.740 | 53.510 | −92.658 | 1.00 | 20.41 | N |
| ATOM | 1155 | CA | ILE | | 156 | 20.163 | 53.774 | −92.886 | 1.00 | 19.70 | C |
| ATOM | 1156 | CB | ILE | | 156 | 20.837 | 52.631 | −93.669 | 1.00 | 17.68 | C |
| ATOM | 1157 | CG2 | ILE | | 156 | 22.342 | 52.777 | −93.591 | 1.00 | 14.29 | C |
| ATOM | 1158 | CG1 | ILE | | 156 | 20.460 | 51.279 | −93.049 | 1.00 | 21.05 | C |
| ATOM | 1159 | CD1 | ILE | | 156 | 20.992 | 50.088 | −93.800 | 1.00 | 19.12 | C |
| ATOM | 1160 | C | ILE | | 156 | 20.311 | 55.070 | −93.680 | 1.00 | 20.31 | C |
| ATOM | 1161 | O | ILE | | 156 | 19.692 | 55.247 | −94.730 | 1.00 | 17.38 | O |
| ATOM | 1162 | N | HIS | | 157 | 21.141 | 55.980 | −93.187 | 1.00 | 15.36 | N |
| ATOM | 1163 | CA | HIS | | 157 | 21.351 | 57.278 | −93.799 | 1.00 | 19.59 | C |
| ATOM | 1164 | CB | HIS | | 157 | 21.103 | 58.405 | −92.773 | 1.00 | 15.60 | C |
| ATOM | 1165 | CG | HIS | | 157 | 19.860 | 58.253 | −91.945 | 1.00 | 18.46 | C |
| ATOM | 1166 | CD2 | HIS | | 157 | 19.552 | 57.393 | −90.943 | 1.00 | 20.86 | C |
| ATOM | 1167 | ND1 | HIS | | 157 | 18.815 | 59.147 | −92.011 | 1.00 | 22.45 | N |
| ATOM | 1168 | CE1 | HIS | | 157 | 17.921 | 58.853 | −91.081 | 1.00 | 20.64 | C |
| ATOM | 1169 | NE2 | HIS | | 157 | 18.347 | 57.793 | −90.418 | 1.00 | 19.46 | N |
| ATOM | 1170 | C | HIS | | 157 | 22.765 | 57.519 | −94.341 | 1.00 | 17.69 | C |
| ATOM | 1171 | O | HIS | | 157 | 23.737 | 56.881 | −93.928 | 1.00 | 16.12 | O |
| ATOM | 1172 | N | ILE | | 158 | 22.873 | 58.463 | −95.269 | 1.00 | 15.78 | N |
| ATOM | 1173 | CA | ILE | | 158 | 24.070 | 59.033 | −95.878 | 1.00 | 16.49 | C |
| ATOM | 1174 | CB | ILE | | 158 | 24.154 | 58.934 | −97.398 | 1.00 | 16.75 | C |
| ATOM | 1175 | CG2 | ILE | | 158 | 25.438 | 59.652 | −97.849 | 1.00 | 23.51 | C |
| ATOM | 1176 | CG1 | ILE | | 158 | 24.216 | 57.481 | −97.861 | 1.00 | 18.01 | C |
| ATOM | 1177 | CD1 | ILE | | 158 | 24.221 | 57.346 | −99.372 | 1.00 | 16.12 | C |
| ATOM | 1178 | C | ILE | | 158 | 23.825 | 60.502 | −95.554 | 1.00 | 15.26 | C |
| ATOM | 1179 | O | ILE | | 158 | 22.805 | 61.068 | −95.945 | 1.00 | 16.89 | O |
| ATOM | 1180 | N | ILE | | 159 | 24.733 | 61.117 | −94.818 | 1.00 | 13.23 | N |
| ATOM | 1181 | CA | ILE | | 159 | 24.524 | 62.487 | −94.422 | 1.00 | 10.27 | C |
| ATOM | 1182 | CB | ILE | | 159 | 24.641 | 62.637 | −92.881 | 1.00 | 11.54 | C |
| ATOM | 1183 | CG2 | ILE | | 159 | 24.301 | 64.089 | −92.466 | 1.00 | 9.66 | C |
| ATOM | 1184 | CG1 | ILE | | 159 | 23.701 | 61.646 | −92.182 | 1.00 | 16.43 | C |
| ATOM | 1185 | CD1 | ILE | | 159 | 23.886 | 61.567 | −90.655 | 1.00 | 16.06 | C |
| ATOM | 1186 | C | ILE | | 159 | 25.568 | 63.388 | −95.046 | 1.00 | 15.79 | C |
| ATOM | 1187 | O | ILE | | 159 | 26.755 | 63.049 | −95.054 | 1.00 | 18.13 | O |
| ATOM | 1188 | N | LEU | | 160 | 25.144 | 64.537 | −95.568 | 1.00 | 14.81 | N |
| ATOM | 1189 | CA | LEU | | 160 | 26.045 | 65.525 | −96.161 | 1.00 | 15.28 | C |
| ATOM | 1190 | CB | LEU | | 160 | 25.636 | 65.837 | −97.606 | 1.00 | 15.05 | C |
| ATOM | 1191 | CG | LEU | | 160 | 26.395 | 66.977 | −98.297 | 1.00 | 14.56 | C |
| ATOM | 1192 | CD1 | LEU | | 160 | 27.879 | 66.636 | −98.400 | 1.00 | 16.31 | C |
| ATOM | 1193 | CD2 | LEU | | 160 | 25.829 | 67.213 | −99.682 | 1.00 | 15.15 | C |
| ATOM | 1194 | C | LEU | | 160 | 25.938 | 66.800 | −95.335 | 1.00 | 13.83 | C |
| ATOM | 1195 | O | LEU | | 160 | 24.860 | 67.364 | −95.228 | 1.00 | 12.93 | O |
| ATOM | 1196 | N | ARG | | 161 | 27.028 | 67.234 | −94.706 | 1.00 | 11.70 | N |
| ATOM | 1197 | CA | ARG | | 161 | 26.882 | 68.484 | −93.950 | 1.00 | 15.67 | C |
| ATOM | 1198 | CB | ARG | | 161 | 27.849 | 68.527 | −92.748 | 1.00 | 15.48 | C |
| ATOM | 1199 | CG | ARG | | 161 | 27.844 | 69.888 | −92.016 | 1.00 | 12.44 | C |
| ATOM | 1200 | CD | ARG | | 161 | 28.879 | 69.989 | −90.872 | 1.00 | 13.73 | C |
| ATOM | 1201 | NE | ARG | | 161 | 28.952 | 71.381 | −90.413 | 1.00 | 14.86 | N |
| ATOM | 1202 | CZ | ARG | | 161 | 29.900 | 71.884 | −89.626 | 1.00 | 16.53 | C |
| ATOM | 1203 | NH1 | ARG | | 161 | 30.877 | 71.127 | −89.172 | 1.00 | 17.91 | N |
| ATOM | 1204 | NH2 | ARG | | 161 | 29.885 | 73.175 | −89.326 | 1.00 | 18.77 | N |
| ATOM | 1205 | C | ARG | | 161 | 27.155 | 69.666 | −94.881 | 1.00 | 14.40 | C |
| ATOM | 1206 | O | ARG | | 161 | 28.216 | 69.757 | −95.511 | 1.00 | 17.51 | O |
| ATOM | 1207 | N | THR | | 162 | 26.206 | 70.584 | −94.978 | 1.00 | 16.61 | N |
| ATOM | 1208 | CA | THR | | 162 | 26.365 | 71.718 | −95.870 | 1.00 | 14.81 | C |
| ATOM | 1209 | CB | THR | | 162 | 25.098 | 71.930 | −96.691 | 1.00 | 17.28 | C |
| ATOM | 1210 | OG1 | THR | | 162 | 24.004 | 72.204 | −95.803 | 1.00 | 20.82 | O |
| ATOM | 1211 | CG2 | THR | | 162 | 24.791 | 70.655 | −97.545 | 1.00 | 17.29 | C |
| ATOM | 1212 | C | THR | | 162 | 26.739 | 73.028 | −95.178 | 1.00 | 19.93 | C |
| ATOM | 1213 | O | THR | | 162 | 27.244 | 73.941 | −95.821 | 1.00 | 18.38 | O |
| ATOM | 1214 | N | GLU | | 163 | 26.470 | 73.147 | −93.878 | 1.00 | 16.73 | N |
| ATOM | 1215 | CA | GLU | | 163 | 26.871 | 74.366 | −93.170 | 1.00 | 20.53 | C |
| ATOM | 1216 | CB | GLU | | 163 | 25.732 | 75.389 | −93.122 | 1.00 | 19.36 | C |
| ATOM | 1217 | CG | GLU | | 163 | 25.258 | 75.885 | −94.476 | 1.00 | 25.89 | C |
| ATOM | 1218 | CD | GLU | | 163 | 23.995 | 76.742 | −94.388 | 1.00 | 31.59 | C |
| ATOM | 1219 | OE1 | GLU | | 163 | 23.615 | 77.146 | −93.266 | 1.00 | 32.44 | O |
| ATOM | 1220 | OE2 | GLU | | 163 | 23.388 | 77.015 | −95.447 | 1.00 | 28.55 | O |
| ATOM | 1221 | C | GLU | | 163 | 27.266 | 73.994 | −91.751 | 1.00 | 19.50 | C |
| ATOM | 1222 | OT1 | GLU | | 163 | 26.719 | 72.981 | −91.267 | 1.00 | 16.44 | O |
| ATOM | 1223 | OT2 | GLU | | 163 | 28.093 | 74.713 | −91.146 | 1.00 | 19.10 | O |
| TER | 1224 | | GLU | | 163 | | | | | | |
| ATOM | 1225 | S | SO4 | C | 300 | 10.832 | 58.369 | −89.899 | 1.00 | 59.99 | S |
| ATOM | 1226 | O1 | SO4 | C | 300 | 9.735 | 58.019 | −90.841 | 1.00 | 58.45 | O |
| ATOM | 1227 | O2 | SO4 | C | 300 | 10.461 | 59.556 | −89.093 | 1.00 | 56.01 | O |
| ATOM | 1228 | O3 | SO4 | C | 300 | 12.040 | 58.721 | −90.692 | 1.00 | 52.29 | O |
| ATOM | 1229 | O4 | SO4 | C | 300 | 11.077 | 57.212 | −88.983 | 1.00 | 49.56 | O |
| TER | 1230 | | SO4 | C | 300 | | | | | | |
| ATOM | 1231 | OH2 | TIP | S | 1 | 33.624 | 62.416 | −93.953 | 1.00 | 13.46 | O |
| ATOM | 1232 | OH2 | TIP | S | 2 | 33.443 | 63.553 | −97.305 | 1.00 | 11.91 | O |
| ATOM | 1233 | OH2 | TIP | S | 3 | 27.531 | 62.996 | −111.177 | 1.00 | 12.94 | O |
| ATOM | 1234 | OH2 | TIP | S | 4 | 21.605 | 71.005 | −96.545 | 1.00 | 20.70 | O |
| ATOM | 1235 | OH2 | TIP | S | 5 | 33.646 | 54.141 | −99.258 | 1.00 | 25.24 | O |
| ATOM | 1236 | OH2 | TIP | S | 6 | 34.774 | 64.187 | −90.113 | 1.00 | 17.57 | O |
| ATOM | 1237 | OH2 | TIP | S | 7 | 32.547 | 56.679 | −96.719 | 1.00 | 21.44 | O |
| ATOM | 1238 | OH2 | TIP | S | 8 | 40.310 | 59.942 | −104.478 | 1.00 | 20.39 | O |
| ATOM | 1239 | OH2 | TIP | S | 9 | 33.606 | 61.941 | −91.367 | 1.00 | 19.97 | O |
| ATOM | 1240 | OH2 | TIP | S | 10 | 30.021 | 52.315 | −98.178 | 1.00 | 25.61 | O |
| ATOM | 1241 | OH2 | TIP | S | 11 | 29.638 | 63.906 | −82.537 | 1.00 | 21.88 | O |
| ATOM | 1242 | OH2 | TIP | S | 12 | 28.576 | 76.352 | −96.247 | 1.00 | 26.20 | O |
| ATOM | 1243 | OH2 | TIP | S | 13 | 33.433 | 62.460 | −106.954 | 1.00 | 22.81 | O |
| ATOM | 1244 | OH2 | TIP | S | 14 | 16.214 | 63.023 | −98.007 | 1.00 | 18.03 | O |
| ATOM | 1245 | OH2 | TIP | S | 15 | 32.611 | 53.476 | −94.398 | 1.00 | 24.43 | O |
| ATOM | 1246 | OH2 | TIP | S | 16 | 45.221 | 56.603 | −86.357 | 1.00 | 26.68 | O |
| ATOM | 1247 | OH2 | TIP | S | 17 | 32.778 | 68.650 | −86.760 | 1.00 | 23.69 | O |
| ATOM | 1248 | OH2 | TIP | S | 18 | 18.503 | 51.029 | −89.491 | 1.00 | 26.24 | O |
| ATOM | 1249 | OH2 | TIP | S | 19 | 35.858 | 63.545 | −95.799 | 1.00 | 19.85 | O |
| ATOM | 1250 | OH2 | TIP | S | 20 | 30.427 | 51.871 | −100.966 | 1.00 | 25.25 | O |
| ATOM | 1251 | OH2 | TIP | S | 21 | 15.199 | 54.092 | −100.856 | 1.00 | 22.99 | O |
| ATOM | 1252 | OH2 | TIP | S | 22 | 43.472 | 61.797 | −104.102 | 1.00 | 41.98 | O |
| ATOM | 1253 | OH2 | TIP | S | 23 | 34.789 | 69.355 | −97.060 | 1.00 | 23.71 | O |
| ATOM | 1254 | OH2 | TIP | S | 24 | 26.784 | 58.231 | −85.337 | 1.00 | 23.86 | O |
| ATOM | 1255 | OH2 | TIP | S | 25 | 16.950 | 52.895 | −102.548 | 1.00 | 25.87 | O |
| ATOM | 1256 | OH2 | TIP | S | 26 | 24.011 | 60.851 | −113.522 | 1.00 | 35.27 | O |
| ATOM | 1257 | OH2 | TIP | S | 27 | 27.573 | 64.900 | −104.931 | 1.00 | 26.37 | O |
| ATOM | 1258 | OH2 | TIP | S | 28 | 26.155 | 47.941 | −103.462 | 1.00 | 32.78 | O |
| ATOM | 1259 | OH2 | TIP | S | 29 | 23.969 | 71.985 | −80.582 | 1.00 | 25.34 | O |
| ATOM | 1260 | OH2 | TIP | S | 30 | 46.046 | 52.449 | −86.698 | 1.00 | 28.42 | O |
| ATOM | 1261 | OH2 | TIP | S | 31 | 44.980 | 64.821 | −97.038 | 1.00 | 23.59 | O |
| ATOM | 1262 | OH2 | TIP | S | 32 | 27.369 | 75.652 | −88.614 | 1.00 | 25.98 | O |
| ATOM | 1263 | OH2 | TIP | S | 33 | 15.836 | 74.710 | −95.002 | 1.00 | 42.42 | O |
| ATOM | 1264 | OH2 | TIP | S | 34 | 37.752 | 47.469 | −94.615 | 1.00 | 32.91 | O |
| ATOM | 1265 | OH2 | TIP | S | 35 | 19.971 | 66.798 | −83.183 | 1.00 | 25.63 | O |
| ATOM | 1266 | OH2 | TIP | S | 36 | 17.777 | 59.078 | −80.149 | 1.00 | 33.52 | O |
| ATOM | 1267 | OH2 | TIP | S | 37 | 31.523 | 58.777 | −97.386 | 1.00 | 28.40 | O |
| ATOM | 1268 | OH2 | TIP | S | 38 | 36.322 | 58.943 | −100.118 | 1.00 | 24.34 | O |
| ATOM | 1269 | OH2 | TIP | S | 39 | 15.825 | 72.327 | −89.613 | 1.00 | 38.46 | O |
| ATOM | 1270 | OH2 | TIP | S | 40 | 13.147 | 69.171 | −95.515 | 1.00 | 39.16 | O |
| ATOM | 1271 | OH2 | TIP | S | 41 | 11.741 | 52.749 | −101.001 | 1.00 | 27.40 | O |
| ATOM | 1272 | OH2 | TIP | S | 42 | 22.580 | 51.781 | −104.804 | 1.00 | 27.61 | O |
| ATOM | 1273 | OH2 | TIP | S | 43 | 22.909 | 53.071 | −84.603 | 1.00 | 36.42 | O |
| ATOM | 1274 | OH2 | TIP | S | 44 | 14.727 | 47.575 | −103.065 | 1.00 | 35.46 | O |
| ATOM | 1275 | OH2 | TIP | S | 45 | 22.983 | 61.755 | −109.764 | 1.00 | 32.54 | O |
| ATOM | 1276 | OH2 | TIP | S | 46 | 14.213 | 69.101 | −90.816 | 1.00 | 31.85 | O |
| ATOM | 1277 | OH2 | TIP | S | 47 | 25.424 | 54.174 | −87.143 | 1.00 | 24.66 | O |
| ATOM | 1278 | OH2 | TIP | S | 48 | 6.174 | 47.654 | −101.478 | 1.00 | 45.67 | O |
| ATOM | 1279 | OH2 | TIP | S | 49 | 26.636 | 59.294 | −114.674 | 1.00 | 28.54 | O |
| ATOM | 1280 | OH2 | TIP | S | 50 | 11.492 | 47.729 | −99.939 | 1.00 | 26.40 | O |
| ATOM | 1281 | OH2 | TIP | S | 51 | 7.989 | 59.804 | −96.941 | 1.00 | 26.35 | O |
| ATOM | 1282 | OH2 | TIP | S | 52 | 35.219 | 60.110 | −96.333 | 1.00 | 31.04 | O |
| ATOM | 1283 | OH2 | TIP | S | 53 | 32.546 | 75.259 | −90.321 | 1.00 | 50.35 | O |
| ATOM | 1284 | OH2 | TIP | S | 54 | 15.740 | 73.401 | −99.059 | 1.00 | 50.75 | O |
| ATOM | 1285 | OH2 | TIP | S | 55 | 25.802 | 69.620 | −106.768 | 1.00 | 31.52 | O |
| ATOM | 1286 | OH2 | TIP | S | 56 | 33.591 | 55.160 | −105.709 | 1.00 | 21.08 | O |
| ATOM | 1287 | OH2 | TIP | S | 57 | 32.410 | 53.576 | −101.626 | 1.00 | 23.66 | O |
| ATOM | 1288 | OH2 | TIP | S | 58 | 31.752 | 53.983 | −97.070 | 1.00 | 26.60 | O |
| ATOM | 1289 | OH2 | TIP | S | 59 | 16.928 | 61.686 | −87.052 | 1.00 | 28.95 | O |
| ATOM | 1290 | OH2 | TIP | S | 60 | 21.761 | 72.709 | −98.615 | 1.00 | 26.20 | O |
| ATOM | 1291 | OH2 | TIP | S | 61 | 23.030 | 78.507 | −82.123 | 1.00 | 45.86 | O |
| ATOM | 1292 | OH2 | TIP | S | 62 | 38.057 | 60.682 | −81.243 | 1.00 | 38.07 | O |
| ATOM | 1293 | OH2 | TIP | S | 63 | 39.979 | 69.532 | −89.463 | 1.00 | 29.68 | O |
| ATOM | 1294 | OH2 | TIP | S | 64 | 24.971 | 67.277 | −104.180 | 1.00 | 28.03 | O |
| ATOM | 1295 | OH2 | TIP | S | 65 | 23.768 | 72.218 | −100.559 | 1.00 | 44.54 | O |
| ATOM | 1296 | OH2 | TIP | S | 66 | 45.647 | 55.907 | −97.518 | 1.00 | 27.28 | O |
| ATOM | 1297 | OH2 | TIP | S | 67 | 35.889 | 60.423 | −102.426 | 1.00 | 32.60 | O |
| ATOM | 1298 | OH2 | TIP | S | 68 | 31.595 | 65.895 | −82.526 | 1.00 | 24.82 | O |
| ATOM | 1299 | OH2 | TIP | S | 69 | 35.344 | 67.549 | −86.389 | 1.00 | 32.02 | O |
| ATOM | 1300 | OH2 | TIP | S | 70 | 30.247 | 62.047 | −80.680 | 1.00 | 27.48 | O |
| ATOM | 1301 | OH2 | TIP | S | 71 | 20.361 | 69.071 | −100.372 | 1.00 | 27.20 | O |
| ATOM | 1302 | OH2 | TIP | S | 72 | 22.042 | 65.496 | −77.382 | 1.00 | 29.97 | O |
| ATOM | 1303 | OH2 | TIP | S | 73 | 12.236 | 46.514 | −102.427 | 1.00 | 38.80 | O |
| ATOM | 1304 | OH2 | TIP | S | 74 | 17.843 | 55.955 | −83.395 | 1.00 | 36.25 | O |
| ATOM | 1305 | OH2 | TIP | S | 75 | 9.252 | 53.272 | −105.277 | 1.00 | 43.26 | O |
| ATOM | 1306 | OH2 | TIP | S | 76 | 30.884 | 49.459 | −102.139 | 1.00 | 37.04 | O |
| ATOM | 1307 | OH2 | TIP | S | 77 | 32.986 | 60.705 | −97.818 | 1.00 | 37.86 | O |
| ATOM | 1308 | OH2 | TIP | S | 78 | 16.726 | 43.073 | −89.889 | 1.00 | 37.74 | O |
| ATOM | 1309 | OH2 | TIP | S | 79 | 18.451 | 52.713 | −106.654 | 1.00 | 24.54 | O |
| ATOM | 1310 | OH2 | TIP | S | 80 | 19.988 | 45.906 | −102.158 | 1.00 | 28.78 | O |
| ATOM | 1311 | OH2 | TIP | S | 81 | 23.184 | 49.126 | −105.053 | 1.00 | 35.49 | O |
| ATOM | 1312 | OH2 | TIP | S | 82 | 38.323 | 51.793 | −97.814 | 1.00 | 33.61 | O |
| ATOM | 1313 | OH2 | TIP | S | 83 | 46.674 | 61.705 | −98.219 | 1.00 | 31.39 | O |
| ATOM | 1314 | OH2 | TIP | S | 84 | 44.907 | 64.486 | −101.868 | 1.00 | 21.78 | O |
| ATOM | 1315 | OH2 | TIP | S | 85 | 21.673 | 46.977 | −104.347 | 1.00 | 34.61 | O |
| ATOM | 1316 | OH2 | TIP | S | 86 | 48.170 | 56.733 | −96.137 | 1.00 | 39.04 | O |
| ATOM | 1317 | OH2 | TIP | S | 87 | 38.152 | 54.106 | −99.004 | 1.00 | 38.82 | O |
| ATOM | 1318 | OH2 | TIP | S | 88 | 9.652 | 61.249 | −93.918 | 1.00 | 38.10 | O |
| ATOM | 1319 | OH2 | TIP | S | 89 | 27.680 | 51.058 | −106.961 | 1.00 | 36.02 | O |
| ATOM | 1320 | OH2 | TIP | S | 90 | 16.104 | 62.439 | −89.578 | 1.00 | 32.35 | O |
| ATOM | 1321 | OH2 | TIP | S | 91 | 48.624 | 54.282 | −86.848 | 1.00 | 43.61 | O |
| ATOM | 1322 | OH2 | TIP | S | 92 | 30.414 | 63.685 | −106.119 | 1.00 | 26.23 | O |
| ATOM | 1323 | OH2 | TIP | S | 93 | 10.583 | 57.242 | −81.318 | 1.00 | 49.38 | O |
| ATOM | 1324 | OH2 | TIP | S | 94 | 38.482 | 45.035 | −93.987 | 1.00 | 42.80 | O |
| ATOM | 1325 | OH2 | TIP | S | 95 | 35.766 | 50.265 | −96.694 | 1.00 | 41.57 | O |
| ATOM | 1326 | OH2 | TIP | S | 96 | 26.498 | 70.606 | −101.555 | 1.00 | 42.87 | O |
| ATOM | 1327 | OH2 | TIP | S | 97 | 30.405 | 68.631 | −100.404 | 1.00 | 37.73 | O |
| ATOM | 1328 | OH2 | TIP | S | 98 | 20.715 | 60.273 | −111.290 | 1.00 | 39.77 | O |
| ATOM | 1329 | OH2 | TIP | S | 99 | 11.764 | 64.250 | −104.686 | 1.00 | 39.57 | O |
| ATOM | 1330 | OH2 | TIP | S | 100 | 9.441 | 58.277 | −94.784 | 1.00 | 34.45 | O |
| ATOM | 1331 | OH2 | TIP | S | 101 | 9.294 | 63.424 | −103.120 | 1.00 | 34.32 | O |
| ATOM | 1332 | OH2 | TIP | S | 102 | 28.524 | 70.397 | −99.562 | 1.00 | 36.91 | O |
| ATOM | 1333 | OH2 | TIP | S | 103 | 14.658 | 58.070 | −110.465 | 1.00 | 41.16 | O |
| ATOM | 1334 | OH2 | TIP | S | 104 | 11.844 | 58.618 | −107.224 | 1.00 | 38.48 | O |
| ATOM | 1335 | OH2 | TIP | S | 105 | 39.784 | 47.036 | −83.888 | 1.00 | 27.15 | O |
| ATOM | 1336 | OH2 | TIP | S | 106 | 6.825 | 62.380 | −93.801 | 1.00 | 45.64 | O |
| ATOM | 1337 | OH2 | TIP | S | 107 | 26.696 | 55.628 | −85.549 | 1.00 | 34.99 | O |
| ATOM | 1338 | OH2 | TIP | S | 108 | 40.949 | 73.520 | −82.929 | 1.00 | 36.83 | O |
| ATOM | 1339 | OH2 | TIP | S | 109 | 25.830 | 62.651 | −113.066 | 1.00 | 39.53 | O |
| ATOM | 1340 | OH2 | TIP | S | 110 | 39.542 | 71.660 | −77.458 | 1.00 | 47.32 | O |
| ATOM | 1341 | OH2 | TIP | S | 111 | 15.230 | 51.153 | −104.661 | 1.00 | 42.28 | O |
| ATOM | 1342 | OH2 | TIP | S | 112 | 25.219 | 53.671 | −83.383 | 1.00 | 38.56 | O |
| ATOM | 1343 | OH2 | TIP | S | 113 | 30.063 | 76.485 | −92.035 | 1.00 | 41.34 | O |
| ATOM | 1344 | OH2 | TIP | S | 114 | 18.849 | 40.166 | −96.895 | 1.00 | 31.89 | O |
| ATOM | 1345 | OH2 | TIP | S | 115 | 35.538 | 57.907 | −82.044 | 1.00 | 36.76 | O |
| ATOM | 1346 | OH2 | TIP | S | 116 | 38.672 | 69.108 | −87.122 | 1.00 | 41.95 | O |
| ATOM | 1347 | OH2 | TIP | S | 117 | 14.022 | 64.070 | −89.908 | 1.00 | 42.64 | O |
| ATOM | 1348 | OH2 | TIP | S | 118 | 45.133 | 43.081 | −93.847 | 1.00 | 34.46 | O |
| ATOM | 1349 | OH2 | TIP | S | 119 | 43.908 | 53.216 | −103.035 | 1.00 | 44.77 | O |
| ATOM | 1350 | OH2 | TIP | S | 120 | 37.939 | 52.430 | −104.394 | 1.00 | 44.43 | O |
| ATOM | 1351 | OH2 | TIP | S | 121 | 11.198 | 51.875 | −103.725 | 1.00 | 38.89 | O |
| ATOM | 1352 | OH2 | TIP | S | 122 | 24.894 | 55.208 | −112.221 | 1.00 | 46.22 | O |
| ATOM | 1353 | OH2 | TIP | S | 123 | 1.858 | 50.042 | −94.694 | 1.00 | 44.61 | O |
| ATOM | 1354 | OH2 | TIP | S | 124 | 4.264 | 49.200 | −100.989 | 1.00 | 39.39 | O |
| ATOM | 1355 | OH2 | TIP | S | 125 | 39.354 | 74.056 | −88.257 | 1.00 | 47.31 | O |
| ATOM | 1356 | OH2 | TIP | S | 126 | 19.020 | 49.810 | −87.071 | 1.00 | 45.96 | O |
| ATOM | 1357 | OH2 | TIP | S | 127 | 39.383 | 58.767 | −83.146 | 1.00 | 46.21 | O |
| ATOM | 1358 | OH2 | TIP | S | 128 | 32.211 | 52.643 | −85.436 | 1.00 | 56.70 | O |
| ATOM | 1359 | OH2 | TIP | S | 129 | 43.300 | 69.494 | −88.460 | 1.00 | 52.03 | O |
| ATOM | 1360 | OH2 | TIP | S | 130 | 18.349 | 65.905 | −111.111 | 1.00 | 38.89 | O |
| ATOM | 1361 | OH2 | TIP | S | 131 | 29.674 | 63.945 | −101.042 | 1.00 | 30.20 | O |
| ATOM | 1362 | OH2 | TIP | S | 132 | 47.466 | 42.579 | −94.983 | 1.00 | 37.96 | O |
| ATOM | 1363 | OH2 | TIP | S | 133 | 17.274 | 76.019 | −91.497 | 1.00 | 37.78 | O |
| ATOM | 1364 | OH2 | TIP | S | 134 | 16.447 | 49.153 | −87.341 | 1.00 | 40.38 | O |
| ATOM | 1365 | OH2 | TIP | S | 135 | 4.402 | 49.176 | −93.389 | 1.00 | 40.75 | O |
| ATOM | 1366 | OH2 | TIP | S | 136 | 25.457 | 56.786 | −114.458 | 1.00 | 48.82 | O |
| ATOM | 1367 | OH2 | TIP | S | 137 | 30.020 | 52.962 | −108.379 | 1.00 | 55.52 | O |
| ATOM | 1368 | OH2 | TIP | S | 138 | 23.558 | 75.580 | −97.544 | 1.00 | 47.99 | O |
| ATOM | 1369 | OH2 | TIP | S | 139 | 15.424 | 65.313 | −109.132 | 1.00 | 42.83 | O |
| ATOM | 1370 | OH2 | TIP | S | 140 | 32.874 | 65.532 | −99.350 | 1.00 | 47.30 | O |
| ATOM | 1371 | OH2 | TIP | S | 141 | 21.251 | 56.004 | −110.175 | 1.00 | 54.62 | O |
| ATOM | 1372 | OH2 | TIP | S | 142 | 33.605 | 59.178 | −79.630 | 1.00 | 45.97 | O |
| ATOM | 1373 | OH2 | TIP | S | 143 | 11.311 | 54.812 | −90.050 | 1.00 | 40.07 | O |
| ATOM | 1374 | OH2 | TIP | S | 144 | 30.878 | 47.564 | −99.480 | 1.00 | 48.96 | O |
| ATOM | 1375 | OH2 | TIP | S | 145 | 17.233 | 78.437 | −89.302 | 1.00 | 48.29 | O |
| ATOM | 1376 | OH2 | TIP | S | 146 | 40.325 | 48.149 | −81.373 | 1.00 | 30.14 | O |
| ATOM | 1377 | OH2 | TIP | S | 147 | 17.402 | 72.405 | −101.114 | 1.00 | 42.97 | O |
| ATOM | 1378 | OH2 | TIP | S | 148 | 31.430 | 70.657 | −102.411 | 1.00 | 51.94 | O |
| ATOM | 1379 | OH2 | TIP | S | 149 | 27.590 | 51.806 | −86.092 | 1.00 | 42.38 | O |
| ATOM | 1380 | OH2 | TIP | S | 150 | 21.836 | 54.107 | −108.467 | 1.00 | 42.58 | O |
| ATOM | 1381 | OH2 | TIP | S | 151 | 28.691 | 51.493 | −91.043 | 1.00 | 41.39 | O |
| ATOM | 1382 | OH2 | TIP | S | 152 | 24.751 | 57.200 | −81.204 | 1.00 | 50.45 | O |
| ATOM | 1383 | OH2 | TIP | S | 153 | 19.531 | 39.797 | −99.555 | 1.00 | 48.68 | O |
| ATOM | 1384 | OH2 | TIP | S | 154 | 44.408 | 61.227 | −83.670 | 1.00 | 41.74 | O |
| ATOM | 1385 | OH2 | TIP | S | 155 | 35.593 | 42.509 | −91.788 | 1.00 | 46.15 | O |
| ATOM | 1386 | OH2 | TIP | S | 156 | 11.588 | 66.104 | −93.159 | 1.00 | 48.33 | O |
| ATOM | 1387 | OH2 | TIP | S | 157 | 34.091 | 64.608 | −102.596 | 1.00 | 44.75 | O |
| ATOM | 1388 | OH2 | TIP | S | 158 | 37.856 | 50.362 | −102.876 | 1.00 | 52.65 | O |
| ATOM | 1389 | OH2 | TIP | S | 159 | 14.023 | 63.119 | −87.005 | 1.00 | 52.18 | O |
| TER | 1390 | | TIP | S | 159 | | | | | | |
| END | | | | | | | | | | | |

### Example 6

### Dephosphorylation Assay

The following assay can be used to test Pin1 modulators for the ability to inhibit the dephosphorylation of Pin1. Specifically, the following assay may be used to test Pin1 modulators for the ability to inhibit the desphosphorylation of pPin1 (Ser71).

The standard isomerase assay as described previously Fisher et al. (Biomed. Biochim. Acta, (1984) 43: 1101-1111) is used to determine the ability of Pin1 to inhibit dephosphorylation. Briefly, the standard assays is performed as follows: the enzyme (112 ng) is preincubated with 72 mM substrate at 4° C. for 30 minutes in an 80 μL reaction volume containing 0.02 mg/μL BSA, 0.8 mM DTT, and 35 mM HEPES (pH 7.8). Proteolysis of the substrate is initiated by the addition of 80 μL of trypsin at 0.4 mg/mL in 35 mM HEPES (pH 7.8) and the release of p-nitroaniline is monitored every 10 seconds at 390 nm using a microplate reader (MRD/8V/DIAS, Dynex Technologies).

To test for the ability of modulators to inhibit dephosphorylation, the enzyme used in the assay is phosphorylated as described in Example 4 and phosphatase is added to the reaction mix. Pin1 modulators such as those described herein can be tested for the ability to inhibit the dephosphorylation process by using this assay and monitoring the rate of release of p-nitroaniline. This assay can be performed using Pin1 that is phosphorylated at position 16, 71, or both.

### Equivalents

Those skilled in the art will recognize, or be able to ascertain using no more than routine experimentation, many equivalents to the specific embodiments and methods described herein. Such equivalents are intended to be encompassed by the scope of the following claims.

We claim:

1. A crystallized Pin1 polypeptide that is phosphorylated at position 71.

2. A crystallized Pin1 polypeptide having a mutation in the phosphokinase A recognition site for serine 16, wherein said polypeptide is phosphorylated at position 71.

3. The crystallized polypeptide of claim 2, wherein said mutation is at position 13, 14, 15, or 16.

4. The crystallized polypeptide of claim 3, wherein said mutation is at position 14.

5. The crystallized polypeptide of claim 4, wherein said mutation is an R14A mutation.

6. The crystallized polypeptide of claim 5, which has its sequence set forth in SEQ ID NO: 2.

7. The crystallized polypeptide of claim 5, which has its coordinates set forth in Table 2.

8. A crystallized Pin1 polypeptide, wherein said polypeptide is phosphorylated at position 16 and 71.

9. The crystallized polypeptide of claim 8, which has its sequence set forth in SEQ ID NO: 1.

10. The crystallized polypeptide of claim 8, which has its coordinates set forth in Table 3.

11. A purified polypeptide having a mutation in the phosphokinase A recognition site for serine 16, wherein said polypeptide is phosphorylated at position 71.

12. The purified polypeptide of claim 1 1, wherein said mutation is at position 13, 14, 15, or 16.

13. The purified polypeptide of claim 12, wherein said mutation is at position 14.

14. The purified polypeptide of claim 13, wherein said mutation is an R14A mutation.

15. The purified polypeptide of claim 14, which has its sequence set forth in SEQ ID NO: 2.

16. A Pin1 polypeptide having a mutation at position 71.

17. The Pin1 polypeptide of claim 16, wherein said mutation is selected from the group consisting of S71A, S71P, S71L, S71T, and S91 W.

18. An antibody that is specific for pPin1(71).

19. The antibody of claim 18, wherein said antibody is a monoclonal antibody.

20. The antibody of claim 18, wherein said antibody is a polyclonal antibody.

21. A method of determining if a subject has a cell proliferative disorder comprising the steps of:
obtaining a biological sample from a subject;
evaluating said sample for the presence of pPin1(71); wherein an elevated level of pPin1(71) as compared to a control sample is indicative that the subject has a cell proliferative disorder.

22. A method of determining if a subject has a cell proliferative disorder comprising the steps of:
obtaining a biological sample from a subject;
evaluating said sample for the presence of pPin1(71); wherein a decreased level of pPin1(71) as compared to a control sample is indicative that the subject has a cell proliferative disorder.

23. A method for determining the prognosis of a subject having a cell proliferative disorder comprising the steps of:
determining the levels of pPin1(71) in a biological sample;
wherein an elevated level of pPin1(71) in the sample compared to the statistical mean of a population having a cell proliferative disorder is indicative of a good prognosis.

24. A method for determining the prognosis of a subject having a cell proliferative disorder comprising the steps of:
determining the levels of pPin1(71) in a biological sample;
wherein a decreased level of pPin1(71) in the sample compared to the statistical mean of a population having a cell proliferative disorder is indicative of a poor prognosis.

25. The method of any one of claims 21, 22, 23, or 24, wherein said levels are determined using an antibody specific for pPin1(71).

26. The method of any one of claims 21, 22, 23, or 24, wherein said method further comprises determining the level of phosphorylation at position 16.

27. The method of claim 25 or 26, wherein said levels of pPin1 are determined using FISH.

28. The method of claim 25 or 26, wherein said levels of pPin1 are determined using IHC.

29. A method of determining the prognosis of a subject having a cell proliferative disorder comprising:
obtaining a first biological sample from said subject and determining the level of pPin1(71) in said sample;
obtaining a second biological sample from said subject at a time after collection of said first biological sample and determining the level of pPin1(71) in said sample;
wherein an increase in the level of pPin1(71) is indicative of good prognosis.

30. A method of determining the prognosis of a subject having a cell proliferative disorder comprising:
obtaining a first biological sample from said subject and determining the level of pPin1(71) in said sample;
obtaining a second biological sample from said subject at a time after collection of said first biological sample and determining the level of pPin1(71) in said sample;
wherein a decrease in the level of pPin1(71) is indicative of poor prognosis.

31. The method of claim 29 or 30, further comprising determining the levels of pPin1(16) in the biological samples.

32. The method of any one of claims 21-31, wherein said biological sample is from the group consisting of: breast tissue, uterine tissue, ovarian tissue, brain tissue, endometrium tissue, cervical tissue, colon tissue, esophagus tissue, hepatocellular tissue, kidney tissue, mouth tissue, prostate tissue, liver tissue, lung tissue, skin tissue, or testicular, endocrine tissue, thyroid tissue, blood, ascites and brain fluid.

33. A kit for determining the prognosis of a subject having a cell proliferative disorder comprising an antibody specific for pPin1(71) and instructions for use.

34. The kit of claim 33 further comprising an antibody specific for pPin1(16).

35. The kit of claim 33 or 34, wherein said antibody is a monoclonal antibody.

36. The kit of claim 33 or 34, wherein said antibody is a polyclonal antibody.

37. The kit of claim 33 or 34, further comprising an antibody specific for a second cancer marker.

38. A method for determining the course of treatment for a subject having a cell proliferative disorder comprising determining the level of pPin1(71 ) in a biological sample from said subject, wherein the lower the level of pPin1(71) the more aggressive the treatment of said subject with an anticancer agent.

39. The method of claim 38, wherein said cell proliferative disorder is cancer.

40. The method of claim 39, wherein said cancer is selected from the group consisting of: oligodendroglioma, astrocytoma, glioblastomamultiforme, cervical carcinoma, endometriod carcinoma, endometrium serous carcenoma, ovary endometroid cancer, ovary Brenner tumor, ovary mucinous cancer, ovary serous cancer, uterus carcinosarcoma, breast lobular cancer, breast ductal cancer, breast medullary cancer, breast mucinous cancer, breast tubular cancer, thyroid adenocarcinoma, thyroid follicular cancer, thyroid medullary cancer, thyroid papillary carcinoma, parathyroid adenocarcinoma, adrenal gland adenoma, adrenal gland cancer, pheochromocytoma, colon adenoma mild dysplasia, colon adenoma moderate dysplasia, colon adenoma severe dysplasia, colon adenocarcinoma, esophagus adenocarcinoma, hepatocelluar carcinoma, mouth cancer, gall bladder adenocarcinoma, pancreatic adenocarcinoma, small intestine adenocarcinoma, stomach diffuse adenocarcinoma, prostate (hormone-refract), prostate (untreated), kidney chromophobic carcinoma, kidney clear cell carcinoma, kidney oncocytoma, kidney papillary carcinoma, testis non-seminomatous cancer, testis seminoma, urinary bladder transitional carcinoma, lung adenocarcinoma, lung large cell cancer, lung small cell cancer, lung squamous cell carcinoma, Hodgkin lymphoma, MALT lymphoma, non-hodgkins lymphoma (NHL) diffuse large B, NHL, thymoma, skin malignant melanoma, skin basolioma, skin squamous cell cancer, skin merkel cell cancer, skin benign nevus, lipoma, liposarcoma abnormal cell growth.

41. The method of claim 38, wherein said anticancer agent is a Pin1 inhibitor.

42. A nucleic acid molecule encoding the polypeptide of any one of claims 1-5 and 8.

43. A vector comprising the nucleic acid molecule of claim 42.

44. A host cell comprising the vector of claim 43.

45. A modulator of Pin1 that inhibits dephosphorylation of serine 71.

46. A modulator of Pin1 that interacts with serine 71 and inhibits catalytic activity.

47. The modulator of claims 46 wherein said modulator mimics a phosphate moiety.

48. A method for determining if a subject is at risk of developing a Pin1-associated state comprising:
obtaining an biological sample from the subject; and
determining if the subject has a mutation in Pin1;
whereby a mutation that produces a constitutively active Pin1 is indicative that the subject is at risk of developing a Pin1-associated state.

49. The method of claim 48, wherein said mutant is a mutation that inhibits the ability of Pin1 to be phosphorylated at position 71.

50. The method of claim 49, wherein said mutation is at position 71.

51. The method of claim 46, wherein said mutant is selected from the group consisting of, S71P, S71A, S71W, and S71L.
